(19) United States
(12) Patent Application Publication
(10) Pub. No.: US 2021/0330693 A1
(43) Pub. Date: Oct. 28, 2021

(54) HEPARANASE INHIBITORS AND THEIR USE AS ANTI-CANCER COMPOUNDS

(71) Applicants: Wayne State University, Detroit, MI (US); Technion, Israel Institute of Technology, Haifa (IL); University of Iowa Research Foundation, Iowa City, IA (US)

(72) Inventors: Hien M. Nguyen, Bloomfield Hills, MI (US); Ravi Sankar Loka, Detroit, MI (US); Eric Sletten, Royal Oak, MI (US); Israel Vlodavsky, Haifa (IL)

(73) Assignees: Wayne State University, Detroit, MI (US); Technion, Israel Institute of Technology, Haifa (IL); University of Iowa Research Foundation, Iowa City, IA (US)

(21) Appl. No.: 17/311,088

(22) PCT Filed: Dec. 5, 2019
(86) PCT No.: PCT/US19/64771

§ 371 Date: Jun. 4, 2021

Related U.S. Application Data
(60) Provisional application No. 62/775,800, filed on Dec. 5, 2018.

(51) Int. Cl. A61K 31/727; A61P 35/00

(52) U.S. Cl. CPC A61K 31/727; A61P 35/00

(57) ABSTRACT

Anti-heparanase compounds for the treatment of cancer are described. The anti-heparanase compounds are high affinity, synthetic glycopolymers that result in minimal anticoagulant activity. Stereoselective fluorinated forms of these compounds are also provided.

### CROSS-REFERENCE TO RELATED APPLICATION

This application claims priority to U.S. Provisional Patent Application No. 62/775,800 filed Dec. 5, 2018 the entire contents of which are incorporated by reference herein as if fully set forth herein.

### STATEMENT REGARDING FEDERALLY SPONSORED RESEARCH OR DEVELOPMENT

This invention was made with government support under grant GM098285 awarded by the National Institutes of Health. The government has certain rights in the invention.

### FIELD OF THE DISCLOSURE

The current disclosure provides anti-heparanase compounds for the treatment of cancer. The anti-heparanase compounds are high affinity, synthetic glycopolymers that result in minimal anticoagulant activity. Stereoselective fluorinated forms of these compounds are also provided.

### BACKGROUND OF THE DISCLOSURE

Cancer is a ubiquitous disease which has pandemic and destructive effects on living organisms. In 2018, the World Health Organization estimated cancer to be the second leading cause of death in humans globally, with an estimated total number of deaths recorded as 9.6 million. Cancer occurs when abnormal cell growth (pre-cancerous lesion) transforms into malignant tumors and metastasize throughout the human body.

Some common forms of cancer include: bladder cancer, breast cancer, colon and/or rectal cancer, endometrial cancer, kidney cancer, leukemia, liver cancer, lung cancer, melanoma, Non-Hodgkin lymphoma, pancreatic cancer, prostate cancer, and thyroid cancer. Some leading risk factors reported to be the main cause for developing cancer include: smoking, high exposure to ultraviolet radiation, unhealthy food and beverage consumption (i.e. unhealthy diet intake), over consumption of alcohol, environmental conditions, and obesity.

Glycosidase enzymes have emerged as a potential target for anticancer drug development due to the glycosidases' ability to catalyze the hydrolysis of glycosidic bonds in complex sugars and the vital role the enzymes play in cellular functions. The hydrolysis of polysaccharides can lead to a range of diseases including diabetes, lysosomal disorders, cystic fibrosis, influenza, Alzheimer's and cancers. Considering these findings, the inhibition of heparanase, a type of glycosidase enzyme, has been targeted as a viable cancer therapeutic.

Heparanase is an endolytic enzyme that degrades heparan sulfate polysaccharide chains, which are widely distributed in tissues and have important regulatory and structural functions in the extracellular matrix and at the cell surface. Heparanase has been shown to cleave heparan sulfate chains at specific sulfation patterns along the internal β-(1,4)-glycosidic bond, between glucuronic acid and N-sulfated glucosamine. Increased levels of heparanase expression have been associated with disease progression, increased tumor growth, increased angiogenesis, metastatic spread, and poor patient prognosis for both hematological and solid tumor malignancies. Therefore, the inhibition of the heparanase enzyme provides an attractive target in the development of anticancer therapeutics.

Classes of molecules have been developed to control heparanase activity. These molecules include: PI-88 and analogues; oligomannurarate JG3; small molecule inhibitors; carbohydrate molecules; saccharide-functionalized glycopolymers; the anticoagulant, heparin; and macromolecules including polysaccharides. In addition, anti-heparanase antibodies that inhibit heparanase activity and subsequent cellular responses have been reported.

The use of these anti-heparanase molecules and antibodies are not without drawbacks. For instance, PI-88, although the most clinically advanced heparanase inhibitor, has a complex mode of action inhibiting both heparanase activity and the binding of growth factors to heparan sulfate. PI-88's clinical trials were ended, as patients developed antibody-induced thrombocytopenia (Rivara et al., Future Med Chem 8:67, 2016; Vlodaysky et al., Drug Resist Updates. 29:54, 2016; Maxhimer et al., Surgery 132:326, 2002; Elkin et al., FASEB J. 15:1661, 2001; Cohen et al., Cancer 113:1004, 2008; Ramani et al., Matrix Biol. 55; 22, 2016; Kudchadkar et al., Expert Opin Invest Drugs 17:1769, 2008). Heparin's anticoagulant activity has limited its use for cancer treatment due to the risk of bleeding complications. Carbohydrate anti-heparanase molecules are heterogeneous in size and sulfation pattern leading to nonspecific binding and unforeseen adverse effects, thus halting their translation into clinical use. Macromolecule anti-heparanases are still met with the challenge of developing an inhibiting epitope (inhitope) that can gain access to the active site of heparanase.

### SUMMARY OF THE DISCLOSURE

The current disclosure provides use of anti-heparanase compounds as anti-cancer agents. In particular embodiments, the anti-heparanase compounds include high affinity, synthetic glycopolymers with minimal anticoagulant activity. In particular embodiments, the anti-heparanase compounds are heparan sulfate mimicking glycopolymers containing disulfated disaccharide. In particular embodiments, the anti-heparanase compounds are stereoselective fluorinated forms of glycopolymer compounds.

### BRIEF DESCRIPTION OF THE SEVERAL VIEWS OF THE DRAWINGS

Many of the drawings submitted herein are better understood in color. Applicant considers the color versions of the drawings as part of the original submission and reserves the right to present color images of the drawings in later proceedings.

FIG. 1: Heparanase cleavage at explicit sulfation pattern. The process of deriving a sulfated glycopolymer inhibitor from natural heparan sulfate (HS) binding to the positively charged binding domains (HBD-1 and HBD-2) of heparanase. Heparanase has been shown to specifically cleave at an explicit sulfation pattern, GlcAβ(1,4)GlcNS(6S), along the HS polysaccharide chain. Advantages of this process include: Homogenous sulfation; strong and specific binding; adjustable valency; and the quick exchange of the saccharide motif.

FIG. 2. C2A-C2G Disaccharides with sulfation patterns varying at the C(6)-O, C(3)-O, and C(2)-N positions. C2A-C2G show the rational design of disaccharide motifs bearing the sulfation patterns at the C(6)-O, C(3)-O, and C(2)-N positions of the glucosamine unit. Disaccharides C2B and C2C will examine whether C(6)-O—SO₃⁻ located at the −2 subsite is critical for recognition. C2B and C2D will determine whether the sulfate group located at the C(6) or C(3) position of the glucosamine unit is more important. Disaccharides C2E and C2F will provide a clear picture of whether N—SO₃⁻ groups located at the −2 subsite of heparanase could be critically important for heparanase-HS interaction. The highly sulfated C2G could have a negative or positive impact on HS-heparanase interactions. The letter “C”, designated for each disaccharide, means Compound.

FIG. 3: The schematic synthesis of protected disaccharide motifs C3E-C3I. The synthesis of protected disaccharide motif C3E includes: N—CF₃ aceylation; O-deacetylation; C-6 and C-3 sulfation; and NAP deprotection. The synthesis of protected disaccharide motif C3F includes: N-sulfation; NAP removal; and O-deacetylation. The synthesis of protected disaccharide motif C3G includes: benzylidene removal; C-6 and N-sulfation; and NAP deprotection. The synthesis of protected disaccharide motif C3H includes: benzylidene removal; C-6 and N-sulfation; C-3 deacetylation; C-3 sulfation; and NAP deprotection. The synthesis of protected disaccharide motif C3I includes: C-6 NAP protection; benzylidene removal; N-sulfation; C-3 deacetylation; C-3 sulfation; and NAP deprotection.

FIG. 4: Synthesis of HS-mimicking glycopolymers via click chemistry followed by ring-open metathesis polymerization (ROMP). Protected disaccharide motifs C3E-C3I are partly composed in the structure of C4A. Disaccharide C5A-05F are partly composed in the ring opening structure in C4D.

FIG. 5: Glycopolymer inhibition pattern of heparanase by HS mimicking glycopolymers using a TR-FRET assay. ^{a}DP and molecular weights (Mₙ) were determined via ¹H-NMR end group analysis. ^{b}Inhibition of heparanase was assessed by in vitro TR-FRET assay against fluorescent-tagged heparan sulfate.

FIG. 6: Positioning of the natural HS substrate, GlcNS(6S)α(1,4)GlcAβ(1,4)GlcNS(6S)α(1,4)GlcA, in the active site of human heparanase. This tetrasaccharide was docked into the apo crystal structure of heparanase (PDB code: 5E8M) using the Autodock Vina suite in YASARA program (Wu, et al., Nat. Struct. Mol. Biol. 2015, 22, 1016-1022; Krieger, et al., Bioinformatics 2014, 30 (20), 2981-2982; Trott, et al., J. Comput. Chem. 2010, 31 (2), 455-461). FIG. 6 was generated using LigPlot⁺ (Laskowski, et al., J. Chem. Inf. Model. 2011, 51 (10), 2778-2786; Wallace, et al., Protein Eng. Des. Sel. 1995, 8 (2), 127-134).

FIG. 7: Binding affinity of GlcNS(6S)α(1,4)GlcA glycopolymer to various HS-binding proteins. The binding affinity was calculated using Equation 1 as referenced in Chai, et al. (Anal. Biochem. 2009, 395 (2), 263-264).

FIGS. 8A-8C: Cross bioactivity studies. (8A) The biolayer interferometry (BLI) trace for the binding of various concentrations (0.016-50 μM) of GlcNS(6S)α(1,4)GlcA glycopolymer (DP=12) to FGF-2. (8B) shows HUVEC cell growth when incubated at 3000 cells/well/100 μL with FGF-2 or FGF-2 plus GlcNS(6S)α(1,4)GlcA glycopolymer (DP=12) at varying concentrations for three days. Absorbance of living cells was measured using CellTiter 96® (Promega Corp., Madison, Wis.) AQueous One Solution at 490 nm. Data were normalized to cells incubated with medium alone (set to 100%). Background absorbance from the polymer at each concentration and medium alone were subtracted from the respective polymer containing samples. Only the medium background absorbance was subtracted from the rest of the samples. The experiment was repeated three times with at least triplicates of each sample per experiment, error bars represent standard deviation. Statistical analysis was done using Welch's t-test. *p<0.01 compared to cells plus FGF-2. (8C) shows the overlay comparing the critical micelle concentration (CMC) data of GlcNS(6S)α(1,4)GlcA glycopolymer (DP=12) with the HUVEC proliferation data.

FIG. 9: Effect of glycopolymer on 4T1 experimental metastasis. Luciferase-labeled 4T1 breast carcinoma cells (1×10⁵/mouse) were injected i.v (n=6 mice/group) with vehicle alone (control, PBS), with positive control (heparin), or with GlcNS(6S)α(1,4)GlcA glycopolymer (DP=12, 100 μg/mouse) injected (i.p) 20 min prior to cell inoculation and also together with the cells. IVIS bioluminescent imaging was performed on day 7 after cell inoculation. For IVIS imaging, mice were injected intraperitoneally with D-luciferin substrate at 150 mg/kg and anesthetized with continuous exposure to isoflurane (EZAnesthesia, Palmer, Pa.). Light emitted from the bioluminescent cells is detected by the IVIS camera system with images quantified for tumor burden using a log-scale color range set at 5×10⁴ to 1×10⁷ and measurement of total photon counts per second (PPS) using Living Image software (Xenogen). The experiment was repeated 3 times with similar results.

FIG. 10: The structure of compound 5A (C5A).

FIG. 11: The synthetic route for the synthesis of trisulfated glycopolymer (C5D).

FIG. 12: The synthetic route for synthesis of C(3)-SO₃ N—SO₃ disulfated glycopolymer (C5C).

FIG. 13: The synthesis for the removal of N-benzylidene for disaccharide (C3B).

FIG. 14: The synthetic route for N-acetylated disulfated glycopolymer (C5E).

FIG. 15: The synthetic route for free amine disulfated glycopolymer (C5F).

FIG. 16: The synthetic route for N-sulfated glycopolymer (C5B).

FIGS. 17A-17F: Computational docking study. For the docking studies, the disclosure used the apo heparanase structure (PDB code: 5E8M) (Wu, et al., Nat. Struct. Mol. Biol. 2015, 22, 1016-1022.). (17A) shows C(6)-SO₃ N—SO₃ disulfated monomer docked into heparanase. (17B) shows trisulfated monomer docked into heparanase. (17C) shows N-acetylated disulfated monomer docked into heparanase. (17D) shows free amine disulfated monomer docked into heparanase. (17E) shows N-sulfated monomer docked into heparanase. (17F) shows C(3)-SO₃ N—SO₃ disulfated monomer docked into heparanase.

FIGS. 18A-18F: Biological assay protocols. The inhibition of heparanase by polymers of different sulfation patterns. (18A) shows the inhibition of heparanase by C(6)-SO₃ N—SO₃ disulfated glycopolymer (C5A). (18B) shows the inhibition of heparanase by N-sulfated glycopolymer (C5B). (18C) shows the inhibition of heparanase by C(3)-SO₃ N—SO₃ disulfated glycopolymer (C5C). (18D) shows the inhibition of heparanase by trisulfated glycopolymer (C5D). (18E) shows the inhibition of heparanase by N-acetylated disulfated glycopolymer (C5E). (18F) shows the inhibition of heparanase by free amine disulfated glycopolymer (C5F).

FIGS. 19A-19U: FGF-2 induced cell proliferation assay. The BLI sensorgrams and fitted response curves. FIGS. 19A)-(19C) show BLI sensorgrams and fitted response curves for the analysis of FGF-1 and heparin. Analysis of stoichiometry for FGF-1/heparin was fitted for a segmented linear regression equation. FIGS. 19D) and (19E) show a BLI sensorgram and fitted response curve for the analysis of FGF-1 and glycopolymer (C5A). FIGS. 19F) and (19G) show a BLI sensorgram and fitted response curve for the analysis of FGF-2 and heparin. FIGS. 19H) and (19I) show a BLI sensorgram and fitted response curve for the analysis of FGF-2 and glycopolymer (C5A). FIGS. 19J) and (19K) show a BLI sensorgram and fitted response curve for the analysis of VEGF and heparin. FIGS. 19L) and (19M) show a BLI sensorgram and fitted response curve for the analysis of VEGF and glycopolymer (C5A). FIGS. 19N) and (19O) show a BLI sensorgram and fitted response curve for the analysis of PF4 and heparin. FIGS. 19P) and (19Q) show a BLI sensorgram and fitted response curve for the analysis of PF4 and glycopolymer (C5A). FIGS. 19R) and (19S) show a BLI sensorgram and fitted response curve for the analysis of P-selectin and heparin. FIGS. 19T) and (19U) show a BLI sensorgram and fitted response curve for the analysis of P-selectin and glycopolymer (C5A).

FIG. 20: The strategies for substrate-controlled glycosylation. (A) shows the general structures of 1,2-trans-, 1,2-cis-, and α-2-deoxy carbohydrates. (B) shows the influence of C-2 neighboring group on 1,2-trans glycoside formation. (C) shows the Influence of C-2 non-participatory group on 1,2-cis glycoside formation.

FIG. 21: The strategies for catalyst-controlled glycosylation. (A) shows the retaining glycosyltransferases-catalyzed α-glycosylation. (B) shows the proposed mechanism for phenanthroline-catalyzed α-stereoretentive glycosylation for access axial 1,2-cis glycosides.

FIGS. 22A-22C: The catalytic glycosylations. (22A) shows the reaction development with phenanthroline catalyst. (22B) shows a standard setup for construction of disaccharide 3. (22C) shows a gram-scale glycosylation reaction. Yields were determined by isolation after chromatographic purification. Diastereomeric (α/β) ratios were determined through analysis by proton nuclear magnetic resonance (¹H NMR) spectroscopy.

FIG. 23: Screening of small-molecule catalysts.

FIG. 24: Screening of hydrogen bromide (HBr) scavengers.

FIG. 25: Increase catalyst loading in the reaction to obtain disaccharide 3 and 1.

FIG. 26: The effect of concentration by introducing a range of concentration parameters to obtain disaccharide 3.

FIG. 27: The effect of various solvents when added to the reaction to obtain disaccharide 3.

FIG. 28: The effect of reaction temperature when a specific temperature is added to the reaction.

FIG. 29: Scope with respect to glucose electrophiles. While acetyl-protected electrophiles were conducted at 50° C., fully protected benzyl-derived electrophiles were conducted at 25° C. Yields were determined by isolation after chromatographic purification. Diastereomeric (α/β) ratios were analyzed by ¹H NMR spectroscopy.

FIG. 30: Mechanistic studies. In (A) the Identification of β-phenanthrolium ion was accomplished by using mass spectroscopy. (B) shows the effect of glycosyl bromide configuration. (C) and (D) show the kinetics of the reaction of isopropanol with glycosyl bromide. (E) and (F) show the intermediate structure calculated using the B3LYP/6-31+G(d,p) level with the solvent model density (SMD) solvent model. (G) and (H) show the non-covalent interactions plot (reduced density gradient isosurface=0.3) for the optimized structure at B3LYP/6-31+G(d,p). The nitrogen surfaces represent attractive interactions, and the carbon surfaces represent repulsive interactions.

FIG. 31: Synthesis of octasaccharide. (a) shows the reactants used were: 5-15 mol % of catalyst 4, IBO (2 equiv.), MTBE (2 M), 50° C., 24 h, 34: 89%, α:β>25:1; 37: 86%, α:β>25:1; 40: 77%, α:β>25:1. (b) shows various solvents, temperature, and disaccharides percentages used in the reaction includes: NaOMe, MeOH, CH₂Cl₂, 25° C., 35: 99%, 38: 70%. (c) shows that glycosyl bromides 36 and 39 were prepared from 34 and 37, respectively, using the following conditions: PTSA, Ac₂O, 70° C., 2 h then HBr/AcOH, CH₂Cl₂, 0° C., 15 min.

FIG. 32: Synthesis of disaccharide 41 and 3 using various reaction conditions.

FIG. 33: Anomerization of β-bromide to α-bromide for disaccharide 1 using various reaction conditions.

FIG. 34: Attempted isomerization of disaccharide 3 using various reaction conditions (including the addition of disaccharide 2 in the reaction).

FIG. 35: Example spectra array for a kinetic experiment.

FIG. 36: Example rate plot for a kinetic experiment showing product concentration versus time.

FIG. 37: Rate of reaction versus catalyst concentration.

FIG. 38: Rate of reaction versus acceptor concentration.

FIG. 39: Product formation versus time at different equivalent of isobutylene oxide (IBO).

FIG. 40: Phenanthroline-catalyzed glycosylation reactions carried out using various reacting conditions.

FIG. 41: Gram scale synthesis of disaccharide 3.

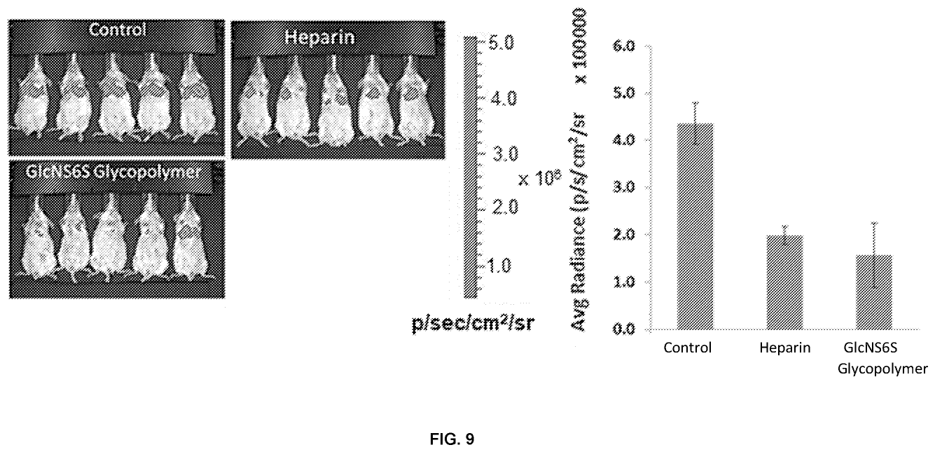

FIGS. 9A8-104: Synthesis of octasaccharides and NMR analysis of desired disaccharides that participate in the synthesis of octasaccharides 40.

FIG. 42: Synthesis of disaccharide 34.

FIG. 43: Synthesis of disaccharide 35.

FIG. 44: Synthesis of tetraccharide 37.

FIG. 45: Synthesis of disaccharide 38.

FIG. 46: Synthesis of disaccharide 40.

FIG. 47: Synthesis of product 1P.

FIG. 48: Rate equation derivation.

FIGS. 49A-49N: Optimized structures and corresponding cartesian coordinates.

### DETAILED DESCRIPTION

Cancer is a ubiquitous disease which has pandemic and destructive effects on living organisms. In 2018, the World Health Organization estimated cancer to be the second leading cause of death in humans globally, with an estimated total number of deaths recorded as 9.6 million (Who. int. (2019), [Accessed 12 Nov. 2019]). Cancer occurs when abnormal cell growth (pre-cancerous lesion) transforms into malignant tumors and metastasize throughout the human body (Who. int. (2019), [Accessed 12 Nov. 2019]).

Some common forms of cancer include: bladder cancer, breast cancer, colon and/or rectal cancer, endometrial cancer, kidney cancer, leukemia, liver cancer, lung cancer, melanoma, Non-Hodgkin lymphoma, pancreatic cancer, prostate cancer, and thyroid cancer (National Cancer Institute. (2019), [Accessed 12 Nov. 2019]). Some leading risk factors reported to be the main cause for developing cancer include: smoking, high exposure to ultraviolet radiation, unhealthy food and beverage consumption (i.e. unhealthy diet intake), over consumption of alcohol, environmental conditions, and obesity (Cdc.gov. (2019) [Accessed 12 Nov. 2019]; Who. int. (2019), [Accessed 12 Nov. 2019).

Glycosidase enzymes have emerged as potential targets for anticancer drug development (Compain, et al., ChemBioChem 2014, 15 (9), 1239-1251) due to the glycosidases' ability to catalyze the hydrolysis of glycosidic bonds in complex sugars and the vital role the enzymes play in cellular functions (Vocadlo, et al., Curr. Opin. Chem. Biol. 2008, 12 (5), 539-555;). The hydrolysis of polysaccharides can lead to a range of diseases including diabetes, lysosomal disorders, cystic fibrosis, influenza, Alzheimer's and cancers (Lillelund, et al., Chem Rev 2002, 102, 515-83; Kajimoto, et al., Curr Top Med Chem 2009, 9, 13-33, Gloster, et al., Org. Biomol Chem 2010, 8, 305-20, Ghani, et al., Eur J Med Chem 2015, 103, 133-62; Singha, A., et al., Med. Chem 2015, 15, 933-946; Bras, et al., Expert Opinion on Therapeutic Patents 2014, 24, 857-874). Considering these findings, the inhibition of heparanase, a type of glycosidase enzyme, has been targeted as a viable cancer therapeutic (Rivara, et al., Future Medicinal Chemistry, 2016, 8, 647-680).

Heparanase is an endolytic enzyme that degrades heparan sulfate polysaccharide chains, which are widely distributed in tissues and have important regulatory and structural functions in the extracellular matrix and at the cell surface. Heparanase has been shown to cleave heparan sulfate chains at specific sulfation patterns along the internal β-(1,4)-glycosidic bond, between glucuronic acid and N-sulfated glucosamine (Rivara, et al., Future Med. Chem. 2016, 8 (6), 647-680; Vlodaysky, et al., Drug Resist. Updates 2016, 29, 54-75; Pisano, et al., Biochem. Pharmacol. 2014, 89 (1), 12-19; Vlodaysky, et al., Nat. Med. 1999, 5, 793). Increased levels of heparanase expression have been associated with disease progression, increased tumor growth, increased angiogenesis, metastatic spread, and poor patient prognosis for both hematological and solid tumor malignancies (Ilan, et al., Int. J. Biochem. Cell Biol. 2006, 38 (12), 2018-2039; Barash, et al., FEBS J. 2010, 277 (19), 3890-3903; Arvatz, et al., Cancer Metastasis Rev. 2011, 30 (2), 253-268; Vlodaysky, et al., Rambam Maimonides Med. J. 2011, 2 (1), e0019; Vlodaysky, et al., Cancer Microenviron. 2012, 5 (2), 115-132; Knelson, et al., Trends Biochem. Sci. 2014, 39 (6), 277-288; Sanderson, et al., Semin. Cell Dev. Biol. 2001, 12 (2), 89-98; US20100233154A1). Therefore, the inhibition of the heparanase enzyme provides an attractive target in the development of anticancer therapeutics.

Classes of molecules have been developed to control heparanase activity. These molecules include: PI-88 and analogues (Karoli et al, J Med Chem 48 8229-8236 2005); oligomannurarate JG3 (Zhao et al, Cancer Res 66 8779-8787 2006); small molecule inhibitors (Ishida et al, Mol Cancer Therap 3 1069-1077 2004; J Org Chem 70 8884-8889 2005; Xu et al, Bioorg Med Chem Lett 16 404-408 2006; Pan et al, Bioorg Med Chem Lett 16 409-412 2006); carbohydrate molecules (Rivara, et al., Future Med. Chem. 2016, 8 (6), 647-680; Kudchadkar, et al., Expert Opin. Invest. Drugs 2008, 17 (11), 1769-1776; Cassinelli, et al., Oncotarget 2016, 7 (30), 47848-47863; Cassinelli, et al., Biochem. Pharmacol. 2013, 85 (10), 1424-1432; Naggi, et. al., J. Biol. Chem. 2005, 280 (13), 12103-12113; Vlodaysky, et al., Curr. Pharm. Des. 2007, 13 (20), 2057-2073; Bar-Ner, et al., Blood 1987, 70 (2), 551-557; Jia, et al., Eur. J. Med. Chem. 2016, 121, 209-220; Lanzi, et al., Curr. Med. Chem. 2017, 24 (26), 2860-2886; Weissmann, et al., Proc. Natl. Acad. Sci. U.S.A 2016, 113 (3), 704-709; Mitsiades, et al., Clin. Cancer. Res. 2009, 15 (4), 1210-1221); saccharide-functionalized glycopolymers (Lundquist, et al., Chem. Rev. 2002, 102 (2), 555-578.); the anticoagulant, heparin (Naggi, et al., J. Bio. Chem. 280, 12103-12113); and macromolecules including polysaccharides (Hosseinkhani, et al., J. Nanopart. Res. 2013, 15 (1), 1345-1355; Abedini, et al., Polym. Adv. Technol. 2018, 29 (10), 2564-2573; Ghadiri, et al., J. Biomed. Mater. Res., Part A 2017, 105 (10), 2851-2864; Khan, et al., Acta Biomater. 2012, 8 (12), 4224-4232; Hosseinkhani, et al., Gene Ther. 2004, 11 (2), 194-203). In addition, anti-heparanase antibodies that inhibit heparanase activity and subsequent cellular responses have been reported (US20100233154A1).

The use of these anti-heparanase molecules and antibodies are not without drawbacks. For instance, PI-88, although the most clinically advanced heparanase inhibitor, has a complex mode of action inhibiting both heparanase activity and the binding of growth factors to heparan sulfate. PI-88's clinical trials were ended, as patients developed antibody-induced thrombocytopenia (Rivara et al., Future Med Chem 8:67, 2016; Vlodaysky et al., Drug Resist Updates. 29:54, 2016; Maxhimer et al., Surgery 132:326, 2002; Elkin et al., FASEB J. 15:1661, 2001; Cohen et al., Cancer 113:1004, 2008; Ramani et al., Matrix Biol. 55; 22, 2016; Kudchadkar et al., Expert Opin Invest Drugs 17:1769, 2008). Heparin's anticoagulant activity has limited its use as a cancer treatment due to the risk of bleeding complications (Letai, et al., The Oncologist, December 1999 vol. 4 no. 6 443-449). Carbohydrate anti-heparanase molecules are heterogeneous in size and sulfation pattern leading to nonspecific binding and unforeseen adverse effects, thus halting their translation into clinical use. Macromolecule anti-heparanases are still met with the challenge of developing an inhibiting epitope (inhitope) that can gain access to the active site of heparanase (Compain, et al., ChemBioChem 2014, 15 (9), 1239-1251).

The current disclosure provides use of anti-heparanase compounds as anti-cancer agents. In particular embodiments, the anti-heparanase compounds include high affinity, synthetic glycopolymers with minimal anticoagulant activity. In particular embodiments, the anti-heparanase compounds are heparan sulfate mimicking glycopolymers containing disulfated disaccharide. In particular embodiments, the anti-heparanase compounds are stereoselective fluorinated forms of glycopolymer compounds. As shown herein, the described compounds provided significant anti-cancer effects. For example, in a mouse model of cancer, the sulfated glycopolymer effectively prohibited carcinoma cells from metastasizing and migrating to the lungs.

As indicated, in particular embodiments, the anti-heparanase glycopolymers disclosed herein have high bonding affinity to various heparan sulfate-binding proteins and minimal anti-coagulant activity compared to the anticoagulating molecule heparin.

In particular embodiments, high affinity refers to a higher apparent dissociation constant of the anti-heparanase glycopolymer when bound to various heparan sulfate-binding proteins, as compared to heparin dissociation constant. For example, heparin naturally binds the proteins FGF-1, FGF-2, VEGF, and PF4. When measured by a solution-based biolayer interferometry (BLI) assay, heparin was found to have a dissociation constant (in nM) of 4.6±3.3, 0.15±0.11, 4.91±1.55, and 0.31±0.028, respectively, when calculated using the Equation:

$$F = {F}_{0} + ( {F}_{MAX} - {F}_{0} )$$

$$\frac{( {n [ P ]}_{T} + {[ M ]}_{T} + {K}_{D} ) - \sqrt{{( {n [ P ]}_{T} + {[ M ]}_{T} + {K}_{D} )}^{2} - 4 {{n [ P ]}_{T} [ M ]}_{T}}}{2 {n [ P ]}_{T}}$$

where F is the fluorescence signal, F₀ is the signal from a blank, F_{MAX} corresponds to the maximal fluorescence intensity, K_{D} is the dissociation constant and n is the number of independent binding sites. Using the same assay, anti-heparanase compounds described herein (e.g., glycopolymer 20) was found to have dissociation constants (in nM) of 2000, 691±162, 281±162, 281±162, and 45±5.11, respectively. Accordingly, “high affinity” can be at least 2× higher binding affinity, at least 4× higher binding affinity, at least 8× higher binding affinity, at least 16× higher binding affinity, at least 32× higher binding affinity, at least 64× higher binding affinity, at least 85× higher binding affinity, at least 100× higher binding affinity or more when compared to heparin's binding to the same heparan sulfate-binding protein.

In particular embodiments, minimal anti-coagulant activity is measured by the anti-heparanase glycopolymer's binding affinity to antithrombin III (ATIII), compared to the anticoagulating molecule heparin's binding affinity to ATIII. In particular embodiments, minimal anti-coagulant activity means that the glycopolymer's binding affinity to ATIII is reduced compared to heparin's binding affinity to ATIII. The reduction can be at least a 10% reduction, at least a 20% reduction, at least a 30% reduction, at least a 50% reduction, or more. In particular embodiments, minimal anti-coagulant activity means that the anti-heparanase glycopolymer has no detectable binding to ATIII. In particular embodiments, minimal anti-coagulant activity means that no coagulant activity is detected in a coagulation assay.

Aspects of the current disclosure are now described with additional detail and options as follows: (i) Compounds for Use as Anti-Cancer Agents; (ii) Compositions for Administration; (iii) Methods of Use; (iv) Experimental Examples; (v) Additional Xenograft Models; and (vi) Closing Paragraphs.

(i) Compounds for Use as Anti-Cancer Agents. In one aspect the present disclosure describes use of compounds that are useful for inhibiting heparanase for the treatment of cancer. In particular embodiments, the disclosure provides use of a compound of formula II:

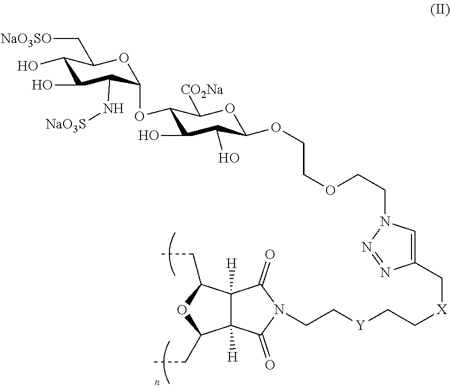

wherein:

### X is —O— or

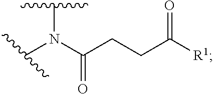

Y is —O— or —CH₂—; n is an integer from 2-100 inclusive; R¹ is OH or —N(H)-L-R^{a}; L is a linking group; R^{a} is a saccharide or disaccharide, which saccharide or disaccharide includes one or more —SO₃Na groups; and the dash bond --- is a single bond or a double bond; or a salt thereof.

The following definitions are used, unless otherwise described: halo is fluoro, chloro, bromo, or iodo. Alkyl, alkoxy, alkenyl, alkynyl, etc. denote both straight and branched groups; but reference to an individual radical such as propyl embraces only the straight chain radical, a branched chain isomer such as isopropyl being specifically referred to. Aryl denotes a phenyl radical or an ortho-fused bicyclic carbocyclic radical having nine to ten ring atoms in which at least one ring is aromatic. Heteroaryl encompasses a radical of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of eight to ten ring atoms including one to four heteroatoms each selected from the group consisting of non-peroxide oxygen, sulfur, and N(X).

The term “alkyl”, by itself or as part of another substituent, means, unless otherwise stated, a straight or branched chain hydrocarbon radical, having the number of carbon atoms designated (i.e., C₁₋₈ means one to eight carbons). Examples include (C₁-C₈)alkyl, (C₂-C₈)alkyl, C₁-C₆)alkyl, (C₂-C₆)alkyl and (C₃-C₆)alkyl. Examples of alkyl groups include methyl, ethyl, n-propyl, iso-propyl, n-butyl, t-butyl, iso-butyl, sec-butyl, n-pentyl, n-hexyl, n-heptyl, n-octyl, and higher homologs and isomers.

The term saccharide includes monosaccharides, disaccharides, trisaccharides and polysaccharides. The term includes glucose, galactose, glucosamine, galactosamine, glucuronic acid, idouronic acid, sucrose fructose and ribose, as well as 2-deoxy sugars such as deoxyribose and the like or 2-fluoro-2-deoxy-sugar. Saccharide derivatives can conveniently be prepared as described in International Patent Applications Publication Numbers WO 96/34005 and 97/03995. A saccharide can conveniently be linked to the remainder of a compound of formula I through an ether bond.

Linker. As described herein, the targeting element can be bonded (connected) to the remainder of the targeted conjugate agent through an optional linker. In particular embodiments the linker is absent (e.g., the targeting element can be bonded (connected) directly to the remainder of the targeted conjugate). The linker can be variable provided the targeting conjugate functions as described herein. The linker can vary in length and atom composition and for example can be branched or non-branched or cyclic or a combination thereof. The linker may also modulate the properties of the targeted conjugate such as solubility, stability and aggregation.

Since the linkers used in the targeted conjugates (e.g., linkers including polyethylene glycol (PEG)) can be highly variable, it is possible to use different sizes and types of targeting elements and still maintain the desired and/or optimal pharmacokinetic profile for the targeted conjugate.

In particular embodiments the linker includes 3-5000 atoms. In particular embodiments the linker includes 3-4000 atoms. In particular embodiments the linker includes 3-2000 atoms. In particular embodiments the linker includes 3-1000 atoms. In particular embodiments the linker includes 3-750 atoms. In particular embodiments the linker includes 3-500 atoms. In particular embodiments the linker includes 3-250 atoms. In particular embodiments the linker includes 3-100 atoms. In particular embodiments the linker includes 3-50 atoms. In particular embodiments the linker includes 3-25 atoms.

In particular embodiments the linker includes 10-5000 atoms. In particular embodiments the linker includes 10-4000 atoms. In particular embodiments the linker includes 10-2000 atoms. In particular embodiments the linker includes 10-1000 atoms. In particular embodiments the linker includes 10-750 atoms. In particular embodiments the linker includes 10-500 atoms. In particular embodiments the linker includes 10-250 atoms. In particular embodiments the linker includes 10-100 atoms. In particular embodiments the linker includes 10-50 atoms. In particular embodiments the linker includes 10-25 atoms.

In particular embodiments the linker includes atoms selected from H, C, N, S and O.

In particular embodiments the linker includes atoms selected from H, C, N, S, P and O.

In particular embodiments the linker includes a branched or unbranched, saturated or unsaturated, hydrocarbon chain, having from 1 to 1000 (or 1-750, 1-500, 1-250, 1-100, 1-50, 1-25, 1-10, 1-5, 5-1000, 5-750, 5-500, 5-250, 5-100, 5-50, 5-25, 5-10 or 2-5 carbon atoms) wherein one or more of the carbon atoms is optionally replaced independently by —O—, —S, —N(R^{a})—, 3-7 membered heterocycle, 5-6-membered heteroaryl or carbocycle and wherein each chain, 3-7 membered heterocycle, 5-6-membered heteroaryl or carbocycle is optionally and independently substituted with one or more (e.g. 1, 2, 3, 4, 5 or more) substituents selected from (C₁-C₆)alkyl, (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, —N(R^{a})₂, hydroxy, oxo (═O), carboxy, aryl, aryloxy, heteroaryl, and heteroaryloxy, wherein each R^{a} is independently H or (C₁-C₆)alkyl. In particular embodiments the linker includes a branched or unbranched, saturated or unsaturated, hydrocarbon chain, having from 1 to 1000 (or 1-750, 1-500, 1-250, 1-100, 1-50, 1-25, 1-10, 1-5, 5-1000, 5-750, 5-500, 5-250, 5-100, 5-50, 5-25, 5-10 or 2-5 carbon atoms) wherein one or more of the carbon atoms is optionally replaced independently by —O—, —S, —N(R^{a}), wherein each R^{a} is independently H or (C₁-C₆) alkyl.

In particular embodiments the linker includes a polyethylene glycol. In particular embodiments the linker includes a polyethylene glycol linked to the remainder of the targeted conjugate by a carbonyl group. In particular embodiments the polyethylene glycol includes 1 to 500 or 5 to 500 or 3 to 100 repeat (e.g., —CH₂CH₂O—) units (Greenwald, R. B., et al., Poly (ethylene glycol) Prodrugs: Altered Pharmacokinetics and Pharmacodynamics, Chapter, 2.3.1., 283-338; Filpula, D., et al., Releasable PEGylation of proteins with customized linkers, Advanced Drug Delivery, 60, 2008, 29-49; Zhao, H., et al., Drug Conjugates with Poly(Ethylene Glycol), Drug Delivery in Oncology, 2012, 627-656).

In particular embodiments the linker is —NH(CH₂CH₂O)₄CH₂CH₂C(═O)—. In particular embodiments the linker is —NH(CH₂CH₂O)ₙCH₂CH₂C(═O)— wherein n is 1-500, 5-500, 3-100, 5-50, 1-50, 1-20, 1-10, 1-5, 2-50, 2-20, 2-10, 2-5, 3-50, 3-20, 3-10, 3-5, 4-50, 4-20, 4-10, 4-5. In particular embodiments the linker is —(CH₂CH₂O)₄CH₂CH₂C(═O)—.

It will be appreciated by those skilled in the art that compounds of the disclosure having a chiral center may exist in and be isolated in optically active and racemic forms. Some compounds may exhibit polymorphism. It is to be understood that the present disclosure encompasses any racemic, optically-active, polymorphic, or stereoisomeric form, or mixtures thereof, of a compound of the disclosure, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In particular embodiments, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95 the absolute stereoisomer depicted. In another embodiment, the compound may be at least 99% the absolute stereoisomer depicted.

In particular embodiments, X is —O— and Y is —O—; or X is

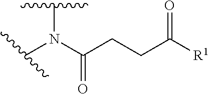

### and Y is —CH₂—.

In particular embodiments, the compound of formula II is a compound of formula

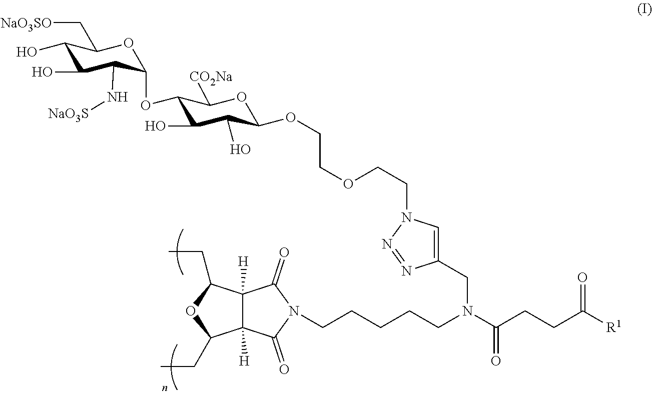

wherein: n is an integer from 2-100 inclusive; R¹ is OH or a salt or —N(H)-L-R^{a}; Lisa linking group; and R^{a} is a saccharide or disaccharide, which saccharide or disaccharide includes one or more —SO₃H groups; or a salt thereof.

In particular embodiments, the compound of formula II is a compound of formula (Ia):

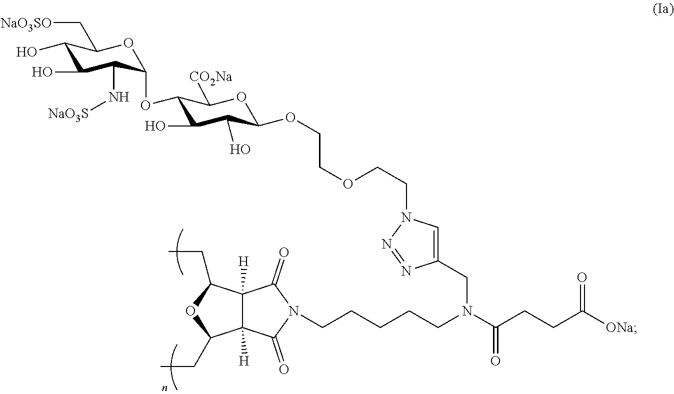

In particular embodiments, the compound of formula II is a compound of formula (Ib):

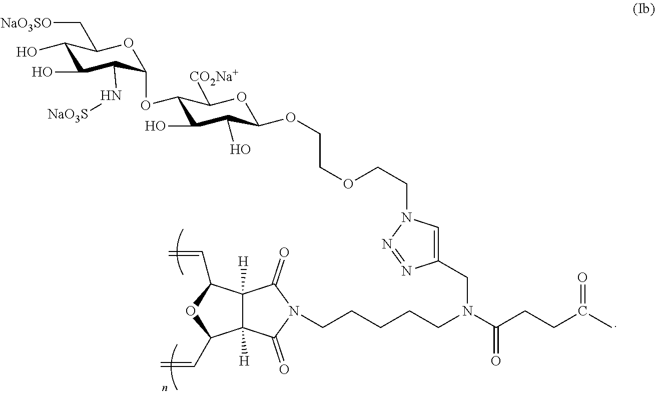

In particular embodiments, the compound of formula II is a compound of formula (Ic):

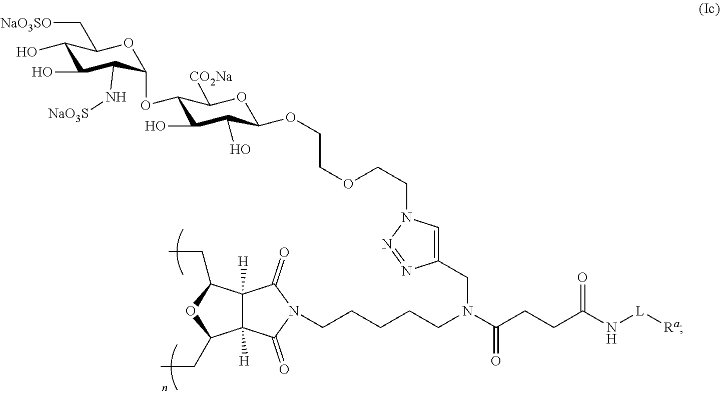

In particular embodiments, the compound of formula II is a compound of formula (Id):

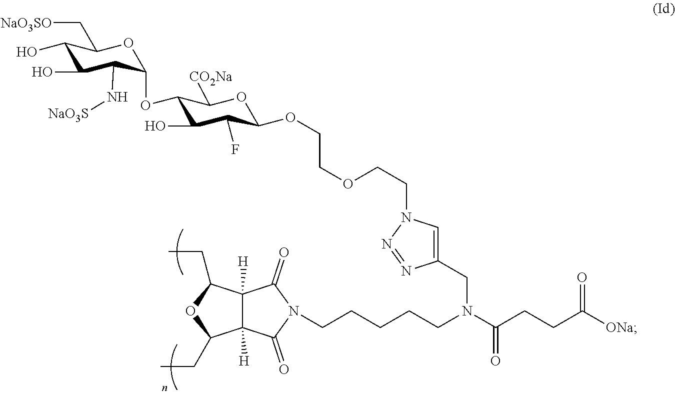

wherein: n is an integer from 2-100 inclusive; the saccharide or disaccharide includes one or more —SO₃H groups, one or more F⁻ groups.

In particular embodiments, the compound of formula II is a compound of formula (Ie):

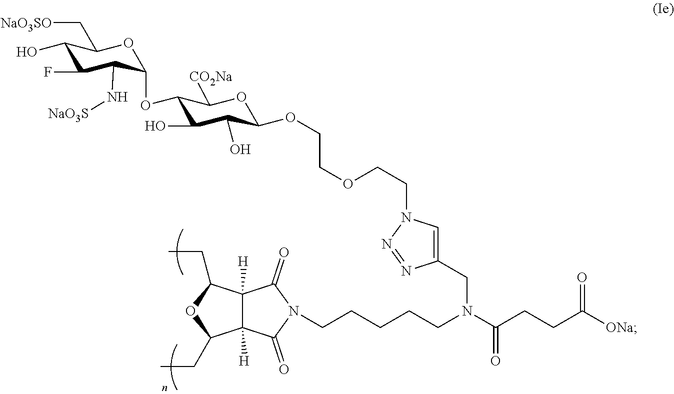

In particular embodiments, the compound of formula II is a compound of formula (If):

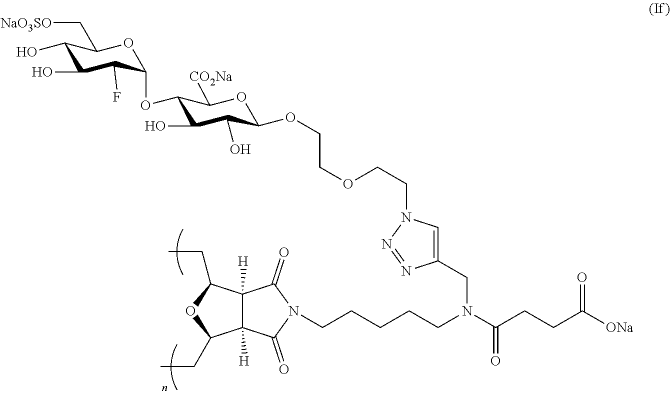

In particular embodiments, the compound of formula II is a compound of formula (Ig):

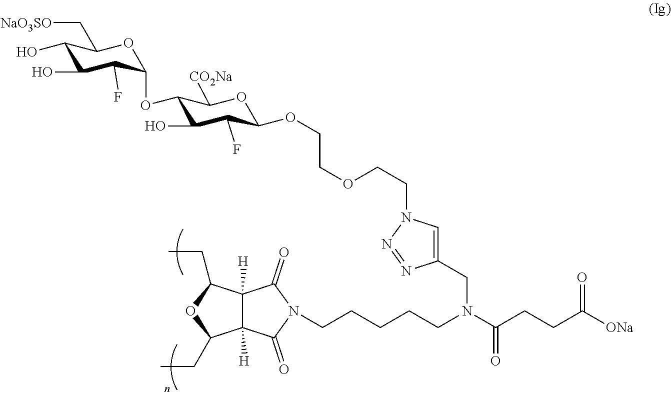

In particular embodiments, the compound of formula II is a compound of formula (IIa):

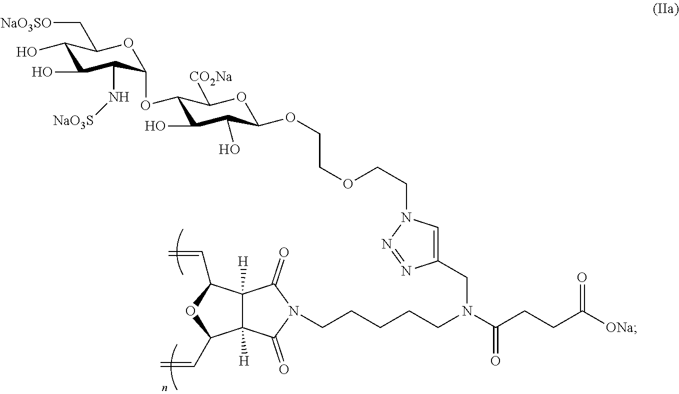

In particular embodiments, the compound of formula II is a compound of formula (IIb):

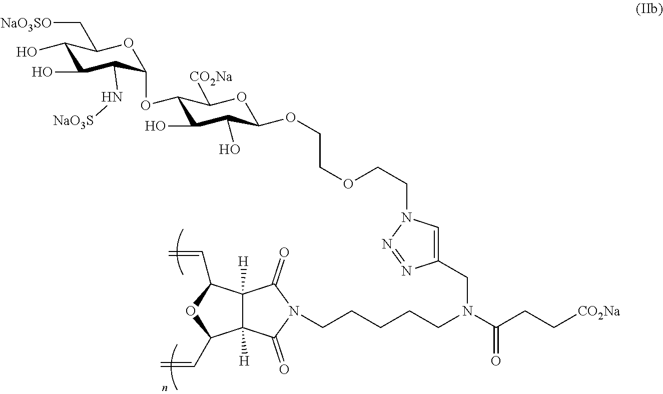

In particular embodiments, the compound of formula II is a compound of formula (IIc):

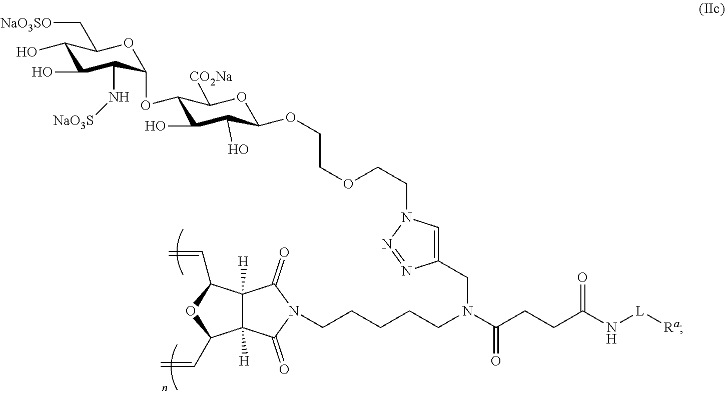

In particular embodiments, the compound of formula II is a compound of formula (IId):

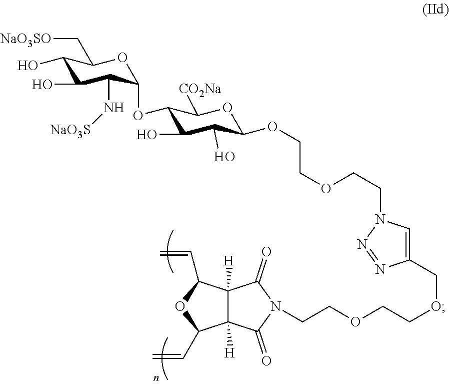

When n is an integer from 2-100 inclusive, this means n can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100. In particular embodiments, n can be more than 100. “When n is an integer from 2-100 inclusive” has the same meaning as “wherein n=2-100 repeating units”.

In particular embodiments, L is between 5 and 75 Angstroms inclusive in length. In particular embodiments, L is between 5 and 50 Angstroms inclusive in length. In particular embodiments, L is between 10 and 30 Angstroms inclusive in length. In particular embodiments, L includes an ether containing chain. In particular embodiments, L is a branched or unbranched, saturated or unsaturated, hydrocarbon chain, having from 1 to 20 carbon atoms, wherein one or more of the carbon atoms is optionally replaced independently by —O—, —S, —N(R^{x})—, wherein each Rx is independently H or (C₁-C₆)alkyl, wherein the hydrocarbon chain is optionally substituted with one or more groups selected from -oxo-, halo and hydroxy. In particular embodiments, L is —CH₂CH₂OCH₂CH₂— or —NHCH₂CH₂OCH₂CH₂—. In particular embodiments, L is —CH₂CH₂OCH₂CH₂—. In particular embodiments, L is —NHCH₂CH₂OCH₂CH₂—. In particular embodiments, R^{a} is a saccharide. In particular embodiments, R^{a} is a disaccharide.

In particular embodiments, R^{a} is selected from:

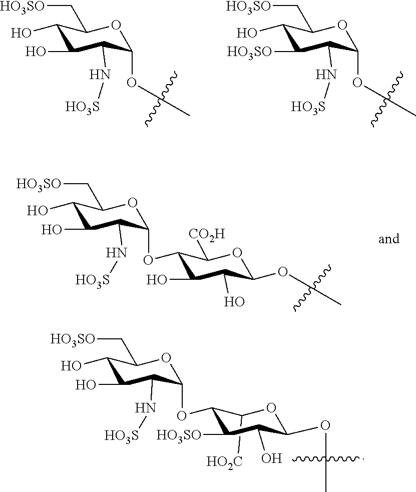

In particular embodiments, R^{a} is selected from:

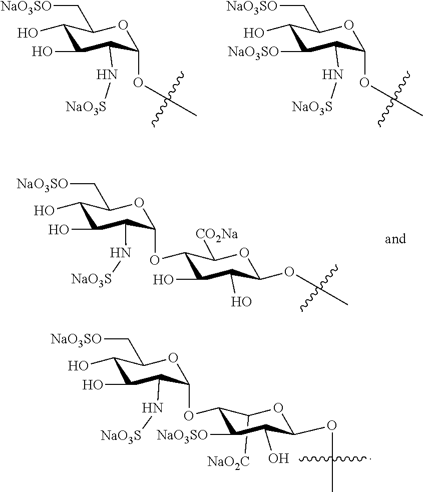

In particular embodiments, R^{a} is:

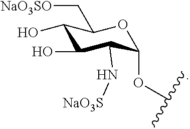

In particular embodiments, the compound of formula II is:

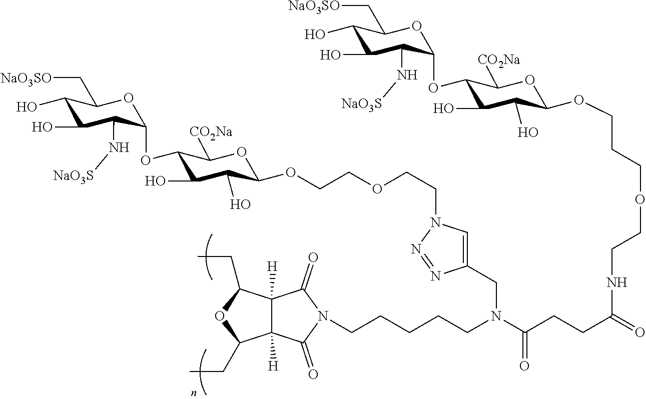

In particular embodiments, the compound of formula II is:

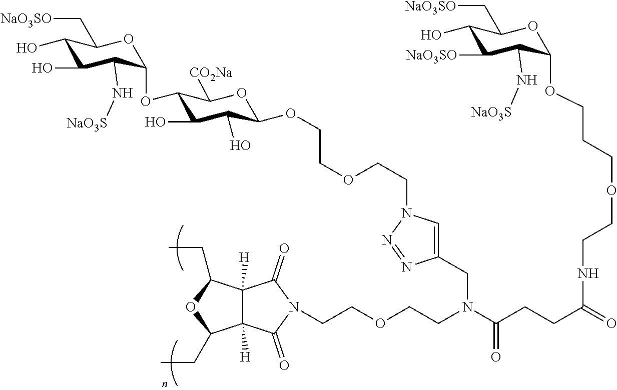

In particular embodiments, the compound of formula II is:

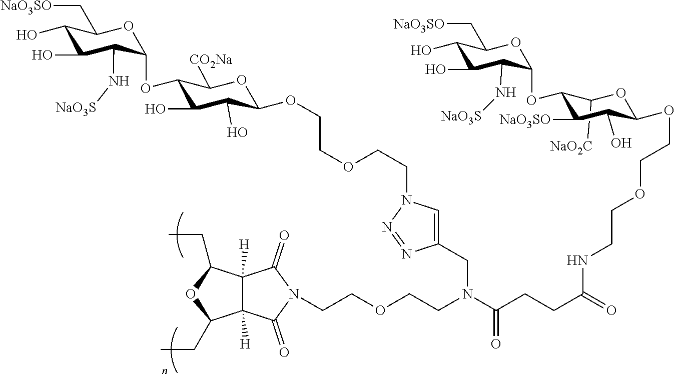

In particular embodiments, n is an integer from 5-100 inclusive. In particular embodiments, n is an integer from 2-75 inclusive. In particular embodiments, n is an integer from 5-75 inclusive. In particular embodiments, n is an integer from 5-15 inclusive. In particular embodiments, n is an integer from 10-100 inclusive. In particular embodiments, n is an integer from 10-75 inclusive. In particular embodiments, n is an integer from 10-55 inclusive. In particular embodiments, n is 12, 27, or 51. In particular embodiments, n is 5, 8, 9, or 12.

In particular embodiments, the compound of formula II is:

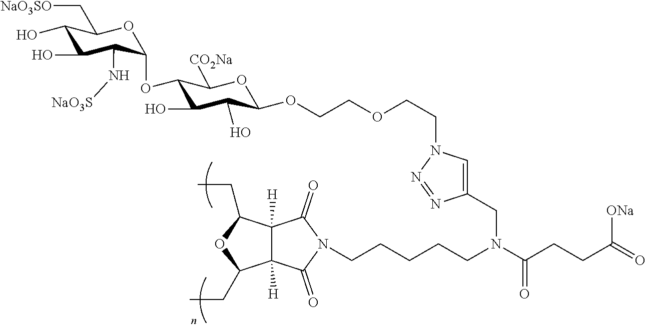

wherein n is 5, 9, or 12.

In particular embodiments, the compound of formula II is:

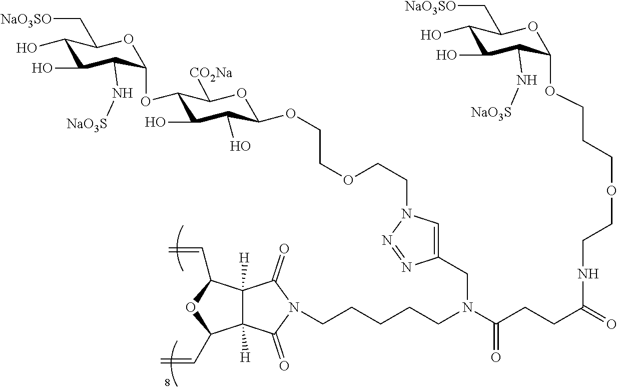

In particular embodiments, the compound of formula II is:

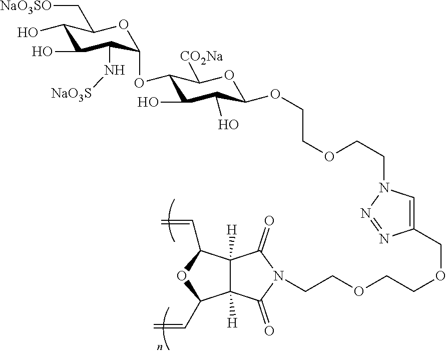

wherein n is 5 or 9.

In another aspect, the disclosure provides use of a polymer including one or more units of the following formula (III):

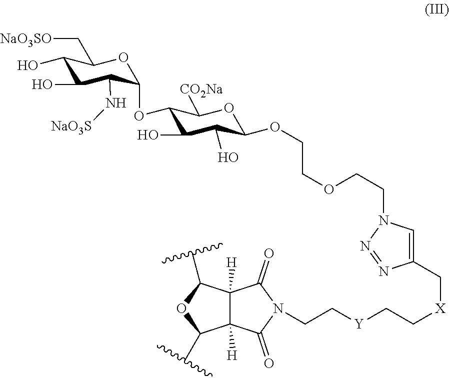

wherein:

### X is —O— or

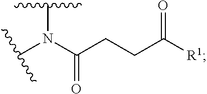

Y is —O— or —CH₂—; R¹ is OH or —N(H)-L-R^{a}; L is a linking group; and R^{a} is a saccharide or disaccharide, which saccharide or disaccharide includes one or more —SO₃H groups.

In another aspect, the disclosure provides use of a polymer including one or more units of the following formula (IIIa):

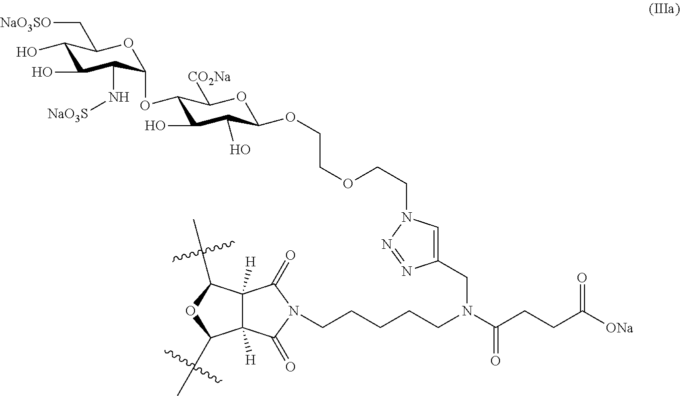

In another aspect, the disclosure provides use of a polymer including one or more units of the following formula (IIIb):

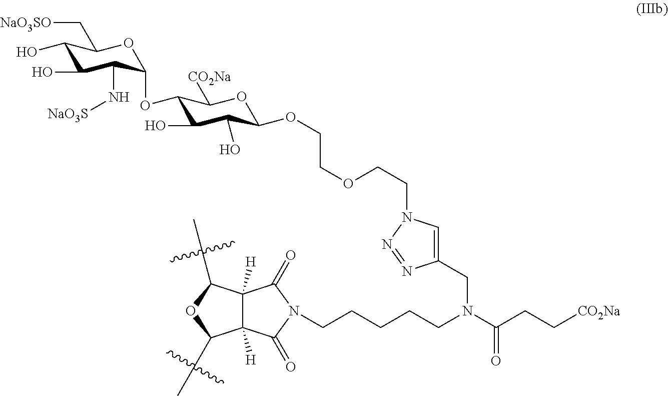

In another aspect, the disclosure provides use of a polymer including one or more units of the following formula (IIIc):

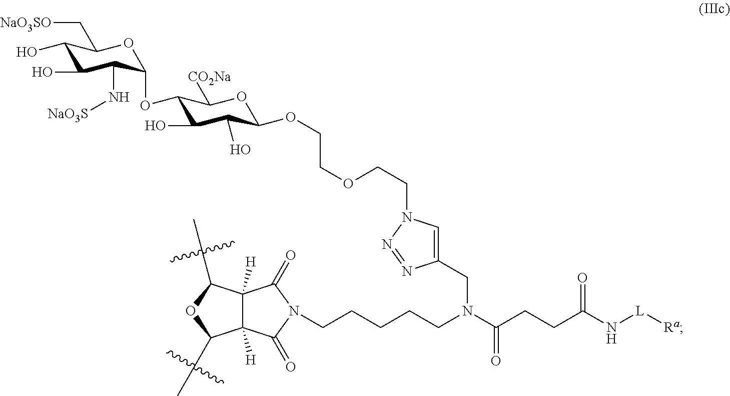

wherein L is a linking group; and R^{a} is a saccharide or disaccharide, which saccharide or disaccharide includes one or more —SO₃H groups.

In another aspect, the disclosure provides use of a polymer including one or more units of the following formula (IIId):

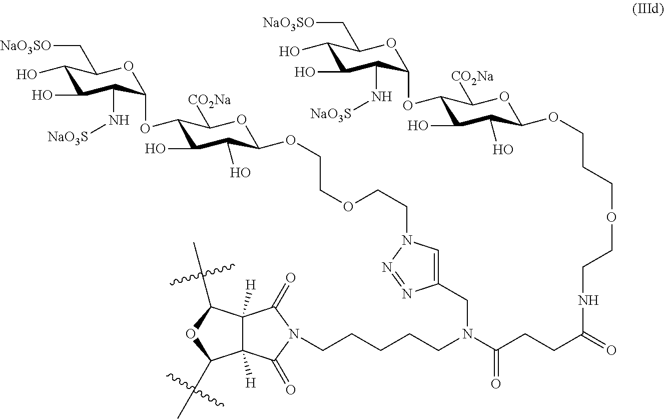

In another aspect, the disclosure provides use of a polymer including one or more units of the following formula (IIIe):

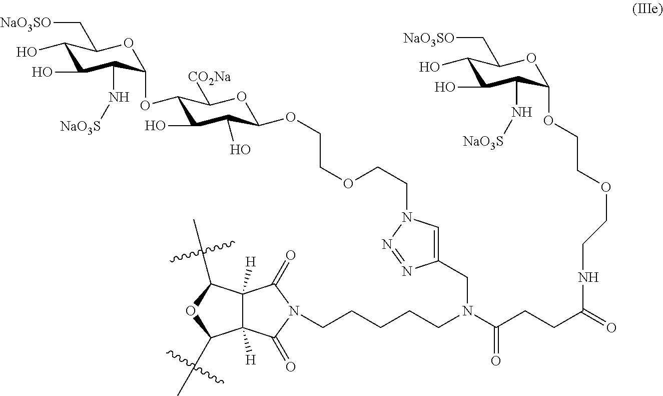

In another aspect, the disclosure provides use of a A polymer including one or more units of the following formula (IIIf):

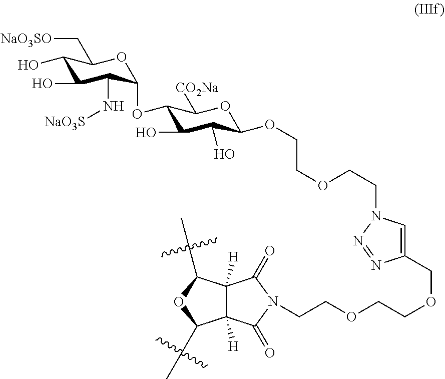

In particular embodiments, the disclosure provides use of a salt of formula II which is a sodium salt.

In particular embodiments, the disclosure provides use of a salt of formula II which is which is a lithium salt.

In another aspect, the disclosure provides a method to inhibit the activity of a heperanase including contacting the heperanase with a compound of formula II, or a salt thereof for the purpose of treating cancer.

Processes for preparing compounds of formula I are provided as further embodiments of the disclosure and are illustrated by the procedures described herein in which the meanings of the generic radicals are as given above unless otherwise qualified. An intermediate useful for preparing a compound of formula I is a compound selected from:

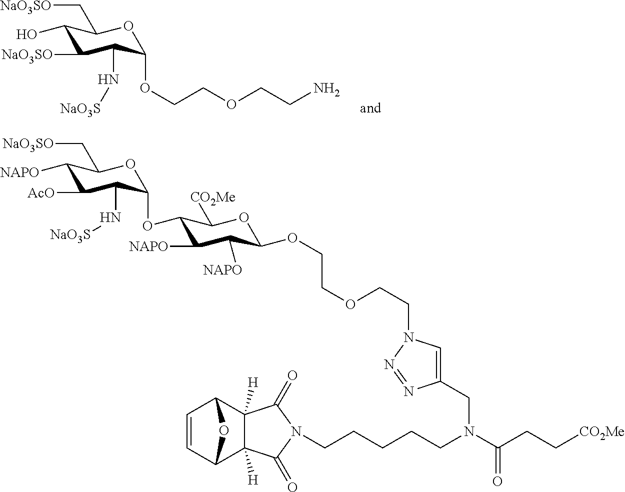

Compound (Ia) can be prepared using the method described in Loka, et al. ACS Appl Mater Interfaces (2019; 11(1):244-254. doi:10.1021/acsami.8b17625). Compounds (1f) and (1g) are described in further detail in the section “Experimental Example 2” listed below. Additional methods that can be considered in synthesizing the described compounds are found in, for example, Loka et al., Chem Commun (Camb). 2017 Aug. 10; 53(65): 9163-9166; Sletten et al., Biomacromolecules 2017, 18, 3387-3399; Ittah, C. P. J. Glaudemans, Carbohydr. Res. 1981, 95, 189-194; Shelling, D. Dolphin, P. Wirz, R. E. Cobbledick, F. W. B. Einstein, Carbohydr. Res. 1984, 132, 241-259; McCarter, et al., Carbohydr. Res. 1993, 249, 77-90; McCarter, et al., J. Am. Chem. Soc. 1997, 119, 5792-5797; Burton, et al., J. Chem. Soc. Perkin Trans. 1 1997, 2375-2382; Burkart, et al., J. Am. Chem. Soc. 1997, 119, 11743-11746; Hayashi, et al., Bioorg. Med. Chem. 1997, 5, 497-500; U.S. Pat. No. 5,770,407; Albert, et al., Tetrahedron 1998, 54, 4839-4848; Albert, et al., Synlett 1999, 1483-1485; Vincent, et al., J. Org. Chem. 1999, 64, 5264-5279; Barlow, et al., Carbohydr. Res. 2000, 328, 473-480; Burkart, et al., Bioorg. Med. Chem. 2000, 8, 1937-1946; Zhang & Liu, J. Am. Chem. Soc. 2001, 123, 6756-6766; Blanchard, et al., Carbohydr. Res. 2001, 333, 7-17; Ly, et al., Biochemistry 2002, 41, 5075-5085; Gonzalez, et al., Eur. J. Org. Chem. 2005, 3279-3285; Kasuya, et al., J. Fluorine Chem. 2007, 128, 562-565; Allman, et al., ChemBioChem 2009, 10, 2522-2529; Errey, et al., Org. Biomol. Chem. 2009, 7, 1009-1016; Mersch, et al., Synlett 2009, 13, 2167-2171; Boutureira, et al., Chem. Commun. 2010, 46, 8142-8144; Wagner, et al., Chem. Eur. J. 2010, 16, 7319-7330; Johannes, et al., Org. Biomol. Chem. 2011, 9, 5541-5546; Ioannou, et al., Chem. Eur. J. 2018, 24, 2832-2836; and Kieser, et al., Chem. Neurosci 2018, 9, 1159-1165.

While sodium salt forms of the compounds are depicted, the disclosure encompasses other salt forms which includes salt forming cations (e.g., potassium salt forms, ammonium salt forms, calcium salt forms, lithium salt forms, iron salt forms, magnesium salt forms, sodium salt forms, copper salt forms, pyridinium salt forms, or quaternary ammonium salt forms) as well as protonated forms of the depicted compounds.

In cases where compounds are sufficiently basic or acidic, a salt of a compound of formula I can be useful as an intermediate for isolating or purifying a compound of formula I. Additionally, administration of a compound of formula I as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

(ii) Compositions for Administration. Compounds described herein can be formulated for administration to subjects in one or more pharmaceutically acceptable carriers. Exemplary carriers include saline, buffered saline, physiological saline, water, Hanks' solution, Ringer's solution, Nonnosol-R (Abbott Labs), glycerol, ethanol, and combinations thereof.

In particular embodiments, a carrier for infusion includes buffered saline with 5% HSA or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent compound adherence to container walls. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as HSA, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Exemplary oral formulations include capsules, coated tablets, edibles, elixirs, emulsions, gels, gelcaps, granules, gums, juices, liquids, oils, pastes, pellets, pills, powders, rapidly-dissolving tablets, sachets, semi-solids, sprays, solutions, suspensions, syrups, tablets, etc.

Particular embodiments include swallowable compositions. Swallowable compositions are those that do not readily dissolve when placed in the mouth and may be swallowed whole without chewing or discomfort. U.S. Pat. Nos. 5,215,754 and 4,374,082 describe methods for preparing swallowable compositions. In particular embodiments, swallowable compositions may have a shape containing no sharp edges and a smooth, uniform and substantially bubble free outer coating.

Therapeutically effective amounts of compounds within a composition can include at least 0.1% w/v or w/w compound; at least 1% w/v or w/w compound; at least 10% w/v or w/w compound; at least 20% w/v or w/w compound; at least 30% w/v or w/w compound; at least 40% w/v or w/w compound; at least 50% w/v or w/w compound; at least 60% w/v or w/w compound; at least 70% w/v or w/w compound; at least 80% w/v or w/w compound; at least 90% w/v or w/w compound; at least 95% w/v or w/w compound; or at least 99% w/v or w/w compound.

(iii) Methods of Use. Methods disclosed herein include treating subjects (humans, veterinary animals (dogs, cats, reptiles, birds, etc.) livestock (horses, cattle, goats, pigs, chickens, etc.) and research animals (monkeys, rats, mice, fish, etc.) with compositions disclosed herein. Treating subjects includes delivering therapeutically effective amounts. Therapeutically effective amounts include those that provide effective amounts, prophylactic treatments and/or therapeutic treatments.

An “effective amount” is the amount of a composition necessary to result in a desired physiological change in the subject. For example, an effective amount can provide an anti-cancer effect. Effective amounts are often administered for research purposes. Effective amounts disclosed herein can cause a statistically-significant effect in an animal model or in vitro assay relevant to the assessment of a cancer's development or progression.

A “prophylactic treatment” includes a treatment administered to a subject who does not display signs or symptoms of a cancer or displays only early signs or symptoms of a cancer such that treatment is administered for the purpose of diminishing or decreasing the risk of developing the cancer further. Thus, a prophylactic treatment functions as a preventative treatment against a cancer. In particular embodiments, prophylactic treatments reduce, delay, or prevent metastasis from a primary a cancer tumor site from occurring.

A “therapeutic treatment” includes a treatment administered to a subject who displays symptoms or signs of a cancer and is administered to the subject for the purpose of diminishing or eliminating those signs or symptoms of the cancer. The therapeutic treatment can reduce, control, or eliminate the presence or activity of the cancer and/or reduce control or eliminate side effects of the cancer.

Function as an effective amount, prophylactic treatment or therapeutic treatment are not mutually exclusive, and in particular embodiments, administered dosages may accomplish more than one treatment type.

In particular embodiments, therapeutically effective amounts provide anti-cancer effects. Anti-cancer effects include a decrease in the number of cancer cells, decrease in the number of metastases, a decrease in tumor volume, an increase in life expectancy, induced chemo- or radiosensitivity in cancer cells, inhibited angiogenesis near cancer cells, inhibited cancer cell proliferation, inhibited tumor growth, prevented or reduced metastases, prolonged subject life, reduced cancer-associated pain, and/or reduced relapse or re-occurrence of cancer following treatment.

A “tumor” is a swelling or lesion formed by an abnormal growth of cells (called neoplastic cells or tumor cells). A “tumor cell” is an abnormal cell that grows by a rapid, uncontrolled cellular proliferation and continues to grow after the stimuli that initiated the new growth cease. Tumors show partial or complete lack of structural organization and functional coordination with the normal tissue, and usually form a distinct mass of tissue, which may be benign, pre-malignant or malignant.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of condition, type of cancer, stage of cancer, previous or concurrent therapeutic interventions, idiopathy of the subject and route of administration.

Useful doses can range from 0.1 to 5 μg/kg or from 0.5 to 1 μg/kg. In other examples, a dose can include 1 μg/kg, 15 μg/kg, 30 μg/kg, 50 μg/kg, 55 μg/kg, 70 μg/kg, 90 μg/kg, 150 μg/kg, 350 μg/kg, 500 μg/kg, 750 μg/kg, 1000 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly).

As indicated, the compositions and formulations disclosed herein can be administered by, e.g., injection or oral administration.

In certain embodiments, compositions are administered to a patient in conjunction with (e.g., before, simultaneously or following) any number of relevant treatment modalities. In particular embodiments, compositions may be used in combination with chemotherapy, radiation, immunosuppressive, agents, such as cyclosporin, azathioprine, methotrexate, mycophenolate, and FK506, antibodies, or other immunoablative agents.

(iv) Experimental Examples. Experimental Example 1. Abstract: Heparanase, the sole heparan sulfate polysaccharide degrading endoglycosidase enzyme has been correlated to tumor angiogenesis and metastasis and therefore has become a potential target for anticancer drug development. In this systematic study, the sulfation pattern of pendant disaccharide moiety on synthetic glycopolymers was synthetically manipulated to achieve optimal heparanase inhibition. Further, the most potent glycopolymer inhibitor of heparanase (IC50=0.10±0.36 nM) was examined for cross-bioactivity, using a solution based competitive BLI assay, with other HS-binding proteins (growth factors, platelet factor 4, and P-selectin) which are responsible for mediating angiogenic activity, antibody-induced thrombocytopenia, and cell metastasis. The synthetic glycopolymer has low affinity for these HS-binding proteins in comparison to natural heparin. In addition, the glycopolymer possessed no proliferative properties towards human umbilical endothelial cells (HUVEC) and a potent antimetastatic effect against 4T1 mammary carcinoma cells. Thus, the present disclosure not only establishes a specific inhibitor of heparanase with high affinity, but also demonstrates the high effectiveness of this multivalent heparanase inhibitor in inhibiting experimental metastasis in vivo.

Introduction. Glycosidases, a class of enzymes which catalyze the hydrolysis of glycosidic bonds in complex sugars play a vital role in cellular function (Vocadlo, et al., Curr. Opin. Chem. Biol. 2008, 12 (5), 539-555). As a result, modulation of the biological activity of glycosidases is a major target for drug discovery (Compain, et al., ChemBioChem 2014, 15 (9), 1239-1251). Heparanase is an endolytic enzyme that cleaves the internal β-(1,4)-glycosidic bond between glucuronic acid (GlcA) and N-sulfated glucosamine (GlcNS) along heparan sulfate (HS) saccharide chains which constitute the extracellular matrix (ECM) and basement membranes (Rivara, et al., Future Med. Chem. 2016, 8 (6), 647-680; Vlodaysky, et al., Drug Resist. Updates 2016, 29, 54-75; Pisano, et al., Biochem. Pharmacol. 2014, 89 (1), 12-19; Vlodaysky, et al., Nat. Med. 1999, 5, 793). Clinical studies have demonstrated that high levels of heparanase expression correlate with increased tumor growth and angiogenesis, enhanced metastasis, and poor patient prognosis for both hematological and solid malignancies, and thus it has become a target for cancer therapeutics (Ilan, et al., Int. J. Biochem. Cell Biol. 2006, 38 (12), 2018-2039; Barash, et al., FEBS J. 2010, 277 (19), 3890-3903; Arvatz, et al., Cancer Metastasis Rev. 2011, 30 (2), 253-268; Vlodaysky, et al., Rambam Maimonides Med. J. 2011, 2 (1), e0019; Vlodaysky, et al., Cancer Microenviron. 2012, 5 (2), 115-132; Knelson, et al., Trends Biochem. Sci. 2014, 39 (6), 277-288; Sanderson, et al., Semin. Cell Dev. Biol. 2001, 12 (2), 89-98). These studies emphasize the need for heparanase inhibitors of high specificity.

Several molecules have been developed to target heparanase activity, but only carbohydrate molecules have advanced to clinical trials for cancer patients (Rivara, et al., Future Med. Chem. 2016, 8 (6), 647-680; Kudchadkar, et al., Expert Opin. Invest. Drugs 2008, 17 (11), 1769-1776; Cassinelli, et al., Oncotarget 2016, 7 (30), 47848-47863; Cassinelli, et al., Biochem. Pharmacol. 2013, 85 (10), 1424-1432; Naggi, et. al., J. Biol. Chem. 2005, 280 (13), 12103-12113; Vlodaysky, et al., Curr. Pharm. Des. 2007, 13 (20), 2057-2073; Bar-Ner, et al., Blood 1987, 70 (2), 551-557; Jia, et al., Eur. J. Med. Chem. 2016, 121, 209-220; Lanzi, et al., Curr. Med. Chem. 2017, 24 (26), 2860-2886; Weissmann, et al., Proc. Natl. Acad. Sci. U.S.A 2016, 113 (3), 704-709; Mitsiades, et al., Clin. Cancer. Res. 2009, 15 (4), 1210-1221). Except for compound PG545 (pixatimod, a highly sulfated tetrasaccharide bound to a lipophilic cholestanol aglycone), the carbohydrate-based heparanase inhibitors are heterogeneous in size and sulfation pattern leading to nonspecific binding and unforeseen adverse effects, thus halting their translation into clinical use (Kudchadkar, et al., Expert Opin. Invest. Drugs 2008, 17 (11), 1769-1776; Cassinelli, et al., Oncotarget 2016, 7 (30), 47848-47863; Cassinelli, et al., Biochem. Pharmacol. 2013, 85 (10), 1424-1432; Naggi, et. al., J. Biol. Chem. 2005, 280 (13), 12103-12113; Vlodaysky, et al., Curr. Pharm. Des. 2007, 13 (20), 2057-2073; Bar-Ner, et al., Blood 1987, 70 (2), 551-557; Dredge, et al., Br. J. Cancer 2011, 104 (4), 635-642; O'Reilly et al., Oncologist. 2017 December; 22(12):1429-e139. doi: 10.1634/theoncologist.2017-0472. Epub 2017 Nov. 20; National Institute of Health. US National Library of Medicine. [Accessed 24 Oct. 2019]). Alternatively, saccharide-functionalized glycopolymers (Lundquist, et al., Chem. Rev. 2002, 102 (2), 555-578.), which have been shown to retain the key biological properties of the natural HS polysaccharides, could be an approach for the development of heparanase inhibitors with high specificity and affinity (Spaltenstein, et al., J. Am. Chem. Soc. 1991, 113 (2), 686-687; Mortell, et al., J. Am. Chem. Soc. 1994, 116 (26), 12053-12054; Oh, et al., Angew. Chem. Int. Ed. 2013, 52 (45), 11796-11799; Sheng, et al., J. Am. Chem. Soc. 2013, 135 (30), 10898-10901; Mammen, et al., Angew. Chem. Int. Ed. 1998, 37 (20), 2754-2794; Gestwicki, et al., J. Am. Chem. Soc. 2002, 124 (50), 14922-14933; Kiessling, et al., Curr. Opin. Chem. Biol. 2000, 4 (6), 696-703). As well, macromolecules including polysaccharides have been utilized in targeted cancer therapies (Hosseinkhani, et al., J. Nanopart. Res. 2013, 15 (1), 1345-1355; Abedini, et al., Polym. Adv. Technol. 2018, 29 (10), 2564-2573; Ghadiri, et al., J. Biomed. Mater. Res., Part A 2017, 105 (10), 2851-2864; Khan, et al., Acta Biomater. 2012, 8 (12), 4224-4232; Hosseinkhani, et al., Gene Ther. 2004, 11 (2), 194-203). This approach, however, is still met with the challenge of developing an inhibiting epitope (inhitope) that can gain access to the active site of heparanase (Compain, et al., ChemBioChem 2014, 15 (9), 1239-1251). In comparison to lectins, like many glycosidase enzymes, heparanase is monomeric and possesses a single deep binding groove (Rivara, et al., Future Med. Chem. 2016, 8 (6), 647-680; Wu, et al., Nat. Struct. Mol. Biol. 2015, 22, 1016-1022). Until 2009, these features deterred the use of multivalent scaffolds as glycosidase inhibiting motifs (Diot, et al., Org. Biomol. Chem. 2009, 7 (2), 357-363). Soon thereafter, several more examples were developed for the inhibition of other glycosidases (Compain, et al., ChemBioChem 2014, 15 (9), 1239-1251; Lepage, et al., Chem. Eur. J. 2016, 22 (15), 5151-5155; Decroocq, et al., Chem. Eur. J. 2011, 17 (49), 13825-13831; Gouin, et al., Chem. Eur. J. 2014, 20 (37), 11616-11628; Ortiz Mellet, et al., J. Mater. Chem. B 2017, 5 (32), 6428-6436; Nierengarten, et al., Chem. Eur. J. 2017, 24 (10), 2483-2492; Brissonnet, et al., Bioconjugate Chem. 2015, 26 (4), 766-772; Compain, et al., Angew. Chem. Int. Ed. 2010, 49 (33), 5753-5756; Abelian Flos, et al., Chem. Eur. J. 2016, 22 (32), 11450-11460; Bonduelle, et al., Chem. Commun. 2014, 50 (25), 3350-3352; Alvarez-Dorta, et al., Chem. Eur. J. 2017, 23 (38), 9022-9025; Siriwardena, et al., RSC Advances 2015, 5 (122), 100568-100578). These studies propose that the valency and relative arrangement of the carbohydrate moieties are critical parameters for governing the multivalent effect toward a given glycosidase, thus allowing for extension to other glycosidases, including heparanase if the right inhitope was selected (Sletten, et al., Biomacromolecules 2017, 18 (10), 3387-3399; Loka, et al., Chem. Commun. 2017, 53 (65), 9163-9166).

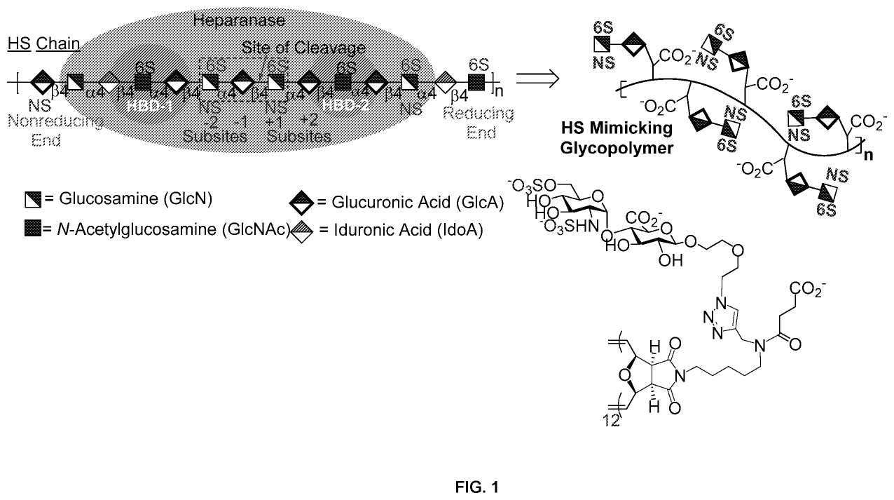

Recently, research reported the use of computational studies and the crystal structure of human heparanase to extract the natural HS-heparanase interactions as a template to design HS mimicking glycopolymers containing the disulfated disaccharide component for maximal inhibition and minimal anticoagulant activity (FIG. 1) (Wu, et al., Nat. Struct. Mol. Biol. 2015, 22, 1016-1022; Sletten, et al., Biomacromolecules 2017, 18 (10), 3387-3399; Loka, et al., Chem. Commun. 2017, 53 (65), 9163-9166). Upon evaluation, glycopolymer 1 with 12 repeating units was determined to be the most potent heparanase inhibitor with a picomolar inhibitory concentration and tight-binding characteristics. Further, removal of the scissile GlcAβ(1,4)GlcN glycosidic bond prevented degradation by heparanase (Sletten, et al., Biomacromolecules 2017, 18 (10), 3387-3399; Loka, et al., Chem. Commun. 2017, 53 (65), 9163-9166; Johnson, et al., J. Am. Chem. Soc. 2011, 133 (3), 559-566; Johnson, et al., Macromolecules 2010, 43 (24), 10326-10335; Loka, et al., Biomacromolecules 2015, 16 (12), 4013-4021). Yet, questions still remained on how inhibition of a glycosidase, specifically heparanase, will be affected by changes in the inhibiting epitope (inhitope) on a multivalent scaffold and how glycopolymer inhibition will translate in vivo.

Herein, the disclosure reports a systematic study on the modulation of multivalent inhibition of heparanase by varying the sulfation pattern of the pendant disaccharide moiety on synthetic glycopolymers. The homogeneity of the research approach allows the research to dissect the contribution of an individual sulfation to the inhibition of heparanase. The disclosure results indicate that heparanase is capable of recognizing subtle changes on differently sulfated glycopolymers. To ensure heparanase specificity, the most potent glycopolymer inhibitor of heparanase was examined with a solution based competitive BLI assay for cross-bioactivity to other HS-binding proteins (growth factors, platelet factor 4, P-selectin) which are responsible for mediating angiogenic activity, antibody-induced thrombocytopenia, and tumor cell metastasis (Pellegrini, et al., Nature 2000, 407, 1029-1034; Arepally, et al., New Engl. J. Med. 2006, 355 (8), 809-817; Läubli, et al., Semin. Cancer Biol. 2010, 20 (3), 169-177). Compared to heparin, the research designed synthetic glycopolymer has a much lower affinity for these proteins. Additionally, the synthetic glycopolymer was shown to have antiproliferative properties when analyzed using a HUVEC cell assay and an anti-metastatic effect in a 4T1 mammary carcinoma model (Cassinelli, et al., Oncotarget 2016, 7 (30), 47848-47863; Cassinelli, et al., Biochem. Pharmacol. 2013, 85 (10), 1424-1432; Naggi, et. al., J. Biol. Chem. 2005, 280 (13), 12103-12113).

Experimental Section. Materials. All commercial chemical reagents used for synthesis were used as received from Sigma Aldrich, Alfa Aesar, TCI, and Combi-Blocks, unless otherwise mentioned. Other reagents and materials were purchased from the following: heparanase, FGF-1, FGF-2, P-selectin, and ATIII were all carrier-free (R&D Systems), HUVECs and reagents (Lonza), Heparin-biotin (Creative PEGworks), Streptavidin BLI biosensors (fortéBIO), CellTiter 96 Aqueous One Solution Cell Proliferation Assay (Fisher Scientific), TR-FRET heparanase inhibition kit (Cis-bio).

Instrumentation. All new compounds were analyzed by NMR spectroscopy and High-Resolution Mass spectrometry. All ¹H NMR spectra were recorded on either a Bruker 400 or 500 MHz spectrometer. All ¹³C NMR spectra were recorded on either a Bruker 100 or 126 MHz NMR spectrometer. All ¹⁹F NMR spectra were recorded on a Bruker 471 MHz NMR spectrometer. High resolution (ESI-TOF) mass spectrometry were acquired at Wayne State University. CMC fluorescence measurements were performed on an Aligent Technologies Cary Eclipse Fluorescence Spectrophotometer. Homogeneous time-resolved fluorescence (HTRF) emissions were measured using a SpectraMax i3x Microplate Reader (Molecular Devices). Number of cells were determined using a Beckman coulter counter. BLI assays were performed on an Octet Red Instrument (fortéBIO).

Glycopolymer Formation. Glycomonomer was placed into 10 mL Shlenk flask under inert atmosphere and dissolved in degassed 2,2,2-trifluoroethanol:1,2-dichloroethane solution. A solution of Grubbs 3rd generation catalyst was added and the reaction heated to 55° C. After 1 h the reaction was monitored for completion by NMR and then triturated from methanol by diethyl ether. Glycopolymer was then deprotected by LiOH in a water:THF mixture. After 24 h the glycopolymer was dialyzed (3.5K MWCO) against 0.9% NaCl solution (3 buffer changes) and DI water (3 buffer changes).

Computational Docking Study. For the docking studies the apo heparanase structure (PDB code: 5E8M) was utilized (Wu, et al., Nat. Struct. Mol. Biol. 2015, 22, 1016-1022). Global docking with each ligand was performed separately on the heparanase structure using Autodock VINA in the YASARA molecular modelling program.

Biolayer Interferometry Cross-Bioactivity Assay. BLI assays were performed on an Octet Red Instrument (fortéBIO) at 25° C. Immobilization and binding analysis were carried out at 1000 rpm using HBS-EP buffer.

HUVEC Culturing. HUVECs were cultured at 37° C. in a humidified atmosphere of 5% CO2 using protocols and reagents supplied by Lonza. At 70-80% confluence cells were harvested with 0.025% trypsin in phosphate buffered saline (PBS) and reseeded into new vessel with fresh growth medium at seeding densities of 2500-5000 cells/cm2 of vessel surface area.

HUVEC Proliferation Assay. Endothelial basal medium (EBM-2) containing only 2% FBS and gentamicin was used for cell proliferation. Cells were resuspended in proliferation medium and 100 μL was seeded on to 96-well microplate at 3000 cells/well. After incubating for one day, FGF-2 and C(6)-SO₃ N—SO₃ polymer 5A in proliferation medium were added to each well maintaining final volume of 200 μL. Each concentration was done in triplicate. After incubating for 70 h, 20 μl of the CellTiter 96 Aqueous One Solution Cell Proliferation Assay was added to each well and absorbance at 490 nm was measured 2 h later. The entire assay was repeated three times.

Critical Micelle Concentration (CMC) Protocol: A stock solution of C(6)-SO₃ N—SO₃ polymer 5A was serially diluted in 1.5 mL Eppendorf tubes at 16 different concentrations with deionized water from 0 to 1 mg/mL. To each tube pyrene stock solution was added and tubes were then covered in aluminum foil and mechanically agitated by an orbital shaker for 2 h and then allowed to equilibrate for 18 hours (h). Fluorescence emission spectra of the polymer solutions containing pyrene were recorded in a 400 μL microcuvette using an excitation wavelength of 335 nm, and the intensities I1 and I3 were measured at the wavelengths corresponding to the first and third vibronic bands located near 373 (I1) and 384 (I3) nm.

TR-FRET Heparanase Inhibition Assay. The inhibitor in Milli-Q water and heparanase (R&D Systems) solution in pH 7.5 triz buffer were added into microtubes and pre-incubated at 37° C. for 10 min. Next, biotin-heparan sulfate-Eu cryptate in pH 5.5 0.2 M NaCH₃CO₂ buffer was added to the microtubes, and the resulting mixture was incubated for 60 min at 37° C. The reaction mixture was stopped by adding Streptavidin-XLent! solution in pH 7.5 dilution buffer made of 0.1 M NaPO₄, 0.8 M KF, 0.1% BSA. After the mixture had been stirring at room temperature (RT) for 15 min, 100 μL (per well) of the reaction mixture was transferred to a 96 well microplate in triplicate and HTRF emissions at 616 nm and 665 nm were measured by exciting at 340 nm using a SpectraMax i3x Microplate Reader (Molecular Devices).

4T1 Metastasis Assay. Luciferase-labeled 4T1 mammary carcinoma cells (1×10⁵/mouse) were injected i.v. (n=6 mice/group) with vehicle alone (control, PBS), with positive control (heparin), or with GlcNS(6S)α((1,4)GlcA glycopolymer (DP=12, 100 μg/mouse) into BALB/c mice (i.p) 20 min prior to cell inoculation and also together with the cells. IVIS bioluminescent imaging was performed on day 7 after cell inoculation. For IVIS imaging, mice were injected intraperitoneally with D-luciferin substrate at 150 mg/kg and anesthetized with continuous exposure to isoflurane (EZAnesthesia, Palmer, Pa.). The experiment was repeated 3 times with similar results.

Results and Discussion. Rational Design of Glycopolymers. In studies with HS oligosaccharides, heparanase has been shown to specifically cleave at an explicit sulfation pattern, GlcAβ(1,4)GlcNS(6S), along the HS polysaccharide chain (FIG. 1) (Peterson, et al., Matrix Biol. 2013, 32 (5), 223-227). During HS biosynthesis, there is no set blueprint, leaving the epimerization of the uronic acid, sulfation, and acetylation patterns to be randomly generated in domains of heavy sulfation and nonsulfated portions (Sarrazin, et al., Cold Spring Harb Perspect Biol 2011, 3 (7)). The heterogeneity of HS leads to enormous amount of information to be contained within the HS “glyco-code”, allowing HS to bind to a wide variety of proteins (Capila, et al., Angew. Chem. Int. Ed. 2002, 41 (3), 390-412). These proteins are involved in diverse physiological processes, including cell-cell communication, wound healing, immune response, and regulation of cell proliferation (Capila, et al., Angew. Chem. Int. Ed. 2002, 41 (3), 390-412). This promiscuity is what has led to the deleterious cross bioactivity of the previously reported heparanase inhibitors which are heparin/HS derivatives or mimetics (Rivara, et al., Future Med. Chem. 2016, 8 (6), 647-680).

The goal to achieve minimal cross-bioactivity while maintaining strong binding to heparanase is difficult because rational design and predictable efficiency of a neo-glycoconjugate toward a specific lectin and even more so glycosidase remains a challenge (Deniaud, et al., Org. Biomol. Chem. 2011, 9 (4), 966-979). Research has previously reported that multivalent glycosidase inhibitors can be rationally designed through computational modeling and by looking at previous oligosaccharide cleavage studies and ligand-protein co-crystal structures, to extract a high-affinity disaccharide motif (Sletten, et al., Biomacromolecules 2017, 18 (10), 3387-3399; Loka, et al., Chem. Commun. 2017, 53 (65), 9163-9166). Yet, some ambiguity remains from both the HS oligosaccharide and the crystal structure studies, with most of the uncertainty being with the glucosamine (GlcN) unit in the −2 binding subsite (Wu, et al., Nat. Struct. Mol. Biol. 2015, 22, 1016-1022; Peterson, et al., Matrix Biol. 2013, 32 (5), 223-227; Davies, et al., Biochem. J 1997, 321 (Pt 2), 557-559). Unfortunately, these questions remain unsolved because the GlcN unit at the +1/−2 subsites cannot be differentiated through enzymatic oligosaccharide synthesis or through the use of isolated heparin oligosaccharide mixtures (Peterson, et al., Matrix Biol. 2013, 32 (5), 223-227). With the ability to systematically synthesize different saccharide motifs from the same building blocks, research rationalized that use of the glycopolymer system was suited for answering these questions. Knowing that the disaccharide moiety had a strong preference for binding to the −2 and −1 subsites (Loka, et al., Chem. Commun. 2017, 53 (65), 9163-9166), a disaccharide having the −2 GlcN unit that could be orthogonally manipulated and then attached to the polymerizable scaffold to be polymerized subsequently was designed.

When designing which disaccharides to place onto the glycopolymers, previous studies and conclusions about the −2 GlcN unit were taken into consideration. The following trends were assessed: (1) Inspection of GlcNS6S at the −2 subsite crystal structure complexes revealed that the electron density for 6-O-sulfate is significantly weaker than that for N-sulfate, indicating that this subsite was occupied by a mixture of GlcNS and GlcNS6S (Wu, et al., Nat. Struct. Mol. Biol. 2015, 22, 1016-1022). As such, this data shows that heparanase can accommodate a variety of sulfated GlcNX sugars at the −2 position, but it is unknown which has a higher binding affinity; (2) For −2 GlcNS6S, the crystal structure of heparanase-HS trisaccharide ligand indicates that the C(6)-O-sulfate participates in electrostatic interactions with the side chain of Lys159. Therefore, preference at the −2 subsite is likely to be GlcNS6S>>GlcNS>GlcNAc because of the formation of additional electrostatic and hydrogen-bonding interactions (Wu, et al., Nat. Struct. Mol. Biol. 2015, 22, 1016-1022); (3) Structurally, the −2 N-sulfate appears to be one of the main determinants for recognition because it is directly in contact with the enzyme through hydrogen bonding networks (Wu, et al., Nat. Struct. Mol. Biol. 2015, 22, 1016-1022); (4) The −2 C(6)-O-sulfate and +1 N-sulfate may further stabilize the heparanase-bound trisaccharide through electrostatic interactions with basic residues lining the active site cleft (Wu, et al., Nat. Struct. Mol. Biol. 2015, 22, 1016-1022); and (5) What effects do addition of a C(3)-O-sulfate at the −2 subsite have on the recognition of heparanase (Peterson, et al., J. Biol. Chem. 2010, 285 (19), 14504-14513).

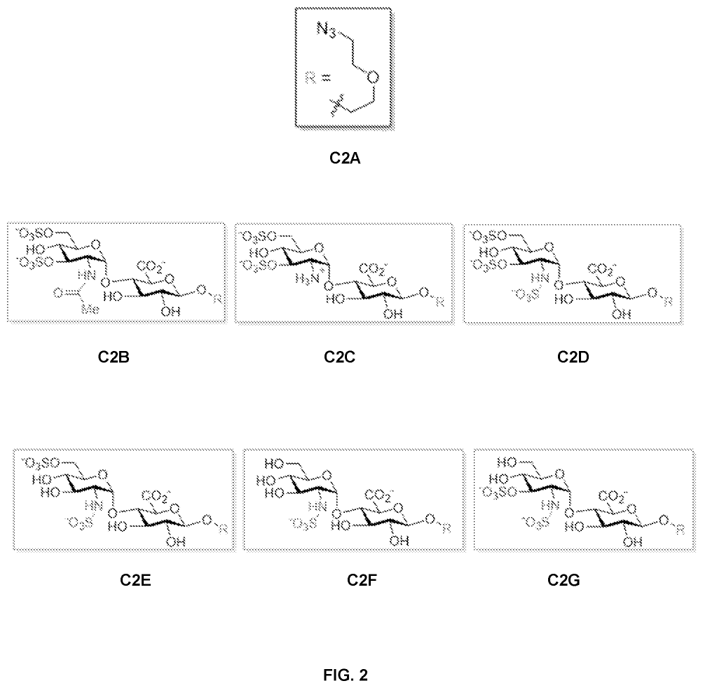

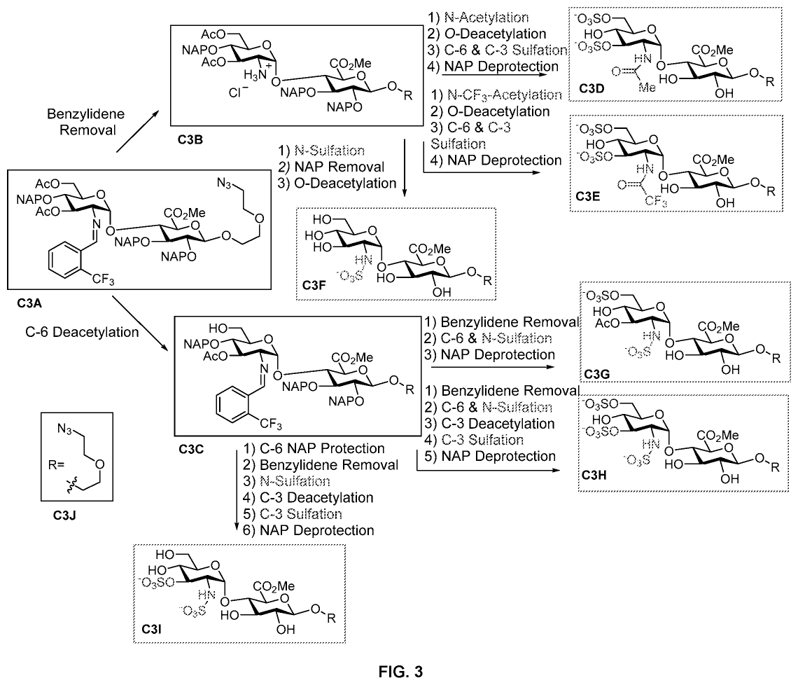

Synthesis of Designed Glycopolymers. To resolve the aforementioned questions, six disaccharides compounds C2B-C2G with sulfation patterns varying at the C(6)-O, C(3)-O, and C(2)-N positions were envisaged (FIG. 2). Based on the crystal structure of HS substrate-heparanase complex, it was hypothesized that N-, 3-O-, and 6-O—SO₃⁻ groups located at −2 subsite of heparanase could be critically important for heparanase-HS interaction. While disaccharides C2B and C2C examine whether C(6)-O—SO₃ located at the −2 subsite is critical for recognition, C2B and C2D determine whether the sulfate group located at C(6) or C(3) position of the glucosamine unit is more important. On the other hand, disaccharides C2E and C2F will provide a clear picture whether N—SO₃ groups located at −2 subsite of heparanase could be critically important for heparanase-HS interaction. Highly sulfated C2G could have a negative or positive impact on HS-heparanase interactions. This study provides a systematic understanding of substrate binding specificity and sulfate-recognition motifs.

With these intended disaccharides in mind, an orthogonal deprotection and selective sulfation strategy to synthesize the six differently sulfated −2 glucosamine units was developed, starting with a common and properly protected disaccharide building block C3A with a pendant azido linker, under a standard set of reaction conditions. A schematic strategy for the construction of the disaccharide fragments is displayed in FIG. 3. Disaccharide C3A, which had been previously synthesized (Loka, et al., Chem. Commun. 2017, 53 (65), 9163-9166), could be quickly diversified by either selective N-benzylidene removal under acidic conditions to provide disaccharide C3B or selective C(6)-deacetylation using sodium methoxide in methanol to yield disaccharide C3C. It was observed that the selective C(6)-deacetylation can only take place when the N-benzylidene group of the glucosamine moiety remains intact (FIG. 3) (Loka, et al., Chem. Commun. 2017, 53 (65), 9163-9166). Disaccharides C3B and C3C would be further functionalized to generate the corresponding six disaccharide intermediates C3D-C3I. In the first series of disaccharide synthesis, disaccharide C3B could be modified by N-acetylation, N—CF₃-acetylation, and selective sulfation, followed by removal of the napthylmethyl (NAP) ether protecting group, to construct the three intermediates (C3D)-(C3F) in overall good yields. The labile CF₃-acyl group is hydrolyzed after polymerization to reveal the free amine.

On the other hand, disaccharide C3C could be functionalized by N-benzylidene removal, followed by simultaneous C(6) and N-sulfation, to produce C3G. Furthermore, the C(3)-acetyl group of C3C can be deprotected and then sulfated eventually constructing C3H. In the steps leading to the synthesis of C3H, the following trends were observed. First, for the deacetylation process to proceed smoothly, it was essential for the N-sulfate counterions to be sodium cation (Na⁺) as opposed to the triethylammonium (Et₃NH⁺). It was discovered that exchange of triethylammonium for sodium reduced the elimination product that forms through deprotonation of the GlcA C(5)-hydrogen. Also, the elimination of the C(5)-hydrogen occurs if there is a free C(2)-amine present during the deacetylation step (Tiruchinapally, et al., Chem. Eur. J. 2011, 17 (36), 10106-10112). For the synthesis of (C3I), the primary C(6)-hydroxyl of C3C is first protected as the napthylmethyl ether, followed by sequential N-benzylidene removal and N-sulfation. After counterion exchange, the disaccharide intermediate is C(3)-deacetylated and then sulfated. Global NAP-deprotection with DDQ produces the corresponding disaccharide 031.

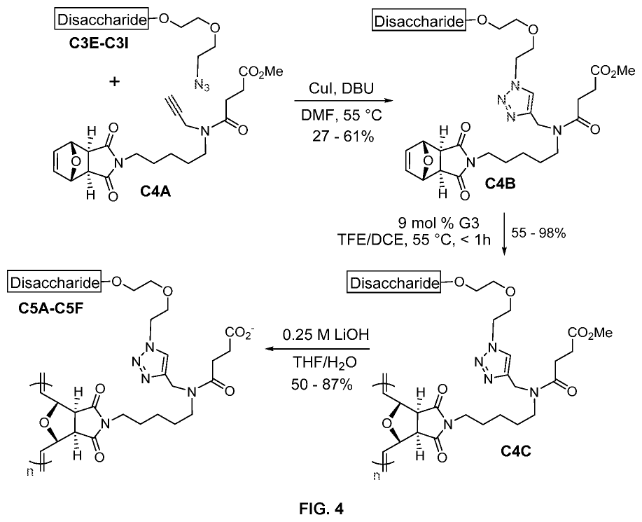

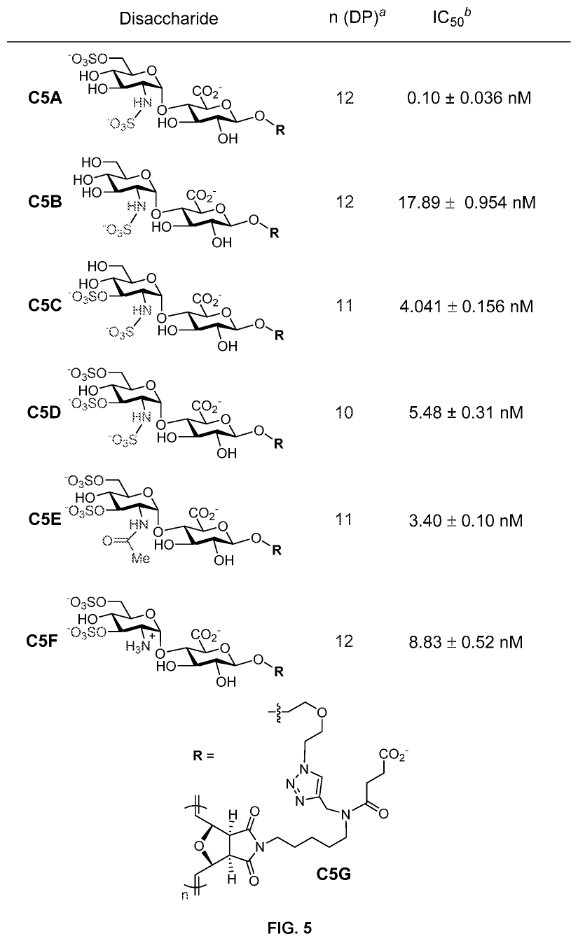

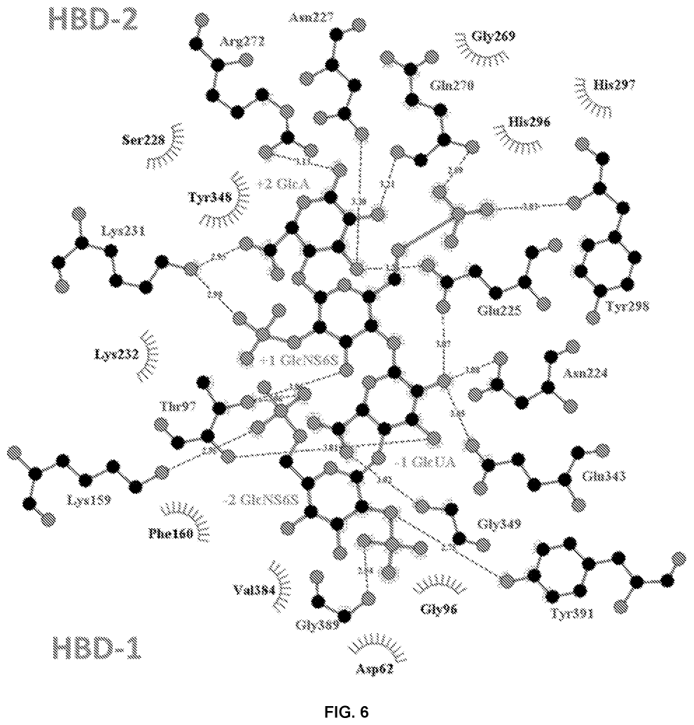

With the six differently sulfated deprotected disaccharides (C3E)-(C3I) in hand, they could now be individually coupled to the ROMP-capable monomer unit C4A via a CuAAC “click” reaction (FIG. 4) (Kolb, et al., Drug Discovery Today 2003, 8 (24), 1128-1137; Rostovtsev, et al., Angew. Chem. Int. Ed. 2002, 41 (14), 2596-2599; Tornøe, et al., J. Org. Chem. 2002, 67 (9), 3057-3064). The newly formed glycomonomers were obtained in moderate yield (27-61%) and then underwent polymerization using Grubbs' third generation catalyst (G3) in a mixture of 1,2-dichloroethane/2,2,2-trifluoroethanol as solvent (Rankin, et al., J. Polym. Sci., Part A: Polym. Chem. 2007, 45 (11), 2113-2128; Choi, et al., Angew. Chem. Int. Ed. 2003, 42 (15), 1743-1746). The unique solvent mixture was necessary to ameliorate the solubility of the polar sulfated monomer unit and to prevent the ruthenium catalyst decomposition, which has been reported with utilization of nucleophilic polar solvents such as methanol. The solvent ratio was adjusted according to the number of sulfates and free hydroxyls present on the disaccharide portion. Previous results show that the ideal degree of polymerization (DP) for inhibition of heparanase by a glycopolymer was 11-12 repeating units (Sletten, et al., Biomacromolecules 2017, 18 (10), 3387-3399; Loka, et al., Chem. Commun. 2017, 53 (65), 9163-9166). As a result, each differently sulfated monomer unit was independently polymerized with 9 mol % Grubbs' catalyst (G3) to provide high yields of the six differently sulfated glycopolymers within 1 h, all with similar optimal degrees of polymerization (Loka, et al., Chem. Commun. 2017, 53 (65), 9163-9166). Due to their amphiphilic nature, these glycopolymers aggregate to form micelles after polymerization. As such, they cannot be analyzed by gel permeation chromatography (GPC); instead, both the DP and molecular weight (Mₑ) of the six glycopolymers were determined by ¹H-NMR end group analysis. Following polymerization, the resulting glycopolymers were fully deprotected using 0.25 M LiOH in THF/H₂O and then purified by dialysis to remove impurities, affording the corresponding polymers FIG. 5A-5F (Johnson, et al., J. Am. Chem. Soc. 2011, 133 (3), 559-566).

In Vitro Testing. Heparanase Inhibition: After purification, the glycopolymers FIG. 5 were evaluated on how their varied sulfation patterns altered their heparanase inhibitory capabilities. Employing a TR-FRET assay against fluorescent labeled-HS, it was ultimately found that there is a direct correlation between sulfation pattern of the −2 GlcN and heparanase inhibition (FIG. 5) (Roy, et al., J. Med. Chem. 2014, 57 (11), 4511-4520). Specifically, it was observed that the −2 GlcN must be sulfated at both the C(6) and C(2)-N positions in order to induce the highest inhibitory effects on heparanase (CSA, IC₅₀=0.10±0.036 nM). Removal of the C(6)-sulfate (C5B) drastically reduced the inhibitory activity against heparanase (IC₅₀ to 17.89±0.954 nM). While previous report has demonstrated that heparanase can recognize glucosamine unit (GlcN) carrying either C(6)- or C(3)-O-sulfate (Peterson, et al., Matrix Biol. 2013, 32 (5), 223-227; Peterson, et al., J. Biol. Chem. 2010, 285 (19), 14504-14513), it was found that glycopolymer C5C bearing C(3)-O-sulfate (C5C, IC₅₀=4.041±0.156 nM) is less effective at inhibiting heparanase than glycopolymer C5A bearing C(6)-O-sulfate (5A). The addition of a third sulfate to the GlcNS6S moiety, forming polymer C5D (5D, IC₅₀=5.48±0.31 nM), did not prove to be advantageous. This result suggests that although the interactions are not purely electrostatic, heparanase recognizes the pendant saccharide. Moreover, the utilization of oversulfated saccharide compounds have been reported to increase nonspecific binding leading to unforeseen adverse effects (Sarrazin, et al., Cold Spring Harb Perspect Biol 2011, 3 (7); Guerrini, et al., Nat. Biotechnol. 2008, 26 (6), 669-675; Warkentin, et al., New Engl. J. Med. 1995, 332 (20), 1330-1335; Sun, et al., Biomacromolecules 2002, 3 (5), 1065-1070). Exchanging the N-sulfate (C5D) for a N-acetyl (C5E: IC50=3.40±0.10 nM) or ammonium (CSF: IC₅₀=8.83±0.52 nM) did not have a significant impact on the binding affinity. Overall, these results suggest that although −2 N-sulfate is important for heparanase recognition, it is not as important as −2 C(6)-O-sulfate.

These results obtained with glycopolymers C5A-C5F in FIG. 5 are in accordance with an in silico docking study with the glycomonomer substrates and the apo crystal structure of heparanase (PDB code: 5E8M) using the Autodock Vina suite in the YASARA program (Wu, et al., Nat. Struct. Mol. Biol. 2015, 22, 1016-1022; Krieger, et al., Bioinformatics 2014, 30 (20), 2981-2982; Trott, et al., J. Comput. Chem. 2010, 31 (2), 455-461). The investigation was initiated by docking the natural HS substrate, GlcNS(6S)α(1,4)GlcAβ(1,4)GlcNS(6S)α(1,4)GlcA tetrasaccharide, into human heparanase to obtain a benchmark for comparison with synthetically designed compounds. Currently, there are no computational programs that could manage the docking of glycopolymers, and so the monomeric precursors were investigated in the computational studies. When both the C(6) and C(2)-N positions were sulfated (polymer C5A, compounds C5A-C5G.), there was a strong network of interactions (ionic and hydrogen bonding) formed (Johnson, et al., J. Am. Chem. Soc. 2011, 133 (3), 559-566). The N-sulfate interacted with Lys159 and Arg303, while the C(6)-O-sulfate from a trivalent network with Asn64, Gly389, and Tyr 391. When the C(3)-O-sulfate for the trisulfate saccharide (C5C), compounds C5A-C5G) was introduced, it added an additional ionic interaction with Lys98; however, the interaction pulled the C(6)-O-sulfate away from Tyr391 and the N-sulfate from Arg303. This docking result is consistent with the experimental data wherein polymer C5C (IC₅₀=5.48±0.31 nM) is less effective at inhibiting heparanase than polymer C5A (IC₅₀=0.10±0.036 nM).

Finally, the prediction for recognition importance at the C(2)-N position (GlcNS6S>>GlcNS>GlcNAc) was partially upheld (Wu, et al., Nat. Struct. Mol. Biol. 2015, 22, 1016-1022). Heparanase strongly recognized the GlcNS6S motif (FIG. 5, C5A), but the preference between GlcNS and GlcNAc (C5B and C5E) were actually reversed. As previously mentioned, the orientation of the saccharide is vital and it was found that a hydrophobic pocket in the −2 subsite (Gly389, Asp62, Val34, Tyr391) accommodated the methyl of the acetyl group and provided the right orientation for the C(6)-sulfate to potentially interact with Lys232. The GlcNS only made it to the outer periphery of the binding site groove with little interactions. Removal of all substitution at the C(2)-N position still yielded fair inhibition (C5F); however, when looking at the docked compound, the disaccharide unit was found in the +2/+1 subsites with the reducing end directed towards HBD-1, opposite of the natural substrate and the other glycomonomer compounds. This docking result supports the findings of previous studies, that the N-sulfate is necessary for recognition in the −2 subsite. Overall, it is concluded that the combinatory effect of having both the C(6)- and C(2)-N positions sulfated presents the saccharide in the proper orientation for optimal binding at the −1, −2 subsite of heparanase. Any additional sulfates or changes in the pattern disrupt the positioning of the saccharide, reducing the number of ionic salt bridges and hydrogen bonding interactions.

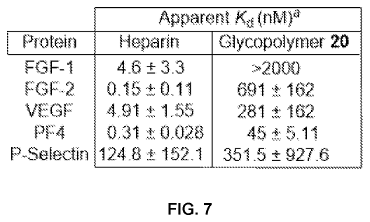

Cross-bioactivity Studies. After discovering that the GlcNS(6S)α(1,4)GlcA glycopolymer C5A (DP=12) is the most potent inhibitor of heparanase, the specificity of this synthetic glycopolymer was next sought to be found since HS polysaccharides are typically promiscuous (Capila, et al., Angew. Chem. Int. Ed. 2002, 41 (3), 390-412). It was previously established that glycopolymer C5A presented no anticoagulant activity in the presence of ATIII (Anti-FXa: IC₅₀>4500 nm and Anti-FIIa: IC₅₀>4500 nm) (Oh, et al., Angew. Chem. Int. Ed. 2013, 52 (45), 11796-11799; Loka, et al., Chem. Commun. 2017, 53 (65), 9163-9166). The ability of C5A to bind to a variety of HS-binding proteins was next screened (C5A-C5G). To achieve this goal, a solution-based BLI assay was utilized to determine the apparent K_{d} of the glycopolymer to HS-binding proteins in comparison to biotinylated-heparin (18 kDa) attached to the BLI streptavidin-probe (FIG. 7) (Cochran, et al., Glycoconjugate J. 2009, 26 (5), 577-587). The study was initiated by testing the validity of the assay by employing heparin (18 kDa) as the ligand. The apparent K_{d} found for several HS-binding proteins (FIG. 7) was similar to previously reported data obtained with a variety of methods (Cochran, et al., Glycoconjugate J. 2009, 26 (5), 577-587). Once the binding of heparin to HS-binding proteins has been established, the protein screening process was initiated by determining the K_{d} for synthetic glycopolymer C5A to three angiogenic growth factors (FGF-1, FGF-2, and VEGF) which are released during degradation of the ECM's HS by heparanase and are responsible for promoting tumor growth (Rivara, et al., Future Med. Chem. 2016, 8 (6), 647-680). The glycopolymer exhibited very low affinity to these three growth factors with K_{d} several orders of magnitude greater than the standard 18 kDa heparin utilized in the assay (FIG. 7). Next, focus was placed on the binding of C5A to platelet factor-4 (PF4), which is responsible for causing thrombocytopenia, the main reason why clinical trials for other carbohydrate-based heparanase inhibitors were halted (Rivara, et al., Future Med. Chem. 2016, 8 (6), 647-680; Arepally, et al., New Engl. J. Med. 2006, 355 (8), 809-817). Again, the K_{d} for the GlcNS(6S)α(1,4)GlcA glycopolymer (45±5.11 nM) was 150 times weaker than that of heparin (0.31±0.028 nM) and three times weaker than that of PI-88 (16.0±1.9 nm), a known heparanase inhibitor (Cochran, et al., Glycoconjugate J. 2009, 26 (5), 577-587). Lastly, P-selectin was tested as it plays a vital role in tumor cell metastasis, and the process can be attenuated by heparin (Stevenson, et al., Thromb. Res. 2007, 120, S107-S111; Manning, et al., Tetrahedron 1997, 53 (35), 11937-11952). Glycopolymer C5A (K_{d}=351.5±927.6 nM) presented a similar affinity to that of heparin (K_{d}=124.8±152.1 nM). The data obtained with P-selectin suggests that inhibiting heparanase and P-selectin simultaneously allows the glycopolymer to suppress both selectin-mediated tumor cell adhesion to endothelial cells and heparanase mediated extravasation through the subendothelial basement membrane.

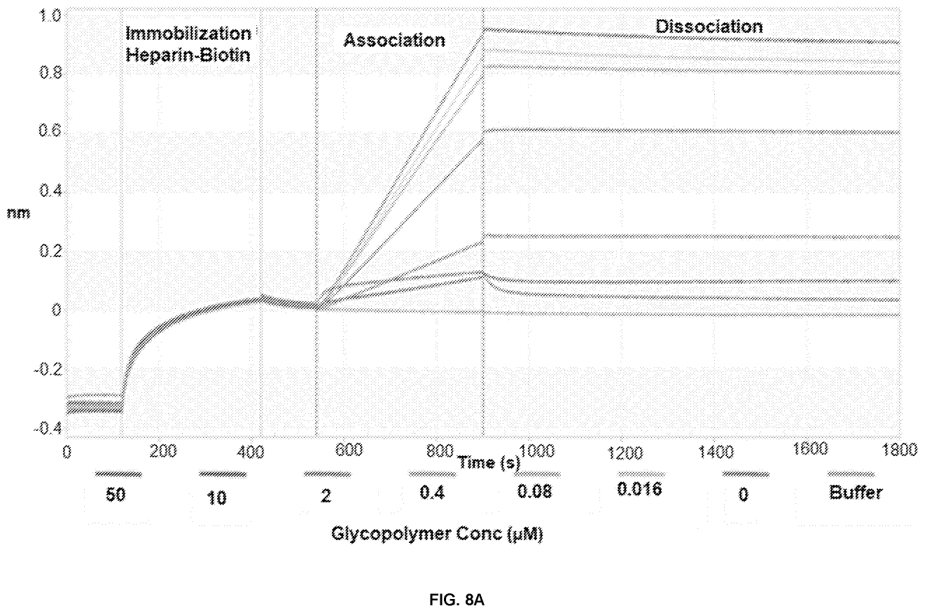

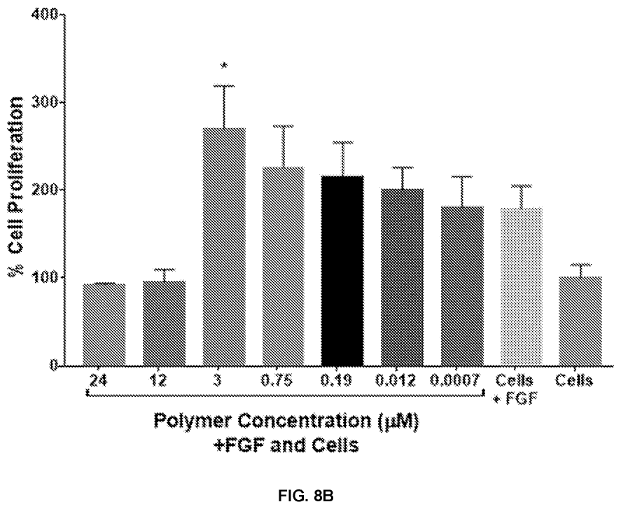

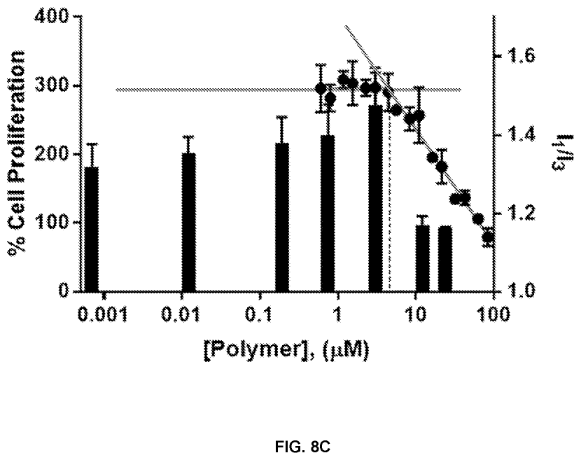

Interestingly, a biphasic behavior was found in all the binding studies. At lower concentrations of polymer C5A (<3 μM) the binding was linear; however, at concentrations above 3 μM there was a drastic change in binding (FIG. 8A). These concentrations directly correlate to the previously found 3.3 μM critical micelle concentration (CMC) for 5A (Loka, et al., Chem. Commun. 2017, 53 (65), 9163-9166). It was determined that at the higher concentrations, glycopolymer C5A exists in its micellar form and begins to tightly sequester the proteins, resulting in that there was no protein available to bind to the heparin attached to the BLI probe (Koide, et al., Nat. Chem. 2017, 9, 715-722; Belair, et al., Chem. Commun. 2014, 50 (99), 15651-15668). The biphasic behavior of the GlcNS(6S)α(1,4)GlcA glycopolymer was also observed in the human umbilical vascular endothelial cell (HUVEC) proliferation assay using FGF-2 (FIG. 8B). Again, at concentrations below the CMC (0.0007-0.75 μM), there was statistically no cell proliferation compared to the control without glycopolymer. These results support the BLI data for FGF-2 to the glycopolymer, in which very little binding occurred at low concentrations (FIG. 8A). It was not until polymer C5A reached 3 μM concentration that a small change in HUVEC proliferation was observed (FIG. 8B). As previously seen with the BLI data, at concentrations above 3 μM, there was a strong decrease in cell proliferation, down to the exact same level as that of the control without FGF-2 (FIG. 8B). As shown in FIG. 8C, there is a direct correlation between cell proliferation and the formation of micelle. It was hypothesized that sequestering FGF-2 by the newly formed micelles does not allow the protein to bind to the FGF-receptor on the HUVEC surface, either from steric repulsion or improper binding orientation of the ternary complex (Pellegrini, et al., Nature 2000, 407, 1029-1034). It is important to note that these concentrations are much greater than the inhibitory concentration of the synthetic GlcNS(6S)α(1,4)GlcA glycopolymer C5A against heparanase.

In Vivo Model. Metastasis is the leading cause of death of cancer patients (Mina, et al., Nat. Rev. Clin. Oncol. 2011, 8 (6), 325-332). Although the metastatic cascade is complex, it is well documented that degradation of the ECM's HS by heparanase is a major contributing factor in the dissemination of malignant tumors (Rivara, et al., Future Med. Chem. 2016, 8 (6), 647-680; Sanderson, et al., Semin. Cell Dev. Biol. 2001, 12 (2), 89-98). Breast cancer is the leading cause of female mortality worldwide and accounts for 25% of the total number of cancer cases and 15% of all cancer-associated female mortality (Torre, et al., Ca-Cancer J. Clin. 2015, 65 (2), 87-108). With the ultimate objective of understanding if the in vitro inhibition of heparanase by sulfated glycopolymers would translate in vivo, the GlcNS(6S)α(1,4)GlcA glycopolymer C5A (DP=12) was subjected to a 4T1 mammary carcinoma model of experimental metastasis (FIG. 9) (Pulaski, et al. Curr. Protoc. Immunol. 2001, 39 (1), 20.22.21-20.22.16; Menhofer, et al., PLOS ONE 2014, 9 (11), e112542). As a positive control, heparin was also subjected to in vivo studies.

Looking at the antimetastatic properties for these two compounds, heparin consistently reduced the size of the metastasized lung tumor by half (FIG. 9). When the GlcNS(6S)α(1,4)GlcA glycopolymer C5A (DP=12) was subjected to the same assay, 4 out of the 5 mice presented almost no metastatic spread into the lungs. As demonstrated in FIG. 9, GlcNS(6S)α(1,4)GlcA glycopolymer C5A (DP=12) markedly inhibited the extravasion of 4T1 cells and their subsequent colonization in the mouse lungs. This effect was similar to that exerted by Roneparstat, N-acetylated, glycol-split heparin, (not shown) (Cassinelli, et al., Oncotarget 2016, 7 (30), 47848-47863; Cassinelli, et al., Biochem. Pharmacol. 2013, 85 (10), 1424-1432; Rossini, et al., Hematol. Oncol. 2017, 36 (1), 360-362), a drug that recently ended a Phase I clinical trial in myeloma patients. These results indicate that glycopolymer inhibits the ability of blood-borne carcinoma cells to extravasate through the subendothelial basement membrane due to combined effect of modulating P-selectin and heparanase activities (Menhofer, et al., PLOS ONE 2014, 9 (11), e112542).

Conclusion. The rational design and synthesis of a powerful multivalent inhibitor of heparanase that translates from in vitro inhibition of the enzyme to an effective in vivo anticancer agent is described in this Example. A synthetically designed glycopolymer of 12 repeating units bearing a pendant GlcNS(6S)α(1,4)GlcA saccharide unit affords tight-binding inhibition of the cancer-promoting endoglycosidase, heparanase was shown. Advantageously, the glycopolymer has minimal cross-bioactivity with serine proteases in the coagulation cascade as well as several HS-binding proteins including angiogenic growth factors and platelet factor 4. The present disclosure also shows that the synthetic glycopolymer could act against P-selectin, which in conjunction with heparanase inhibition provides a dual mechanism underlying the potent inhibition of malignant cell dissemination from metastasizing throughout the body. Inhibition of metastasis has been clearly demonstrated in a mouse 4T1 carcinoma cell model, in which the sulfated glycopolymer effectively prohibited the carcinoma cells to extravasate and colonize in the lungs. Overall, the disclosure presented a high affinity, synthetic glycopolymer inhibitor of heparanase that overcomes the limitations associated with the lack of specificity noted with previously developed heparin-based inhibitors.

Supporting Information. General information: Methods and Reagents. All reactions were performed in dried flasks fitted with glass stopper under a positive pressure of nitrogen atmosphere unless otherwise noted. Organic solutions were concentrated using a Buchi rotary evaporator below 40° C. at 25 torr. Analytical thin-layer chromatography (TLC) was routinely utilized to monitor the progress of the reactions and performed using pre-coated glass plates with 230-400 mesh silica gel impregnated with a fluorescent indicator (250 nm). Visualization was achieved using UV light, iodine, or ceric ammonium molybdate stain. Flash column chromatography was performed using 40-63 μm silica gel (SiliaFlash® F60 from Silicycle) or by a Redisep Rf Gold column on a Teledyne ISCO Flash Purification System. Dry solvents were obtained from a SG Waters solvent system utilizing activated alumina columns under an argon pressure.

Instrumentation. All NMR spectra were taken at 25° C. in deuterated solvent (Cambridge Isotope Laboratories) unless stated otherwise. Chemical shifts are expressed in parts per million (δ scale) relative to the NMR solvent for ¹H and ¹³C NMR (CDCl₃: δ 7.27 ppm, δ 77.16 ppm; D₂O: δ 4.79 ppm; and MeOD (d-4): δ 3.31 ppm, δ 49.00 ppm) or CF₃-toluene (−63.72 ppm) for ¹⁹F NMR. Spectra were processed using the automatic phasing and polynomial baseline correction features of the MestReNova software. Data are presented as follows: chemical shift, multiplicity (s=singlet, d=doublet, t=triplet, q=quartet, m=multiplet), integration, and coupling constant in hertz (Hz). High resolution (ESI-TOF) mass spectrometry were acquired at Wayne State University.

General Synthetic Procedures and Characterization.

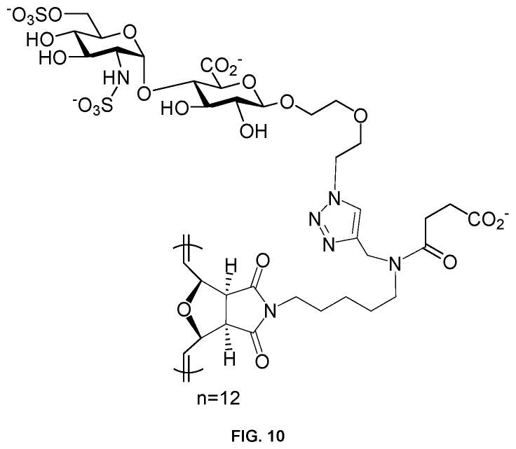

FIG. 10 shows the structure of compound CSA. Compound C5A was prepared as described in Loka, et al., Chem. Commun. 2017, 53, 9163-9166; Sletten, et al., Biomacromolecules 2017, 18, 3387-3399.

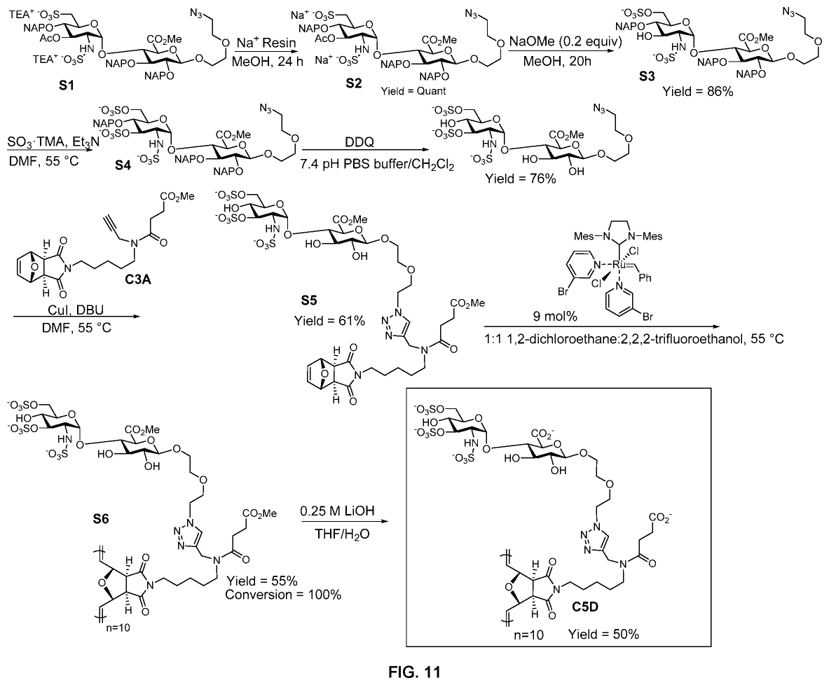

FIG. 11 shows the synthetic route for the synthesis of trisulfated glycopolymer C5D. Compound 51 was prepared as described in Loka, et al., Chem. Commun. 2017, 53, 9163-9166; Sletten, et al., Biomacromolecules 2017, 18, 3387-3399.

A 20 mL scintillation vial was charged with S1 (35 mg) in 1.5 mL of methanol. To the vial, 1 g of Na⁺ exchange resin was added. The reaction was stirred vigorously at 1000 RPM for 24 h. After 24 h the reaction was filtered and concentrated by rotary evaporation to quantitatively yield the sodium salt S2 (35 mg). Full conversion to the sodium salt was then analyzed by ¹H NMR by looking for the disappearance of the triethlyamine associated resonances: ¹H NMR (500 MHz, MeOD) δ 7.88-7.81 (m, 5H), 7.75 (d, J=8.3 Hz, 2H), 7.70 (dd, J=12.8, 7.4 Hz, 4H), 7.58 (d, J=8.1 Hz, 1H), 7.50-7.43 (m, 7H), 7.41-7.33 (m, 2H), 5.59 (d, J=3.5 Hz, 1H), 5.31-5.25 (m, 1H), 5.14 (dd, J=11.4, 7.7 Hz, 2H), 5.01 (d, J=11.2 Hz, 1H), 4.95 (d, J=11.4 Hz, 1H), 4.82 (d, J=11.3 Hz, 1H), 4.73 (dd, J=16.2, 9.5 Hz, 2H), 4.42 (d, J=9.6 Hz, 1H), 4.29 (d, J=10.4 Hz, 1H), 4.22-4.13 (m, 2H), 4.00-3.95 (m, 1H), 3.89 (t, J=8.1 Hz, 1H), 3.83 (s, 3H), 3.81-3.76 (m, 2H), 3.67-3.63 (m, 2H), 3.59-3.54 (m, 3H), 3.47 (dd, J=10.8, 3.5 Hz, 1H), 3.22 (dd, J=5.5, 4.3 Hz, 2H), 1.94 (s, 3H).

In a 1 mL conical Schlenk flask, under nitrogen, compound S2 (35 mg, 0.032 mmol, 1 equiv.) was dissolved in a NaOMe (0.34 mg, 0.0063 mmol, 0.2 equiv.) in anhydrous Methanol (0.5 mL) solution. The reaction mixture was stirred overnight at RT. Reaction completion was monitored by the disappearance of the starting material by ESI mass spectrometry in negative mode. Upon completion, the reaction mixture was directly loaded using minimal methanol onto a brand new 12 g Redisep Rf Gold column and purified by silica gel flash chromatography on a Teledyne ISCO Flash Purification System (A-CH₂Cl₂ B-Methanol 0→40% B over 25 CV) to afford disaccharide S3 (28.4 mg, 86%).

The NMR results were: ¹H NMR (500 MHz, MeOD) δ 7.90 (s, 1H), 7.87-7.85 (m, 1H), 7.84-7.80 (m, 4H), 7.77-7.69 (m, 6H), 7.66 (d, J=7.9 Hz, 1H), 7.61 (d, J=8.5 Hz, 1H), 7.48-7.42 (m, 7H), 7.40-7.35 (m, 2H), 5.52 (d, J=3.6 Hz, 1H), 5.17-5.08 (m, 4H), 4.93 (d, J=8.6 Hz, 2H), 4.79 (d, J=11.6 Hz, 1H), 4.72 (d, J=7.4 Hz, 1H), 4.36 (dd, J=10.5, 3.1 Hz, 1H), 4.24 (d, J=8.8 Hz, 1H), 4.17 (d, J=8.2 Hz, 1H), 4.13 (d, J=8.5 Hz, 1H), 4.00-3.94 (m, 1H), 3.90 (t, J=7.9 Hz, 1H), 3.83-3.79 (m, 1H), 3.76 (s, 3H), 3.70-3.60 (m, 10H), 3.60-3.55 (m, 4H), 3.25-3.20 (m, 2H).

¹³C NMR (126 MHz, MeOD) δ 171.0, 137.7, 137.4, 137.1, 134.8, 134.7, 134.7, 134.5, 134.4, 134.4, 129.2, 129.0, 128.9, 128.9, 128.8, 128.7, 128.7, 128.6, 128.5, 128.1, 127.9, 127.7, 127.6, 127.3, 127.0, 126.8, 126.7, 126.7, 126.6, 104.7, 99.8, 82.8, 82.7, 79.2, 77.2, 76.1, 75.8, 75.2, 75.1, 74.6, 71.4, 70.9, 70.2, 67.3, 59.8, 55.1, 53.4, 51.7.

Purification elution fractions were analyzed for product by ESI mass spectrometry in negative mode: HRMS (ESI⁻) calc. for C₅₀H₅₂N₄O₁₈S₂ (M+Na)⁻¹: 1083.2615; found: 1083.2621.

A 5 mL vial was sequentially charged with disaccharide S3 (26 mg, 0.0311 mmol, 1 equiv.), DMF (0.160 mL), SO₃.Me₃N (130 mg, 0.933 mmol, 30 equiv.), and triethylamine (0.087 mL, 0.622 mmol, 20 equiv.). The reaction mixture was stirred at 50° C. for 3 d. The reaction progress was monitored by ESI negative mode mass spectrometry. The white solid was filtered off using cotton plug washing with CH₂Cl₂. The reaction was then concentrated in vacuo. The residue was purified using C-18 reverse phase silica gel flash chromatography (0→80% acetonitrile/water) to afford S4 (26 mg, 74%).

The NMR results were: ¹H NMR (500 MHz, MeOD) δ 8.00 (s, 1H), 7.87 (dd, J=10.6, 7.3 Hz, 2H), 7.80 (dd, J=10.2, 7.2 Hz, 3H), 7.76-7.72 (m, 2H), 7.72-7.63 (m, 5H), 7.53 (dd, J=8.4, 1.4 Hz, 1H), 7.44-7.33 (m, 7H), 5.73 (d, J=3.1 Hz, 1H), 5.42 (d, J=11.3 Hz, 1H), 5.34 (d, J=9.7 Hz, 1H), 5.11 (d, J=11.5 Hz, 1H), 4.98 (d, J=11.3 Hz, 1H), 4.85-4.80 (m, 3H), 4.80-4.74 (m, 1H), 4.70 (d, J=7.7 Hz, 1H), 4.43 (d, J=9.4 Hz, 1H), 4.24-4.12 (m, 3H), 4.00-3.92 (m, 2H), 3.81-3.76 (m, 6H), 3.67 (t, J=4.7 Hz, 2H), 3.59 (t, J=5.0 Hz, 2H), 3.52-3.43 (m, 2H), 3.22 (dd, J=5.4, 4.0 Hz, 2H).

¹³C NMR (126 MHz, MeOD) δ 171.2, 138.3, 137.7, 137.6, 134.8, 134.8, 134.7, 134.5, 134.4, 134.3, 129.3, 129.2, 129.0, 128.9, 128.8, 128.6, 128.5, 128.5, 128.5, 128.0, 127.9, 127.5, 127.2, 126.9, 126.8, 126.6, 126.6, 126.5, 104.7, 99.2, 84.2, 82.7, 78.8, 77.9, 77.8, 76.0, 75.8, 75.4, 72.0, 71.4, 71.0, 70.2, 67.0, 59.1, 53.5, 51.7, 45.6.

Purification elution fractions were analyzed for product by ESI mass spectrometry in negative mode: HRMS (ESI⁻) calc. for C₅₀H₅₁N₄O₂₁S₃ (M+2Na)⁻¹: 1185.2003; found: 1185.1987.

A 20 mL scintillation vial was charged with 2-naphthylmethyl protected sulfated disaccharide S4 (34 mg, 0.03 mmol, 1 equiv.), CH₂Cl₂ (0.45 mL), pH 7.4 1×PBS buffer (0.45 mL) and recrystallized 2,3-dichloro-5,6-dicyano-1,4-benzoquinone (54.5 mg, 0.24 mmol, 8 equiv.). An oversized stir bar was added and the vial was wrapped in aluminum foil. The biphasic reaction mixture was vigorously stirred overnight at RT. Reaction completion was monitored by disappearance of the starting material by ESI mass spectrometry in negative mode. Upon completion the reaction mixture was directly loaded onto a brand new 40 g Redisep Rf Gold column using minimal methanol and purified by on a Teledyne ISCO Flash Purification System (A-CH₂Cl₂ B-Methanol 0→20% B over 5 CV then 20→40% B over 20 CV) to afford the disaccharide C3H (16.5 mg, 76%).

The NMR results were: ¹H NMR (500 MHz, MeOD) δ 5.56 (d, J=3.4 Hz, 1H), 4.45 (d, J=7.8 Hz, 1H), 4.35 (dd, J=10.7, 8.6 Hz, 1H), 4.23 (dt, J=18.9, 6.3 Hz, 2H), 4.03-3.98 (m, 1H), 3.97-3.91 (m, 1H), 3.90-3.80 (m, 4H), 3.79-3.65 (m, 9H), 3.42-3.37 (m, 3H).

¹³C NMR (126 MHz, MeOD) δ 170.6, 104.6, 101.0, 81.1, 79.3, 77.2, 76.2, 74.2, 72.4, 71.3, 71.0, 70.2, 70.1, 67.3, 58.6, 53.6, 51.8.

Purification elution fractions were analyzed for product by ESI mass spectrometry in negative mode: HRMS (ESI⁻) calc. for C₁₇H₂₇N₄O₂₁S₃ (M+2Na)⁻¹: 765.01254; found: 765.0131.

An oven dried 10 mL Schlenk flask was charged with a solution of polymerizable scaffold C (7.8 mg, 0.0195 mmol 1.2 equiv.) in CH₂Cl₂ and a solution of deprotected sulfated disaccharide C3H (11.7 mg, 0.016 mmol, 1 equiv.) in methanol. The mixture was then concentrated by rotary evaporation and placed in vacuo for 30 min. Under N₂, copper(I) iodide (3 mg, 0.016 mmol, 1 equiv.) was added followed by anhydrous DMF (0.2 mL). Lastly the addition of DBU (3 μL, 0.0195 mmol, 1.2 equiv.) was performed by a microsyringe. The resulting mixture was stirred overnight at 55° C. The reaction mixture was monitored by ESI mass spectrometry in negative mode for complete consumption of C3H. Upon completion, the reaction mixture was directly loaded onto a brand new 24 g Redisep Rf Gold column using minimal methanol and purified by silica gel flash chromatography on a Teledyne ISCO Flash Purification System (A-CH₂Cl₂ B-Methanol 0→60% B over 20 CV) to afford the diantennary glycomonomer S5 (11 mg, 61%), after click reaction.

The NMR results were: ¹H NMR (500 MHz, MeOD) δ 8.14 (s, 1H), 7.99 (s, 1H), 6.48 (dd, J=11.3, 5.8 Hz, 2H), 5.60-5.55 (m, 1H), 5.40-5.30 (m, 2H), 5.02 (d, J=21.7 Hz, 1H), 4.65-4.51 (m, 3H), 4.42 (d, J=7.8 Hz, 1H), 4.38-4.32 (m, 1H), 4.25-4.15 (m, 2H), 4.04-3.97 (m, 1H), 3.88 (d, J=4.8 Hz, 4H), 3.82 (s, 3H), 3.69-3.63 (m, 9H), 3.46-3.38 (m, 3H), 3.35 (d, J=2.1 Hz, 1H), 3.19-3.06 (m, 1H), 2.90-2.82 (m, 1H), 2.75 (s, 1H), 2.66 (dt, J=9.5, 4.8 Hz, 3H), 2.57 (d, J=7.4 Hz, 1H), 1.60 (dd, J=32.8, 20.3 Hz, 4H), 1.36-1.21 (m, 2H).

¹³C NMR (126 MHz, MeOD) δ 173.9, 169.2, 136.7, 136.2, 103.3, 99.4, 80.5, 80.4, 79.8, 78.7, 77.9, 76.0, 75.9, 74.6, 72.8, 71.0, 69.9, 68.9, 68.8, 65.9, 57.1, 52.2, 50.9, 50.1, 49.7, 41.6, 39.3, 29.3, 28.7, 28.0, 27.7, 26.6, 26.3, 26.3, 23.8, 23.7.

Purification elution fractions were analyzed for product by ESI mass spectrometry in negative mode: HRMS (ESI⁻) calc. for C₃₈H₅₃N₆O₂₇S₃ (M+2Na⁺2H)⁻¹: 1169.2072; found: 1169.2051.

Into an oven dried 10 mL Schlenk flask under N₂ a solution of diantennary monomer S5 (4.5 mg, 0.0044 mmol) in a degassed mixture of 1:1 1,2-dichloroethane:2,2,2-trifluoroethanol (DCE:TFE) (1 mL) was transferred in. (Note: Solvent mixture was degassed in bulk by freeze-pump-thaw method prior to dissolving monomer. Degassing was repeated at least 5 times until bubbles subsided.) The mixture was then concentrated by rotary evaporation and placed in vacuo for 30 min. In a glove box under an inert N₂ atmosphere a 1 mL oven dried, conical Schlenk flask was charged with 3.3 mg of catalyst [(H₂IMes)(3-Br-py)₂(Cl)₂Ru═CHPh] (G3), then sealed with glass stopper and removed from the glove box. The G3 was then dissolved in 0.485 mL of degassed 2.5:1 DCE:TFE under N₂, to make a stock solution. Under N₂, monomer S5 was redissolved in the degassed 2.5:1 DCE:TFE (0.100 mL) mixture and a magnetic stir bar was added. 0.100 mL of the G3 stock solution was then rapidly injected to the monomer solution Schlenk under N₂ and then sealed with a glass stopper (final concentration=0.025 M). The resulting solution was then lowered into a 55° C. oil bath and allowed to stir. After the solution became cloudy (1 h) the conversion of the monomer was monitored by ¹H NMR of a reaction aliquot in CD₃OD by observing the disappearance of the strained alkene peak at 6.4 ppm. Upon full conversion the reaction was cooled to RT and stirred for 5 min. The reaction mixture was quenched with ethyl vinyl ether (5 drops) and allowed to stir for 30 min. The reaction mixture was then transferred into a 20 mL scintillation vial and concentrated in vacuo. The crude product was dissolved in a minimal amount of methanol and precipitated with an excess of diethyl ether. Precipitate was allowed to settle and the liquid was then decanted off. Note: If the precipitant was very fine, this solution was centrifuged, and the liquid was decanted. The precipitate was then redissolved in excess methanol (2 mL) and reconcentrated until the polymer was in a minimal amount of methanol. This process was repeated two more times. The final residual precipitate was dried in vacuo to yield trisulfated polymer S6, after polymerization, as an off white solid (1.7 mg, yield=55%, conversion=100%, DP=10). The ratio of the GlcN anomeric peak (5.5 ppm) and the phenyl end group (7.4 ppm) were used to find the DP.

The NMR results were: ¹H NMR (500 MHz, D₂O) δ 8.12-7.82 (m, 1H), 7.41 (s, 1H), 5.94 (s, 1H), 5.53 (s, 1H), 5.42 (s, 1H), 4.60 (s, 3H), 4.40-4.12 (m, 3H), 4.01-3.58 (m, 16H), 3.47-3.30 (m, 5H), 3.19 (s, 1H), 2.81 (d, J=27.8 Hz, 2H), 2.66 (s, 3H), 1.53 (s, 4H), 1.28 (s, 2H).

Trisulfated polymer S6 (1.7 mg) was charged into a 5 mL vial along with 0.137 mL, 0.25 M LiOH aqueous solution, 1.5 mL water, and 0.377 mL THF and allowed to stir at RT for 24 h. The reaction mixture was then frozen using liquid nitrogen and lyophilized to completion. Remaining solid was then dissolved in water and placed inside a dialysis cartridge (Slide-A-Lyzer G2 Dialysis Cassettes, 3.5K MWCO, 3 mL, Cat. #: 87723) and dialyzed against 0.9% NaCl solution for 24 h (3 buffer changes) then against DI water for 24 h (3 buffer changes). Finally, the sample was transferred into a 5 mL vial and frozen by liquid nitrogen. The sample was then lyophilized to obtain fully deprotected trisulfated polymer C5D, after saponification, as a white solid (0.9 mg, 50%).

The NMR results were: ¹H NMR (500 MHz, D₂O) δ 8.14-7.86 (m, 1H), 7.56-7.25 (m, 1H), 6.02-5.69 (m, 1H), 5.58 (s, 1H), 5.14 (s, 1H), 4.66-4.45 (m, 5H), 4.34 (d, J=10.3 Hz, 2H), 4.18 (d, J=11.5 Hz, 1H), 4.11-4.03 (m, 1H), 3.99-3.60 (m, 12H), 3.56 (t, J=9.2 Hz, 1H), 3.45-3.33 (m, 4H), 3.31-3.24 (m, 1H), 2.90-2.50 (m, 4H), 1.73-1.38 (m, 4H), 1.37-1.09 (m, 2H).

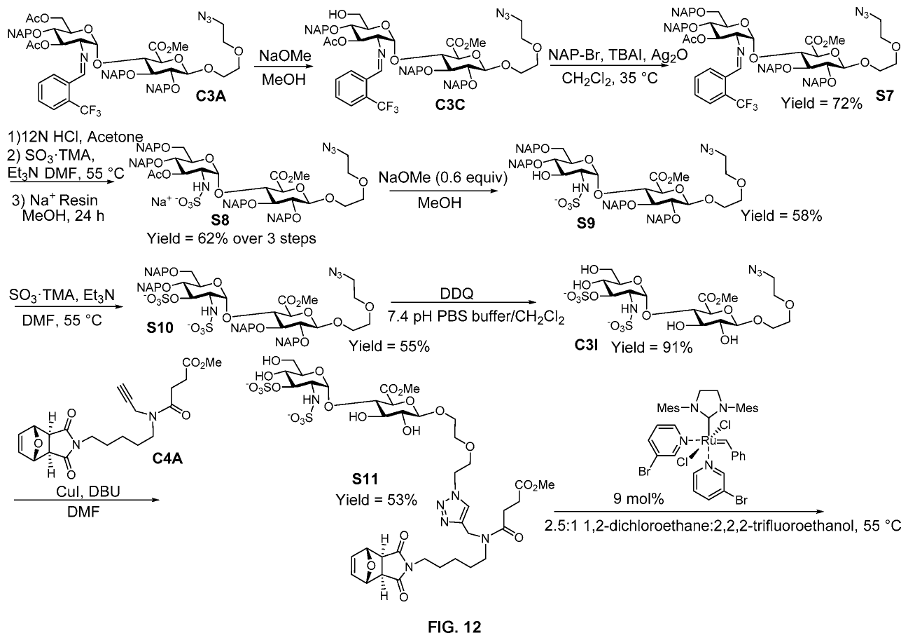

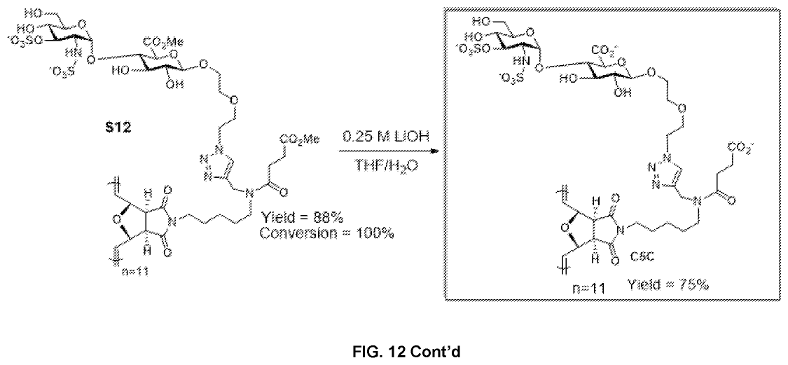

FIG. 12 shows the synthetic route for synthesis of C(3)-SO₃N—SO₃ disulfated glycopolymer CSC.

A 25 mL oven dried Schlenk flask was charged with disaccharide C3 A (128 mg, 0.112 mmol, 1 equiv.), anhydrous methanol (0.52 mL), and CH₂Cl₂ (0.15 mL). NaOMe (14.2 mg, 0.26 mmol, 1 equiv.) was added and stirred at RT for 1 h. The reaction was monitored for completion by TLC (1:1 hexanes:ethyl acetate). Upon completion, the reaction was diluted with CH₂Cl₂ and neutralized by Amberlyst® (Rohm & Haas, Co., West Philadelphia, Pa.) 15 hydrogen form, filtered, and concentrated to yield disaccharide C3C (103 mg, 83%).

Disaccharide C3C (87 mg, 0.078 mmol, 1 equiv.) was charged under N₂ into an oven dried 10 mL Schlenk flask along with 2-(bromomethyl)naphthalene (346 mg, 20 equiv.), tetrabutylammonium iodide (5.8 mg, 0.2 equiv.), and 4 Å activated molecular sieves (52 mg, 100 mg/mL). Contents were then dissolved in dry CH₂Cl₂ (0.52 mL) under N₂ and stirred at RT. After 1 h, Ag₂O (18.5 mg, 0.078 mmol, 1 equiv.) was added under N₂ and the reaction was allowed to stir overnight at 35° C. The reaction was monitored by TLC (1:1 hexanes:ethyl acetate). Upon completion, the reaction was filtered through a Celite® 545 plug and concentrated. The reaction mixture was then dissolved in 0.5 mL of toluene loaded directly on to a silica gel column and purified by flash chromatography (10 g of silica, ½in ID×12 in column, 5:1→3:1→2:1→1:1 hexanes:ethyl acetate) to provide the desired S7 (50.6 mg, yield=72% based on recovered starting material).

The NMR results were: ¹H NMR (500 MHz, CDCl₃) δ 8.41 (d, J=1.8 Hz, 1H), 8.26 (d, J=7.5 Hz, 1H), 7.84 (t, J=7.5 Hz, 3H), 7.75 (t, J=8.2 Hz, 3H), 7.68-7.61 (m, 3H), 7.59 (t, J=7.8 Hz, 3H), 7.55-7.46 (m, 6H), 7.46-7.32 (m, 11H), 7.16 (dd, J=8.4, 1.3 Hz, 1H), 6.90 (dd, J=8.4, 1.1 Hz, 1H), 5.67 (t, J=9.8 Hz, 1H), 5.62 (d, J=3.5 Hz, 1H), 5.08 (d, J=11.2 Hz, 1H), 5.00 (d, J=11.7 Hz, 1H), 4.91-4.79 (m, 3H), 4.69-4.60 (m, 3H), 4.56 (d, J=11.6 Hz, 1H), 4.31 (t, J=9.2 Hz, 1H), 4.13 (d, J=9.5 Hz, 1H), 4.11-4.04 (m, 1H), 3.96-3.81 (m, 4H), 3.81-3.67 (m, 8H), 3.67-3.60 (m, 3H), 3.51 (dd, J=10.3, 3.5 Hz, 1H), 3.31 (td, J=4.8, 2.4 Hz, 2H), 1.68 (s, 3H).

¹³C NMR (126 MHz, CDCl₃) δ 169.6, 169.3, 160.2, 136.0, 135.8, 135.7, 135.6, 133.4, 133.3, 133.3, 133.3, 133.2, 133.1, 134.0, 132.8, 132.1, 130.6, 128.6, 128.4, 128.1, 128.1, 128.1, 128.0, 128.0, 127.9, 127.9, 127.7, 127.7, 127.6, 127.1, 127.0, 126.4, 126.3, 126.3, 126.3, 126.1, 126.1, 125.9, 125.9, 125.8, 125.8, 125.6, 125.0, 124.7, 104.1, 99.0, 84.1, 81.6, 75.9, 75.7, 74.8, 74.6, 74.5, 73.9, 73.6, 72.5, 71.4, 70.5, 70.1, 69.4, 68.1, 52.7, 50.8, 20.7.

Purification elution fractions were analyzed for product by ESI mass spectrometry: HRMS (ESI⁺) calc. for C₇₁H₆₇F₃N₄O₁₃ (M)⁺: 1240.4657; found: 1240.4663.

Into a 2.5 mL vial containing S7 (153 mg, 0.123 mmol, 1 equiv.) 0.6 mL of acetone was added followed by 12 N HCl (0.153 mL, 15 equiv.) and stirred at RT for 8 min, with monitoring by TLC (1:1 hexanes:ethyl acetate). Upon completion, the reaction mixture was then diluted with acetone and concentrated in vacuo. The crude was passed through a silica plug using 1:1 hexanes:ethyl acetate→100% ethyl acetate→20:1 CH₂Cl₂:methanol. The residue in a 10 mL oven dried Schlenk flask was sequentially charged with anhydrous DMF (0.6 mL), SO₃.Me₃N (513 mg, 3.69 mmol, 30 equiv.), and triethylamine (0.34 mL, 2.46 mmol, 20 equiv.) under nitrogen. The reaction mixture was stirred at 55° C. for 3 d. The reaction progress was monitored by ESI negative mode mass spectrometry. The white solid was filtered off using cotton plug washing with CH₂Cl₂. The reaction was then concentrated in vacuo. The residue was purified using C-18 reverse phase silica gel flash chromatography (0→80% acetonitrile/water) to afford the triethylammonium salt form of S8.

To a 25 mL round bottom charged with the triethylammonium salt of S8, 5 mL of methanol followed by 5 g of Na⁺ exchange resin was added. The reaction was stirred vigorously at 1000 RPM for 24 h. After 24 h the reaction was filtered and concentrated by rotary evaporation to quantitatively yield the sodium salt S8 (89 mg, 62% over 3 steps).

The NMR results were: ¹H NMR (400 MHz, MeOD) δ 7.82-7.65 (m, 14H), 7.56 (d, J=9.4 Hz, 2H), 7.48-7.37 (m, 11H), 7.33 (dd, J=13.3, 4.1 Hz, 2H), 7.20 (dd, J=8.4, 1.5 Hz, 1H), 5.72 (d, J=3.4 Hz, 1H), 5.26-5.20 (m, 1H), 5.11 (dd, J=11.4, 7.0 Hz, 2H), 4.94 (d, J=12.1 Hz, 1H), 4.77-4.66 (m, 4H), 4.61 (dd, J=11.8, 2.8 Hz, 2H), 4.19-4.15 (m, 2H), 4.00-3.88 (m, 2H), 3.81-3.60 (m, 11H), 3.55 (dd, J=10.9, 6.4 Hz, 3H), 3.48 (dd, J=10.8, 3.5 Hz, 1H), 3.22-3.17 (m, 2H), 1.96 (s, 3H).

¹³C NMR (126 MHz, MeOD) δ 173.1, 171.0, 137.4, 137.2, 137.0, 137.0, 134.7, 134.7, 134.6, 134.5, 134.4, 134.3, 129.2, 129.1, 129.0, 129.0, 128.9, 128.9, 128.7, 128.7, 128.7, 128.6, 128.5, 127.9, 127.9, 127.7, 127.5, 127.3, 127.3, 127.2, 127.1, 127.1, 127.0, 127.0, 127.0, 126.9, 126.8, 126.7, 104.8, 99.2, 83.2, 82.7, 77.7, 76.7, 75.8, 75.5, 75.2, 75.1, 74.4, 74.3, 72.8, 71.3, 70.7, 70.2, 69.4, 58.4, 55.1, 53.3, 51.7, 21.5.

Purification elution fractions were analyzed for product by ESI mass spectrometry: HRMS (ESI) calc. for C₆₃H₆₃N₄O₁₅S (M)⁻¹: 1163.3965; found: 1163.3960.

In a 10 mL Schlenk flask, under nitrogen, compound S8 (40 mg, 0.034 mmol, 1 equiv.) was dissolved in a NaOMe (1.1 mg, 0.024 mmol, 0.6 equiv.) in anhydrous Methanol (0.7 mL) solution. The reaction mixture was stirred overnight at RT. Reaction completion was monitored by the disappearance of the starting material by ESI mass spectrometry in negative mode. After 24 h an additional 0.3 equiv. (0.55 mg) of NaOMe was added in 0.1 mL of anhydrous methanol. Upon completion, the reaction mixture was directly loaded using minimal methanol onto a brand new 12 g Redisep Rf Gold column and purified by silica gel flash chromatography on a Teledyne ISCO Flash Purification System (A-CH₂Cl₂ B-Methanol 0→40% B over 25 CV) to afford disaccharide S9 (22 mg, 58%), after acetate deprotection. Purification elution fractions were analyzed for product by ESI mass spectrometry in negative mode.

The NMR results were: ¹H NMR (500 MHz, MeOD) δ 7.79 (d, J=7.2 Hz, 1H), 7.77-7.71 (m, 9H), 7.69-7.60 (m, 5H), 7.56 (s, 1H), 7.47-7.33 (m, 11H), 7.26 (dd, J=8.4, 1.4 Hz, 1H), 5.65 (d, J=3.6 Hz, 1H), 5.14-5.02 (m, 3H), 4.94 (d, J=11.0 Hz, 1H), 4.78 (d, J=11.6 Hz, 1H), 4.73-4.62 (m, 3H), 4.54 (d, J=12.2 Hz, 1H), 4.19-4.12 (m, 2H), 4.00-3.89 (m, 2H), 3.82-3.70 (m, 4H), 3.68-3.62 (m, 6H), 3.61-3.58 (m, 1H), 3.58-3.54 (m, 4H), 3.39 (dd, J=10.3, 3.6 Hz, 1H), 3.21 (dd, J=5.5, 3.9 Hz, 2H).

¹³C NMR (126 MHz, MeOD) δ 171.1, 137.6, 137.5, 137.2, 137.0, 134.8, 134.7, 134.7, 134.5, 134.4, 134.4, 129.1, 129.1, 129.0, 129.0, 128.9, 128.8, 128.7, 128.7, 128.6, 128.5, 128.0, 127.8, 127.7, 127.5, 127.4, 127.3, 127.2, 127.1, 127.1, 127.0, 126.9, 126.9, 126.8, 126.7, 126.7, 126.6, 104.7, 99.1, 83.3, 82.8, 79.4, 76.0, 76.0, 75.5, 75.2, 75.1 74.9, 74.3, 72.2, 71.4, 71.0, 70.2, 69.7, 59.9, 53.1, 51.7.

Purification elution fractions were analyzed for product by ESI mass spectrometry: HRMS (ESI) calc. for C₆₁H₆₁N₄O₁₅S (M)⁻¹: 1121.3854; found: 1121.3860.

A 10 mL oven dried Schlenk flask containing S9 (36 mg, 0.032 mmol, 1 equiv.) was sequentially charged with anhydrous DMF (0.160 mL), SO₃.Me₃N (179 mg, 1.28 mmol, 40 equiv.), and triethylamine (0.059 mL, 0.8 mmol, 25 equiv.) under nitrogen. The reaction mixture was stirred at 55° C. for 3 d. The reaction progress was monitored by ESI negative mode mass spectrometry. The white solid was filtered off using cotton plug washing with CH₂Cl₂. The reaction was then concentrated in vacuo. The residue was purified using C-18 reverse phase silica gel flash chromatography (0→80% acetonitrile/water) to afford the triethylammonium salt form of S10 (21 mg, 55%), after sulfation.

The NMR results were: ¹H NMR (500 MHz, MeOD) δ 7.83 (s, 1H), 7.74 (ddd, J=21.7, 15.1, 8.4 Hz, 10H), 7.65-7.60 (m, 5H), 7.48 (dd, J=8.5, 1.4 Hz, 1H), 7.45-7.37 (m, 10H), 7.31 (dd, =11.0, 4.0 Hz, 1H), 5.86 (d, =3.0 Hz, 1H), 5.35 (d, =11.3 Hz, 1H), 5.27 (d, =11.0 Hz, 1H), 5.10 (d, J=11.6 Hz, 1H), 4.98 (d, J=11.4 Hz, 1H), 4.82-4.74 (m, 2H), 4.71 (d, J=7.6 Hz, 1H), 4.64 (dd, J=16.3, 11.6 Hz, 2H), 4.50 (d, J=12.1 Hz, 1H), 4.23-4.17 (m, 2H), 4.02-3.92 (m, 2H), 3.82-3.69 (m, 8H), 3.66 (dd, J=9.8, 7.4 Hz, 3H), 3.57 (t, J=5.0 Hz, 2H), 3.52-3.46 (m, 2H), 3.21 (dd, J=5.5, 4.0 Hz, 2H).

¹³C NMR (126 MHz, MeOD) δ 171.5, 138.2, 137.6, 137.6, 137.2, 134.8, 134.8, 134.7, 134.7, 134.5, 134.4, 134.2, 129.2, 129.1, 129.0, 129.0, 128.9, 128.8, 128.7, 128.6, 128.6, 128.5, 128.5, 128.1, 127.9, 127.7, 127.7, 127.7, 127.5, 127.2, 127.1, 127.1, 127.0, 126.9, 126.8, 126.8, 126.7, 126.6, 126.5, 84.2, 82.8, 79.0, 78.1, 76.5, 75.7, 75.7, 75.3, 74.2, 72.9, 71.4, 71.0, 70.2, 69.6, 59.1, 53.4, 51.7.

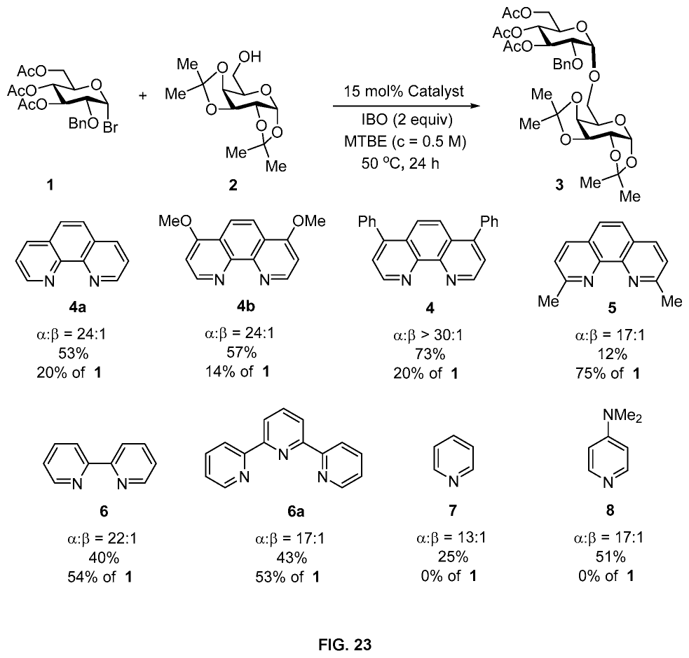

Purification elution fractions were analyzed for product by ESI mass spectrometry in negative mode. HRMS (ESL) calc. for C₆₁H₆₁N₄O₁₅S (M+Na)⁻¹: 1223.3241; found: 1223.3247 (FIG. 23A).

A 5 mL vial was charged with 2-naphthylmethyl protected sulfated disaccharide S10 (21 mg, 0.017 mmol, 1 equiv.), CH₂Cl₂ (0.17 mL), pH 7.4 1×PBS buffer (0.17 mL) and recrystallized 2,3-dichloro-5,6-dicyano-1,4-benzoquinone (31.7 mg, 0.14 mmol, 8 equiv.). An oversized stir bar was added and the vial was wrapped in aluminum foil. The biphasic reaction mixture was vigorously stirred overnight at RT. Reaction completion was monitored by disappearance of the starting material by ESI mass spectrometry in negative mode. Upon completion the reaction mixture was directly loaded onto a brand new 12 g Redisep Rf Gold column using minimal methanol and purified by silica gel flash chromatography on a Teledyne ISCO Flash Purification System (A-CH₂Cl₂ B-Methanol 0→20% B over 5 CV then 20→50% B over 20 CV) to afford the disaccharide C3I (9.8 mg, 91%), after napthyl deprotection.

The NMR results were: ¹H NMR (400 MHz, MeOD) δ 5.56 (d, J=3.4 Hz, 1H), 4.38 (d, J=7.8 Hz, 1H), 4.28 (dd, J=10.7, 8.9 Hz, 1H), 3.95 (d, J=9.6 Hz, 1H), 3.87 (dt, J=10.5, 4.1 Hz, 1H), 3.80 (t, J=9.1 Hz, 1H), 3.75-3.57 (m, 12H), 3.38 (dd, J=9.9, 2.7 Hz, 1H), 3.32 (dd, J=10.0, 4.3 Hz, 3H), 3.16 (d, J=7.1 Hz, 1H).

¹³C NMR (101 MHz, MeOD) δ 170.7, 104.6, 100.5, 80.2, 79.5, 77.3, 75.9, 74.2, 74.0, 71.3, 71.0, 70.2, 70.2, 61.7, 58.4, 53.3, 51.8.

Purification elution fractions were analyzed for product by ESI mass spectrometry in negative mode. HRMS (ESI⁻) calc. for C₁₇H₂₈N₄O₁₈S₂ (M+Na)⁻¹: 663.0737; found: 663.0734.

An oven dried 10 mL Schlenk flask was charged with a solution of polymerizable scaffold C4A (7.4 mg, 0.018 mmol 1.2 equiv.) in CH₂Cl₂ and a solution of deprotected sulfated disaccharide C3I (9.8 mg, 0.015 mmol, 1 equiv.) in methanol. The mixture was then concentrated by rotary evaporation and placed in vacuo for 30 min. Under N₂, copper (I) iodide (2.8 mg, 0.015 mmol, 1 equiv.) was added followed by anhydrous DMF (0.160 mL). Lastly the addition of DBU (2.5 μL, 0.015 mmol, 1.2 equiv.) was performed by a microsyringe. The resulting mixture was stirred overnight at 55° C. The reaction mixture was monitored by ESI mass spectrometry in negative mode for complete consumption of C3I. Upon completion, the reaction mixture was directly loaded onto a brand new 12 g Redisep Rf Gold column using minimal methanol and purified by silica gel flash chromatography on a Teledyne ISCO Flash Purification System (A-CH₂Cl₂ B-Methanol 0→50% B over 20 CV) to afford the diantennary glycomonomer S11 (8.2 mg, 53%), after click reaction.

The NMR results were: ¹H NMR (400 MHz, MeOD) δ 8.14 (s, 1H), 7.93 (s, 1H), 6.53-6.43 (m, 2H), 5.67 (d, J=3.5 Hz, 1H), 5.39-5.30 (m, 2H), 5.05 (s, 1H), 4.67-4.51 (m, 3H), 4.44 (d, J=7.8 Hz, 1H), 4.36 (dd, J=10.7, 8.9 Hz, 1H), 4.03 (d, J=9.4 Hz, 1H), 3.88 (m, 4H), 3.82-3.62 (m, 15H), 3.42 (m, 5H), 3.16 (td, J=13.7, 6.7 Hz, 1H), 2.88 (t, J=6.3 Hz, 1H), 2.79-2.70 (m, 2H), 2.69-2.61 (m, 3H), 2.58 (t, J=7.5 Hz, 1H), 1.75-1.49 (m, 4H), 1.41-1.24 (m, 2H).

¹³C NMR (101 MHz, MeOD) δ 175.3, 174.5, 170.8, 138.1, 137.7, 104.7, 100.3, 81.9, 81.8, 81.1, 80.1, 80.0, 79.9, 79.5, 77.4, 75.8, 74.2, 73.9, 71.3, 70.3, 70.3, 61.7, 58.4, 53.3, 52.3, 52.2, 51.4, 51.1, 43.0, 40.7, 30.1, 29.4, 29.1, 28.9, 27.7, 25.2, 25.1.

Purification elution fractions were analyzed for product by ESI mass spectrometry in negative mode. HRMS (ESI⁻) calc. for C₁₇H₂₈N₄O₁₈S₂ (M+Na)⁻¹: 663.0737; found: 663.0734.

Into an oven dried 10 mL Schlenk flask under N₂ a solution of diantennary monomer S11 (8.2 mg, 0.0078 mmol) in a degassed mixture of 2.5:1 1,2-dichloroethane:2,2,2-trifluoroethanol (DCE:TFE) (1 mL) was transferred in. (Note: Solvent mixture was degassed in bulk by freeze-pump-thaw method prior to dissolving monomer. Degassing was repeated at least 5 times until bubbles subsided.) The mixture was then concentrated by rotary evaporation and placed in vacuo for 30 min. In a glove box under an inert N₂ atmosphere a 1 mL oven dried, conical Schlenk flask was charged with 4.9 mg of catalyst [(H₂IMes)(3-Br-py)₂(Cl)₂Ru═CHPh] (G3), then sealed with glass stopper and removed from the glove box. The G3 was then dissolved in 0.79 mL of degassed 2.5:1 DCE:TFE under N₂, to make a stock solution. Under N₂, monomer S11 was redissolved in the degassed 2.5:1 DCE:TFE (0.214 mL) mixture and a magnetic stir bar was added. 0.100 mL of the G3 stock solution was then rapidly injected to the monomer solution Schlenk under N. and then sealed with a glass stopper (final concentration=0.025 M). The resulting solution was then lowered into a 55° C. oil bath and allowed to stir. After the solution became cloudy (1 h) the conversion of the monomer was monitored by ¹H NMR of a reaction aliquot in CD₃OD by observing the disappearance of the strained alkene peak at 6.4 ppm. Upon full conversion the reaction was cooled to RT and stirred for 5 min. The reaction mixture was quenched with ethyl vinyl ether (5 drops) and allowed to stir for 30 min. The reaction mixture was then transferred into a 20 mL scintillation vial and concentrated in vacuo. The crude product was dissolved in a minimal amount of methanol and precipitated with an excess of diethyl ether. Precipitate was allowed to settle and the liquid was then decanted off. Note: If the precipitant was very fine, this solution was centrifuged, and the liquid was decanted. The precipitate was then redissolved in excess methanol (2 mL) and reconcentrated until the polymer was in a minimal amount of methanol. This process was repeated two more times. The final residual precipitate was dried in vacuo to yield disulfated polymer S12 as an off white solid (7.2 mg, yield=88%, conversion=100%, DP=11), after polymerization.

The NMR results were: ¹H NMR (500 MHz, D₂O) δ 8.08 (s, 1H), 7.91 (s, 1H), 7.57-7.24 (m, 2H), 5.93 (s, 2H), 5.73 (s, 1H), 5.58 (s, 2H), 5.43 (d, J=10.2 Hz, 2H), 4.57 (d, J=33.2 Hz, 3H), 4.32 (t, J=9.9 Hz, 1H), 4.17 (s, 1H), 3.97-3.61 (m, 17H), 3.47 (d, J=10.8 Hz, 2H), 3.11 (s, 3H), 2.76 (s, 3H), 2.64 (s, 3H), 1.54 (s, 4H), 1.25 (s, 2H).

Disulfated polymer S12 (7.2 mg) was charged into a 20 mL vial along with 0.579 mL 0.25 M LiOH aqueous solution, 6.3 mL water, and 1.57 mL THF and allowed to stir at RT for 24 h. The reaction mixture was then frozen using liquid nitrogen and lyophilized to completion. Remaining solid was then dissolved in water and placed inside a dialysis cartridge (Slide-A-Lyzer G2 Dialysis Cassettes, 3.5K MWCO, 3 mL, Cat. #: 87723) and dialyzed against 0.9% NaCl solution for 24 h (3 buffer changes) then against DI water for 24 h (3 buffer changes). Finally, sample was transferred into a 5 mL vial and frozen by liquid nitrogen. The sample was then lyophilized to obtain fully deprotected disulfated polymer C5C as a white solid (6.1 mg, 75%), after saponification.

The NMR results were: ¹H NMR (500 MHz, D₂O) δ 8.07 (s, 1H), 7.92 (d, J=16.6 Hz, 1H), 7.34 (t, J=38.9 Hz, 1H), 5.89 (d, J=103.1 Hz, 2H), 5.59 (s, 2H), 5.10 (s, 1H), 4.60 (s, 2H), 4.49 (s, 1H), 4.36 (t, J=9.7 Hz, 1H), 4.00-3.61 (m, 15H), 3.40 (t, J=16.4 Hz, 6H), 3.19 (s, 1H), 2.77 (s, 2H), 2.63 (s, 2H), 1.60 (d, J=35.7 Hz, 4H), 1.27 (s, 1H).

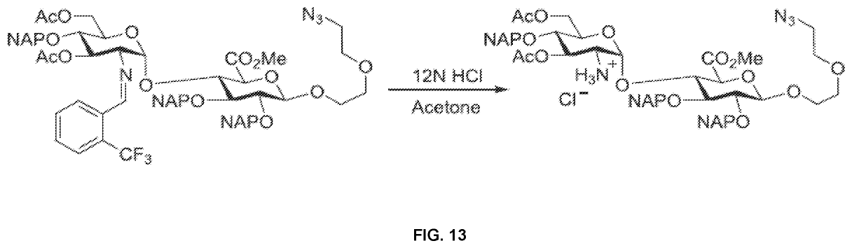

FIG. 13 shows the synthesis for the removal of N-benzylidene for disaccharide C3B. The structure of compound C3B was prepared by literature procedure and crude compound moved forward (Loka, et al., Chem. Commun. 2017, 53, 9163-9166; Sletten, et al., Biomacromolecules 2017, 18, 3387-3399).

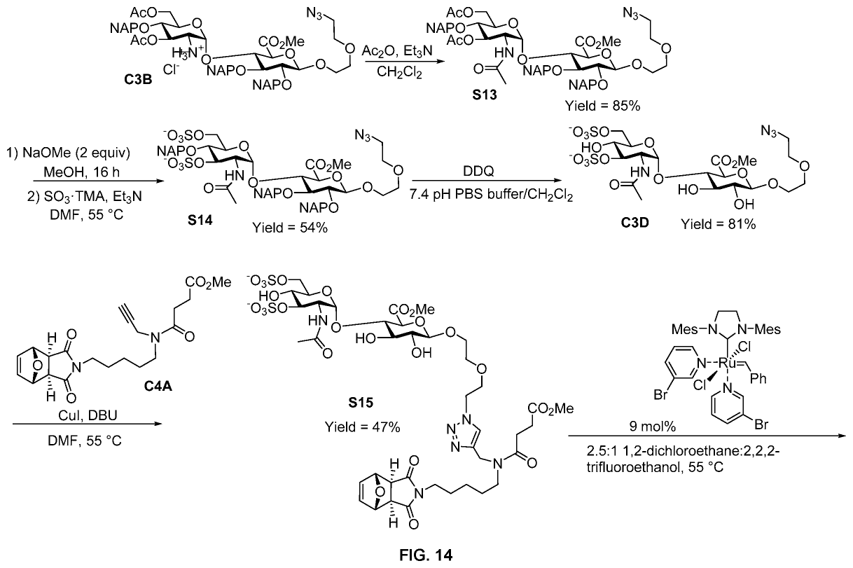

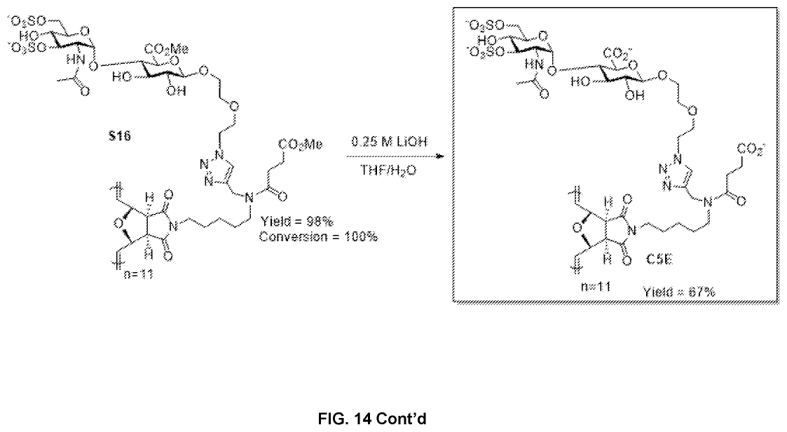

FIG. 14 shows the synthetic route for N-acetylated disulfated glycopolymer (5E).

An oven dried 10 mL Schlenk flask was charged with a solution of disaccharide C3B (45.4 mg, 0.046 mmol 1 equiv.) in anhydrous CH₂Cl₂ and subsequently charged with triethylamine (0.032 mL, 0.23 mmol, 5 equiv.), acetic anhydride (0.022 mL, 0.23 mmol, 5 equiv.), and a few crystals of 4-dimethylaminopyridine. The reaction was stirred at RT for 4 h, with monitoring by TLC (1:1 hexanes:ethyl acetate and 20:1 CH₂Cl₂:methanol). Upon completion, the reaction mixture was loaded directly on to a silica gel column and purified by flash chromatography (10 g of silica, ½in ID×12 in column, 1:1→1:2 hexanes:ethyl acetate). After purification, the fractions containing the product were combined and concentrated to provide the desired S13 (40 mg, 85%), after N-acetylation.

The NMR results were: ¹H NMR (500 MHz, CDCl₃) δ 7.85-7.68 (m, 12H), 7.52-7.40 (m, 7H), 7.39 (dd, J=8.4, 1.5 Hz, 1H), 7.34 (dd, J=8.4, 1.3 Hz, 1H), 5.97 (d, J=9.8 Hz, 1H), 5.39 (dd, J=10.8, 9.2 Hz, 1H), 5.19 (dd, J=15.9, 11.0 Hz, 2H), 4.99 (d, J=3.4 Hz, 1H), 4.89 (d, J=11.3 Hz, 1H), 4.83-4.76 (m, 2H), 4.73 (d, J=11.5 Hz, 1H), 4.64 (d, J=7.1 Hz, 1H), 4.34 (dd, J=12.1, 1.9 Hz, 1H), 4.25-4.14 (m, 2H), 4.07-3.98 (m, 2H), 3.91 (d, J=9.2 Hz, 1H), 3.86-3.79 (m, 2H), 3.77-3.64 (m, 8H), 3.61 (d, J=5.1 Hz, 2H), 3.29 (td, J=4.8, 2.1 Hz, 2H), 1.99 (s, 3H), 1.87 (d, J=7.1 Hz, 3H), 1.07 (s, 3H).

¹³C NMR (126 MHz, CDCl₃) δ 171.0, 170.5, 170.3, 168.2, 135.5, 134.9, 134.8, 133.3, 133.3, 133.2, 133.1, 133.1, 133.0, 128.4, 128.3, 128.2, 128.0, 127.9, 127.7, 127.7, 127.6, 127.1, 127.0, 126.9, 126.3, 126.2, 126.2, 126.2, 126.1, 126.1, 126.0, 126.0, 125.9, 104.2, 99.4, 82.1, 81.6, 78.1, 75.7, 75.2, 74.9, 74.8, 74.5, 73.5, 70.5, 70.3, 70.0, 69.2, 62.0, 52.9, 52.1, 50.7, 22.0, 21.0, 20.5.

Purification elution fractions were analyzed for product by ESI mass spectrometry: HRMS (ESI) calc. for C₅₆H₆₀N₄O₁₅ (M+Na): 1051.3934; found: 1051.3947.

A 10 mL oven dried Schlenk flask was charged with disaccharide S13 (40 mg, 0.039 mmol, 1 equiv.) and anhydrous methanol (0.250 mL). NaOMe (4 mg, 0.08 mmol, 1 equiv.) was added and stirred overnight at RT. The reaction was monitored for completion by TLC (1:2 hexanes:ethyl acetate). Upon completion, the reaction was diluted with CH₂Cl₂:methanol mixture and neutralized by Amberlyst® 15 hydrogen form, filtered, and concentrated.

An oven dried 10 mL Schlenk flask containing deacetylated crude was sequentially charged under N₂ with DMF (0.2 mL), SO₃.Me₃N (217 mg, 1.56 mmol, 40 equiv.), and triethylamine (110 mL, 0.78 mmol, 20 equiv.). The reaction mixture was stirred at 50° C. for 3 d. The reaction progress was monitored by ESI negative mode mass spectrometry. The white solid was filtered off using cotton plug washing with CH₂Cl₂. The reaction mixture was then concentrated in vacuo. The residue was purified using C-18 reverse phase silica gel flash chromatography (0→80% acetonitrile/water) to afford disulfated disaccharide S14 (24 mg, 54%).

The NMR results were: ¹H NMR (500 MHz, MeOD) δ 8.00 (s, 1H), 7.90-7.84 (m, 1H), 7.83-7.67 (m, 10H), 7.63 (d, J=7.9 Hz, 1H), 7.47-7.33 (m, 8H), 5.51 (d, J=3.1 Hz, 1H), 5.33 (d, J=9.7 Hz, 1H), 5.15 (d, J=11.5 Hz, 1H), 5.05 (s, 1H), 4.94 (d, J=11.1 Hz, 1H), 4.84-4.76 (m, 4H), 4.72 (d, J=7.6 Hz, 1H), 4.42 (d, J=10.2 Hz, 1H), 4.20 (d, J=10.3 Hz, 1H), 4.12 (d, J=9.4 Hz, 1H), 4.05-3.91 (m, 3H), 3.87 (t, J=8.8 Hz, 1H), 3.83-3.66 (m, 8H), 3.61 (t, J=4.9 Hz, 2H), 3.54 (t, J=8.3 Hz, 1H), 3.26 (d, J=4.6 Hz, 2H), 1.70 (s, 3H).

¹³C NMR (126 MHz, MeOD) δ 173.8, 170.8, 137.5, 137.4, 137.3, 134.8, 134.8, 134.7, 134.5, 134.4, 134.4, 129.2, 129.1, 128.9, 128.9, 128.8, 128.6, 128.6, 128.6, 128.5, 127.6, 127.3, 127.2, 127.0, 126.9, 126.9, 126.8, 126.8, 126.7, 105.0, 98.7, 84.3, 82.9, 78.7, 77.7, 77.4, 76.1, 76.0, 75.8, 75.5, 72.2, 71.4, 71.0, 70.3, 66.8, 55.3, 53.6 51.8, 22.9.

Purification elution fractions were analyzed for product by ESI mass spectrometry in negative mode: HRMS (ESL) calc. for C₅₂H₅₄N₄O₁₉S₂(M+Na)⁻¹: 1125.2721; found: 1125.2708.

A 5 mL vial was charged with 2-naphthylmethyl protected disulfated disaccharide S14 (23 mg, 0.021 mmol, 1 equiv.), CH₂Cl₂ (0.3 mL), pH 7.4 1×PBS buffer (0.3 mL) and recrystallized 2,3-dichloro-5,6-dicyano-1,4-benzoquinone (28 mg, 0.12 mmol, 6 equiv.). An oversized stir bar was added and the vial was wrapped in aluminum foil. The biphasic reaction mixture was vigorously stirred overnight at RT. Reaction completion was monitored by disappearance of the starting material by ESI mass spectrometry in negative mode. Upon completion the reaction mixture was directly loaded onto a brand new 24 g Redisep Rf Gold column using minimal methanol and purified by silica gel flash chromatography on a Teledyne ISCO Flash Purification System (A-CH₂Cl₂ B-Methanol 0→20% B over 5 CV then 20→50% B over 20 CV) to afford the disaccharide C3D (11.7 mg, 81%), after napthyl deprotection.

The NMR results were: ¹H NMR (500 MHz, MeOD) δ 5.30 (d, J=3.5 Hz, 1H), 4.44-4.39 (m, 2H), 4.23 (s, 2H), 4.04-3.91 (m, 4H), 3.84 (s, 3H), 3.79 (ddd, J=14.8, 13.8, 7.1 Hz, 3H), 3.73-3.60 (m, 8H), 3.43-3.38 (m, 2H), 3.29-3.24 (m, 3H), 2.00 (d, J=5.5 Hz, 3H).

¹³C NMR (126 MHz, MeOD) δ 173.6, 170.6, 104.7, 100.0, 79.8, 79.6, 77.1, 76.1, 75.0, 72.7, 71.4, 71.0, 70.2, 67.2, 53.8, 53.6, 51.8, 48.0, 22.9.

Purification elution fractions were analyzed for product by ESI mass spectrometry in negative mode: HRMS (ESI⁻) calc. for C₁₉H₃₀N₄O₁₉S₂(M+Na)⁻¹: 705.0843; found: 705.0849.

An oven dried 10 mL Schlenk flask was charged with a solution of polymerizable scaffold C4A (8.27 mg, 0.02 mmol 1.2 equiv.) in CH₂Cl₂ and a solution of deprotected disulfated disaccharide C3D (11.7 mg, 0.017 mmol, 1 equiv.) in methanol. The mixture was then concentrated by rotary evaporation and placed in vacuo for 30 min. Under N₂, copper (I) iodide (3.3 mg, 0.017 mmol, 1 equiv.) was added followed by anhydrous DMF (0.2 mL). Lastly the addition of DBU (3 μL, 0.02 mmol, 1.2 equiv.) was performed by a microsyringe. The resulting mixture was stirred overnight at 55° C. The reaction mixture was monitored by ESI mass spectrometry in negative mode for complete consumption of C3D. Upon completion, the reaction mixture was directly loaded onto a brand new 12 g Redisep Rf Gold column using minimal methanol and purified by silica gel flash chromatography on a Teledyne ISCO Flash Purification System (A-CH₂Cl₂ B-Methanol 0→50% B over 20 CV) to afford the diantennary glycomonomer S15 (8.7 mg, 47%), after click reaction.

¹H NMR (500 MHz, MeOD) δ 8.12 (s, 1H), 7.96 (s, 1H), 6.48 (d, J=7.5 Hz, 2H), 5.39-5.34 (m, 1H), 5.31 (d, J=20.3 Hz, 2H), 5.05 (d, J=10.7 Hz, 1H), 4.71 (s, 1H), 4.66-4.53 (m, 3H), 4.47-4.37 (m, 2H), 4.24 (s, 2H), 4.04-3.97 (m, 2H), 3.93-3.87 (m, 3H), 3.83 (d, J=11.0 Hz, 4H), 3.75-3.62 (m, 10H), 3.47-3.35 (m, 4H), 3.25 (d, J=9.6 Hz, 1H), 3.20-3.08 (m, 1H), 2.87 (t, J=6.4 Hz, 1H), 2.74 (d, J=6.2 Hz, 1H), 2.67 (q, J=7.0 Hz, 3H), 2.57 (d, J=7.5 Hz, 1H), 2.01 (s, 3H), 1.74-1.51 (m, 4H), 1.31 (m, 2H).

¹³C NMR (126 MHz, MeOD) δ 175.3, 174.5, 173.9, 173.5, 170.6, 138.0, 137.7, 104.7, 100.1, 82.0, 81.8, 81.2, 80.1, 79.5, 77.0, 76.1, 75.0, 72.8, 71.3, 70.3, 70.2, 70.2, 67.2, 53.9, 53.6, 52.3, 51.4, 51.1, 44.0, 43.1, 40.7, 30.1, 29.4, 29.1, 28.9, 28.0, 27.7, 27.7, 25.2, 25.0, 23.0.

Purification elution fractions were analyzed for product by ESI mass spectrometry in negative mode: HRMS (ESL) calc. for C₄₀H₅₆N₆O₂₅S₂ (M+Na+2H)⁻¹: 1109.2790; found: 1109.2798.

A solution of diantennary monomer S15 (8.7 mg, 0.008 mmol) in a degassed mixture of 2.5:1, 1,2-dichloroethane:2,2,2-trifluoroethanol (DCE:TFE) (1 mL) was transferred into an oven dried 10 mL Schlenk flask under N₂ (Solvent mixture was degassed in bulk by freeze-pump-thaw method prior to dissolving monomer. Degassing was repeated at least 5 times until bubbles subsided.). The mixture was then concentrated by rotary evaporation and placed in vacuo for 30 min. In a glove box under an inert N₂ atmosphere a 1 mL oven dried, conical Schlenk flask was charged with 4.6 mg of catalyst [(H₂IMes)(3-Br-py)₂(Cl)₂Ru═CHPh] (G3), then sealed with glass stopper and removed from the glove box. The G3 was then dissolved in 0.73 mL of degassed 2.5:1 DCE:TFE under N., to make a stock solution. Under N., monomer S15 was redissolved in the degassed 2.5:1 DCE:TFE (0.25 mL) mixture and a magnetic stir bar was added. 0.100 mL of the G3 stock solution was then rapidly injected to the monomer solution Schlenk under N. and then sealed with a glass stopper (final concentration=0.025 M). The resulting solution was then lowered into a 55° C. oil bath and allowed to stir. After the solution became cloudy (1 h) the conversion of the monomer was monitored by ¹H NMR of a reaction aliquot in CD₃OD by observing the disappearance of the strained alkene peak at 6.4 ppm. Upon full conversion the reaction was cooled to RT and stirred for 5 min. The reaction mixture was quenched with ethyl vinyl ether (5 drops) and allowed to stir for 30 min. After, the reaction mixture was then transferred into a 20 mL scintillation vial and concentrated in vacuo. The crude product was dissolved in a minimal amount of methanol and precipitated with an excess of diethyl ether. Precipitate was allowed to settle and the liquid was then decanted off. If the precipitant was very fine, this solution was centrifuged, and the diethyl ether layer was decanted. The precipitate was then re-dissolved in excess methanol (2 mL) and re-concentrated until the polymer was in a minimal amount of methanol. This process was repeated two more times. On the final precipitation the polymer was not re-dissolved in methanol and placed in vacuo to yield disulfated polymer S16 as an off white solid (8.5 mg, yield=98%, conversion=100%, DP=11), after polymerization.

The NMR results were: ¹H NMR (500 MHz, MeOD) δ 8.13 (s, 1H), 7.96 (s, 1H), 7.51-7.21 (m, 1H), 5.96 (s, 1H), 5.69 (s, 1H), 5.41 (s, 1H), 5.30 (s, 1H), 4.66 (d, J=43.9 Hz, 3H), 4.42 (s, 2H), 4.24 (s, 2H), 4.01 (s, 2H), 3.86 (d, J=34.4 Hz, 8H), 3.72 (d, J=46.4 Hz, 11H), 3.43 (s, 2H), 3.04 (s, 1H), 2.88 (s, 1H), 2.78-2.56 (m, 3H), 2.00 (s, 3H), 1.61 (s, 4H), 1.30 (s, 2H).

Disulfated polymer S16 (8.5 mg) was charged into a 20 mL vial along with 0.7 mL 0.25 M LiOH aqueous solution, 7.3 mL water, and 1.9 mL THF and allowed to stir at RT for 24 h. The reaction mixture was then frozen using liquid nitrogen and lyophilized to completion. Remaining solid was then dissolved in water and placed inside a dialysis cartridge (Slide-A-Lyzer G2 Dialysis Cassettes, 3.5K MWCO, 3 mL, Cat. #: 87723) and dialyzed against 0.9% NaCl solution for 24 h (3 buffer changes) then against DI water for 24 h (3 buffer changes). Finally, sample was transferred into a 5 mL vial and frozen by liquid nitrogen. The sample was then lyophilized to obtain fully deprotected disulfated polymer C5E as a white solid (5.3 mg, 67%), after saponification.

The NMR results were: ¹H NMR (500 MHz, D₂O) δ 7.89 (d, J=40.2 Hz, 1H), 7.27 (s, 1H), 5.91 (s, 2H), 5.31 (s, 1H), 5.01 (s, 1H), 4.54-4.20 (m, 6H), 4.12-3.93 (m, 3H), 3.86 (d, J=7.3 Hz, 4H), 3.63 (m, 7H), 3.25 (d, J=6.5 Hz, 5H), 3.08 (s, 1H), 2.60 (d, J=77.5 Hz, 5H), 1.93 (s, 3H), 1.45 (s, 4H), 1.15 (s, 2H).

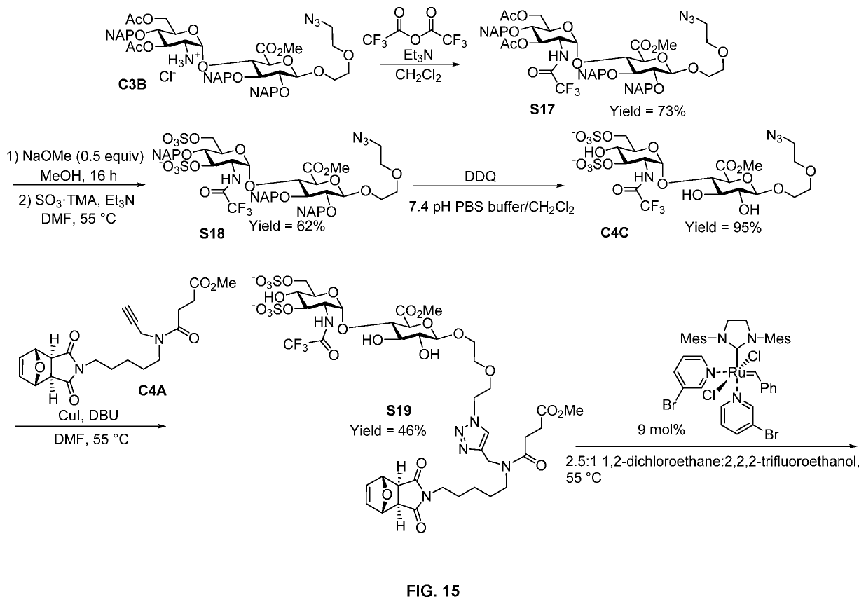

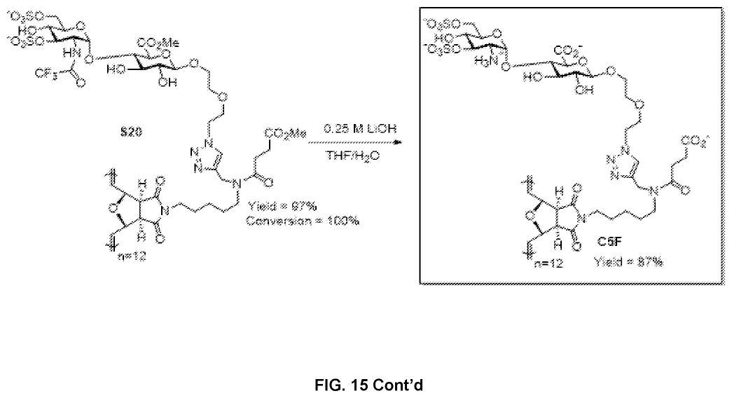

FIG. 15 shows the synthetic route for free amine disulfated glycopolymer CSF.

An oven dried 10 mL Schlenk flask was charged with a solution of disaccharide C3B (88 mg, 0.0892 mmol 1 equiv.) in anhydrous CH₂Cl₂ and subsequently charged with triethylamine (0.124 mL, 0.892 mmol, 10 equiv.), trifluoroacetic anhydride (0.0744 mL, 0.535 mmol, 6 equiv.), and a few crystals of 4-dimethylaminopyridine. The reaction was stirred at RT for 5 h, with monitoring by TLC (1:1 hexanes:ethyl acetate and 20:1 CH₂Cl₂:methanol). Upon completion, the reaction mixture was loaded directly on to a silica gel column and purified by flash chromatography (10 g of silica, ½in ID×12 in column, 4:143:142:142:1 hexanes:ethyl acetate). After purification, the fractions containing the product were combined and concentrated to provide the desired S17 (72 mg, 90%).

The NMR results were: ¹H NMR (400 MHz, CDCl₃) δ 7.84-7.76 (m, 5H), 7.74-7.68 (m, 4H), 7.65 (t, J=4.3 Hz, 2H), 7.55 (s, 1H), 7.51-7.47 (m, 2H), 7.47-7.41 (m, 4H), 7.36 (ddd, J=8.4, 5.1, 1.6 Hz, 2H), 7.24 (dd, J=8.5, 1.6 Hz, 1H), 6.95 (d, J=9.6 Hz, 1H), 5.38-5.30 (m, 2H), 5.09 (t, J=11.1 Hz, 2H), 4.81-4.67 (m, 4H), 4.63 (d, J=7.2 Hz, 1H), 4.31 (d, J=11.1 Hz, 1H), 4.26-4.09 (m, 3H), 4.05-3.98 (m, 1H), 3.97 (d, J=9.2 Hz, 1H), 3.83-3.62 (m, 9H), 3.59 (t, J=5.0 Hz, 2H), 3.27 (td, J=4.7, 1.2 Hz, 2H), 1.95 (s, 3H), 1.85 (s, 3H).

¹⁹F NMR (471 MHz, CDCl₃) δ −75.86.

Purification elution fractions were analyzed for product by ESI mass spectrometry: HRMS (ESI⁺) calc. for C₅₆H₅₇F₃N₄O₁₅ (M+Na): 1105.3665; found: 1105.3665.

A 10 mL oven dried Schlenk flask was charged with disaccharide S17 (70 mg, 0.0646 mmol, 1 equiv.) and anhydrous methanol (0.35 mL). NaOMe (1.75 mg, 0.0323 mmol, 1 equiv.) was added and stirred overnight at RT. The reaction was monitored for completion by TLC (1:2 hexanes:ethyl acetate). Upon completion, the reaction was diluted with CH₂Cl₂:methanol mixture and neutralized with Amberlyst® 15 hydrogen form (registered trademark of The Dow Chemical Company or an affiliated company of Dow), filtered, and concentrated.

An oven dried 10 mL Schlenk flask containing deacetylated crude was sequentially charged under N₂ with DMF (0.35 mL), SO₃.Me₃N (316 mg, 2.58 mmol, 40 equiv.), and triethylamine (0.182 mL, 1.29 mmol, 20 equiv.). The reaction mixture was stirred at 50° C. for 3 d. The reaction progress was monitored by ESI negative mode mass spectrometry. The white solid was filtered off using cotton plug washing with CH₂Cl₂. The reaction was then concentrated in vacuo. The residue was purified using C-18 reverse phase silica gel flash chromatography (0480% acetonitrile/water) to afford disulfated disaccharide S18 (46 mg, 62%).

The NMR results were: ¹H NMR (500 MHz, MeOD) δ 8.00 (s, 1H), 7.90-7.85 (m, 1H), 7.83-7.63 (m, 11H), 7.46-7.38 (m, 7H), 7.33 (dd, J=8.4, 1.3 Hz, 1H), 5.78 (d, J=3.2 Hz, 1H), 5.33 (d, J=9.8 Hz, 1H), 5.13 (d, J=11.5 Hz, 1H), 4.99 (d, J=11.2 Hz, 1H), 4.85-4.82 (m, 3H), 4.78 (d, =11.6 Hz, 2H), 4.72 (d, =7.6 Hz, 1H), 4.42 (dd, =10.6, 2.5 Hz, 1H), 4.20 (dd, =10.6, 1.6 Hz, 1H), 4.16 (d, J=9.4 Hz, 1H), 4.07 (t, J=9.0 Hz, 1H), 4.00-3.91 (m, 2H), 3.85-3.76 (m, 6H), 3.72 (d, J=9.8 Hz, 1H), 3.68-3.64 (m, 2H), 3.59 (t, J=5.0 Hz, 2H), 3.52 (d, J=8.0 Hz, 1H), 3.25-3.19 (m, 2H).

¹³C NMR (126 MHz, MeOD) δ 171.1, 137.4, 137.4, 137.2, 134.8, 134.7, 134.5, 134.4, 134.4, 129.2, 129.1, 128.9, 128.9, 128.8, 128.7, 128.6, 128.6, 128.5, 128.5, 127.6, 127.5, 127.2, 127.1, 127.0, 126.8, 126.8, 126.7, 126.7, 126.7, 104.8, 96.6, 84.5, 82.9, 77.5, 77.0, 76.3, 76.1, 75.8, 75.4, 72.2, 71.4, 71.0, 70.2, 66.7, 56.1, 53.6, 51.7.

¹⁹F NMR (471 MHz, MeOD) δ −76.98.

Purification elution fractions were analyzed for product by ESI mass spectrometry in negative mode: HRMS (ESL) calc. for C₅₂H₅₁F₃N₄O₁₉S₂(M+Na)⁻¹: 1179.2438; found:1179.2438.

A 5 mL vial was charged with 2-naphthylmethyl protected disulfated disaccharide S18 (23 mg, 0.02 mmol, 1 equiv.), CH₂Cl₂ (0.3 mL), pH 7.4 1×PBS buffer (0.3 mL) and recrystallized 2,3-dichloro-5,6-dicyano-1,4-benzoquinone (36 mg, 0.12 mmol, 8 equiv.). An oversized stir bar was added and the vial was wrapped in aluminum foil. The biphasic reaction mixture was vigorously stirred overnight at RT. Reaction completion was monitored by disappearance of the starting material by ESI mass spectrometry in negative mode. Upon completion the reaction mixture was directly loaded onto a brand new 24 g Redisep Rf Gold column using minimal methanol and purified by silica gel flash chromatography on a Teledyne ISCO Flash Purification System (A-CH₂Cl₂ B-Methanol 0→5% B over 3 CV then 5→40% B over 20 CV) to afford the disaccharide C4C (14 mg, 95%), after napthyl deprotection.

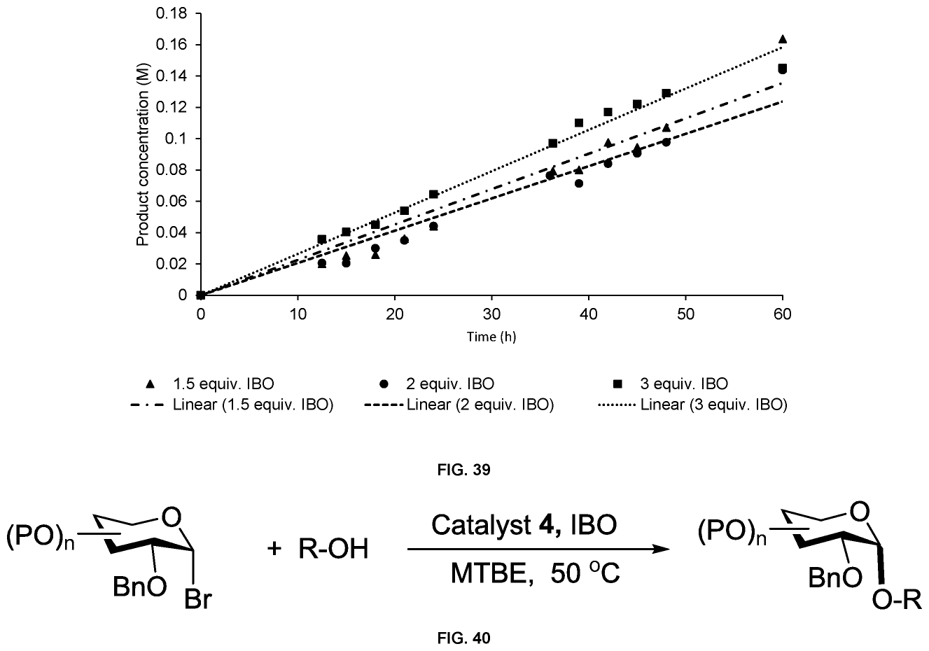

The NMR results were: ¹H NMR (400 MHz, MeOD) δ 5.38 (d, J=3.5 Hz, 1H), 4.55-4.48 (m, 1H), 4.42 (d, J=7.8 Hz, 1H), 4.28-4.17 (m, 2H), 4.04-3.96 (m, 2H), 3.96-3.90 (m, 1H), 3.85-3.66 (m, 12H), 3.59 (t, J=9.1 Hz, 1H), 3.43-3.36 (m, 2H), 3.26 (dd, J=9.3, 7.8 Hz, 2H) (FIG. 39A).

¹³C NMR (101 MHz, MeOD) δ 170.6, 104.6, 99.2, 80.2, 78.5, 76.8, 76.0, 74.9, 72.8, 71.3, 71.0, 70.2, 69.8, 67.0, 54.8, 53.6, 51.8, 9.2.

Purification elution fractions were analyzed for product by ESI mass spectrometry in negative mode: HRMS (ESL) calc. for C₁₉H₂₇F₃N₄O₁₉S₂ (M+Na)⁻¹: 759.0560; found: 759.0559.

An oven dried 10 mL Schlenk flask was charged with a solution of polymerizable scaffold C4A (10 mg, 0.025 mmol 1.2 equiv.) in CH₂Cl₂ and a solution of deprotected disulfated disaccharide 4C (15.3 mg, 0.021 mmol, 1 equiv.) in methanol. The mixture was then concentrated by rotary evaporation and placed in vacuo for 30 min. Under N₂, copper (I) iodide (3.9 mg, 0.021 mmol, 1 equiv.) was added followed by anhydrous DMF (0.25 mL). Lastly the addition of DBU (4 μL, 0.025 mmol, 1.2 equiv.) was performed by a microsyringe. The resulting mixture was stirred overnight at 55° C. The reaction mixture was monitored by ESI mass spectrometry in negative mode for complete consumption of 4C. Upon completion, the reaction mixture was directly loaded onto a brand new 12 g Redisep Rf Gold column using minimal methanol and purified by silica gel flash chromatography on a Teledyne ISCO Flash Purification System (A-CH₂Cl₂ B-Methanol 0→50% B over 20 CV) to afford the diantennary glycomonomer S19 (9.5 mg, 46%), after click reaction.

The NMR results were: ¹H NMR (500 MHz, MeOD) δ 8.11 (s, 1H), 7.94 (s, 1H), 6.48 (d, J=7.6 Hz, 2H), 5.41 (d, J=2.4 Hz, 1H), 5.38-5.30 (m, 2H), 5.05 (s, 1H), 4.70 (s, 1H), 4.64-4.50 (m, 4H), 4.40 (dd, J=7.8, 2.8 Hz, 1H), 4.29-4.19 (m, 2H), 4.01 (dd, J=20.4, 6.3 Hz, 2H), 3.92-3.77 (m, 7H), 3.78-3.57 (m, 10H), 3.42 (dd, J=18.9, 11.1 Hz, 4H), 3.27 (t, J=8.7 Hz, 1H), 3.14 (ddd, J=23.1, 13.6, 6.7 Hz, 1H), 2.87 (dd, J=8.0, 4.6 Hz, 1H), 2.73 (d, J=6.3 Hz, 1H), 2.71-2.62 (m, 3H), 2.58 (t, J=7.5 Hz, 1H), 1.71-1.50 (m, 4H), 1.38-1.25 (m, 2H).

¹³C NMR (126 MHz, MeOD) δ 175.3, 174.5, 174.4, 173.8, 173.8, 170.6, 138.0, 137.7, 104.7, 99.1, 81.9, 81.8, 81.2, 80.1, 80.1, 78.5, 76.9, 76.0, 74.9, 72.9, 71.2, 70.3, 69.9, 67.1, 54.8, 53.7, 52.6, 52.2, 51.4, 51.4, 51.1, 47.2, 44.0, 43.1, 42.0, 40.7, 30.1, 29.4, 29.1, 28.9, 28.0, 27.7, 27.7, 25.2, 25.1.

¹⁹F NMR (471 MHz, MeOD) δ −76.97.

Purification elution fractions were analyzed for product by ESI mass spectrometry in negative mode: HRMS (ESI⁻) calc. for C₄₀H₅₃F₃N₆O₂₅S₂(M+Na+2H)⁻¹: 1163.2508; found: 1163.2489.

Into an oven dried 10 mL Schlenk flask under N₂ a solution of diantennary monomer S19 (9.5 mg, 0.008 mmol) in a degassed mixture of 2.5:1 1,2-dichloroethane:2,2,2-trifluoroethanol (DCE:TFE) (1 mL) was transferred in. (Solvent mixture was degassed in bulk by freeze-pump-thaw method prior to dissolving monomer. Degassing was repeated at least 5 times until bubbles subsided.) The mixture was then concentrated by rotary evaporation and placed in vacuo for 30 min. In a glove box under an inert N₂ atmosphere a 1 mL oven dried, conical Schlenk flask was charged with 4.6 mg of catalyst [(H₂IMes)(3-Br-py)₂(Cl)₂Ru═CHPh] (G3), then sealed with glass stopper and removed from the glove box. The G3 was then dissolved in 0.77 mL of degassed 2.5:1 DCE:TFE under N₂, to make a stock solution. Under N₂, monomer S19 was re-dissolved in the degassed 2.5:1 DCE:TFE (0.25 mL) mixture and a magnetic stir bar was added. 0.100 mL of the G3 stock solution was then rapidly injected to the monomer solution Schlenk under N₂ and then sealed with a glass stopper (final concentration=0.025 M). The resulting solution was then lowered into a 55° C. oil bath and allowed to stir. After the solution became cloudy (1 h) the conversion of the monomer was monitored by ¹H NMR of a reaction aliquot in CD₃OD by observing the disappearance of the strained alkene peak at 6.4 ppm. Upon full conversion the reaction was cooled to RT and stirred for 5 min. The reaction mixture was quenched with ethyl vinyl ether (5 drops) and allowed to stir for 30 min. The reaction mixture was then transferred into a 20 mL scintillation vial and concentrated in vacuo. The crude product was dissolved in a minimal amount of methanol and precipitated with an excess of diethyl ether. Precipitate was allowed to settle and the liquid was then decanted off. Note: If the precipitant was very fine, this solution was centrifuged, and the liquid was decanted. The precipitate was then re-dissolved in excess methanol (2 mL) and re-concentrated until the polymer was in a minimal amount of methanol. This process was repeated two more times. The final residual precipitate was dried in vacuo to yield disulfated polymer S20 as an off white solid (9.3 mg, yield=97%, conversion=100%, DP=12), after polymerization.

The NMR results were: ¹H NMR (500 MHz, MeOD) δ 8.13 (s, 1H), 7.96 (s, 1H), 7.51-7.16 (m, 1H), 5.95 (s, 1H), 5.69 (s, 1H), 5.40 (s, 1H), 4.56 (m, 5H), 4.41 (s, 1H), 4.23 (dd, J=18.7, 9.9 Hz, 2H), 4.01 (d, J=11.6 Hz, 2H), 3.87 (d, J=9.2 Hz, 3H), 3.83 (s, 5H), 3.42 (s, 1H), 3.01 (s, 1H), 2.87 (s, 1H), 2.75 (s, 1H), 2.65 (s, 2H), 1.61 (m, 4H), 1.26 (m, 2H).

¹⁹F NMR (471 MHz, MeOD) δ −76.72.

Disulfated polymer S20 (9.3 mg) was charged into a 20 mL vial along with 0.76 mL 0.25 M LiOH aqueous solution, 7.98 mL water, and 2.1 mL THF and allowed to stir at RT for 24 h. The reaction mixture was then frozen using liquid nitrogen and lyophilized to completion. Remaining solid was then dissolved in water and placed inside a dialysis cartridge (Slide-A-Lyzer G2 Dialysis Cassettes, 3.5K MWCO, 3 mL, Cat. #: 87723) and dialyzed against 0.9% NaCl solution for 24 h (3 buffer changes) then against DI water for 24 h (3 buffer changes). Finally, sample was transferred into a 5 mL vial and frozen by liquid nitrogen. The sample was then lyophilized to obtain fully deprotected disulfated polymer C5F as a white solid (7.8 mg, 87%), after saponification.

The NMR results were: ¹H NMR (500 MHz, D₂O) δ 7.99 (s, 1H), 7.84 (s, 1H), 7.34 (s, 1H), 5.92 (s, 1H), 5.63 (s, 1H), 5.01 (s, 1H), 4.55-4.39 (m, 2H), 4.39-4.23 (m, 2H), 4.11 (d, J=10.7 Hz, 1H), 3.95-3.79 (m, 4H), 3.70 (dd, J=22.1, 12.8 Hz, 6H), 3.61-3.48 (m, 4H), 3.37-2.95 (m, 6H), 2.75-2.34 (m, 4H), 1.41 (s, 4H), 1.12 (s, 2H).

¹⁹F NMR (471 MHz, MeOD) δ No resonance.

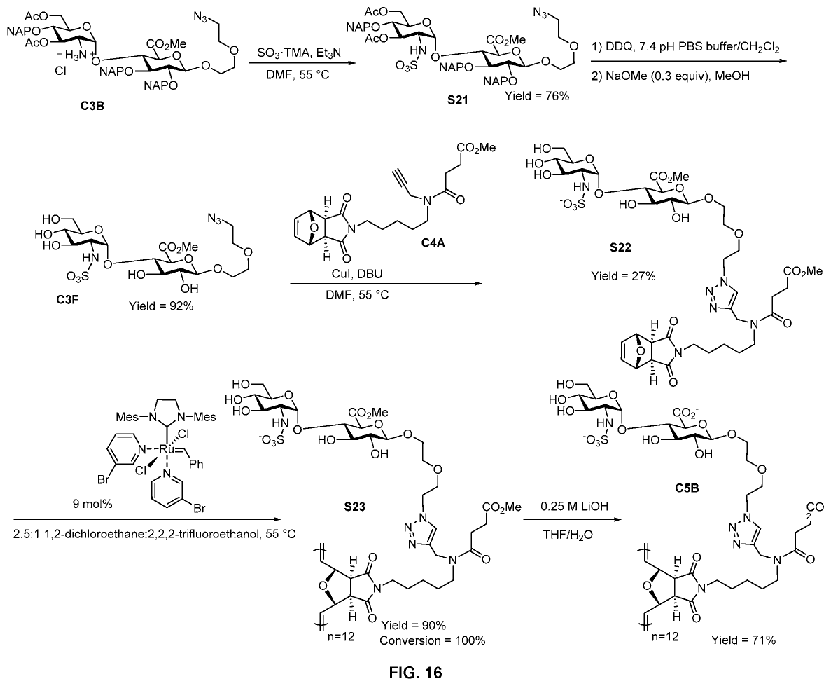

FIG. 16 shows the synthetic route for N-sulfated glycopolymer C5B.

To 10 mL oven dried Schlenk flask containing C3B (47.5 mg, 0.05 mmol, 1 equiv.) was sequentially charged with anhydrous DMF (0.2 mL), SO₃.Me₃N (70 mg, 0.5 mmol, 10 equiv.), and triethylamine (0.07 mL, 1.5 mmol, 30 equiv.) under nitrogen. The reaction mixture was stirred at 55° C. for 3 d. The reaction progress was monitored by ESI negative mode mass spectrometry. The white solid was filtered off using cotton plug washing with CH₂Cl₂. The reaction was then concentrated in vacuo. The residue was purified using C-18 reverse phase silica gel flash chromatography (0→80% acetonitrile/water) to afford S21 (40 mg, 76%), after sulfation.

The NMR results were: ¹H NMR (500 MHz, MeOD) δ 7.86-7.69 (m, 11H), 7.60 (d, J=8.0 Hz, 1H), 7.49-7.36 (m, 9H), 5.62 (d, J=3.4 Hz, 1H), 5.31 (dd, J=10.7, 9.2 Hz, 1H), 5.14 (d, J=11.4 Hz, 2H), 4.95 (d, J=11.3 Hz, 1H), 4.83-4.73 (m, 3H), 4.71 (d, J=7.5 Hz, 1H), 4.38 (dd, J=11.9, 1.8 Hz, 1H), 4.20-4.07 (m, 4H), 3.99-3.93 (m, 1H), 3.91-3.85 (m, 1H), 3.82-3.73 (m, 5H), 3.71 (dd, J=11.0, 8.1 Hz, 1H), 3.66-3.62 (m, 2H), 3.60-3.50 (m, 3H), 3.46 (dd, J=10.8, 3.4 Hz, 1H), 3.22 (dd, J=5.4, 4.3 Hz, 2H), 2.04 (s, 3H), 1.87 (s, 3H).

¹³C NMR (126 MHz, MeOD) δ 173.1, 172.5, 170.9, 137.5, 137.3, 136.8, 134.7, 134.5, 134.4, 134.3, 129.2, 129.1, 129.0, 128.9, 128.9, 128.7, 128.7, 128.7, 128.5, 127.9, 127.9, 127.7, 127.5, 127.3, 127.3, 127.2, 127.1, 127.0, 126.9, 126.7, 126.6, 104.8, 99.4, 83.1, 82.8, 77.2, 77.0, 75.9, 75.5, 75.3, 75.1, 74.4, 71.3, 71.2, 70.9, 70.2, 63.6, 58.3, 53.3, 51.7, 21.5, 20.6.

Purification elution fractions were analyzed for product by ESI mass spectrometry in negative mode: HRMS (ESI) calc. for C₅₄H₅₁N₄O₁₇S (M)⁻¹: 1065.3439; found: 1065.3426.

A 5 mL vial was charged with 2-naphthylmethyl protected disulfated disaccharide S21 (38 mg, 0.034 mmol, 1 equiv.), CH₂Cl₂ (0.5 mL), pH 7.4 1×PBS buffer (0.5 mL) and recrystallized 2,3-dichloro-5,6-dicyano-1,4-benzoquinone (47 mg, 0.21 mmol, 6 equiv.). An oversized stir bar was added and the vial was wrapped in aluminum foil. The biphasic reaction mixture was vigorously stirred overnight at RT. Reaction completion was monitored by disappearance of the starting material by ESI mass spectrometry in negative mode. Upon completion the reaction mixture was directly loaded onto a brand new 24 g Redisep Rf Gold column using minimal methanol and purified by silica gel flash chromatography on a Teledyne ISCO Flash Purification System (A-CH₂Cl₂ B-Methanol 0→5% B over 3 CV then 5→50% B over 20 CV) to afford the disaccharide C3F (17.5 mg, 92%).

¹H NMR (500 MHz, MeOD) δ 5.45 (d, J=3.6 Hz, 1H), 4.38 (d, J=7.8 Hz, 1H), 3.93 (d, J=9.6 Hz, 1H), 3.88 (dt, J=8.7, 4.1 Hz, 1H), 3.82 (t, J=9.2 Hz, 1H), 3.76-3.60 (m, 11H), 3.43 (dt, J=18.2, 8.8 Hz, 2H), 3.39-3.31 (m, 3H), 3.20-3.11 (m, 2H).

¹³C NMR (126 MHz, D₂O) δ 170.4, 102.6, 98.2, 77.1, 75.4, 74.3, 72.7, 72.4, 71.2, 69.7, 69.4, 69.2, 69.2, 60.0, 58.0, 53.5, 50.3.

Purification elution fractions were analyzed for product by ESI mass spectrometry in negative mode: HRMS (ESI⁻) calc. for C₁₇H₂₉N₄O₁₅S (M)⁻¹: 561.1356; found: 561.1356.

An oven dried 10 mL Schlenk flask was charged with a solution of polymerizable scaffold C4A (15 mg, 0.037 mmol 1.2 equiv.) in CH₂Cl₂ and a solution of deprotected disulfated disaccharide C3F (17.5 mg, 0.031 mmol, 1 equiv.) in methanol. The mixture was then concentrated by rotary evaporation and placed in vacuo for 30 min. Under N₂, copper (I) iodide (5.9 mg, 0.031 mmol, 1 equiv.) was added followed by anhydrous DMF (0.25 mL). Lastly the addition of DBU (5.4 μL, 0.037 mmol, 1.2 equiv.) was performed by a microsyringe. The resulting mixture was stirred overnight at 55° C. The reaction mixture was monitored by ESI mass spectrometry in negative mode for complete consumption of (C3F). Upon completion, the reaction mixture was directly loaded onto a brand new 12 g Redisep Rf Gold column using minimal methanol and purified by silica gel flash chromatography on a Teledyne ISCO Flash Purification System (A-CH₂Cl₂ B-Methanol 0→50% B over 20 CV) to afford the diantennary glycomonomer S22 (8 mg, 27%).

¹H NMR (500 MHz, MeOD) δ 8.13 (s, 1H), 7.93 (s, 1H), 6.48 (t, J=7.4 Hz, 2H), 5.55 (d, J=3.7 Hz, 1H), 5.38-5.28 (m, 2H), 5.05 (s, 1H), 4.70 (d, J=8.2 Hz, 1H), 4.65-4.52 (m, 3H), 4.40 (dd, J=7.8, 3.5 Hz, 1H), 3.99 (d, J=9.6 Hz, 1H), 3.88 (dt, J=11.7, 7.7 Hz, 4H), 3.78 (s, 3H), 3.74-3.61 (m, 10H), 3.54-3.37 (m, 6H), 3.29-3.21 (m, 2H), 3.20-3.06 (m, 1H), 2.87 (t, J=6.3 Hz, 1H), 2.73 (d, J=6.0 Hz, 2H), 2.69-2.62 (m, 3H), 2.57 (t, J=7.5 Hz, 1H), 1.72-1.49 (m, 4H), 1.30 (s, 2H).

¹³C NMR (126 MHz, MeOD) δ 175.3, 175.3, 174.5, 174.4, 173.9, 173.8, 170.7, 138.0, 137.7, 104.7, 100.2, 100.2, 83.6, 82.0, 81.8, 81.7, 81.2, 80.1, 79.4, 77.3, 76.0, 74.5, 73.8, 73.4, 71.5, 71.3, 70.3, 70.2, 62.1, 59.9, 53.2, 52.3, 52.2, 51.5, 51.4, 51.1, 50.3, 49.8, 47.1, 44.0, 43.1, 42.0, 40.7, 30.7, 30.12, 29.4, 29.1, 28.9, 28.0, 27.8, 27.7, 25.2, 25.1.

HRMS (ESL) calc. for C₃₈H₅₅N₆O₂₁S (M+2H)¹: 965.3297; found: 965.3303.

Purification elution fractions were analyzed for product by ESI mass spectrometry in negative mode. Into an oven dried 10 mL Schlenk flask under N₂ a solution of diantennary monomer S22 (8 mg, 0.008 mmol) in a degassed mixture of 2.5:1 1,2-dichloroethane:2,2,2-trifluoroethanol (DCE:TFE) (1 mL) was transferred in. (Note: Solvent mixture was degassed in bulk by freeze-pump-thaw method prior to dissolving monomer. Degassing was repeated at least 5 times until bubbles subsided.) The mixture was then concentrated by rotary evaporation and placed in vacuo for 30 min. In a glove box under an inert N₂ atmosphere a 1 mL oven dried, conical Schlenk flask was charged with 4.6 mg of catalyst [(H₂IMes)(3-Br-py)₂(Cl)₂Ru═CHPh] (G3), then sealed with glass stopper and removed from the glove box. The G3 was then dissolved in 0.692 mL of degassed 2.5:1 DCE:TFE under N₂, to make a stock solution. Under N₂, monomer S22 was redissolved in the degassed 2.5:1 DCE:TFE (0.23 mL) mixture and a magnetic stir bar was added. 0.100 mL of the G3 stock solution was then rapidly injected to the monomer solution Schlenk under N₂ and then sealed with a glass stopper (final concentration=0.025 M). The resulting solution was then lowered into a 55° C. oil bath and allowed to stir. After the solution became cloudy (1 h) the conversion of the monomer was monitored by ¹H NMR of a reaction aliquot in CD₃OD by observing the disappearance of the strained alkene peak at 6.4 ppm. Upon full conversion the reaction was cooled to RT and stirred for 5 min. The reaction mixture was quenched with ethyl vinyl ether (5 drops) and allowed to stir for 30 min. After, the reaction mixture was then transferred into a 20 mL scintillation vial and concentrated in vacuo. The crude product was dissolved in a minimal amount of methanol and precipitated with an excess of diethyl ether. Precipitate was allowed to settle and the liquid was then decanted off. If the precipitant was very fine, this solution was centrifuged, and the liquid was decanted. The precipitate was then redissolved in excess methanol (2 mL) and reconcentrated until the polymer was in a minimal amount of methanol. This process was repeated two more times. The final residual precipitate dried in vacuo to yield disulfated polymer S23 as an off white solid (7.2 mg, yield=90%, conversion=100%, DP=12), after polymerization.

The NMR results were: ¹H NMR (500 MHz, D₂O) δ 8.07 (s, 1H), 7.90 (s, 1H), 7.50-7.20 (m, 1H), 5.94 (s, 1H), 5.74 (s, 1H), 5.53 (s, 1H), 4.59 (s, 3H), 4.52 (d, J=7.6 Hz, 1H), 4.13 (d, J=7.5 Hz, 1H), 3.98-3.86 (m, 4H), 3.83-3.70 (m, 7H), 3.65 (s, 5H), 3.58-3.48 (m, 2H), 3.46-3.31 (m, 4H), 3.26-3.03 (m, 2H), 2.84-2.54 (m, 4H), 1.70-1.38 (m, 4H), 1.26 (s, 2H).

Disulfated polymer S23 (7.6 mg) was charged into a 20 mL vial along with 0.63 mL 0.25 M LiOH aqueous solution, 6.6 mL water, and 1.7 mL THF and allowed to stir at RT for 24 h. The reaction mixture was then frozen using liquid nitrogen and lyophilized to completion. Remaining solid was then dissolved in water and placed inside a dialysis cartridge (Slide-A-Lyzer G2 Dialysis Cassettes, 3.5K MWCO, 3 mL, Cat. #: 87723) and dialyzed against 0.9% NaCl solution for 24 h (3 buffer changes) then against DI water for 24 h (3 buffer changes). Finally, sample was transferred into a 5 mL vial and frozen by liquid nitrogen. The sample was then lyophilized to obtain fully deprotected disulfated polymer C5B as a white solid (5.4 mg, 71%), after saponification.

The NMR results were: ¹H NMR (500 MHz, D₂O) δ 7.93 (s, 1H), 7.76 (s, 1H), 7.21 (s, 1H), 5.93-5.52 (m, 2H), 5.48 (s, 1H), 4.99 (s, 1H), 4.45 (s, 2H), 4.32 (s, 1H), 3.81 (s, 3H), 3.75-3.38 (m, 11H), 3.36-3.16 (m, 5H), 3.11-2.91 (m, 2H), 2.70-2.40 (m, 4H), 1.45 (s, 4H), 1.10 (s, 2H).

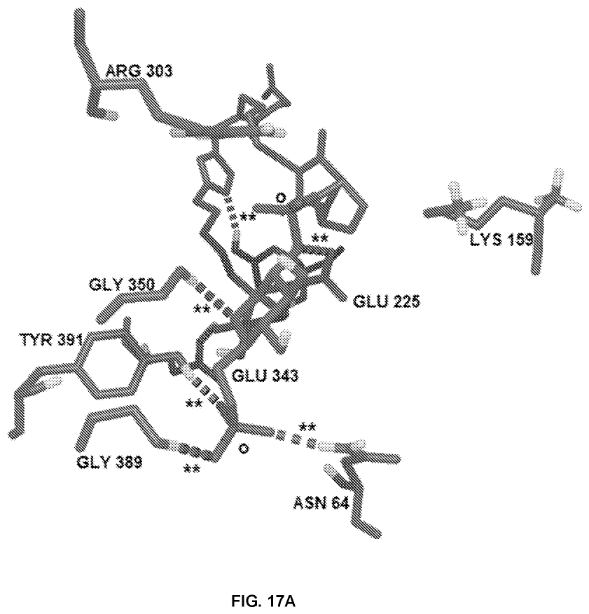

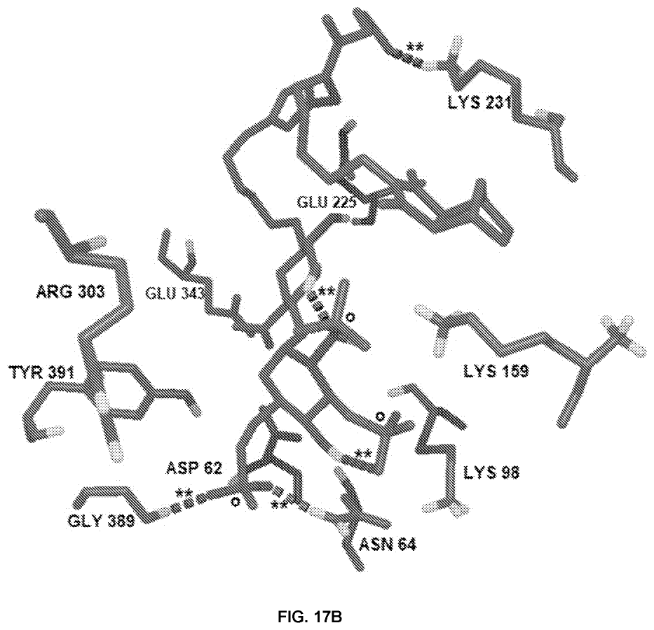

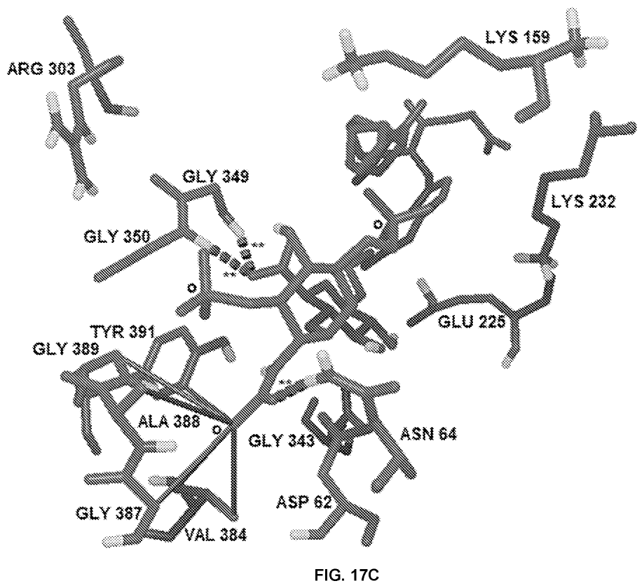

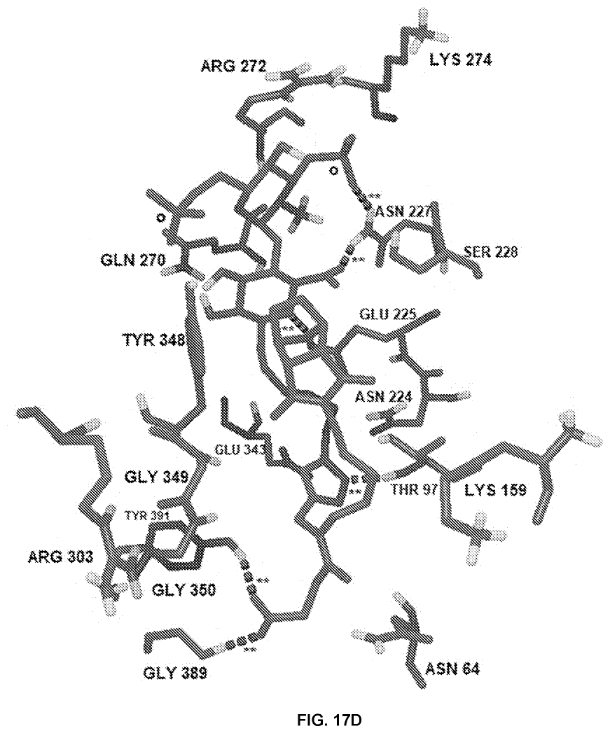

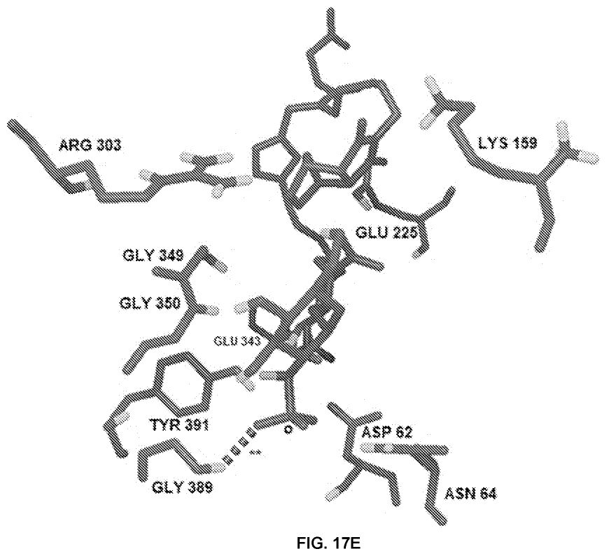

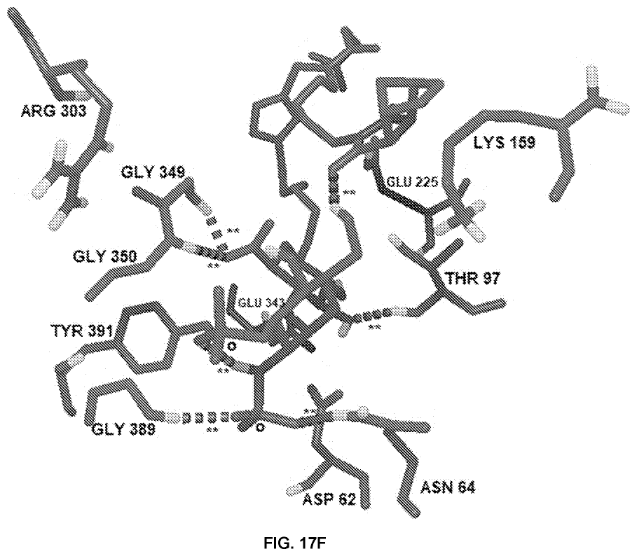

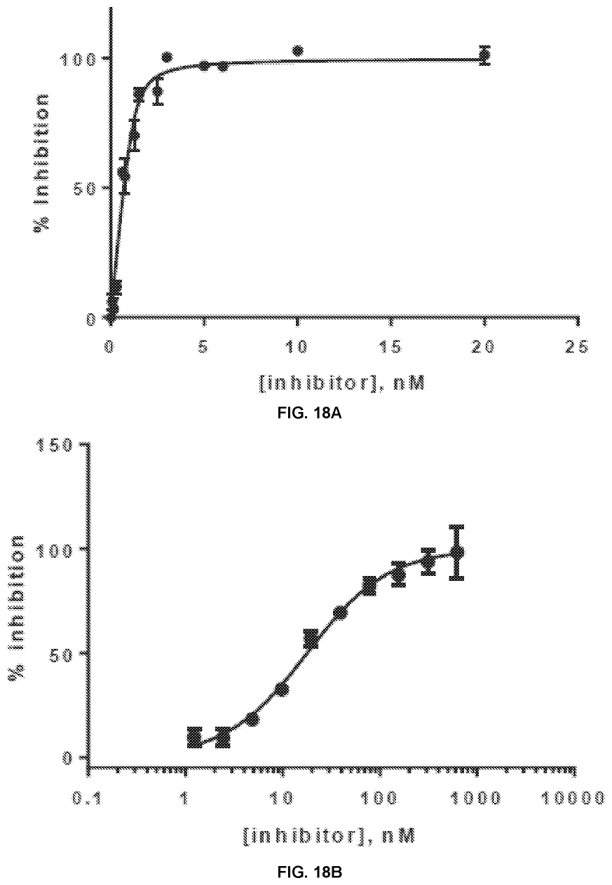

Computational docking study. FIGS. 17A-17F show the computational docking study. For the docking studies, the disclosure used the apo heparanase structure (PDB code: 5E8M) (Wu, et al., Nat. Struct. Mol. Biol. 2015, 22, 1016-1022.). The enzyme structure was imported into Yasara (Krieger, et al., Bioinformatics 2014, 30, 2981-2982.), cleaned, energy minimized in vacuo, and Glu225 was manually protonated. Ligands were constructed in a two-step method. The saccharide portion was first built using the Glycam GAGs builder (Glycam.org. (2019). Available at: http://glycam.org/ [Accessed 29 Oct. 2019]) and then imported into the Avagadro molecular editing software (Avogadro. (2019). Available at: https://avogadro.cc/ [Accessed 29 Oct. 2019]) where the aliphatic portion was added. The ligand was then subjected to a steepest descent energy minimization and saved in the .pdb format. Global docking with each ligand was performed on the heparanase structure separately using the Autodock VINA default parameters in a simulation cell set built at least 10 Å from all the three sides of the enzyme. The set-up was done with the YASARA molecular modeling program (Yasara.org. (2019). Available at: http://www.yasara.org/ [Accessed 29 Oct. 2019].) and the built-in docking simulation macro ‘dock_run.mrc’ for 100 docking runs using the AMBER14 force field for protein (D. A. Case, et al., 2014, AMBER 14, University of California, San Francisco.) and GLYCAM06 (Kirschner, et al., J. Comput. Chem. 2007, 29, 622-655.) and GAFF/Am1BCC for the synthetic saccharide ligand and a pose cluster RMSD of 5 Å for the docking conformations. Ligands and receptor residues were kept flexible during the docking runs. The most populated clusters of the 100 docking runs were subjected to further analysis. Hydrogen bonds are designated with double asterisks (**). Hydrophobic interactions are designated with the letter “o” (see FIGS. 17A-17F).

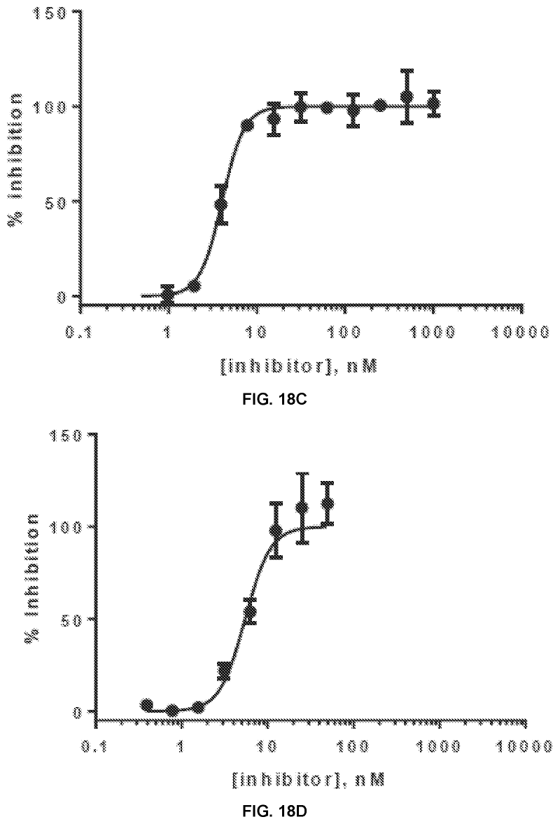

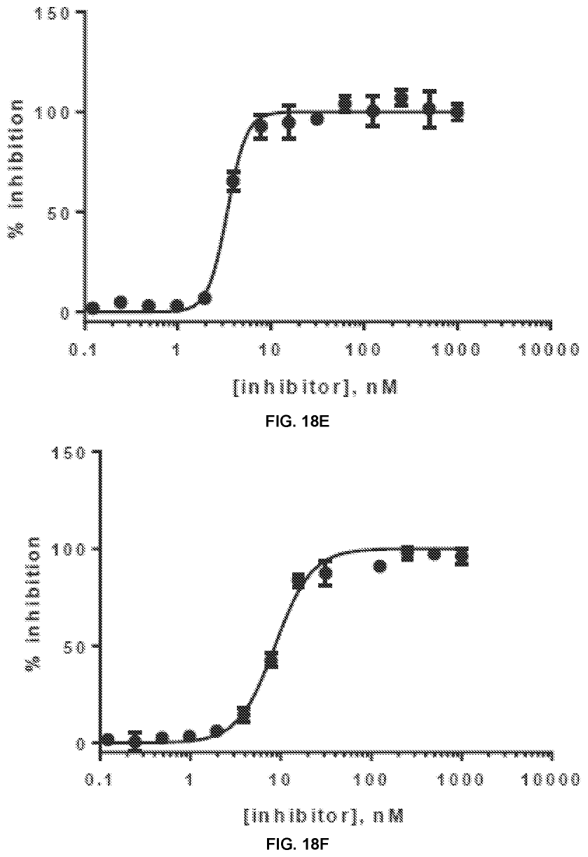

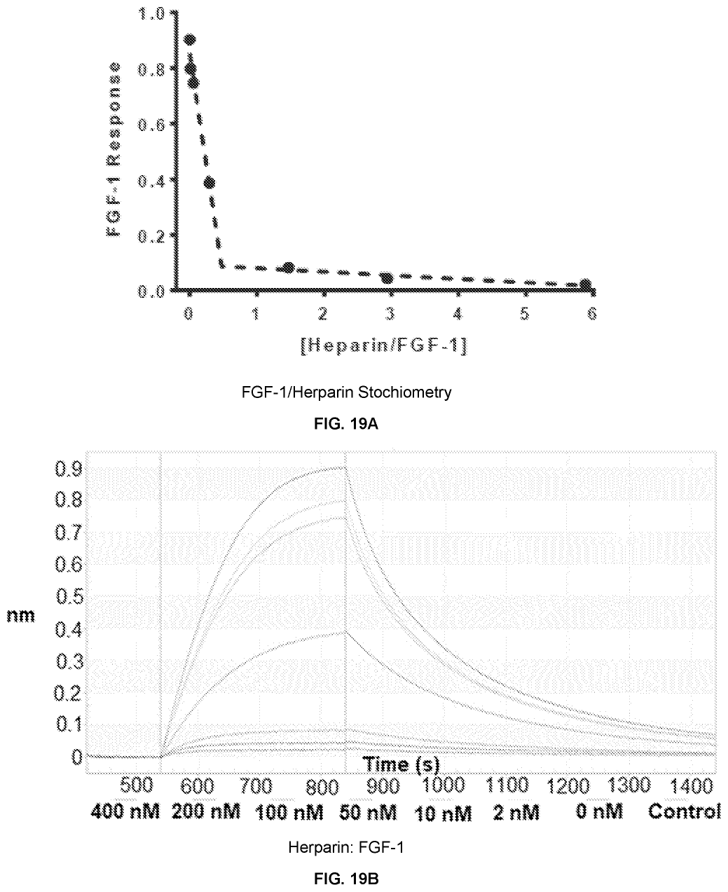

Biological assay protocols. Critical Micelle Concentration (CMC) Protocol (Kalyanasundaram, et al., J. Phys. Chem. 1977, 81, 2176-2180.): FIG. 18 show the inhibition of heparanase by polymers of different sulfation patterns. (A) shows the inhibition of heparanase by C(6)-SO₃ N—SO₃ disulfated glycopolymer C5A. (B) shows the inhibition of heparanase by N-sulfated glycopolymer C5B. (C) shows the inhibition of heparanase by C(3)-SO₃ N—SO₃ disulfated glycopolymer C5C. (D) shows the inhibition of heparanase by trisulfated glycopolymer C5D. (E) shows the inhibition of heparanase by N-acetylated disulfated glycopolymer C5E. (F) shows the inhibition of heparanase by free amine disulfated glycopolymer C5F.

Fluorescence measurements were performed in an Aligent Technologies Cary Eclipse Fluorescence Spectrophotometer. A 15 μM stock solution of pyrene was formed in a 15:85 methanol:water mixture. A stock solution of C(6)-SO₃ N—SO₃ polymer C5A was serially diluted in 1.5 mL Eppendorf tubes to a volume of 420 μL at 16 different concentrations with deionized water from 0 to 1 mg/mL. To each tube 30 μL of the pyrene stock solution were added to bring the final pyrene concentration to 1 μM and a methanol concentration of <1%. Tubes were then covered in aluminum foil and mechanically agitated by an orbital shaker for 2 h and then allowed to equilibrate for 18 h. Fluorescence emission spectra of the polymer solutions containing pyrene were recorded in a 400 μL microcuvette using an excitation wavelength of 335 nm, and the intensities I₁ and I₃ were measured at the wavelengths corresponding to the first and third vibronic bands located near 373 (I₁) and 384 (I₃) nm. A 2.5 nm slit width was used for both excitation and emission. All fluorescence measurements were carried out at 25.0° C. The average ratio of I₁/I₃ for three trials was plotted against the concentration of each polymeric sample using GraphPad Prism 7. The CMC was taken at the intersection of two calculated regression lines.

TR-FRET Heparanase Inhibition Assay (Roy, et al., J. Med. Chem. 2014, 57, 4511-4520.). 42 μl of inhibitor solution in Milli-Q water (0.00016-4000 μM) or just Milli-Q water (as a control), and 42 μl of heparanase (5.3 nM, R&D Systems) solution in pH 7.5 triz buffer (consisting of 20 mM TrisHCl, 0.15 M NaCl and 0.1% CHAPS) or just buffer as blank were added into microtubes and pre-incubated at 37° C. for 10 min bringing the [heparanase] to 0.5 nM. Next, 84 μl of biotin-heparan sulfate-Eu cryptate (Cisbio, Cat #: 61BHSKAA) (58.6 ng in pH 5.5 0.2 M NaCH₃CO₂ buffer) was added to the microtubes, and the resulting mixture was incubated for 60 min at 37° C. The reaction mixture was stopped by adding 168 μl of Streptavidin-XLent! (Cisbio, Cat #: 611SAXLA) (1.0 μg/ml) solution in pH 7.5 dilution buffer made of 0.1 M NaPO₄, 0.8 M KF, 0.1% BSA. After the mixture had been stirring at RT for 15 min, 100 μL (per well) of the reaction mixture was transferred to a 96 well microplate (Corning #3693 96 well, white polystyrene, half-area) in triplicates and HTRF emissions at 616 nm and 665 nm were measured by exciting at 340 nm using a SpectraMax i3x Microplate Reader (Molecular Devices). Due to the IC₅₀ value being the same as the concentration of heparanase in the reaction, glycopolymer C5A had to be fit to a Henderson Tight-Binding Equation:

$$\% Inibition = 100 \frac{{E}_{I}}{{E}_{T}} = 50 ( \frac{{E}_{T} + {K}_{D} + {I}_{o} - \sqrt{{K}_{D}^{2} + {E}_{T}^{2} + {I}_{o}^{2} + 2 {E}_{T} {K}_{D} + 2 {K}_{D} {I}_{o} - 2 {E}_{T} {I}_{o}}}{{E}_{T}} )$$

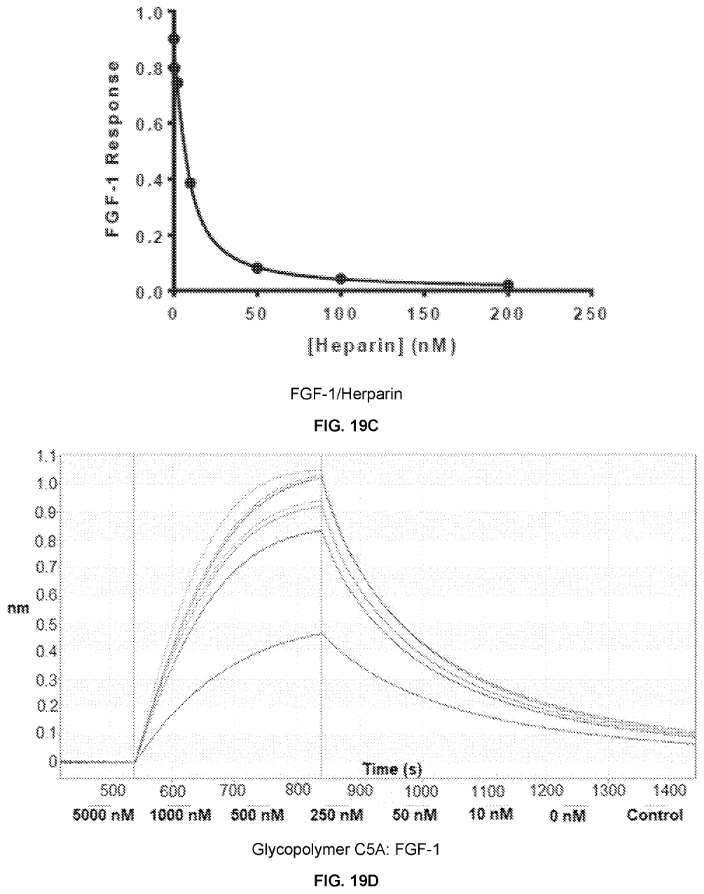

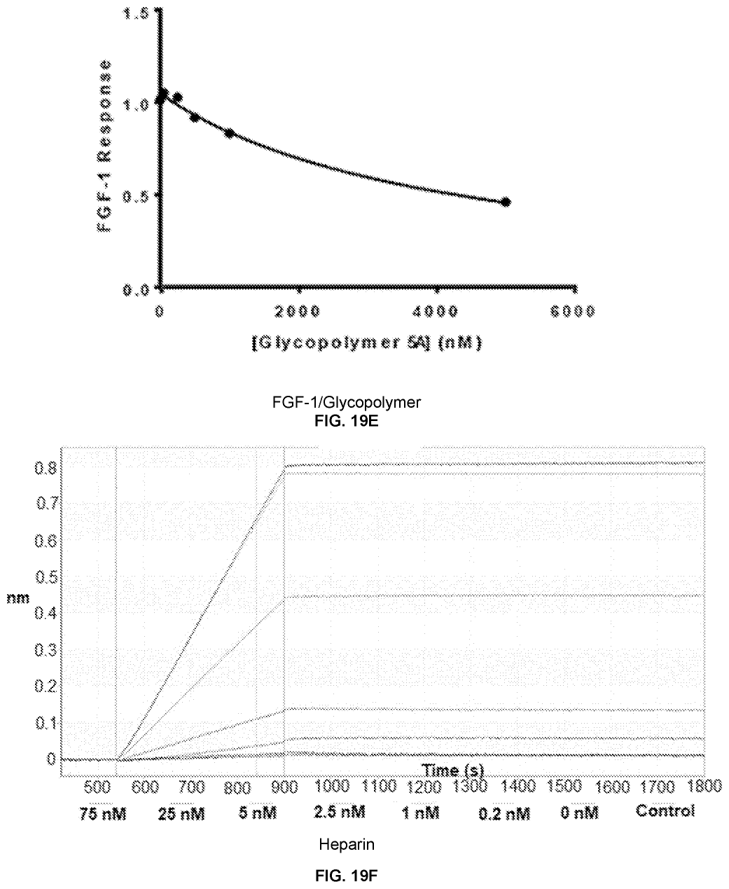

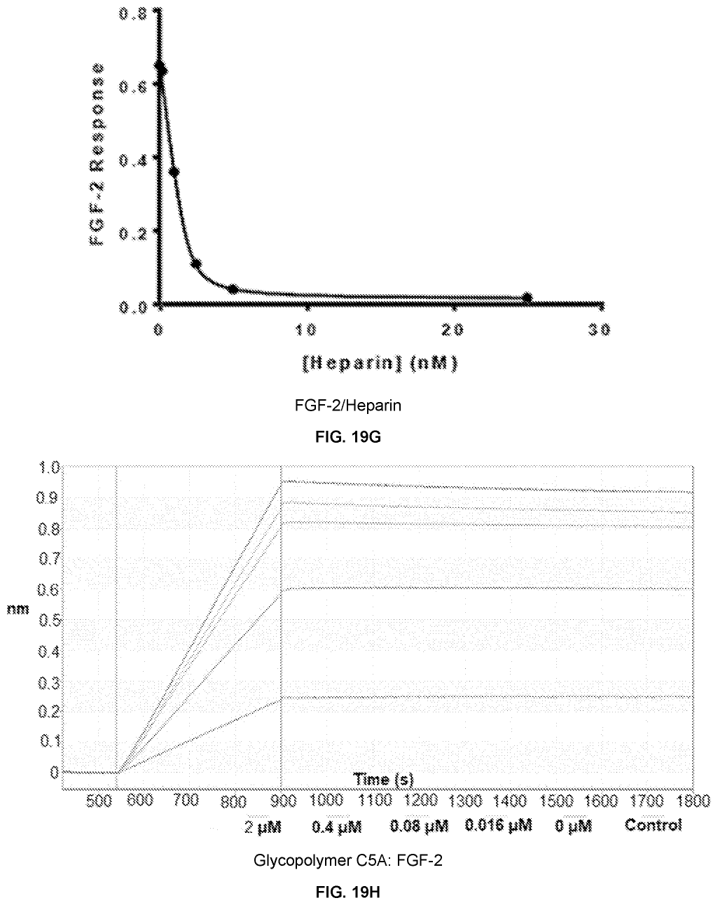

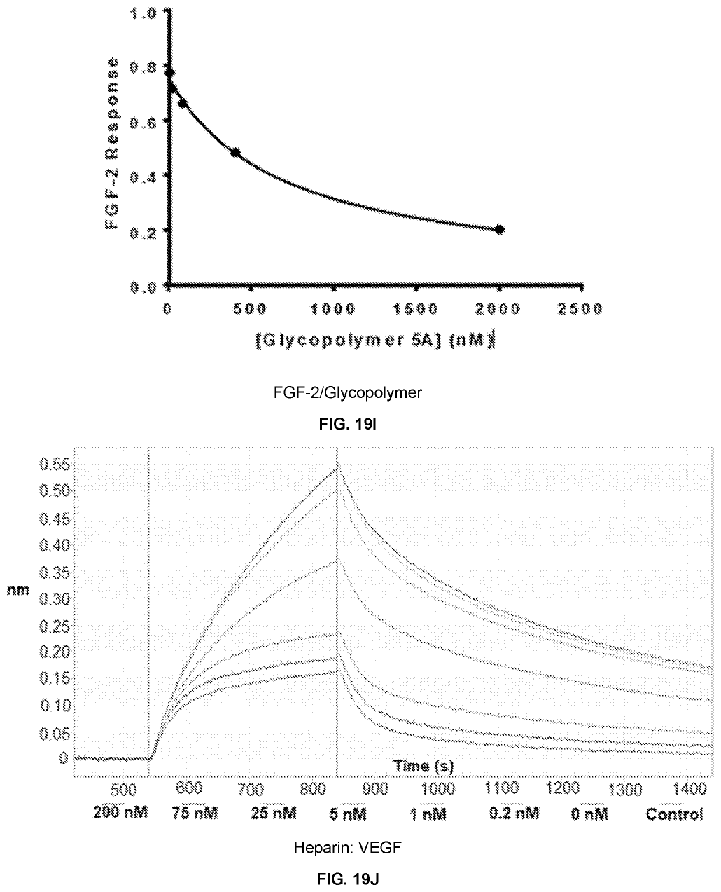

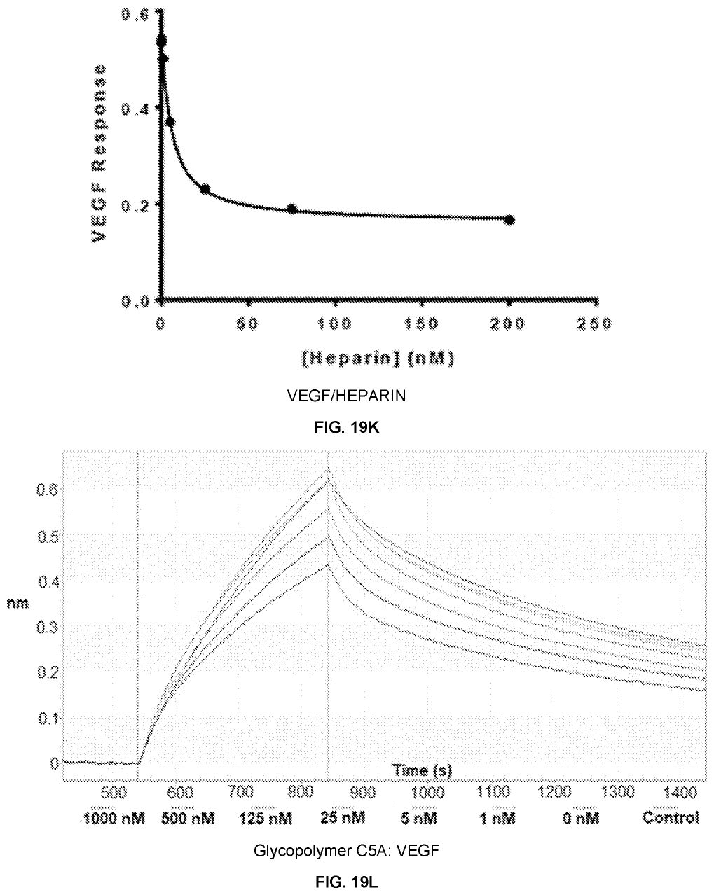

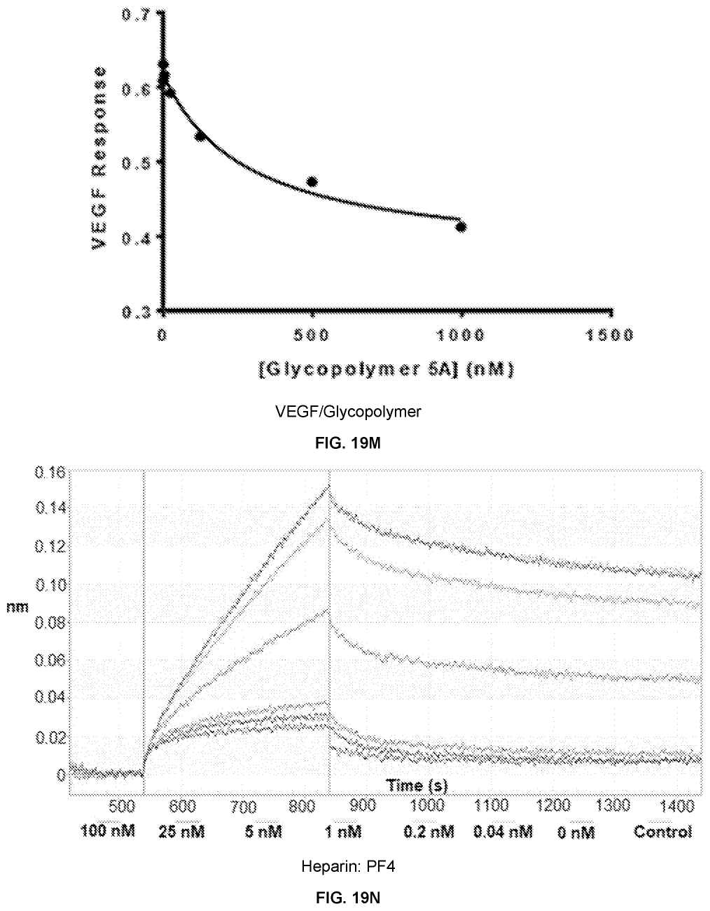

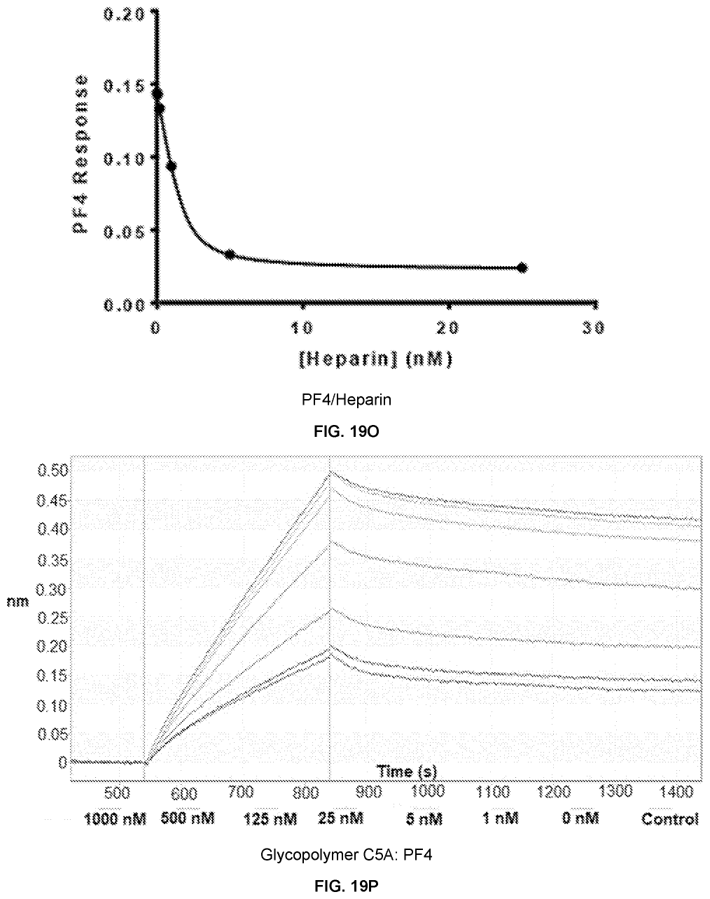

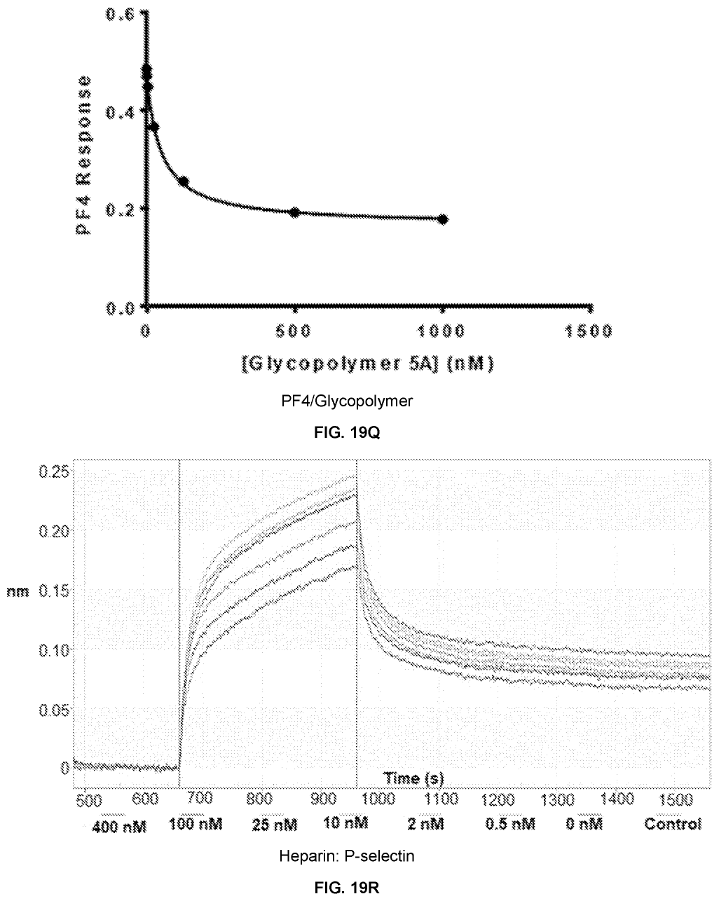

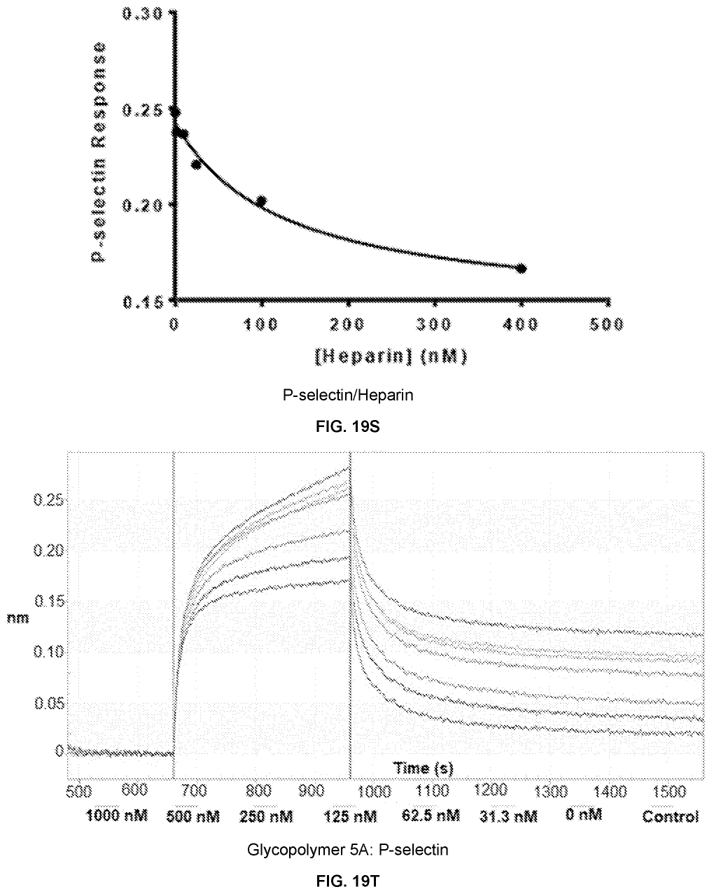

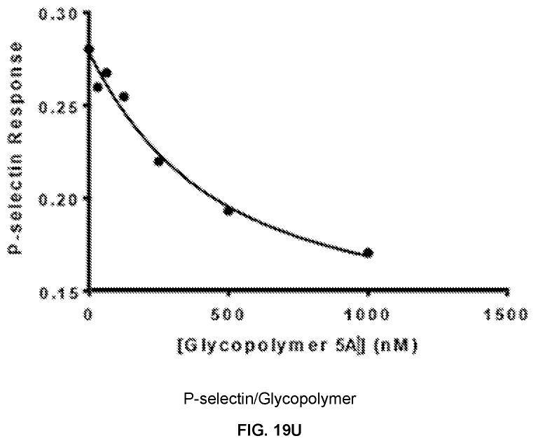

FGF-2 induced cell proliferation assay. FIGS. 19A-19U show BLI sensorgrams and fitted response curves. The association for mass transport of FGF-2 and heparin was carried out for 5 min where as in the solution affinity assay association was performed for 6 min, so responses were recorded at 5 min. FIGS. 19A-19C show BLI sensorgrams and fitted response curves for the analysis of FGF-1 and heparin. Analysis of stoichiometry for FGF-1/heparin was fitted for a segmented linear regression equation. FIGS. 19D and 19E show a BLI sensorgram and fitted response curve for the analysis of FGF-1 and glycopolymer CSA. FIGS. 19F and 19G show a BLI sensorgram and fitted response curve for the analysis of FGF-2 and heparin. FIGS. 19H and 19I show a BLI sensorgram and fitted response curve for the analysis of FGF-2 and glycopolymer CSA. FIGS. 19J and 19K show a BLI sensorgram and fitted response curve for the analysis of VEGF and heparin. FIGS. 19L and 19M show a BLI sensorgram and fitted response curve for the analysis of VEGF and glycopolymer CSA. FIGS. 19N and 19O show a BLI sensorgram and fitted response curve for the analysis of PF4 and heparin. FIGS. 19P and 19Q show a BLI sensorgram and fitted response curve for the analysis of PF4 and glycopolymer C5A. FIGS. 19R and 19S show a BLI sensorgram and fitted response curve for the analysis of P-selectin and heparin. FIGS. 19T and 19U show a BLI sensorgram and fitted response curve for the analysis of P-selectin and glycopolymer CSA.

Cell culture and harvest: HUVECs were cultured at 37° C. in a humidified atmosphere of 5% CO₂ using protocols and reagents supplied by Lonza. Endothelial Growth Medium (EGM), supplemented with hydrocortisone, fetal bovine serum (FBS), ascorbic acid, heparin, gentamicin and growth factors such as VEGF, FGF-2, EFG and IGF was used to maintain the cells. The cell cultures were grown to 70-80% confluence. Once at this confluence the cells were treated with 0.025% trypsin in PBS and incubated for 4-5 min until the cells detached from the flask surface. EGM (8 ml) was added to the harvested cells and the cell suspensions were centrifuged at 190×g for 5 min. The cell pellets were then resuspended in the growth medium and the number of cells were determined using a Beckman coulter counter. After ensuring uniform suspension, cells were reseeded into new vessel with fresh growth medium at seeding densities around 2500-5000 cells/cm² of vessel surface area.

Cell proliferation. Endothelial basal medium (EBM-2), containing only 2% FBS and gentamicin, was used for cell proliferation. Initially, the optimal cell density and concentration of FGF-2, required to induce maximal cell proliferation, were determined. FGF-2 was reconstituted according to manufacturer's protocol and stored at −80° C. FGF-2 stock and C(6)-SO₃ N—SO₃ polymer C5A were diluted by the proliferation medium to the desired concentrations. Cells were resuspended in proliferation medium and 100 μL was seeded on to a 96-well microplate at 3000 cells/well. After incubating for one day, FGF-2(2 nM; 50 μl) and C(6)-SO₃ N—SO₃ polymer C5A (48-0.047 μM; 50 μl) were added to each well, maintaining a final volume of 200 μL. Each concentration was done in triplicate. After incubating for 70 h, 20 μl of the CellTiter 96 Aqueous One Solution Cell Proliferation Assay was added to each well and absorbance, at 490 nm, was measured 2 h later. The entire assay was repeated three times.

Biolayer Interferometry (BLI) Assay. BLI assays were performed on an Octet Red Instrument (fortéBIO) at 25° C. Immobilization and binding analysis were carried out at 1000 rpm using HBS-EP buffer [10 mM HEPES, pH 7.4, 150 mM NaCl, 3.0 mM EDTA, and 0.005% (v/v) surfactant tween20]. A solution affinity assay, used to determine affinities of ligands by SPR analysis, was adopted to BLI (Cochran et al., Glycoconjugate J. 2009, 26, 577-587.). In this method, protein is mixed with various concentrations of ligand (glycopolymer C5A or heparin, 18 kDa). Free protein in this equilibrium mixture is tested for binding against immobilized heparin (all proteins are carrier-free and purchased from R&D Systems). Heparin-biotin (Creative PEGworks, 18 kDa, 1 biotin per HP polymer), 5 μg/mL was immobilized on to streptavidin biosensors (fortéBio) for 5 min. Binding experiments were carried out under conditions of mass transport. Binding was fitted to equation 1 (as taught in reference Chai, et al. Anal. Biochem. 2009, 395, 263-264.) using Graphpad Prism. BLI response was used in place of F and ligand (heparin/glycopolymer C5A) concentration was used in place of [metal]. Binding analysis of P-selectin was carried out with HBS-EP buffer with 2 mM CaCl₂, 2 mM MgCl₂ and 0.5 mg/mL BSA.

4T1 Metastasis Assay (Menhofer, et al., PLOS ONE 2014, 9, el 12542). Luciferase-labeled 4T1 breast carcinoma cells (1×10⁵/mouse) were injected i.v (n=6 mice/group) with vehicle alone (control, PBS), with positive control (heparin), or with GlcNS(6S)α(1,4)GlcA glycopolymer (DP=12, 100 μg/mouse) into BALB/c mice (i.p) 20 min prior to cell inoculation and also together with the cells. IVIS bioluminescent imaging was performed on day 7 after cell inoculation. For IVIS imaging, mice were injected intraperitoneally with D-luciferin substrate at 150 mg/kg and anesthetized with continuous exposure to isoflurane (EZAnesthesia, Palmer, Pa.). Light emitted from the bioluminescent cells is detected by the IVIS camera system with images quantified for tumor burden using a log-scale color range set at 5×10⁴ to 1×10⁷ and measurement of total photon counts per second (PPS) using Living Image software (Xenogen). The experiment was repeated 3 times with similar results.

Heparanase Enzymatic Activity (ECM Degradation Assay) (Vlodaysky, et al., Current Protocols in Cell Biology 2001, 1, 10.14.11-10.14.14). Sulfate [³⁵S] labeled ECM coating the surface of 35 mm tissue culture dishes, is incubated (3-4 h, 37° C., pH 6.0, 1 ml final volume) with recombinant human heparanase (0.5 ng/ml) in the absence and presence of increasing concentrations of the inhibitory compound (for determination of the IC₅₀ in this assay). The reaction mixture contains: 50 mM NaCl, 1 mM DTT, 1 mM CaCl₂), and 10 mM buffer Phosphate-Citrate, pH 6.0. To evaluate the occurrence of proteoglycan degradation, the incubation medium is collected and applied for gel filtration on Sepharose 6B columns (0.9×30 cm). Fractions (0.2 ml) are eluted with PBS and counted for radioactivity. The excluded volume (Vo) is marked by blue dextran and the total included volume (Vt) by phenol red. Degradation fragments of HS side chains are eluted from Sepharose 6B at 0.5<Kay<0.8 (peak II). Sulfate labeled material eluted in peak I (fractions 3-10, just after the void volume) represents nearly intact HSPG released from the ECM due to proteolytic activity residing in the ECM. Results are best represented by the actual gel filtration pattern.

Experimental Example 2. Phenanthroline-Catalyzed Stereoretentive Glycosylations. Carbohydrates are essential components of many bioactive molecules in nature. However, efforts to elucidate their modes of action are often impeded by limitations in synthetic access to well-defined oligosaccharides. Most of the current methods rely on the design of specialized coupling-partners to control selectivity during formation of glycosidic bonds. Here, the present disclosure reports a commercially available phenanthroline that catalyzes stereoretentive glycosylation with glycosyl bromides. The method provides efficient access to a myriad of axial 1,2-cis glycosides as well as axial 2-azido- and 2-fluoro-glycosides. This operationally simple and air- and moisture-tolerant procedure has been performed for the large-scale synthesis of a disaccharide and an octasaccharide adjuvant. Density functional theory calculations predict the anomeric phenanthrolinium ion, which prefers the equatorial orientation, to be stabilized via non-covalent interactions between the C-1 axial hydrogen of glycosyl moiety and phenanthroline nitrogen atom. These calculations, together with kinetic studies, suggest that the reaction proceeds via double S_{N}2-like mechanism.

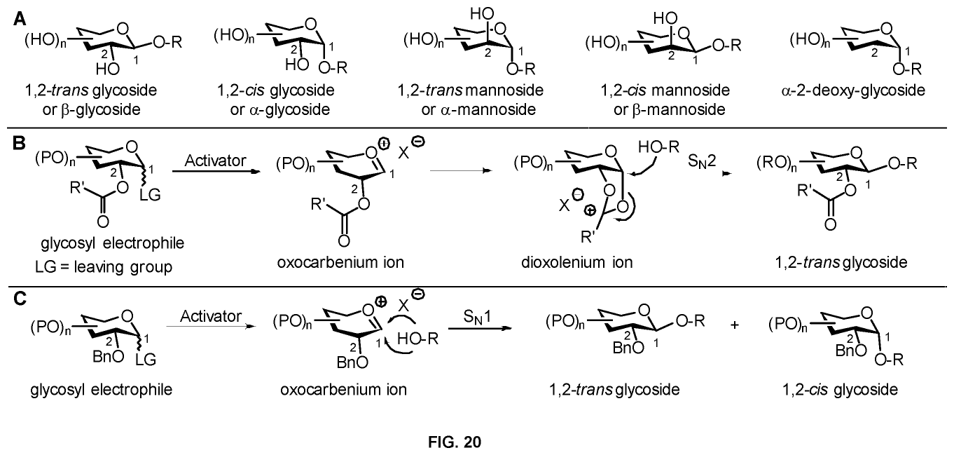

Introduction. Glycosylations are fundamental methods for constructing complex carbohydrates. Key reactions involve glycosidic bond formation that connects glycosyl electrophiles to glycosyl nucleophiles to generate oligosaccharides, which play a critical role in cellular functions and disease processes (Ohtsubo, et al., Cell. 126, 855-867 (2006); Brockhausen, et al., EMBO Rep. 7, 599-604 (2006); Crocker, et. al., Nat. Rev. Immunol. 7, 255-266 (2007); van Kooyk, et al., Nat. Immunol. 9, 593-601 (2008)). As a result, the efficient preparation of well-defined oligosaccharides has been a major focus in carbohydrate synthesis. Despite recent advances (Zhu, et al., Angew. Chem. Int. Ed. 48, 1900-1934 (2009); McKay, et al., ACS Catal. 2, 1563-1595 (2012)., Seeberger, et al., Acc. Chem. Res. 48, 1450-1463 (2015)) the ability to forge C-0 glycosidic bonds (FIG. 20, A) in a stereoselective fashion is not easily predictable due to the reaction's high degree of variables and shifting S_{N}1-S_{N}2 mechanistic paradigm (FIG. 20) (Boltje, et al., Nat. Chem. 1, 611-622 (2009).; Leng, et al., Acc. Chem. Res. 51, 628-639 (2018); Crich, et al., Acc. Chem. Res. 43, 1144-1153 (2010)). Most established methods to achieve stereoselective glycosylation reactions have focused on tuning the steric and electronic nature of the protecting group on the electrophilic partners (Boons, et al., Contemp. Org. Synth. 3, 173-200 (1996); Nigudkar, et al., Chem. Sci. 6, 2687-2704 (2015); Kim, et al., J. Am. Chem. Soc. 127, 12090-12097 (2005); Yasomanee, et al., J. Am. Chem. Soc. 134, 20097-20102 (2012); Yasomanee, et al., Angew. Chem. Int. Ed. 53, 10453-10456 (2014); Crich, et al., J. Org. Chem. 62, 1198-1199 (1997)). The most reliable approach is based on the O-acyl participatory protecting group at C(2) of the glycosyl electrophile for construction of the 1,2-trans glycosidic linkage via an S_{N}2-like pathway (FIG. 20, B) (Boons, et al., Contemp. Org. Synth. 3, 173-200 (1996)). The formation of 1,2-cis glycosides requires an electrophilic partner with a non-participatory ether functionality at C(2) (Nigudkar, et al., Chem. Sci. 6, 2687-2704 (2015)). Use of this type of electrophiles typically engages in an S_{N}1-like pathway, leading to a mixture of two stereoisomers that differ in the configuration of the anomeric center (FIG. 20, C) (Nigudkar, et al., Chem. Sci. 6, 2687-2704 (2015)). Novel methods based on neighboring group participation (Kim, et al., J. Am. Chem. Soc. 127, 12090-12097 (2005)) and remote participation (Yasomanee, et al., J. Am. Chem. Soc. 134, 20097-20102 (2012); Yasomanee, et al., Angew. Chem. Int. Ed. 53, 10453-10456 (2014)) of the protecting groups on glycosyl electrophiles offer a solution for forming 1,2-cis glycosides. These substrate-controlled methods, however, are highly specialized for each electrophilic partner. Alternatively, catalyst-controlled glycosylation has emerged as a way to eliminate the need for specific protecting groups (Geng, et al., Angew. Chem. Int. Ed. 52, 10089-10092 (2013); Sun, et al., Angew. Chem. Int. Ed. 55, 8041-8044 (2016); Kimura, et al., Org. Lett. 18, 3190-3193 (2016); Park, et al., Science. 355, 162-+(2017); Mensah, et al., J. Org. Chem. 74, 1650-1657 (2009). Peng, et al., J. Am. Chem. Soc. 137, 12653-12659 (2015)). However, only limited catalytic examples for forming axial 1,2-cis glycosides are known (Kimura, et al., Org. Lett. 18, 3190-3193 (2016)).

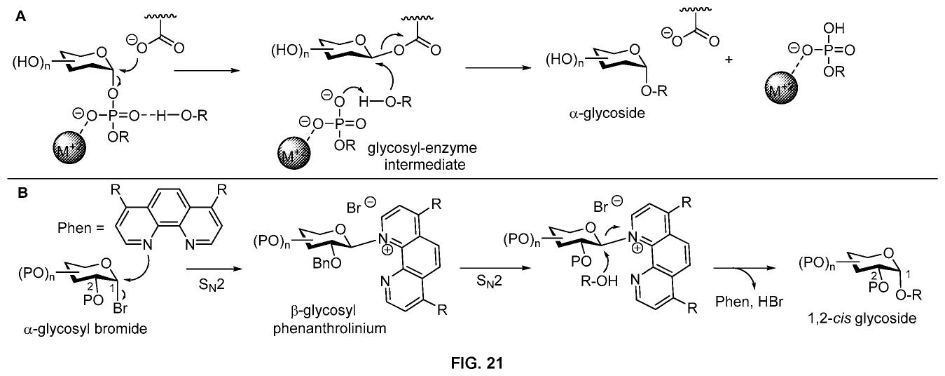

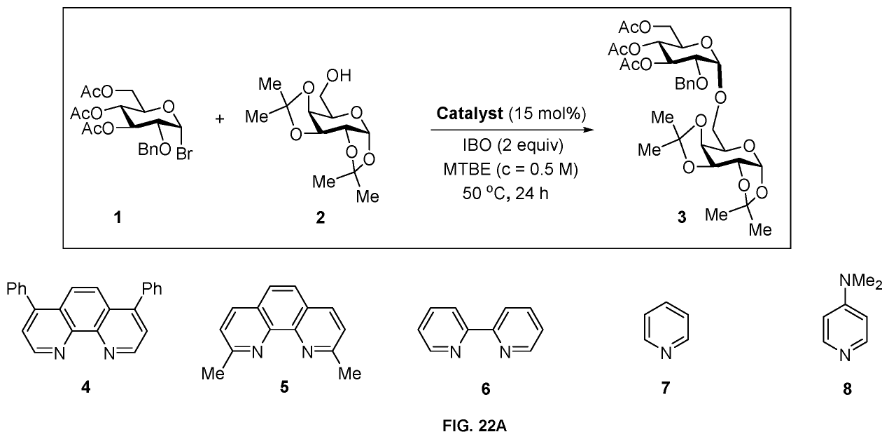

Since the synthesis of oligosaccharides relies on many diverse sugar building blocks, it is uncertain whether the aforementioned catalytic systems would be translated over a range of axial 1,2-cis glycosides. Retaining glycosyltransferases are known to catalyze α-glycosidic bond formation (Lairson, et al., Annu. Rev. Biochem. 77, 521-555 (2008)) with net retention of anomeric configuration (FIG. 21, A). Inspired by the effectiveness of enzymes, it was envisioned that a small molecule catalyst capable of performing stereoretentive glycosylations to provide 1,2-cis glycosides with predictable α-selectivity and in preparatively high yields would likely find broad applications. Pyridine has been reported to serve as a nucleophilic catalyst (Fu, et al., Acc. Chem. Res. 33, 412-420 (2000)). Displacement of the anomeric leaving group of a glycosyl electrophile with pyridine affords an anomeric pyridinium ion intermediate (Mulani, et al., Org. Biomol. Chem. 12, 1184-1197 (2014)), one that prefers the equatorial position (β) to avoid the steric interactions associated with positioning that group in the axial (a) orientation (Frihed, et al., Chem. Rev. 115, 4963-5013 (2015)). Invertive substitution by a nucleophile would then afford an axial 1,2-cis glycoside. Unfortunately, pyridine-mediated reaction proceeds with marginal bias for the α-selectivity as an axial pyridinium ion, which can also be formed to compete for access to a 1,2-trans glycoside (Garcia, et al., J. Am. Chem. Soc. 122, 4269-4279 (2000)). An attractive option would be to use phenanthroline (FIG. 21, B), which has been shown to be a powerful ligand for metal ions and a binding agent for DNA/RNA through non-covalent interactions (Bencini, et al., Chem. Rev. 254, 2096-2180 (2010); Erkkila, et al., Chem. Rev. 99, 2777-2795 (1999)). Phenanthroline is a rigid and planar structure with two fused pyridine rings whose nitrogen atoms are positioned to act cooperatively. The first nitrogen atom could serve as a catalytic nucleophile to react with a glycosyl electrophile to form a covalent β-phenanthrolium ion preferentially (FIG. 21, B), since phenanthroline is more sterically demanding than pyridine. The second nitrogen atom could non-covalently interact with glycosyl moiety or act as a hydrogen-bond acceptor to facilitate invertive substitution by a nucleophile. These unique features of phenanthroline could effectively promote a double displacement mechanism.

Here, the disclosure shows a bathophenanthroline catalyst for the highly selective synthesis of axial 1,2-cis glycoside synthesis. This catalytic-controlled glycosylation methodology allows access a broad range of saccharides bearing C(2)-oxygen, azido, and fluoro functionality and is applicable for construction of potent vaccine adjuvant, α-glycan octasaccharide. Presumably, this is the first reaction reported wherein a phenanthroline serves as a nucleophilic catalyst to control a stereoretentive glycosylation.

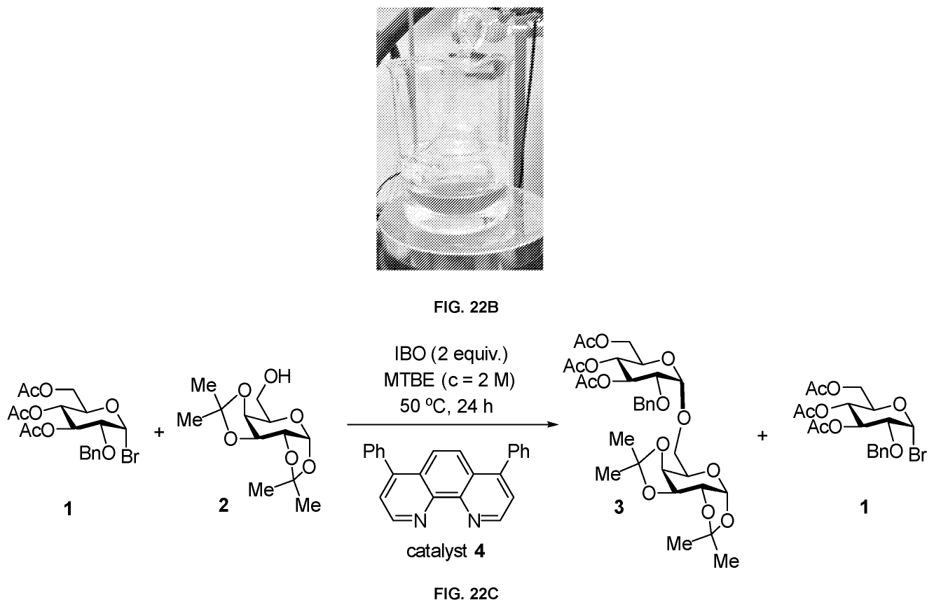

Results and discussion. Reaction development. The realization of the stereoretentive glycosylation concept outlined above is influenced by the anomeric configuration of the electrophilic substrate. In the current reaction development, α-configured glycosyl bromide 1 was chosen as a model electrophilic partner and galactopyranoside 2 as a glycosyl nucleophile to simplify the analysis of coupling product mixtures 22A). Previous reports have documented the ability of glycosyl bromides to function as one of the most common electrophiles under various glycosylation conditions and to generate as α-configured substrates (Koenig, W., et al., Ber. Dtsch. Chem. Ges. 34, 957-981 (1901); Lanz, et al., Eur. J. Org. Chem., 3119-3125 (2016)). The reaction of 2 with glucosyl electrophile 1, having a C(2)-non-participatory benzyl (Bn) group (Nigudkar, et al., Chem. Sci. 6, 2687-2704 (2015)), often proceeds via an S_{N}1-like pathway to provide the coupling product with poor anomeric selectivity. As expected, use of the conventional Lewis acid, silver triflate (AgOTf), provided a 4:1 (α:β) mixture of the desired product 3. Upon exploring a range of reaction parameters (FIGS. 23-28), coupling of 2 with 1 was discovered in the presence of 15 mol % of 4,7-diphenyl-1,10-phennathroline (4) as a catalyst and isobutylene oxide (IBO) as a hydrogen bromide scavenger in tert-butyl methyl ether (MTBE) at 50° C. for 24 h and that this provided the highest yield and α-selectivity of 3 (73% yield, α:β>30:1). In the absence of catalyst 4, no reaction was apparent after 24 h. The reaction was conducted with other catalysts (5-8), and three trends were observed. First, the yield of 3 is correlated with the ability of the catalyst to displace the anomeric bromide. The C(2)- and C(9)-methyl groups of catalyst 5 reduce the accessibility of the pyridine nitrogen atom for displacing the bromide leaving group. Second, the conformation of the catalyst can influence the efficiency and selectivity of the coupling event. For instance, 2,2′-bipyridine (6) is less α-selective than catalyst 4 potentially due the two nitrogen atoms being disrupted by the free-rotation about the bond linking the pyridine rings. Third, the α-selectivity is correlated with the efficiency of the catalyst to promote glycosylation. As expected, pyridine (7) is not as α-selective as phenanthroline catalyst 4. Since 4-(dimethylamino)pyridine (8) is known to be a more effective catalyst than pyridine (7) (Koenig, W., et al., Ber. Dtsch. Chem. Ges. 34, 957-981 (1901)), the product 3 was obtained in higher yield (25% vs. 51%) (FIG. 22A).

A primary roadblock that hinders study of the role of carbohydrates in many biological processes remains the limited availability of reproducible and predictable glycosylation conditions to allow for routine oligosaccharide synthesis in large and pure quantities. In addition, current techniques are limited to specialists who can produce these constructs. Since the phenanthroline-catalyzed reaction is air- and moisture-tolerant and operationally simple by combining coupling partners 1 and 2 with catalyst 4 and IBO in MTBE under an open air in the flask (FIG. 20B), this system could be suitable for a large-scale synthesis. Accordingly, the reaction was conducted on a 4 mmol scale of 1 and 4.4 mmol of 2 (FIG. 22C). Because the reaction was performed on a gram scale at a relatively high concentration (2 M), a catalyst loading of 5 mol % proved sufficient. The product 3 was attained without any effect on the yield and selectivity.

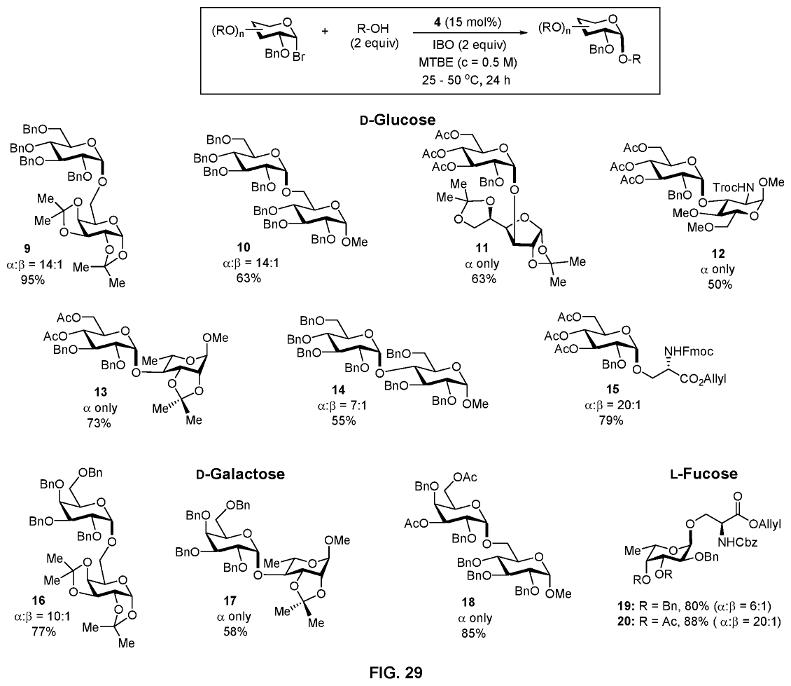

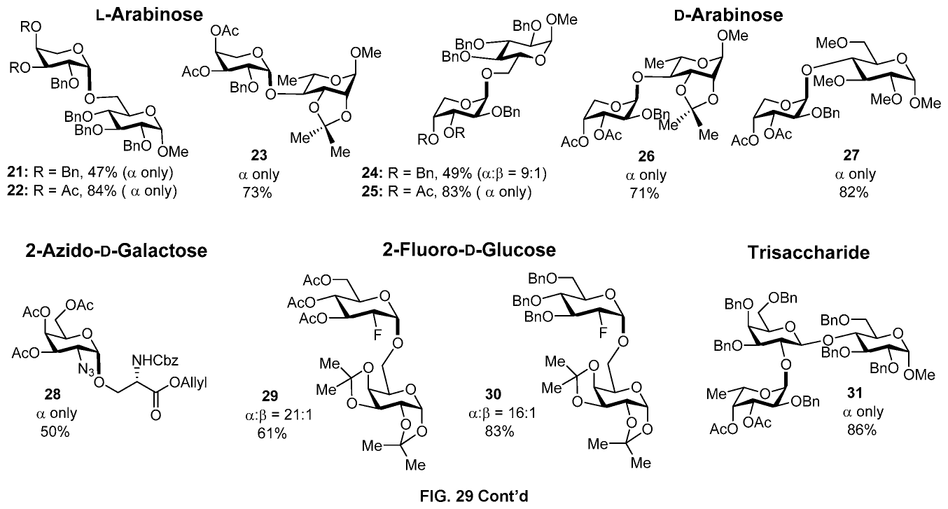

Substrate Scope. In an effort to guide specialists and non-specialists towards optimal phananthroline-catalyzed glycosylation conditions without prior reaction optimizations, general guidelines based on the scope of the coupling partners are needed. There are several underlying factors that could potentially influence the efficiency and the stereochemistry of the products. While the C-2 protecting group of glycosyl electrophile has a direct impact on the selectivity of the product Boons, et al., Contemp. Org. Synth. 3, 173-200 (1996); Kim, et al., J. Am. Chem. Soc. 127, 12090-12097 (2005)), the protecting group nature at other positions are capable of indirectly influencing the reaction (Yasomanee, et al., J. Am. Chem. Soc. 134, 20097-20102 (2012); Yasomanee, et al., Angew. Chem. Int. Ed. 53, 10453-10456 (2014); Baek, et al., J. Am. Chem. Soc. 131, 17705-17713 (2009)). The reactivity of alcohol nucleophiles can also have an impact on the coupling efficiency and selectivity. As such, glucose-derived having electron-withdrawing acyl and electron-donating benzyl groups at C(3), C(4), and C(6) positions, were first explored with primary and secondary hydroxyls of nucleophilic coupling partners. To validate that the phenanthroline catalyst 4 could overturn the “remote” participation of the C(3)-, C(4)-, and/or C(6)-acyl protecting groups (Boons, et al., Contemp. Org. Synth. 3, 173-200 (1996); Yasomanee, et al., J. Am. Chem. Soc. 134, 20097-20102 (2012); Yasomanee, et al., Angew. Chem. Int. Ed. 53, 10453-10456 (2014); Baek, et al., J. Am. Chem. Soc. 131, 17705-17713 (2009)), glucosyl bromide bearing non-participatory benzyl protecting groups were explored with C(6)-hydroxyl of carbohydrate nucleophiles (FIG. 29). Compared to electrophile 1, no significant compromise to the α-selectivity was observed as both disaccharides 9 and 10 with high levels of α-selectivity, suggesting an S_{N}2-type displacement for this catalyst-controlled method. This catalytic protocol is more α-selective than other methods. For example, while the disclosure catalytic system provided 10 with α:β=14:1, reaction with trichloroacetimidate and cyclic difluoroimidate electrophiles with use of TMSOTf as promoter provided 10 with an α:β ratio of 4:1 and 1:1.2, respectively (Nigudkar, et al., J. Am. Chem. Soc. 136, 921-923 (2014); Nguyen, et al., J. Am. Chem. Soc. 123, 8766-8772 (2001)). Glycosyl bromides also act as viable electrophiles to efficiently glycosylate hindered C(3)- and C(4)-secondary hydroxyls. In all cases, the expected α-product (11-13, FIG. 29) was produced predominantly. For the challenging C(4)-hydroxyl of the glucoside nucleophile, the S_{N}1-S_{N}2 reaction paradigm was slightly shifted (14: α:β=7:1). A primary alcohol of a protected serine amino acid also exhibited excellent α-selectivity (15: α:β=20:1).

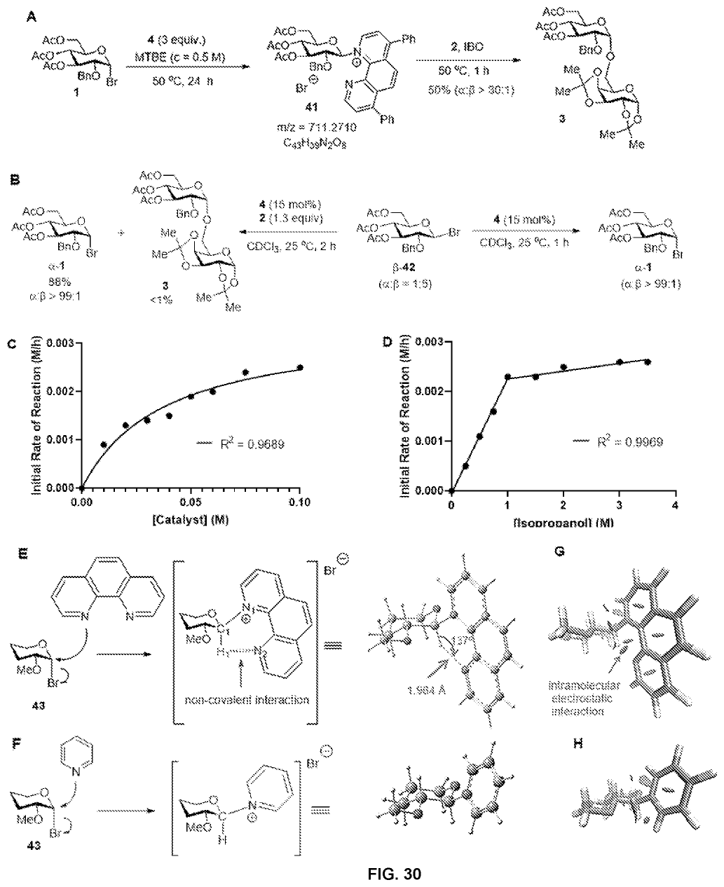

Variation of the structure of the electrophilic reacting partner was also explored (FIG. 29). Compared to D-glucose, the axial C(4)-benzyl protecting group of D-galactose has been reported to favor β-product formation (Chatterjee, et al., J. Am. Chem. Soc. 140, 11942-11953 (2018)). In contrast, the catalyst 4 overturned this intrinsic substrate bias to provide disaccharides 16-18 with excellent α-selectivity. Upon comparison of this catalytic-controlled method with the amide-mediated method (Lu, et al., Angew. Chem. Int. Ed. 50, 7315-7320 (2011)), it is clear that the reaction is α-selective for formation of 16 in the phenanthroline system (α:β=10:1) relative to the amide system (α:β=3:1). The capacity of the phenanthroline system with L-fucose was investigated. While tribenzyl L-fucosyl bromide reacted rapidly to provide 19 in 80% yield with synthetically useful levels of α-selectivity (α:β=6:1), use of an electron withdrawing L-fucose provided 20 exclusively as α-isomer. Both 19 and 20 are key units of a thrombospondin type 1 repeat, which plays a vital role in an autosomal recessive disorder (Vasudevan, et al., Curr. Biol. 25, 286-295 (2015)). The more labile monosaccharides were investigated next. Use of tribenzyl protected L-arabinosyl bromide provided 21 exclusively as α-isomer (FIG. 30), albeit with moderate yield (47%). It was observed that this electron-donating L-arabinose substrate decomposed during the course of the reaction, consequently attenuating the yield of 21. To increase the stability of L-arabinose, the C(3)- and C(4)-acetyl groups were used to produce 22 in high yield (84%). This electron-withdrawing substrate was also compatible with the C(4)-hydroxyl, affording α-product 23, key motif of glycosphingolipid vesparioside B (Gao, et al., J. Am. Chem. Soc. 138, 1684-1688 (2016)). A similar trend was observed with D-arabinose, providing disaccharides 24-27 with good to excellent levels of α-selectivity. To compare, this catalytic protocol to produce 24 (α:β=9:1) is more α-selective than the method using tribenzyl arabinose thioglycoside and NIH/AgOTf as the activating agent (α:β=3:1) (Gao, et al., J. Am. Chem. Soc. 138, 1684-1688 (2016)). The selectivity trends with electrophiles bearing C(2)-azido and C(2)-fluoro groups was also sought to be determined (FIG. 30). Excellent α-selectivity with use of C(2)-azido-D-galactose was observed (28, 50%, α only). To compare, 28, a precursor of tumor-associated mucin T_{N} antigen (Pratt, et al., Chem. Soc. Rev. 34, 58-68 (2005)), could also be prepared in a 4:1 (α:β) mixture using a stoichiometric amount of AgClO₄ as the activating reagent (Kuduk, et al., J. Am. Chem. Soc. 120, 12474-12485 (1998)). The 2-fluoro-D-glucose substrate was observed next. The ability of the C(2)-F bond to have an impact on the stereochemical outcome of the coupling product has been reported (Bucher, et al., Angew. Chem. Int. Ed. 49, 8724-8728 (2010)). While the 2-fluoro-glucose having benzyl protecting groups is β-selective under TMSOTf-mediated conditions (Bucher, et al., Angew. Chem. Int. Ed. 49, 8724-8728 (2010); Durantie, et al., Chem.-Eur. J. 18, 8208-8215 (2012)), the analogous acetyl-0 electrophile affords a 1:1 mixture of α- and β-isomers (Bucher, et al., Angew. Chem. Int. Ed. 49, 8724-8728 (2010); Durantie, et al., Chem.-Eur. J. 18, 8208-8215 (2012)). In contrast to the reported method, both the acetyl- and benzyl-protected 2-fluoro-D-glucose substrates are highly α-selective under the disclosures catalytic conditions (29, α:β=21:1; 30, α:β=16:1). Finally, this catalyst-controlled method is also amendable to the synthesis of a protected human milk α-trisaccharide 31 in high yield (86%) (Xiao, et al., J. Org. Chem. 81, 5851-5865 (2016)).

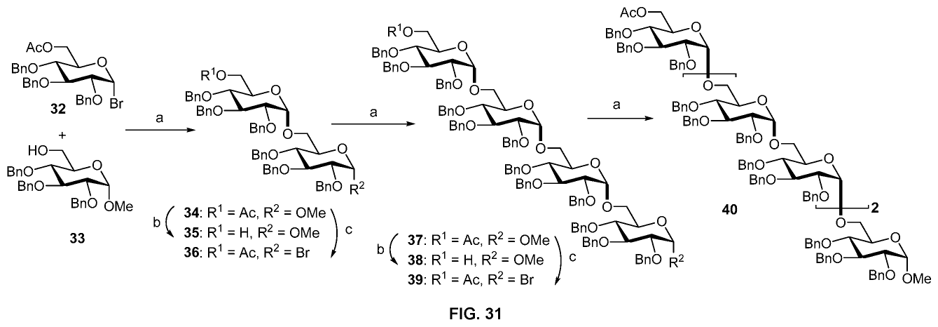

The critical question remains whether this phenanthronline system is applicable for construction of larger oligosaccharides. The α-(1,6)-linked octasaccharide 40 was chosen (FIG. 31), a carbohydrate backbone of the natural α-glucan polysaccharides (Bittencourt, et al., J. Biol. Chem. 281, 22614-22623 (2006); van Bueren, et al., Nat. Struct. Mol. Biol. 14, 76-84 (2007)), which have the potential as vaccine adjuvants. However, these α-glucans are heterogeneous in size and composition. As such, well-defined oligosaccharides are required to study bioactive fragments. In the disclosure, the anomeric methoxy group was chosen for the reducing end of oligosaccharides as nucleophile 33 is comercially available (FIG. 30). Accordingly, a catalyst loading of 5 mol % proved efficient to promote the coupling of 33 with glycosyl bromide 32 to provide disaccharide 34 in good yield and excellent α-selectivity (86%, α:β>25:1). This catalytic method is also suitable for preparing 10 mmol of 34 with comparable yield and selectivity (8.4 g, 89%, α:β>25:1). Acetyl hydrolysis of 34 provided disaccharide nucleophile 35. For the synthesis of electrophile 36, disaccharide 33 was first converted to the glycosyl acetate intermediate (Cao, et al., Carbohydr. Res. 341, 2219-2223 (2006)), which was isolated prior to converting into bromide 36, which was used without further purification in the coupling to 35 to afford tetrasaccharide 37 (86%, α:β>25:1). Compound 37 was further functionalized to generate 38 and 39, under similar conditions for preparation of 35 and 36, for use in another coupling iteration to generate octasaccharide 40 (77%, α:β>25:1). Overall, the synthesis of 40 underscores the ability of the catalyst 4 to construct well-defined large oligosaccharides.

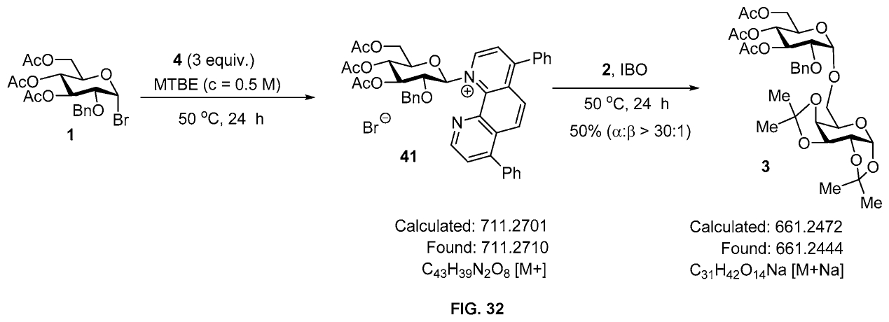

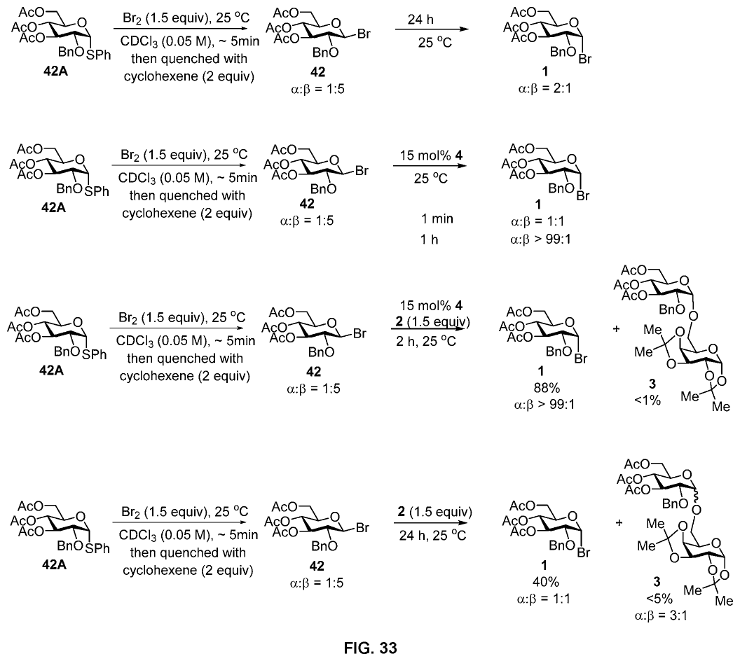

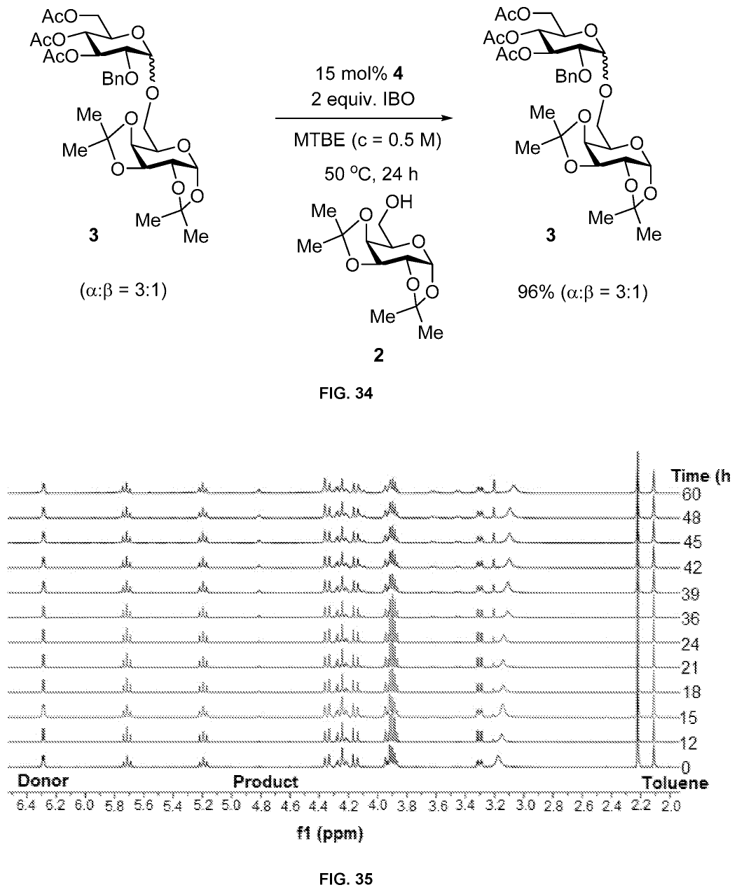

Mechanistic studies. Having obtained 1,2-cis product in high yield and excellent α-selectivity, the mechanism of the phenanthroline-catalyzed stereoselective glycosylation was investigated next. With the possibility that the reaction goes through a transient β-phenanthrolinium intermediate, this putative species was attempted to be detected by using mass spectroscopy. In the event, glycosyl bromide 1 was treated with stoichiometric amount of 4 in MTBE (0.5 M) for 24 h at 50° C. Formation of a phenanthrolinium ion 41 was confirmed using electrospray ionization (ESI) with an m/z ratio of 711.2710 (FIG. 30). Subsequent fragmentation of 41 using collision induced dissociation (CID) led to the formation of the phenanthroline species with an m/z ratio of 333.1396 (FIG. 32). The final step involved the introduction of nucleophile 2 to provide disaccharide 3 with comparable results to those obtained earlier (FIG. 22A-22C). It was next evaluated if the stereochemistry of the 1,2-cis product would be dictated by the anomeric configuration of the electrophile. Consistent with the proposed double inversion S_{N}2 pathway (FIG. 21, B), α-configured glycosyl bromide is the reacting partner. The kinetic β-bromide 42, generated in situ from α-thioglycoside (FIG. 33) (Nigudkar, et al., J. Am. Chem. Soc. 136, 921-923 (2014); Vasudevan, et al., Curr. Biol. 25, 286-295 (2015)), rapidly converted into the thermodynamically stable α-bromide 1 in the presence of catalyst 4 within 1 h at 25° C. (FIG. 30, B). In the absence of 4, β-bromide 42 slowly anomerized to α-bromide 1 at 25° C. (FIG. 34). A conversion of α-bromide, in the presence of added bromide ion, to the more reactive β-bromide, which reacts with a nucleophile to give a 1,2-cis glycoside, has been reported in Lemieux, et al., J. Am. Chem. Soc. 97, 4056-4062 (1975). In contrast, coupling of 2 with β-bromide 42 in the presence of 15 mol % of 4 afforded 1,2-cis product 3 in less than 1% (FIG. 30, B). The α:β ratio of the desired product 3 is kinetically-derived and is not reflective of a thermodynamic distribution arising from post-coupling anomerization (FIG. 33).

To gain further mechanistic insight, the initial rates of phenanthroline-catalyzed glycosylation of a nucleophile, 2-propanol, with glycosyl bromide 1 were also determined using ¹H NMR spectroscopy. The kinetic data suggest that the reaction undergoes S_{N}2-like mechanism (FIGS. 30, C-D and FIGS. 35-39), as the initial rate of the reaction is both catalyst (FIG. 30, C) and nucleophile (FIG. 30, D) dependent. The initiate rate of reaction is quite slow, supporting that there is likely no background reaction in the absence of catalyst 4 (FIG. 30, C). There is a non-linearity downward as the concentration of catalyst 4 increases (FIG. 30, C), probably due to catalyst aggregation as the reaction mixture becomes insoluble at high catalyst concentration. The biphasic kinetic in FIG. 30, D suggests a shift in the rate-determining step (RDS) at different isopropanol concentration. At high concentration of isopropanol, the RDS is the formation of the phenanthrolinium ion (first step, FIG. 21, B). At low concentration of isopropanol, nucleophilic attack (second step) is the RDS.

Finally, to understand the role of the phenanthroline catalyst in controlling high α-selective 1,2-cis glycosylation, the intermediate structures for nucleophilic addition of phenanthroline (FIG. 30, E) or pyridine (FIG. 30, F) to glycosyl bromide 43 have been optimized using density functional theory (DFT) calculations at the B3LYP/6-31+G(d,p) level with the SMD implicit solvent model. DFT calculations predict that the β-phenanthrolinium intermediate is stabilized by intramolecular non-covalent interactions between the C-1 axial hydrogen of glycosyl moiety and the nitrogen atom of phenanthroline (the bond distance of H₁—N₂ is 1.964 Å and the bond angle of C₁—H₁N₂ is 136.9°). The C—N surface in the non-covalent interaction plot (FIG. 30, H) (Johnson, et al., J. Am. Chem. Soc. 132, 6498-6506 (2010)) also indicates that the electrostatic interaction is presented in an anomeric β-phenanthrolinium ion. On the other hand, the non-covalent interactions are not observed for the β-pyridinium ion (FIG. 30, G; 30, H). It appears that a tight phenanthrolinium ion complex shields the β-face of glycosyl moiety, making the β-face more accessible for nucleophilic attack via the S_{N}2 pathway.

Methods. Synthesis. A general procedure for phenanthroline-catalyzed glycosylation is as follows. A 50 mL round-bottom flask was charged with glycosyl bromide 1 (1.83 g, 4.0 mmol, 1.0 equiv.), alcohol 2 (1.25 g, 4.8 mmol, 1.2 equiv.), catalyst 4 (66 mg, 0.2 mmol, 15 mol %), IBO (0.7 mL, 8.0 mmol, 2.0 equiv.) and MTBE (2.0 mL). The resulting solution was stirred at 50° C. for 24 h under open-air atmosphere, diluted with toluene, and purified by silica gel flash chromatography (toluene/ethyl acetate: 5/143/1) to give the desired disaccharide 3 (1.784 g, 70%, α:β>30:1) and recovered 1 (0.515 g, 28%).

Kinetic Study. A 10 mL scintillation vial was charged with glycosyl bromide 1 (fixed amount, 0.25 mmol, 1.0 equiv.), isopropanol (vary amount from 0.5 to 5 equiv.), catalyst 4 (vary amount from 2 to 20 mol %), IBO (vary amount from 1.5 to 3 equiv.), toluene (internal standard, 0.083 mmol, 0.33 equiv.), and C₆D₆ (0.5 mL). The resulting solution was then transferred to a 5 mm NMR tube. ¹H NMR spectrum was acquired on a 400 MHz instrument before heating. Then the mixture in NMR tube was consistently shaken and heated in a 50° C. water bath. Between 3 and 60 h, spectra were obtained depending on the experiment. Example spectra and example rate plot were based on standard conditions: 0.25 mmol glycosyl bromide 1 (1.0 equiv.), 0.75 mmol acceptor (3.0 equiv.), 15 mol % catalyst 4, 0.5 mmol IBO (2 equiv.), 0.083 mmol toluene (0.33 equiv.) as an internal standard, and 0.5 mL C6D₆ (0.5 M).

Calculation. All calculations were carried out with Gaussian 09. Geometry optimization for reactant, intermediates, transition states, and products were computed at the B3LYP/6-31+G(d,p) level of theory with the SMD implicit solvation model in diethyl ether. There is only one imaginary frequency for transition state structures and no imaginary frequency for reactant, intermediates, and products. Non-covalent interactions (NCI) were calculated with the NCI PLOT program.

Conclusions. Overall, the phenanthroline-catalyzed glycosylation strategy provides a general platform for α-selective formation of a range of 1,2-cis glycosides. This catalytic system is not confined to the predetermined nature of glycosyl coupling partners and mimics glycosyltransferase-catalyzed retentive mechanisms, wherein the stereochemistry of the products is influenced by the anomeric α-configuration of the glycosyl electrophiles. This work stands at the underdeveloped intersection of operationally simple conditions, catalytic glycosylation, and stereocontrolled glycosidic bond formation, each of which represents an important theme in the synthesis of well-fined oligosaccharides. Further expanding the scope of the catalytic α-selective glycosylation reaction represents a feasible roadmap towards a general and broadly accessible solution to complex carbohydrate synthesis. This roadmap includes the investigation of bacterial sugar building blocks found in many oligosaccharides and polysaccharides, the development of better conditions for iterative coupling of carbohydrate building blocks, and the advancement of a more generalized automation of oligosaccharide synthesis.

Supporting information. General information. Methods and Reagents: All reactions were performed in oven-dried flasks fitted with septa under a positive pressure of nitrogen atmosphere. Organic solutions were concentrated using a Buchi rotary evaporator below 40° C. at 25 torr. Analytical thin-layer chromatography was routinely utilized to monitor the progress of the reactions and performed using pre-coated glass plates with 230-400 mesh silica gel impregnated with a fluorescent indicator (250 nm). Visualization was then achieved using UV light, iodine, or ceric ammonium molybdate. Flash column chromatography was performed using 40-63 μm silica gel (SiliaFlash F60 from Silicycle). Dry solvents were obtained from a SG Waters solvent system utilizing activated alumina columns under an argon pressure. All other commercial reagents were used as received from Sigma Aldrich, Alfa Aesar, Acros Organics, TCI, and Combi-Blocks, unless otherwise noted.

Instrumentation. All new compounds were characterized by Nuclear Magnetic Resonance (NMR) spectroscopy and High-Resolution Mass spectrometry (HRMS). All ¹H NMR spectra were recorded on either Bruker 400 or 500 MHz spectrometers or DRX-400 (400 MHz) spectrometer. All ¹³C NMR spectra were recorded on either Bruker 100 or 125 MHz spectrometer or DRX-400 (100 MHz) spectrometer. All ¹⁹F NMR spectra were recorded on DRX-400 (376 MHz) spectrometer. Chemical shifts are expressed in parts per million (δ scale) downfield from tetramethylsilane and are referenced to the residual proton in the NMR solvent (CDCl₃: δ 7.26 ppm, δ 77.00 ppm). Data are presented as follows: chemical shift, multiplicity (s=singlet, d=doublet, t=triplet, q=quartet, m=multiplet, and bs=broad singlet), integration, and coupling constant in hertz (Hz).

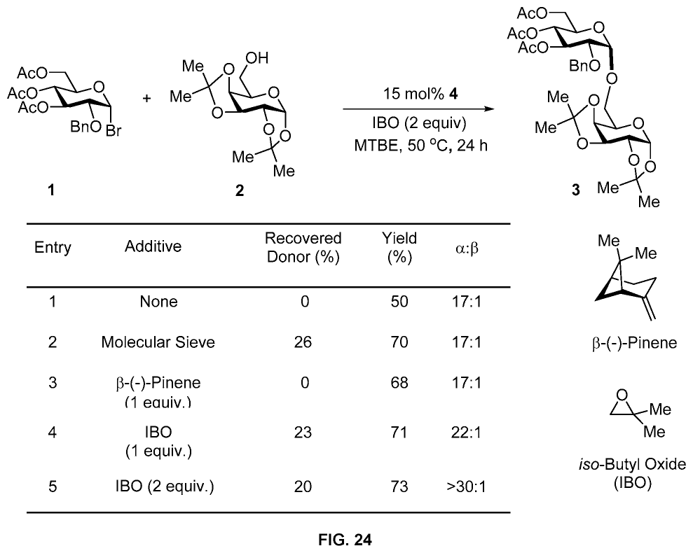

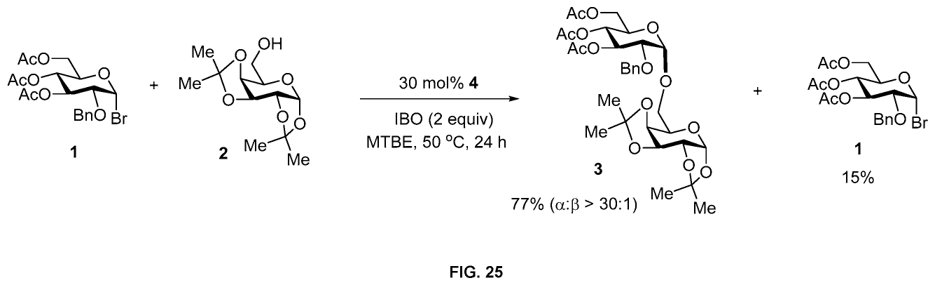

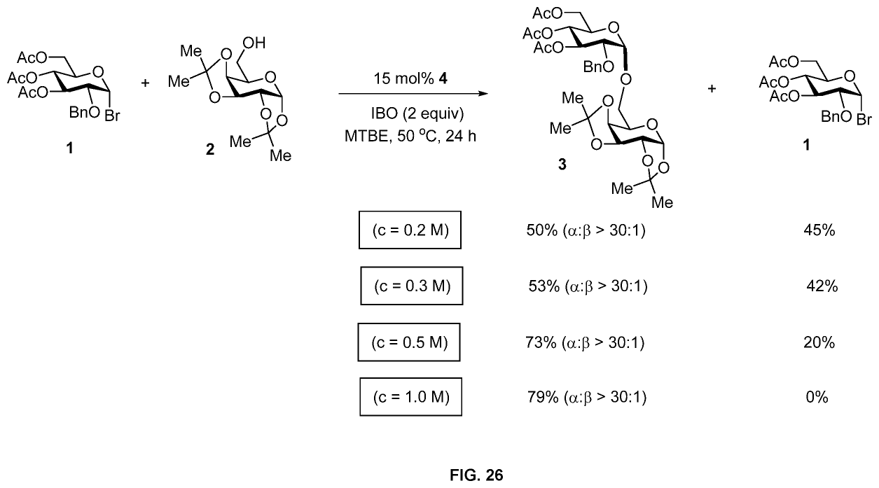

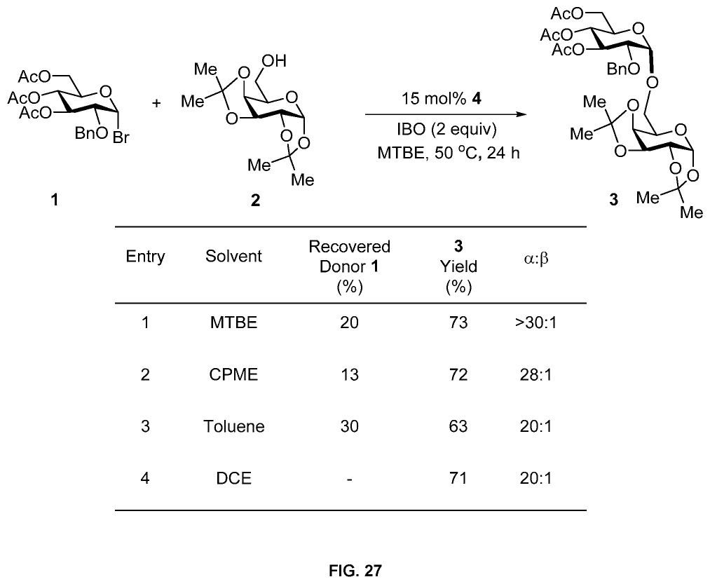

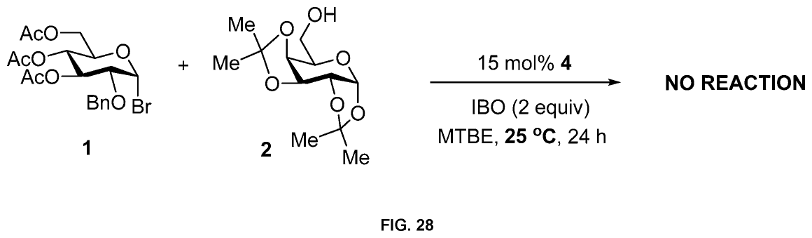

Optimization studies. FIGS. 23-28 show the optimization studies for a range of reaction parameters of various molecules. FIG. 23 shows the screening of small-molecule catalysts. FIG. 24 shows the screening of hydrogen bromide (HBr) scavengers of the reaction. FIG. 25 shows the increasing catalyst loading of the reaction. FIG. 26 shows the effect of various concentrations of the small-molecule catalysts in the reaction. FIG. 27 shows the effect of various solvents when added to the reaction. FIG. 28 shows the effect of the reaction when temperature is added. No reaction occurred when a temperature of 25° C. was added to the reaction.

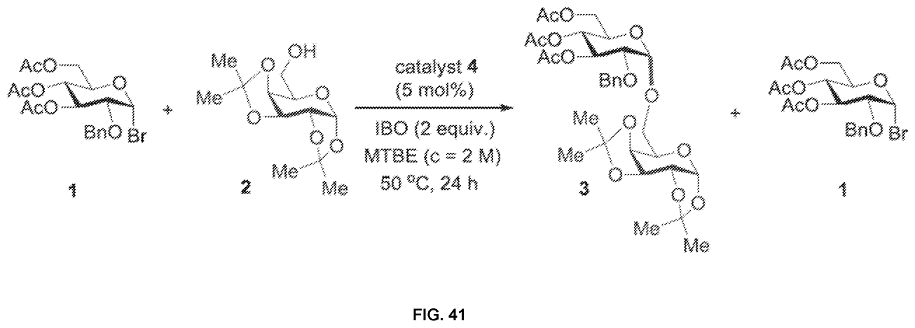

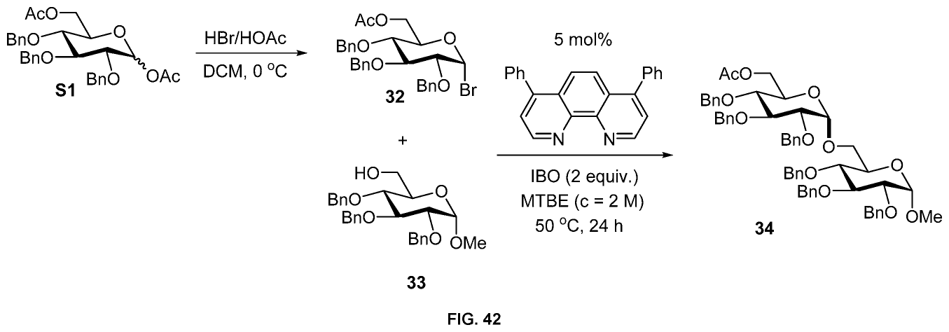

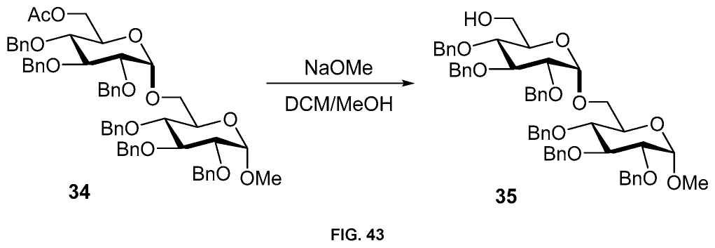

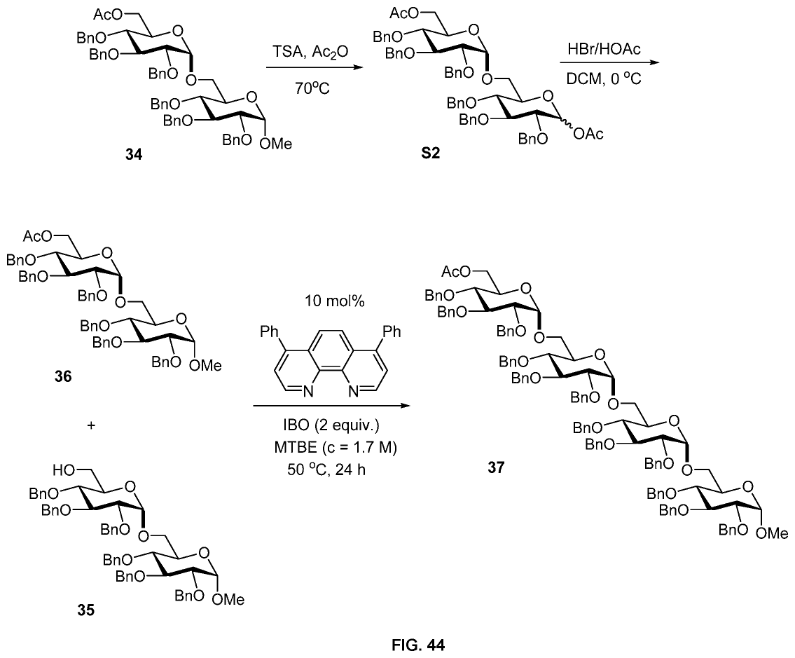

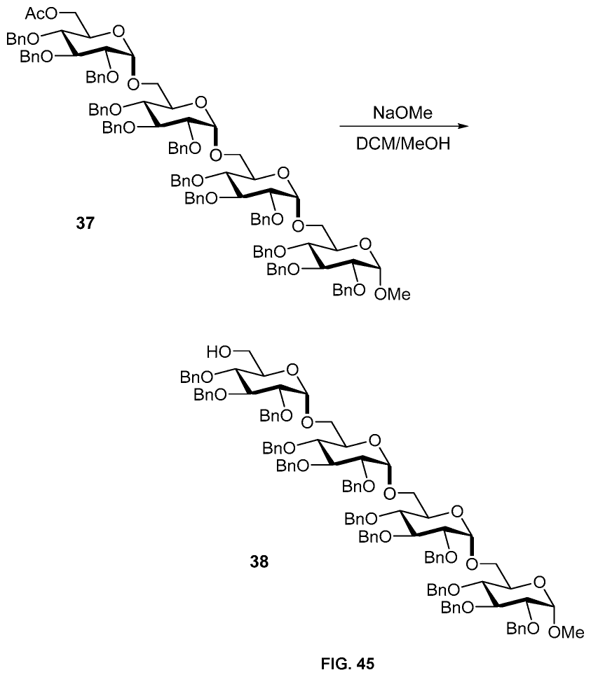

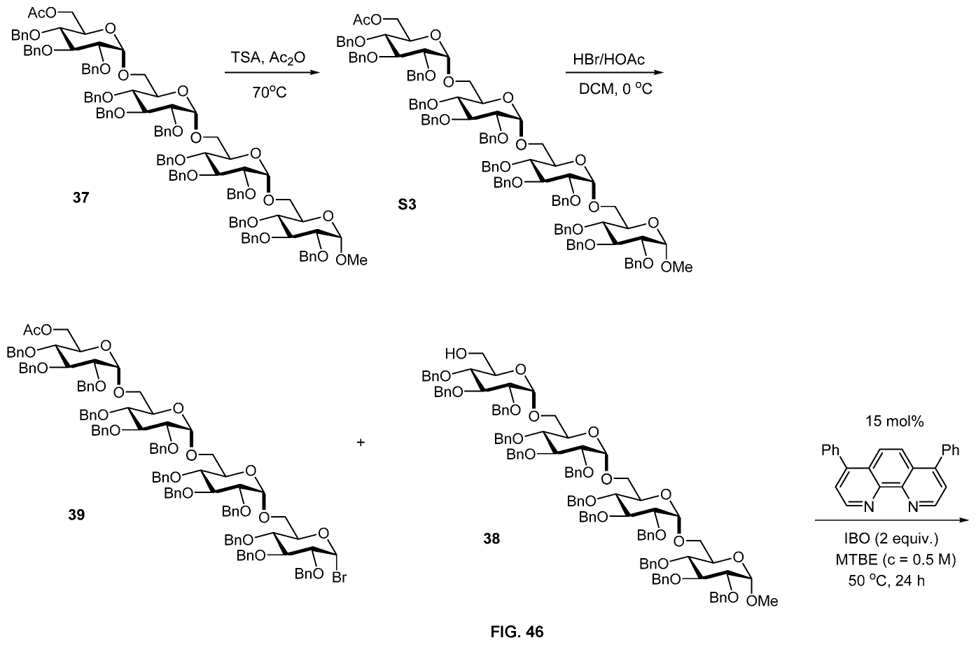

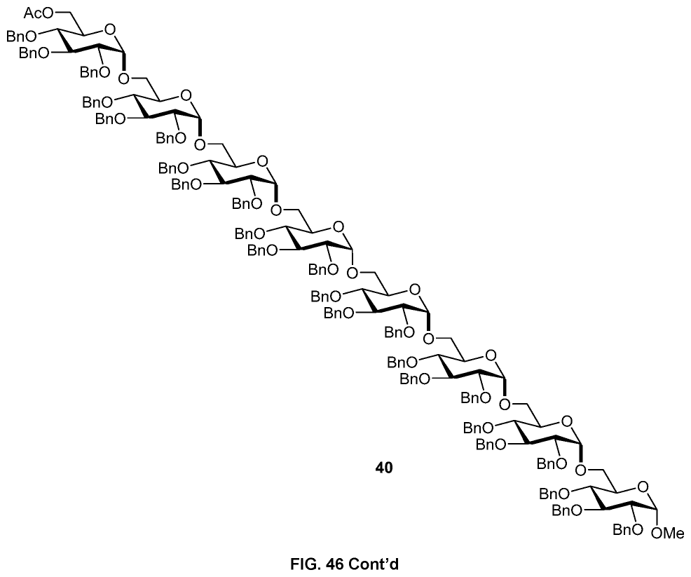

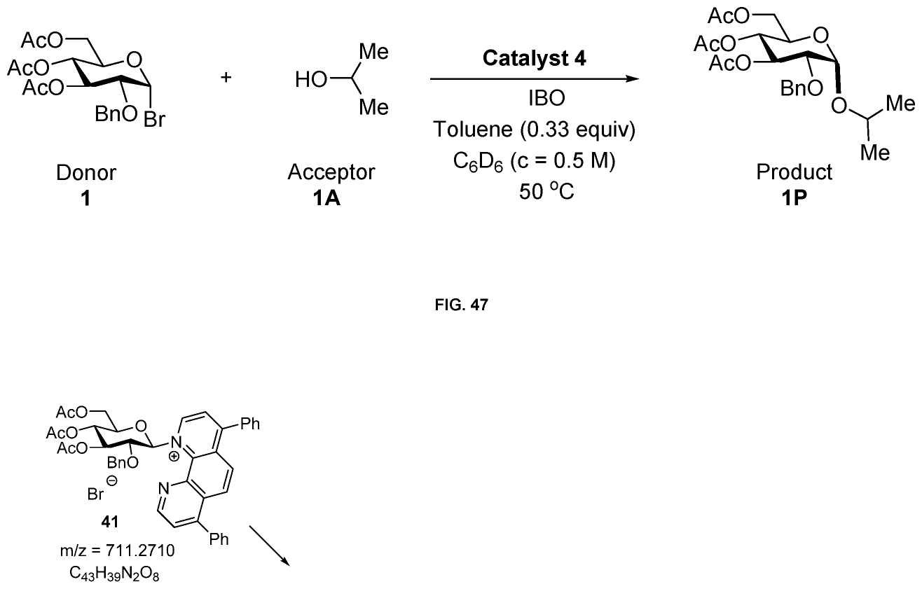

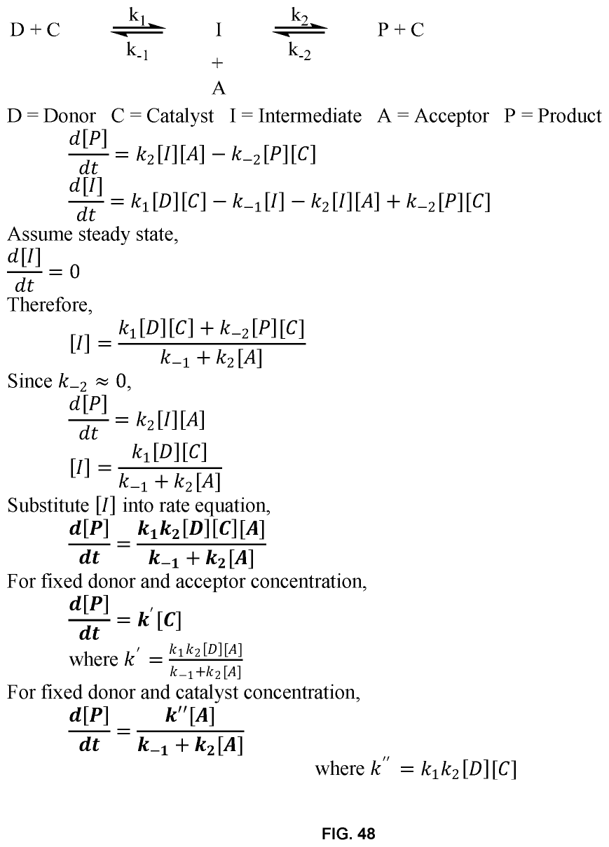

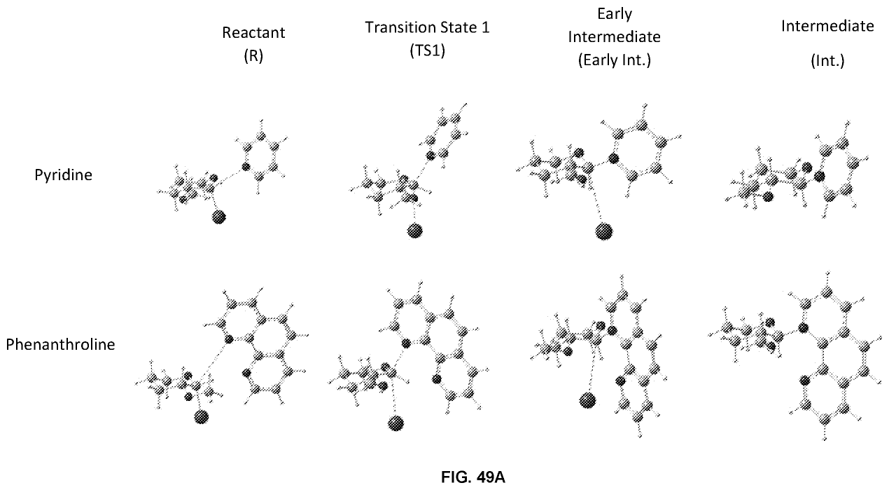

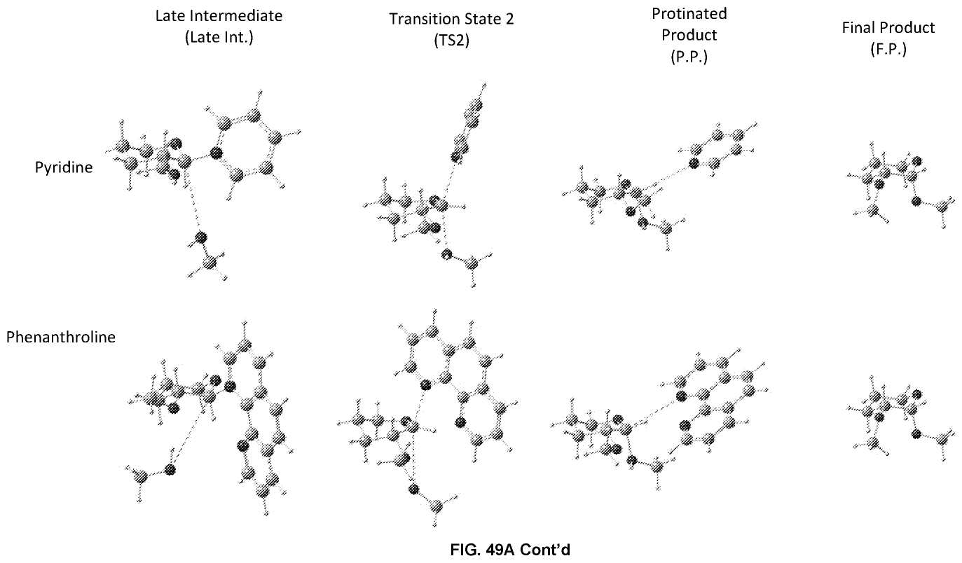

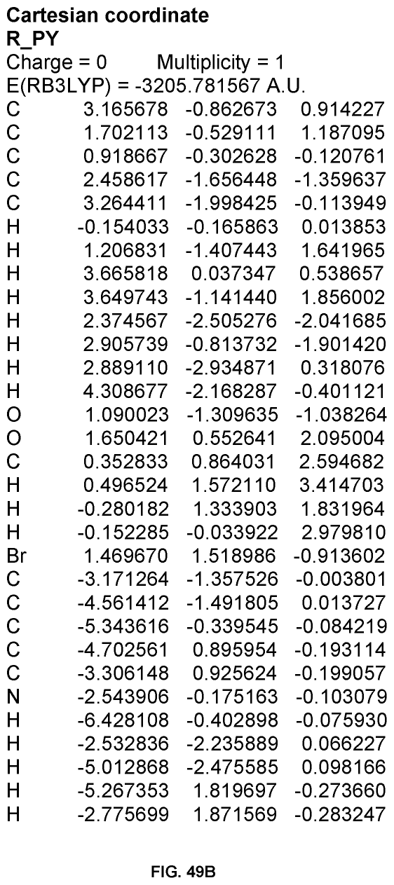

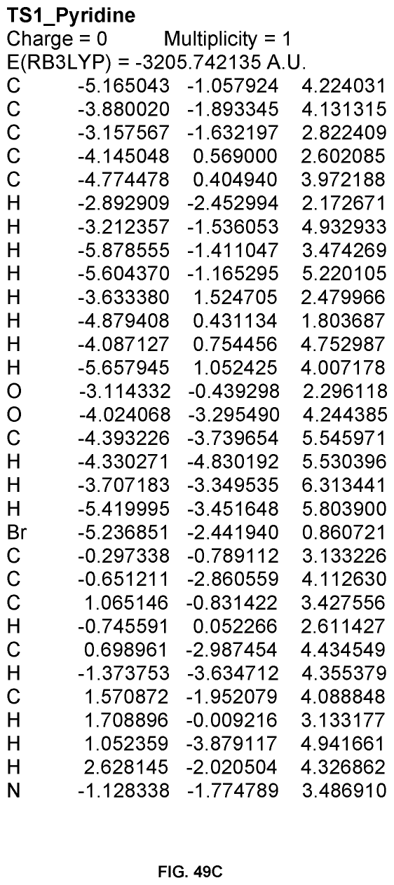

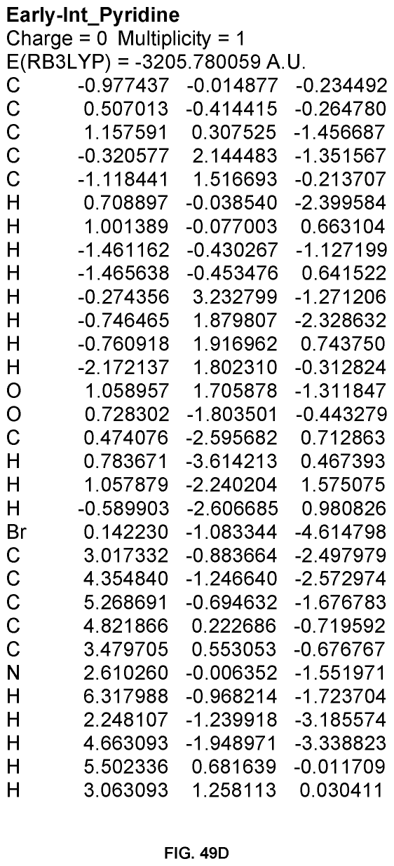

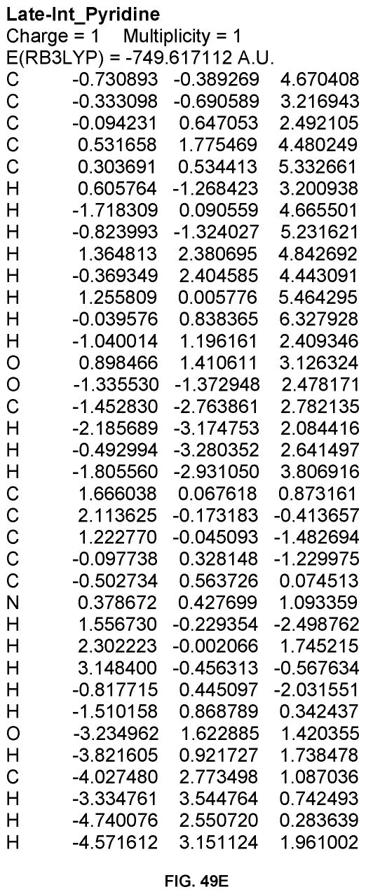

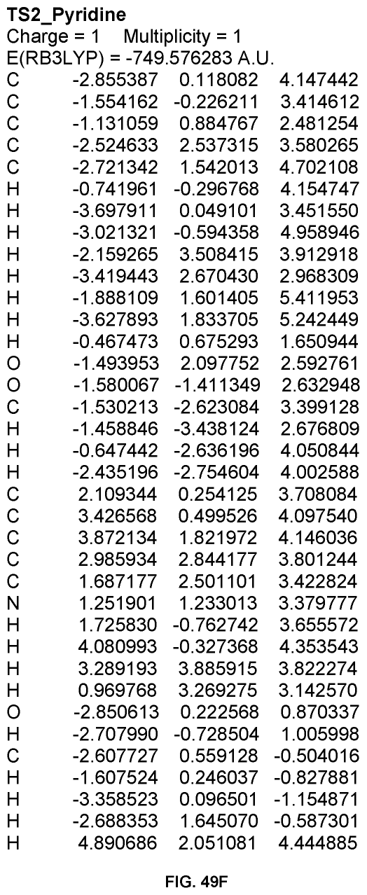

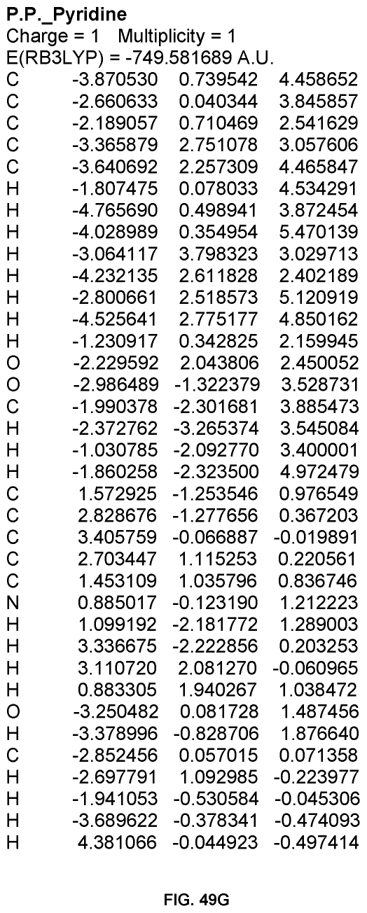

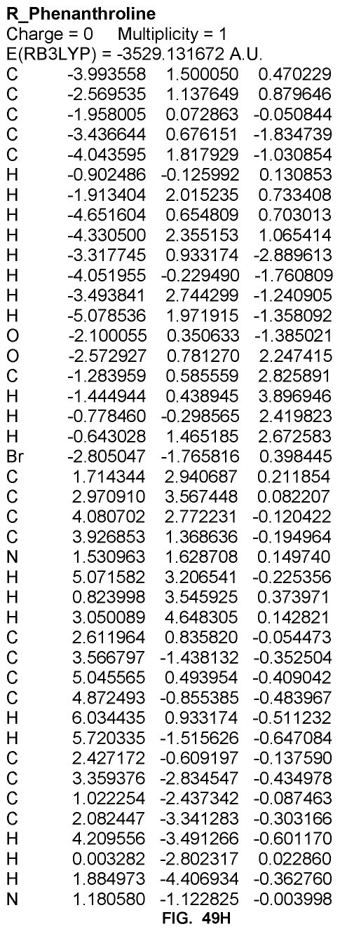

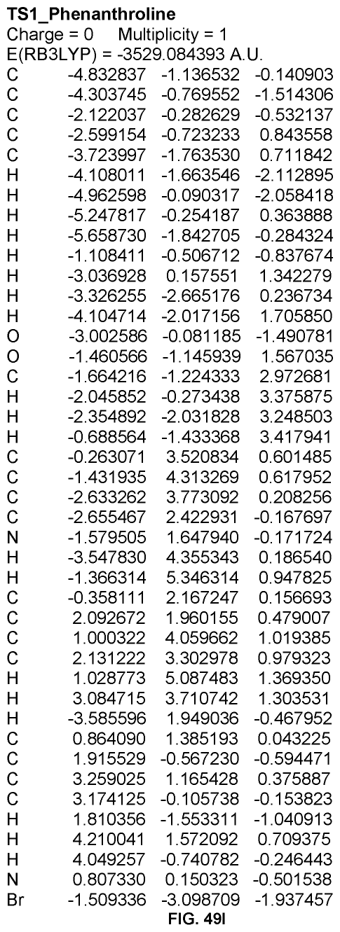

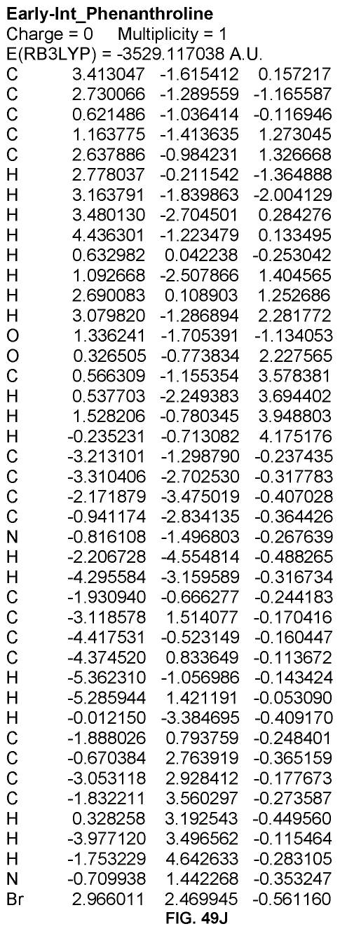

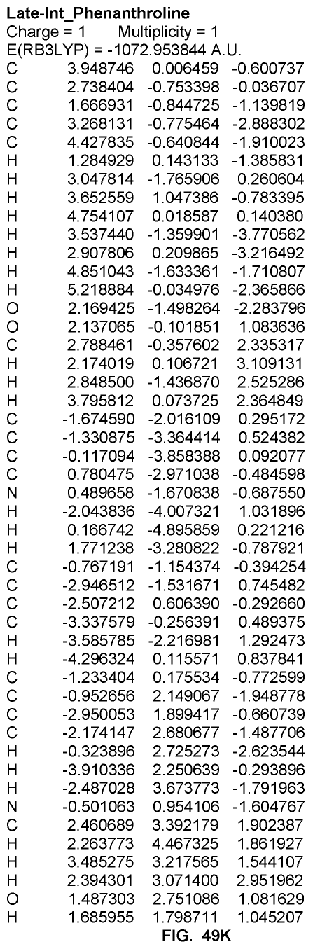

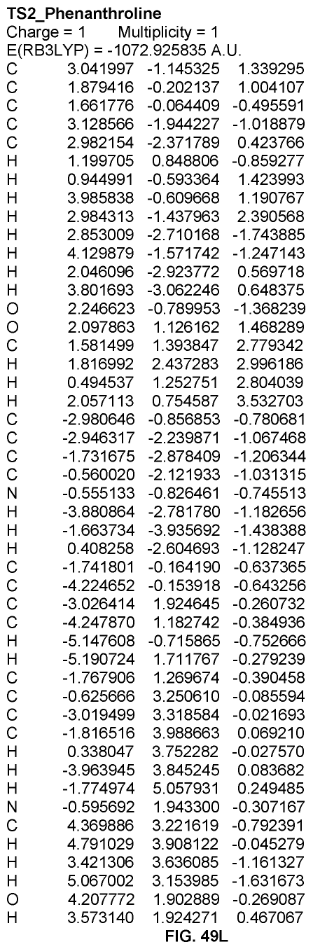

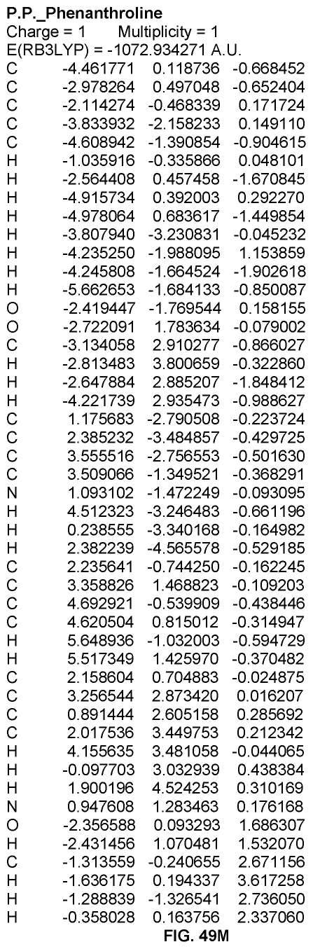

Phenanthroline-catalyzed glycosylation reactions. General Procedure. FIG. 40 shows a phenanthroline-catalyzed glycosylation reaction carried out using various reacting conditions. Under standard conditions A, a 10 mL Schlenk flask was charged with glycosyl bromide (0.2 mmol, 1.0 equiv.), alcohol (0.6 mmol, 3.0 equiv.), catalyst 4 (see FIG. 22, A) (0.03 mmol, 15 mol %), IBO (0.4 mmol, 2.0 equiv.) and MTBE (0.4 mL). The resulting solution was stirred at 50° C. for 24 h, diluted with toluene, and purified by silica gel flash chromatography (toluene/ethyl acetate: 5/1→3/1) to give the desired product. With standard conditions B, a 10 mL Schlenk flask was charged with glycosyl bromide (0.4 mmol, 2.0 equiv.), alcohol (0.2 mmol, 1.0 equiv.), catalyst 4 (0.06 mmol, 30 mol %), IBO (0.4 mmol, 2.0 equiv.) and MTBE (0.2 mL). The resulting solution was stirred at 50° C. for 48 h, diluted with toluene, and purified by silica gel flash chromatography (toluene/ethyl acetate: 5/1→3/1) to give the desired product. In standard conditions B′, a 10 mL Schlenk flask was charged with glycosyl bromide (0.4 mmol, 2.0 equiv.), alcohol (0.2 mmol, 1.0 equiv.), catalyst 4 (0.06 mmol, 30 mol %), IBO (0.4 mmol, 2.0 equiv.) and MTBE (0.4 mL). The resulting solution was stirred at 50° C. for 24 h, diluted with toluene, and purified by silica gel flash chromatography (toluene/ethyl acetate: 9/144/1) to give the desired product. Using standard condition C, a 10 mL Schlenk flask was charged with glycosyl bromide (0.6 mmol, 3.0 equiv.), alcohol (0.2 mmol, 1.0 equiv.), catalyst 4 (0.1 mmol, 50 mol %), IBO (0.6 mmol, 3.0 equiv.) and MTBE (0.2 mL). The resulting solution was stirred at 50° C. for 48 h, diluted with toluene, and purified by silica gel flash chromatography (toluene/ethyl acetate: 5/1→3/1) to give the desired product. In standard condition D, a 10 mL Schlenk flask was charged with glycosyl bromide (0.2 mmol, 2.0 equiv.), alcohol (0.1 mmol, 1.0 equiv.), catalyst 4 (0.02 mmol, 20 mol %), IBO (0.2 mmol, 2.0 equiv.) and MTBE (0.2 mL). The resulting solution was stirred at 25° C. for 24 h, diluted with toluene, and purified by silica gel flash chromatography (toluene/ethyl acetate: 9/144/1) to give the desired product. In standard conditions D′, a 10 mL Schlenk flask was charged with glycosyl bromide (0.2 mmol, 2.0 equiv.), alcohol (0.1 mmol, 1.0 equiv.), catalyst 4 (0.02 mmol, 20 mol %), IBO (0.2 mmol, 2.0 equiv.) and MTBE (0.2 mL). The resulting solution was stirred at 25° C. for 48 h, diluted with toluene, and purified by silica gel flash chromatography (toluene/ethyl acetate: 9/144/1) to give the desired product. In standard condition E, a 10 mL Schlenk flask was charged with glycosyl bromide (0.2 mmol, 1.0 equiv.), alcohol (0.6 mmol, 3.0 equiv.), catalyst 4 (0.04 mmol, 20 mol %), IBO (0.4 mmol, 2.0 equiv.) and MTBE (0.4 mL). The resulting solution was stirred at 25° C. for 24 h, diluted with toluene, and purified by silica gel flash chromatography (toluene/ethyl acetate: 9/144/1) to give the desired product. In standard conditions F, a 10 mL Schlenk flask was charged with glycosyl bromide (0.2 mmol, 1.0 equiv.), alcohol (0.6 mmol, 3.0 equiv.), catalyst 4 (0.04 mmol, 20 mol %), IBO (0.4 mmol, 2.0 equiv.) and MTBE (0.4 mL). The resulting solution was stirred at 50° C. for 24 h, diluted with toluene, and purified by silica gel flash chromatography (toluene/ethyl acetate: 9/144/1) to give the desired product. In standard conditions G, a 10 mL Schlenk flask was charged with glycosyl bromide (0.22 mmol, 1.1 equiv.), alcohol (0.2 mmol, 1.0 equiv.), catalyst 4 (0.03 mmol, 15 mol %), IBO (0.4 mmol, 2.0 equiv.) and MTBE (0.4 mL). The resulting solution was stirred at 50° C. for 24 h, diluted with toluene, and purified by silica gel flash chromatography (toluene/ethyl acetate: 33/149/1) to give the desired product

Under condition A (73% (117 mg), α:β>30:1), the ¹H NMR for disaccharide 3 was: 7.30-7.27 (m, 5H), 5.49 (d, J=5.2 Hz, 1H), 5.43 (t, J=10.0 Hz, 1H), 5.00-4.90 (m, 2H), 4.70-4.55 (m, 3H), 4.34-4.28 (m, 3H), 4.12-4.06 (m, 1H), 4.04-4.00 (m, 2H), 3.80-3.72 (m, 2H), 3.55 (dd, J=10.0, 3.6 Hz, 1H), 2.07 (s, 3H), 2.01 (s, 3H), 2.00 (s, 3H), 1.56 (s, 3H), 1.43 (s, 3H), 1.33 (s, 3H), 1.28 (s, 3H). The ¹H NMR matches what is reported in the literature. The ¹H NMR and ¹³C NMR were reported in the literature (Kamat, et al., J. Org. Chem. 72, 6938-6946 (2007).; Koshiba, et al. Chem.-Asian J. 3, 1664-1677 (2008)).

Under condition D (95% (74.6 mg), α:β=14:1), the ¹H NMR for disaccharide 9 was: δ=7.40-7.09 (m, 20H), 5.53 (d, J=5.0 Hz, 1H), 4.99 (m, 2H), 4.82 (m, 2H), 4.73 (m, 2H), 4.66-4.57 (m, 2H), 4.48 (m, 2H), 4.40-4.29 (m, 2H), 4.09-3.96 (m, 2H), 3.88-3.56 (m, 7H), 1.54 (s, 3H), 1.46 (s, 3H), 1.35 (s, 3H), 1.34 (s, 3H). The ¹H NMR matches what is reported in the literature (Koshiba, et al. Chem.-Asian J. 3, 1664-1677 (2008)).

Under condition E (63% (124.2 mg), α:β=14:1), the ¹H NMR for disaccharide 10 was: δ 7.44-7.16 (m, 35H), 5.08-4.97 (m, 4H), 4.93-4.83 (m, 3H), 4.81-4.70 (m, 4H), 4.67-4.61 (m, 3H), 4.56-4.46 (m, 2H), 4.10-4.01 (m, 2H), 3.93-3.83 (m, 3H), 3.82-3.66 (m, 4H), 3.65-3.58 (m, 2H), 3.52 (dd, J=9.6, 3.6 Hz, 1H), 3.43 (s, 3H). The ¹H NMR matches what is reported in the literature (Koshiba, et al. Chem.-Asian J. 3, 1664-1677 (2008)).

Under condition B (63% (100 mg), α only), the ¹H NMR for disaccharide 11 was: δ=7.38-7.22 (m, 5H), 5.94 (d, J=3.6 Hz, 1H), 5.39 (t, J=10.0 Hz, 1H), 5.31 (d, J=3.6 Hz, 1H), 4.92 (t, J=10.0 Hz, 1H), 4.71 (d, J=12.0 Hz, 1H), 4.60-4.52 (m, 2H), 4.47-4.41 (m, 1H), 4.26-4.4.02 (m, 2H), 4.13-3.97 (m, 5H), 3.57 (dd, J=10.0, 3.6 Hz, 1H), 2.09 (s, 3H), 2.02 (s, 3H), 1.97 (s, 3H), 1.49 (s, 3H), 1.41 (s, 3H), 1.32 (s, 3H), 1.24 (s, 3H). The ¹H NMR matches what is reported in the literature (Demchenko, et al., Org. Lett. 5, 455-458 (2003)).

Under condition B (50% (87 mg), α only), the ¹H NMR and ¹³C NMR for disaccharide 12 was: δ=7.31-7.27 (m, 5H), 5.47-5.35 (m, 3H), 4.91 (t, J=10.0 Hz, 1H), 4.93 (d, J=12.0 Hz, 1H), 4.77-4.60 (m, 4H), 4.33-4.19 (m, 2H), 4.08 (dd, J=12.0, 2.0 Hz, 1H), 3.98-3.90 (m, 1H), 3.85 (t, J=10.0 Hz, 1H), 3.65 (dd, J=10.0, 4.0 Hz, 1H), 3.62-3.59 (m, 3H), 3.57-3.40 (m, 1H), 3.39 (s, 3H), 3.35 (s, 6H), 2.10 (s, 3H), 2.01 (s, 3H), 1.91 (s, 3H). The ¹³C NMR (CDCl3, 100 MHz) was: δ=170.3, 169.8, 154.0, 137.6, 128.4, 128.2, 127.8, 98.3, 98.2, 95.3, 90.9, 80.2, 74.6, 73.7, 71.9, 71.7, 70.7, 70.6, 68.9, 67.1, 62.9, 60.4, 59.1, 55.1, 54.4, 20.74, 20.71, 20.6. The HRMS (ESI) was calculated for C₃₁H₄₃NO₁₅Cl₃ (M+H): 774.1698 (found: 774.1703).

Under condition B (73% (141 mg), α only), the ¹H NMR and ¹³C NMR for disaccharide 13 was: δ=7.40-7.22 (m, 10H), 5.14 (t, J=10.0 Hz, 1H), 4.96 (d, J=4.0 Hz, 1H), 4.90-4.60 (m, 5H), 4.36 (dd, J=12.0, 2.8 Hz, 1H), 4.22-4.09 (m, 3H), 4.00-3.88 (m, 2H), 3.8-3.60 (m, 1H), 3.63 (dd, J=10.0, 3.2 Hz, 1H), 3.34 (s, 3H), 3.38-3.30 (m, 1H), 2.08 (s, 3H), 1.91 (s, 3H), 1.51 (s, 3H), 1.33 (d, J=4.4 Hz, 3H), 1.32 (s, 3H). The ¹³C NMR (CDCl₃, 100 MHz) was: δ=170.9, 169.5, 138.3, 137.5, 128.4, 128.3, 128.2, 128.0, 127.7, 127.6, 109.1, 98.3, 97.7, 81.2, 79.5, 79.4, 76.8, 75.9, 75.3, 74.3, 69.3, 67.6, 64.6, 61.6, 54.6, 28.1, 26.3, 20.73, 20.72, 17.3 (FIG. 77B). The HRMS (ESI) was calculated for C₃₄H₄₄O₁₂Na (M+Na): 667.2730 (found: 667.2735).

Under condition B: (55% (54.3 mg), a:6=7:1), the ¹H NMR for disaccharide 14 was: δ 7.37-7.05 (m, 35H), 5.69 (d, J=3.5 Hz, 1H), 5.03 (d, J=11.6 Hz, 1H), 4.91-4.39 (m, 13H), 4.27 (d, J=12.2 Hz, 1H), 4.11-4.01 (m, 2H), 3.93-3.80 (m, 3H), 3.74-3.69 (m, 1H), 3.67-3.56 (m, 3H), 3.51-3.46 (m, 2H), 3.41-3.39 (m, 1H), 3.37 (s, 3H). The ¹H NMR matches what is reported in the literature (Koshiba, et al. Chem.-Asian J. 3, 1664-1677 (2008)). The ¹H NMR and ¹³C NMR were reported in the literature (Koshiba, et al. Chem.-Asian J. 3, 1664-1677 (2008)).

Under condition B (79% (147 mg), a:6=20:1), the ¹H NMR and ¹³C NMR for disaccharide 15 was: δ=7.76 (d, J=7.6 Hz, 2H), 7.63 (dd, J=7.6, 3.2 Hz, 2H), 7.40-7.23 (m, 9H), 6.04 (d, J=8.8 Hz, 1H), 5.94-5.83 (m, 1H), 5.40 (t, J=9.6 Hz, 1H), 5.32 (d, J=16.0 Hz, 1H), 5.24 (d, J=9.6 Hz, 1H), 4.95 (t, J=10.0 Hz, 1H), 4.77 (d, J=3.2 Hz, 1H), 4.68-4.38 (m, 5H), 4.45-4.40 (m, 2H), 4.26-3.95 (m, 5H), 3.90 (dd, J=10.0, 3.2 Hz, 1H), 3.57 (dd, J=10.0, 3.6 Hz, 1H), 2.05 (s, 3H), 2.02 (s, 6H). The ¹³C NMR (CDCl₃, 100 MHz) was: δ=170.5, 170.0, 169.7, 169.4, 155.9, 143.70, 143.67, 141.2, 137.5, 131.4, 128.9, 129.5, 128.4, 128.1, 128.0, 127.7, 127.6, 127.0, 125.1, 119.9, 119.0, 98.1, 76.6, 72.8, 71.6, 70.2, 68.4, 67.8, 67.2, 66.4, 61.9, 54.5, 47.0, 20.7, 20.59, 20.57. The HRMS (ESI) was calculated for C₄₀H₄₄NO₃ (M+Na): 746.2813 (found: 746.2810).

Under conditions E (77% (120.4 mg), a:6=10:1), the ¹H NMR for disaccharide 16 was: δ 7.43-7.10 (m, 20H), 5.53 (d, J=5.0 Hz, 1H), 5.03 (d, J=3.6 Hz, 1H), 4.95 (d, J=11.4 Hz, 1H), 4.85 (d, J=11.7 Hz, 1H), 4.78-4.72 (m, 3H), 4.62-4.56 (m, 2H), 4.52-4.40 (m, 2H), 4.35-4.29 (m, 2H), 4.10-3.95 (m, 5H), 3.84-3.73 (m, 2H), 3.62-3.51 (m, 2H), 1.54 (s, 3H), 1.45 (s, 3H), 1.35-1.29 (m, 6H). The ¹H NMR matches what is reported in the literature (Lafont, et al., Carbohydr. Res. 341, 695-704 (2006)). The ¹H NMR and ¹³C NMR were reported in the literature (Lafont, et al., Carbohydr. Res. 341, 695-704 (2006)).

Under condition F (58% (86.4 mg), α only), the ¹H NMR for disaccharide 17 was: δ=7.39-7.21 (m, 20H), 4.98-4.92 (m, 2H), 4.87-4.81 (m, 2H), 4.75-4.68 (m, 3H), 4.59 (d, J=11.3 Hz, 1H), 4.48 (d, J=11.9 Hz, 1H), 4.39 (d, J=11.9 Hz, 1H), 4.24 (dd, J=9.2, 4.5 Hz, 1H), 4.16-4.04 (m, 4H), 3.96 (dd, J=10.2, 2.7 Hz, 1H), 3.77-3.60 (m, 2H), 3.50 (dd, J=8.3, 4.6 Hz, 1H), 3.36-3.27 (m, 4H), 1.37 (s, 3H), 1.30 (d, J=6.3 Hz, 3H), 1.25 (s, 3H). The ¹H NMR matches what is reported in the literature (Koshiba, et al. Chem.-Asian J. 3, 1664-1677 (2008)). The ¹H NMR and ¹³C NMR were reported in the literature (Koshiba, et al. Chem.-Asian J. 3, 1664-1677 (2008)).

Under condition A (75% (151 mg), α only), the ¹H NMR and ¹³C NMR for disaccharide 18 was: δ=7.41-7.21 (m, 25H), 5.30 (dd, J=10.8, 3.2 Hz, 1H), 5.05 (d, J=3.2 Hz, 1H), 5.01 (d, J=11.2 Hz, 1H), 4.96 (d, J=11.2 Hz, 1H), 4.85 (d, J=11.2 Hz, 1H), 4.78-4.60 (m, 7H), 4.53 (d, J=11.6 Hz, 1H), 4.17-4.00 (m, 6H), 3.85-3.70 (m, 3H), 3.61 (t, J=9.6 Hz, 1H), 3.45 (dd, J=9.6, 3.6 Hz, 1H), 3.41 (s, 3H), 2.05 (s, 3H), 1.96 (s, 3H). The ¹³C NMR (CDCl₃, 100 MHz) was: δ=170.3, 170.2, 138.6, 138.3, 138.2, 138.0, 137.5, 128.3, 128.25, 128.21, 128.18, 128.0, 127.9, 127.82, 127.77, 127.7, 127.6, 127.5, 127.4, 97.7, 97.3, 81.9, 79.8, 77.8, 75.5, 74.98, 74.95, 74.9, 73.6, 73.1, 72.2, 70.1, 67.8, 66.1, 62.7, 55.0, 20.9, 20.6. The HRMS (ESI) was calculated for C₅₂H₅₈O₁₃Na (M+Na): 913.3775 (found: 913.3787).

Under condition D (80% (55.7 mg), α:β=6:1), the ¹H NMR and ¹³C NMR for disaccharide 19 was: 6=7.42-7.19 (m, 20H), 6.08 (d, J=9.0 Hz, 1H), 5.90-5.80 (m, 1H), 5.29 (d, J=17.2 Hz, 1H), 5.21-5.12 (m, 3H), 4.97 (d, J=11.6 Hz, 1H), 4.85-4.77 (m, 2H), 4.73-4.53 (m, 7H), 4.20 (dd, J=9.9, 2.2 Hz, 1H), 4.01 (dd, J=10.1, 3.6 Hz, 1H), 3.80 (dd, J=10.1, 2.7 Hz, 1H), 3.73 (q, J=6.4 Hz, 1H), 3.60-3.52 (m, 2H), 1.07 (d, J=6.4 Hz, 3H). The ¹³C NMR (CDCl₃, 100 MHz) was: δ=170.0, 156.2, 138.8, 138.5, 138.4, 136.3, 131.6, 128.5, 128.4, 128.3, 128.2, 128.1, 127.8, 127.6, 127.5, 118.6, 98.9, 79.0, 77.6, 76.4, 74.8, 73.3, 73.2, 69.0, 67.0, 66.8, 66.0, 54.4, 16.5. The HRMS (ESI) was calculated for C₄₁H₄₅NO₉Na (M+Na): 718.2987 (found: 718.2967).

Under condition B (88% (107 mg), α:β=20:1), the ¹H NMR and ¹³C NMR for disaccharide 20 was: δ=7.38-7.20 (m, 10H), 6.00-5.93 (m, 2H), 5.40-5.07 (m, 6H), 4.74-4.52 (m, 6H), 4.25 (dd, J=10.0, 6.4 Hz, 1H), 4.04-3.98 (m, 1H), 3.81 (dd, J=10.0, 3.6 Hz, 1H), 3.57 (dd, J=10.0, 3.2 Hz, 1H), 2.13 (s, 3H), 1.97 (s, 3H), 1.08 (d, J=6.4 Hz, 3H). The ¹³C NMR (CDCl₃, 100 MHz) was: δ=170.3, 169.8, 169.5, 156.0, 137.9, 136.2, 131.4, 128.39, 128.36, 128.0, 127.8, 127.6, 119.1, 98.5, 73.4, 73.1, 71.2, 69.9, 69.1, 67.0, 66.2, 64.6, 54.3, 20.7, 20.6, 15.7. The HRMS (ESI) was calculated for C₃₁H₃₇NO₁₁Na (M+Na): 622.2264 (found: 622.2265).

Under condition D (47% (61 mg), α only), the ¹H NMR and ¹³C NMR for disaccharide 21 was: δ=7.42-7.22 (m, 30H), 5.00-4.60 (m, 14H), 4.03-3.96 (m, 2H), 3.88-3.58 (m, 8H), 3.46 (dd, J=12.0, 4.0 Hz, 1H), 3.32 (s, 3H). The ¹³C NMR (CDCl₃, 100 MHz) was: δ=138.82, 138.76, 138.6, 138.4, 138.3, 138.1, 128.32, 128.26, 128.24, 128.18, 127.91, 127.89, 127.8, 127.6, 127.4, 98.3, 97.8, 82.0, 80.0, 77.9, 76.3, 76.2, 75.6, 74.9, 73.9, 73.3, 72.8, 72.4, 71.6, 70.2, 66.4, 60.5, 54.9. The HRMS (ESI) was calculated for C₅₄H₅₈O₁₀Na (M+Na): 889.3922 (found: 889.3943).

Under condition B: (84% (130 mg), α only), the ¹H NMR and ¹³C NMR for disaccharide 22 was: δ=7.42-7.22 (m, 20H), 5.39-5.32 (m, 2H), 5.05 (d, J=3.6 Hz, 1H), 5.02 (d, J=10.8 Hz, 1H), 4.97 (d, J=11.2 Hz, 1H), 4.86 (d, J=11.2 Hz, 1H), 4.76 (d, J=11.2 Hz, 1H), 4.71-4.58 (m, 5H), 4.01 (t, J=10.0 Hz, 1H), 3.96-3.53 (m, 7H), 3.46 (dd, J=9.6, 3.6 Hz, 1H), 3.41 (s, 3H), 2.13 (s, 3H), 2.04 (s, 3H). The ¹³C NMR (CDCl₃, 100 MHz) was: δ=107.1, 169.9, 138.7, 138.3, 138.1, 138.0, 128.3, 128.21, 128.20, 127.9, 127.8, 127.7, 127.6, 127.5, 127.4, 127.2, 97.8, 97.7, 82.0, 79.9, 77.7, 75.5, 74.9, 73.7, 73.2, 72.2, 70.2, 69.4, 69.0, 66.2, 60.3, 55.0, 20.8, 20.7. The HRMS (ESI) was calculated for C₄₄H₅₀O₁₂Na (M+Na): 793.3200 (found: 793.3211).

Under condition B (73% (76 mg), α only), the ¹H NMR and ¹³C NMR for disaccharide 23 was: δ=7.38-7.25 (m, 5H), 5.36-5.30 (m, 2H), 5.08 (d, J=3.6 Hz, 1H), 4.85 (s, 1H), 4.72-4.63 (m, 2H), 4.37 (d, J=13.2 Hz, 1H), 4.19-4.10 (m, 2H), 3.73 (dd, J=10.0, 6.4 Hz, 1H), 3.77-3.55 (m, 2H), 3.40 (dd, J=10.0, 6.4 Hz, 1H), 3.53 (s, 3H), 2.11 (s, 3H), 2.00 (s, 3H), 1.50 (s, 3H), 1.34 (s, 3H), 1.31 (d, J=6.4 Hz, 3H). The ¹³C NMR (CDCl₃, 100 MHz) was: δ=170.2, 169.9, 137.8, 128.3, 127.84, 127.79, 109.0, 98.4, 97.7, 80.2, 76.8, 76.1, 74.0, 73.8, 69.9, 69.4, 64.8, 61.0, 54.6, 27.7, 26.3, 20.9, 20.8, 17.3. The HRMS (ESI) was: calc. for C₂₆H₃₆O₁₁Na (M+Na): 547.2155 (found: 547.2156).

Under condition D (48% (62 mg), a:6=9:1), the ¹H NMR and ¹³C NMR for disaccharide 24 was: δ=7.42-7.22 (m, 30H), 5.00-4.60 (m, 14H), 4.03-3.56 (m, 10H), 3.50 (dd, J=8.0, 4.0 Hz, 1H), 3.32 (s, 3H). The ¹³C NMR (CDCl₃, 100 MHz) was δ=138.7, 138.62, 138.58, 138.4, 138.3, 138.2, 128.4, 128.31, 128.28, 128.2, 128.1, 1287.94, 127.90, 127.83, 127.75, 127.7, 127.6, 127.5, 98.3, 97.9, 82.0, 80.0, 77.7, 76.2, 75.7, 74.9, 73.7, 73.4, 73.2, 72.3, 71.7, 70.0, 66.4, 60.4, 55.0. The HRMS (ESI) was calculated for C₅₄H₅₈O₁₀Na (M+Na): 889.3922 (found: 889.3959).

Under condition A (83% (128 mg), α only), the ¹H NMR and ¹³C NMR for disaccharide 25 was: δ=7.43-7.20 (m, 20H), 5.39-5.36 (m, 2H), 5.02-4.94 (m, 2H), 4.87-4.80 (m, 3H), 4.75-4.60 (m, 5H), 4.07-3.97 (m, 2H), 3.95-3.88 (m, 2H), 3.81 (dd, J=10.0, 3.2 Hz, 1H), 3.76-3.57 (m, 4H), 3.37 (s, 3H), 2.15 (s, 3H), 2.03 (s, 3H). The ¹³C NMR (CDCl₃, 100 MHz) was: δ=170.2, 170.0, 138.7, 138.5, 138.2, 137.9, 128.3, 128.22, 128.19, 128.0, 127.8, 127.7, 127.53, 127.46, 127.41, 127.38, 127.3, 98.0, 97.9, 81.8, 80.2, 77.4, 75.6, 74.8, 73.8, 73.4, 73.0, 69.6, 69.4, 66.6, 60.4, 55.0, 20.9, 20.8. The HRMS (ESI) was: calc. for C₄₄H₅₀O₁₂Na (M+Na): 793.3200 (found: 793.3204).

Under condition B (71% (74 mg), α only), the ¹H NMR and ¹³C NMR for disaccharide 26 was: δ=7.39-7.28 (m, 5H), 5.75 (d, J=3.6 Hz, 1H), 5.34-5.31 (m, 1H), 5.25 (dd, J=10.4, 3.6 Hz, 1H), 4.86 (s, 1H), 4.77 (d, J=12.0 Hz, 1H), 4.65 (d, J=12.0 Hz, 1H), 4.26 (dd, J=6.8, 5.6 Hz, 1H), 4.10 (d, J=5.6 Hz, 1H), 3.99 (dd, J=12.8, 1.2 Hz, 1H), 3.90 (dd, J=10.4, 3.6 Hz, 1H), 3.78-3.70 (m, 1H), 3.67 (dd, J=12.8, 2.0 Hz, 1H), 3.55 (dd, J=10.0, 6.4 Hz, 1H), 3.36 (s, 3H), 2.11 (s, 3H), 2.03 (s, 3H), 1.54 (s, 3H), 1.36 (s, 3H), 1.33 (d, J=6.4 Hz, 3H). The ¹³C NMR (CDCl₃, 100 MHz) was: δ=170.2, 169.9, 138.0, 128.2, 127.7, 109.2, 97.9, 95.7, 78.4, 77.9, 76.0, 73.3, 72.6, 69.4, 68.8, 63.5, 60.7, 54.6, 27.9, 26.3, 20.9, 20.8, 18.2. The HRMS (ESI) was calculated for C₂₆H₃₆O₁₁Na (M+Na): 547.2155 (found: 547.2150).

Under condition A (82% (90 mg), α only), the ¹H NMR and ¹³C NMR for disaccharide 27 was: δ=7.39-7.27 (m, 5H), 5.35-5.30 (m, 2H), 5.08 (d, J=4.0 Hz, 1H), 4.82 (d, J=3.2 Hz, 1H), 4.75-4.63 (m, 2H), 4.29 (d, J=13.2 Hz, 1H), 3.95 (dd, J=10.0, 4.0, Hz, 1H), 3.76-3.40 (m, 6H), 3.59 (s, 3H), 3.49 (s, 3H), 3.40 (s, 3H), 3.27 (dd, J=10.0, 4.0 Hz, 1H), 3.21 (s, 3H), 2.12 (s, 3H), 1.99 (s, 3H). The ¹³C NMR (CDCl₃, 100 MHz) was: δ=170.3, 169.9, 137.8, 128.4, 128.0, 127.9, 98.5, 97.2, 82.5, 81.2, 75.2, 74.2, 74.1, 70.0, 69.8, 69.7, 60.9, 60.7, 58.8, 58.6, 55.1, 20.9, 20.8. The HRMS (ESI) was: calc. for C₂₆H₃₈O₁₂Na (M+Na): 565.2261 (found: 564.2260).

Under condition C (50% (59 mg), α only), the ¹H NMR for disaccharide 28 was: δ=7.40-7.30 (m, 5H), 5.98-5.87 (m, 1H), 5.82 (d, J=8.0 Hz, 1H), 5.46-5.23 (m, 4H), 5.20-5.10 (m, 2H), 4.97 (d, J=3.6 Hz, 1H), 4.70-4.56 (m, 3H), 4.22-4.00 (m, 5H), 3.62 (dd, J=11.2, 3.6 Hz, 1H), 2.14 (s, 3H), 2.05 (s, 3H), 2.02 (s, 3H). The ¹H NMR and ¹³C NMR has been reported in the literature (Friedrichbochnitschek, et al., J. Org. Chem. 54, 751-756 (1989)). The ¹H NMR matches what was reported in the literature (Friedrichbochnitschek, et al., J. Org. Chem. 54, 751-756 (1989).

Under condition C (61% (50 mg), α:β=25:1), the ¹H NMR and ¹⁹F NMR for disaccharide 29 was: δ=5.58-5.45 (m, 2H), 5.11 (d, J=4.0 Hz, 1H), 5.01 (t, J=8.0 Hz, 1H), 4.60 (dd, J=8.0, 4.0 Hz, 1H), 4.48 (ddd, J=48.0, 8.0, 4.0 Hz, 1H), 4.34-4.25 (m, 3H), 4.18-4.00 (m, 3H), 3.90-3.73 (m, 2H), 2.07 (s, 3H), 2.05 (s, 3H), 2.02 (s, 3H), 1.55 (s, 3H), 1.41 (s, 3H), 1.32 (s, 3H), 1.31 (s, 3H). The ¹⁹F NMR (CDCl₃, 100 MHz) was: δ=−201.4. The ¹H NMR and ¹³C NMR were reported in the literature (Vincent, et al., J. Org. Chem. 64, 5264-5279 (1999)). The ¹H NMR matches what was reported in the literature (Vincent, et al., J. Org. Chem. 64, 5264-5279 (1999)).

Under condition D (83% (85 mg), a:6=16:1), the ¹H NMR, ¹³C NMR, and ¹⁹F NMR for disaccharide 30 was: δ=7.39-7.15 (m, 15H), 5.52 (d, J=4.8 Hz, 1H), 5.11 (d, J=4.0 Hz, 1H), 4.90 (d, J=10.8 Hz, 1H), 4.84 (d, J=11.2 Hz, 1H), 4.76 (d, J=10.8 Hz, 1H), 4.66-4.57 (m, 2.5H), 4.51-4.45 (m, 2.5H), 4.33-4.29 (m, 2H), 4.10 (dt, J=12.4, 9.2 Hz, 1H), 4.02 (t, J=6.0 Hz, 1H), 3.90 (dt, J=10.0, 2.0 Hz, 1H), 3.84 (dd, J=10.4, 2.0 Hz, 1H), 3.81-3.66 (m, 4H), 1.55 (s, 3H), 1.45 (5, 3H), 1.35 (5, 3H), 1.34 (5, 3H). The ¹³C NMR (CDCl₃, 100 MHz) was: δ=138.5, 138.2, 137.9, 128.4, 128.3, 127.9, 127.9, 127.8, 127.7, 127.7, 127.7, 109.3, 108.6, 96.8 (d, J_{c-F}=20.9 Hz), 96.3, 91.1 (d, J_{c-F}=191.0 Hz), 80.6 (d, J_{c-F}=16.1 Hz), 76.8 (d, J_{c-F}=8.3 Hz), 75.1 (d, J_{c-F}=2.7 Hz), 75.0, 73.5, 70.74, 70.68, 70.6, 70.2, 68.1, 66.9, 66.2, 26.2, 26.0, 25.0, 24.5. The ¹⁹F NMR (CDCl₃, 100 MHz) was: δ −199.09 (dd, J=49.5, 12.2 Hz). The HRMS (ESI) was calculated for C₃₈H₄₇O₁₀FNa (M+Na): 717.305 (found: 713.3044).

Under condition B (86% 213 mg), α only), the ¹H NMR and ¹³C NMR for disaccharide 31 was: δ=7.42-6.98 (m, 35H), 5.72 (d, J=2.4 Hz, 1H), 5.32 (dd, J=10.8, 2.8 Hz, 1H), 5.18 (s 1H), 5.04 (d, J=9.6 Hz, 1H), 4.90-4.58 (m, 8H), 4.50-4.40 (m, 5H), 4.36-4.25 (m, 3H), 4.17-4.06 (m, 3H), 3.95 (s, 1H), 3.82-3.58 (m, 5H), 3.55-3.45 (m, 2H), 3.43 (s, 3H), 3.39-3.30 (m, 2H), 2.10 (5, 3H), 1.99 (5, 3H), 1.08 (d, J=6.4 Hz, 3H). The ¹³C NMR (CDCl₃, 100 MHz) was δ=170.5, 170.0, 140.0, 138.6, 138.3, 138.03, 138.01, 137.9, 128.5, 128.30, 128.27, 128.2, 128.1, 128.0, 127.8, 127.7, 127.64, 127.58, 127.56, 127.5, 127.31, 127.28, 127.2, 127.02, 126.97, 126.1, 100.2, 98.3, 96.8, 83.8, 80.3, 78.7, 75.6, 75.0, 74.6, 73.7, 73.3, 73.2, 73.03, 72.98, 72.8, 72.2, 71.7, 70.7, 69.7, 69.6, 67.9, 67.6, 64.2, 55.3, 20.7, 20.5, 15.5. The HRMS (ESI) was calculated for O₃₁H₃₇NO₁₁Na (M+Na): 622.2264 (found: 622.2265).

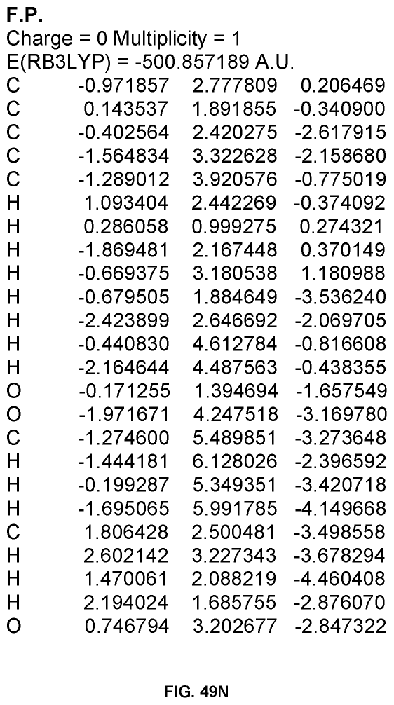

FIG. 41 shows the gram scale synthesis of disaccharide 3. A 50 mL round-bottom flask was charged with glycosyl bromide 1 (1.83 g, 4.0 mmol, 1.0 equiv), alcohol 2 (1.25 g, 4.8 mmol, 1.2 equiv), catalyst 4 (66 mg, 0.2 mmol, 15 mol %), IBO (0.7 mL, 8.0 mmol, 2.0 equiv.) and MTBE (2.0 mL). The resulting solution was stirred at 50° C. for 24 h under open-air atmosphere, diluted with toluene, and purified by silica gel flash chromatography (toluene/ethyl acetate: 5/1→3/1) to give the desired disaccharide 3 (1.784 g, 70%, α:β>30:1) and recovered 1 (0.515 g, 28%).

FIGS. 42-46 show the step-by-step synthesis of octasaccharides 40. In FIG. 42, A 500 mL round-bottom flask was charged with S1 (8.03 g, 15.0 mmol, 1.5 equiv.) and DCM (150 mL). The solution was cooled to 0° C., then HBr/HOAc (33% wt, 15 mL) was added. The solution was stirred at 0° C. for 30 minutes till the reaction was complete as monitored by TLC. The solution was diluted with ethyl acetate, washed with saturated NaHCO₃ solution for two times, dried over Na₂SO₄, concentrated in vacuo, and the afforded glycosyl bromide 32 was used directly.

A 50 mL round-bottom flask was charged with glycosyl bromide 32 (15.0 mmol, 1.5 equiv), alcohol 33 (4.63 g, 10.0 mmol, 1.0 equiv), BPhen (166 mg, 0.5 mmol, 5 mol %), IBO (1.78 mL, 20.0 mmol, 2.0 equiv.) and MTBE (2.0 mL). The resulting solution was stirred at 50° C. for 24 h under open-air atmosphere, diluted with toluene, and purified by silica gel flash chromatography (toluene/ethyl acetate: 20/1→10/1) to give the desired disaccharide 34 (8.36 g, 89%, α:β>25:1).

The ¹H NMR for disaccharide 34 was: δ=7.40-7.28 (m, 30H), 5.00-4.60 (m, 14H), 4.28-4.22 (m, 2H), 4.05-4.00 (m 2H), 3.90-3.80 (m, 3H), 3.78-3.66 (m, 2H), 3.55-3.46 (m, 3H), 3.40 (s, 3H), 2.01 (s, 3H). The ¹H and ¹³C NMR, of disaccharide 34, were reported in the literature (Kovac, et al., Carbohydr. Res. 184, 87-112 (1988)).

FIG. 43 shows the synthesis of disaccharide 34. A 50 mL oven-dried RBF was charged with 34 (350 mg, 0.37 mmol, 1.0 equiv.), MeONa (10 mg, 0.19 mmol, 0.5 equiv.), and CH₂Cl₂/MeOH (1 mL/1 mL). The solution was stirred at RT overnight. When the reaction was complete as monitored by TLC, the reaction mixture was evaporated, and purified by flash chromatography on silica gel (hexane/ethyl acetate: 2/1→1/1) to afford 341 mg (99%) of 35. FIG. The ¹H NMR for disaccharide 35 was: ¹H NMR (CDCl₃, 400 MHz): δ=7.40-7.28 (m, 30H), 5.00-4.52 (m, 14H), 4.28-4.22 (m, 2H), 4.05-3.46 (m, 10H), 3.35 (s, 3H). The ¹H and ¹³C NMR, of disaccharide 35, were reported in the literature (Kovac, et al., Carbohydr. Res. 184, 87-112 (1988)).

FIG. 44 show the synthesis of tetraccharide 37. A 50 mL round-bottom flask was charged with 34 (940 mg, 1.0 mmol, 1.0 equiv.), PTSA H₂O (248 mg, 1.3 mmol, 1.3 equiv.), and Ac₂O (6 mL). The solution was stirred at 70° C. for 2 h. The solution was diluted with ethyl acetate, washed with saturated NaHCO₃ (aq.) for three times, concentrated in vacuo, and the residue was purified by silica gel flash chromatography (hexane/ethyl acetate=4/1-2/1) to afford 572 mg (61%) of S2. The NMR for disaccharide S2 was: ¹H NMR (CDCl₃, 400 MHz): δ=7.40-7.28 (m, 30H), 6.28 (d, J=4.0 Hz, 1H), 5.00-4.60 (m, 13H), 4.28-4.22 (m, 2H), 4.05-3.46 (m, 11H), 2.13 (s, 3H), 1.99 (s, 3H). The ¹H and ¹³C NMR, of disaccharide S2, were reported in the literature (Kovac, et al., Carbohydr. Res. 184, 87-112 (1988)).

A 50 mL round-bottom flask was charged with S2 (500 mg, 0.51 mmol, 1.5 equiv.) and DCM (30 mL). The solution was cooled to 0° C., then HBr/HOAc (33% wt, 0.5 mL) was added. The solution was stirred at 0° C. for 20 minutes till the reaction was complete as monitored by TLC. The solution was diluted with ethyl acetate, washed with saturated NaHCO₃ solution for two times, dried over Na₂SO₄, concentrated in vacuo, and the afforded glycosyl bromide 36 was used directly.

A 50 mL round-bottom flask was charged with glycosyl bromide 36 (0.51 mmol, 1.5 equiv), alcohol 33 (320 mg, 0.34 mmol, 1.0 equiv), BPhen (11 mg, 0.034 mmol, 10 mol %), IBO (0.06 mL, 0.68 mmol, 2.0 equiv.) and MTBE (0.2 mL). The resulting solution was stirred at 50° C. for 24 h under open-air atmosphere, diluted with toluene, and purified by silica gel flash chromatography (toluene/ethyl acetate: 20/1→10/1) to give the desired tetraccharide 37 (520 mg, 86%, α:β>25:1).

The ¹H NMR and ¹³C NMR for tetraccharide 37 was: ¹H NMR (CDCl₃, 400 MHz) δ=7.42-7.28 (m, 60H), 5.11 (d, J=4.0 Hz, 1H), 5.05-4.60 (m, 27H), 4.28-4.22 (m, 2H), 4.08-4.00 (m, 4H), 3.90-3.75 (m 12H), 3.60-3.42 (m, 6H), 3.40 (s, 3H), 2.03 (s, 3H); ¹³C NMR (CDCl₃, 100 MHz) δ=170.6, 138.8, 138.6, 138.5, 138.4, 138.3, 138.1, 137.9, 128.30, 128.25, 128.2, 128.0, 127.94, 127.88, 127.73, 127.70, 127.65, 127.6, 127.5, 127.44, 127.41, 127.37, 127.32, 127.28, 97.9, 97.00, 96.95, 82.0, 81.5, 80.3, 80.2, 80.1, 80.0, 77.6, 77.4, 75.6, 75.5, 75.3, 74.9, 74.8, 73.3, 72.3, 72.2, 72.1, 70.71, 70.64, 70.5, 70.3, 68.6, 65.6, 65.5, 65.5, 62.9, 55.1, 20.8. The HRMS (ESI) was also reported. The ¹H and ¹³C NMR, of disaccharide 37, were reported in the literature (Kovac, et al., Carbohydr. Res. 184, 87-112 (1988)). The HRMS calculation for C₁₁₁H₁₁₈O₂₂Na (M+Na) was: 1825.8007 (found: 1925.8009).

FIG. 45 shows the synthesis of disaccharide 38. A 50 mL oven-dried RBF was charged with 37 (250 mg, 0.14 mmol, 1.0 equiv.), MeONa (4 mg, 0.07 mmol, 0.5 equiv.), and CH₂Cl₂/MeOH (1 mL/1 mL). The solution was stirred at RT. When the reaction was complete as monitored by TLC, the reaction mixture was evaporated, and purified by flash chromatography on silica gel (toluene/ethyl acetate: 5/1→3/1) to afford 170 mg (70%) of 38.

The ¹H NMR and ¹³C NMR for disaccharide 38 was: ¹H NMR (CDCl₃, 400 MHz) δ=7.42-7.28 (m, 60H), 5.05-4.60 (m, 28H), 4.05-3.40 (m, 24H), 3.37 (s, 3H); ¹³C NMR (CDCl₃, 100 MHz) δ=138.8, 128.7, 138.6, 138.54, 138.45, 138.4, 138.3, 138.2, 138.1, 128.33, 128.30, 128.27, 128.2, 127.94, 127.91, 127.8, 127.64, 127.57, 127.5, 127.40, 127.35, 127.1, 98.0, 97.1, 97.0, 82.0, 81.5, 81.4, 77.7, 77.5, 75.6, 75.42, 75.37, 74.9, 73.3, 72.31, 72.25, 72.2, 70.82, 70.75, 70.7, 70.5, 65.8, 65.6, 65.4, 61.8, 55.1. The ¹H and ¹³C NMR, of disaccharide 38, were reported in the literature (Kovac, et al., Carbohydr. Res. 184, 87-112 (1988)).

FIG. 46 shows the synthesis of disaccharide 40. A 50 mL round-bottom flask was charged with 37 (500 mg, 0.27 mmol, 1.0 equiv.), PTSA H₂O (67 mg, 0.35 mmol, 1.3 equiv.), and Ac₂O (3 mL). The solution was stirred at 70° C. for 2 h. The solution was diluted with ethyl acetate, washed with saturated NaHCO₃ (aq.) for three times, concentrated in vacuo, and the residue was purified by silica gel flash chromatography (toluene/ethyl acetate=8/1-5/1) to afford 249 mg (51%) of S3. The ¹H and ¹³C NMR of disaccharide S3 has been reported in the literature (Kovac, et al., Carbohydr. Res. 184, 87-112 (1988)). The ¹H NMR (CDCl₃, 400 MHz) shows: 8=7.40-7.28 (m, 60H), 6.34 (d, J=4.0 Hz, 1H), 5.00-4.60 (m, 27H), 4.28-4.22 (m, 2H), 4.05-3.46 (m, 22H), 2.08 (s, 3H), 2.02 (s, 3H).

A 25 mL round-bottom flask was charged with S3 (110 mg, 0.06 mmol, 1.5 equiv.) and DCM (6 mL). The solution was cooled to 0° C., then HBr/HOAc (33% wt, 0.06 mL) was added. The solution was stirred at 0° C. for 15 minutes until the reaction was complete as monitored by TLC. The solution was diluted with ethyl acetate, washed with saturated NaHCO₃ solution for two times, dried over Na₂SO₄, concentrated in vacuo, and the afforded glycosyl bromide 39 was used directly.

A 50 mL round-bottom flask was charged with glycosyl bromide 39 (0.06 mmol, 1.5 equiv), alcohol 38 (70 mg, 0.04 mmol, 1.0 equiv), BPhen (2 mg, 0.006 mmol, 15 mol %), IBO (0.007 mL, 0.08 mmol, 2.0 equiv.) and MTBE (0.08 mL). The resulting solution was stirred at 50° C. for 24 h under open-air atmosphere, diluted with toluene, and purified by silica gel flash chromatography (toluene/ethyl acetate: 20/1→10/1) to give the desired disaccharide 40 (109 mg, 77%, α:β>25:1).

The ¹H NMR and ¹³C NMR for disaccharide 40 was: ¹H NMR (CDCl₃, 400 MHz) δ=7.42-7.28 (m, 120H), 5.05 (d, J=4.0 Hz, 1H), 5.05-4.40 (m, 54H), 4.25-4.18 (m, 2H), 4.08-4.00 (m, 8H), 3.90-3.30 (m 39H), 3.32 (s, 3H), 1.98 (s, 3H); ¹³C NMR (CDCl3, 100 MHz) δ=170.7, 138.8, 138.6, 138.54, 138.46, 138.4, 138.2, 138.0, 128.38, 128.36, 128.3, 128.2, 128.04, 127.99, 127.9, 127.8, 127.6, 127.5, 127.4, 127.34, 127.27, 98.0, 97.30, 97.25, 97.18, 97.16, 97.1, 97.0, 82.1, 81.5, 80.4, 80.3, 80.2, 80.0, 75.7, 75.5, 75.4, 75.0, 74.9, 73.4, 72.2, 72.13, 72.07, 70.88, 70.87, 70.8, 70.7, 70.6, 68.7, 65.5, 63.0, 55.1, 20.8. The ¹H and ¹³C NMR, of disaccharide 40, were reported in the literature (Kovac, et al., Carbohydr. Res. 184, 87-112 (1988)). The HRMS calculation for C₂₁₉H₂₃₀O₄₂Na (M+Na) was: 3554.5754 (found: 3554.5867).

Mechanistic studies. High resolution mass spectrometry analysis of glycosyl phenanthrolium 34. FIG. 32 shows the synthesis of disaccharide 3. A 10 mL bottle was charged with glycosyl bromide 1 (46 mg, 0.1 mmol, 1.0 equiv), 4 (100 mg, 0.3 mmol, 3.0 equiv.), and MTBE (1.2 mL). The reaction mixture was stirred at 50° C. for 24 h. Formation of the glycosyl phenanthrolinium ion 41 was confirmed using ESI with an m/z ratio of 711.2710 (see below). Subsequent fragmentation of 41 using CID led to the formation of various fragment ions, most notably the phenanthroline species with an m/z ratio of 331.1396 (see below). The mixture was concentrated and dried in vacuo. The resulting residue was mixed with alcohol 2 (39 mg, 0.15 mmol, 1.5 equiv.), and MTBE (0.4 mL). The reaction mixture was stirred at 50° C. for 12 h, formation of the desired disaccharide 3 was confirmed by high resolution ESI, diluted with toluene, and purified by silica gel flash chromatography (toluene/ethyl acetate: 5/1→3/1) to give the desired disaccharide 3 (31 mg, 50%, α:β>30:1).

General experimental procedure for kinetic studies. FIG. 47 shows the synthesis of product 1P. A 10 mL scintillation vial was charged with glycosyl bromide 1 (fixed amount, 0.25 mmol, 1.0 equiv), isopropanol acceptor 1A (vary amount from 0.5 to 5 equiv), catalyst 4 (vary amount from 2 to 20 mol %), IBO (vary amount from 1.5 to 3 equiv), toluene (internal standard, 0.083 mmol, 0.33 equiv), and C₆D₆ (0.5 mL). The resulting solution was then transferred to a 5 mm NMR tube.

¹H NMR spectrum was acquired on a 400 MHz instrument before heating. Then the mixture in NMR tube was then consistently shaken and heated in a 50° C. water bath. Between 3 and 60 h, spectra were obtained depending on the experiment. Example spectra and example rate plot were based on standard condition: 0.25 mmol glycosyl bromide 1 (1.0 equiv), 0.75 mmol acceptor (3.0 equiv), 15 mol % catalyst 4, 0.5 mmol IBO (2 equiv), 0.083 mmol toluene (0.33 equiv) as an internal standard, and 0.5 mL C6D₆ (0.5 M).

Spectra processing. The spectra for each kinetic experiment were processed using MestReNova (v. 6.0.2, Mestrelab Research S.L.). The concentration of product was measured by integration of its H-1 proton against the toluene internal standard, 8=2.1 ppm. Peak fitting or deconvolution algorithms were not used for integration. An example spectra array for a kinetic experiment is shown in FIG. 35.

Rates of the reactions in the disclosure was obtained by using the rate equation derivation (FIG. 48).

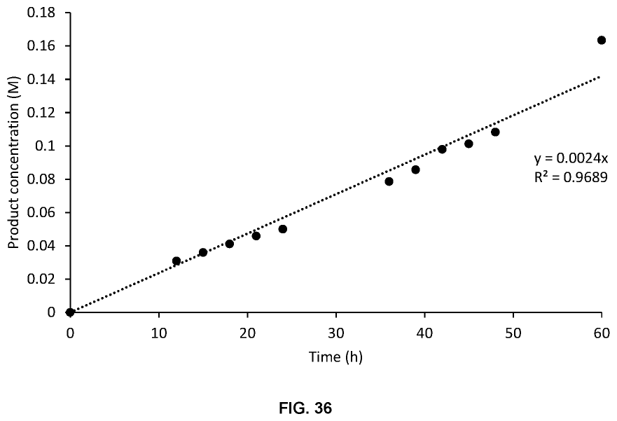

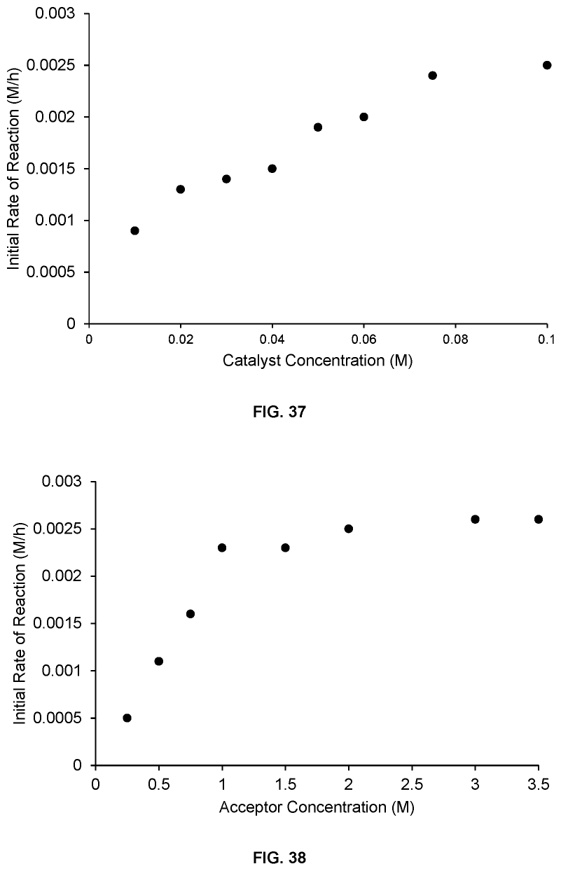

Graphing. For each kinetic experiment, the concentration of product versus time were plotted on Excel 2016. Linear regression was obtained by best fitting with all points (FIG. 36). Slope of the best-fit line represents the initial rate of reaction for each kinetic experiment. The initial rate was then graphed against catalyst concentration for fixed acceptor concentration (FIG. 37), and against acceptor concentration for fixed catalyst concentration (FIG. 38). The product formation versus time was also compared at different equivalent of IBO (FIG. 39).

DFT calculations. All calculations were carried out with Gaussian 09 (Gaussian 09 Rev. E.01 (Wallingford, C T, 2013)). Geometry optimization for reactant, intermediates, transition states, and products were computed at the B3LYP/6-31+G(d,p) level of theory (Stephens, et al., J. Phys. Chem. 98, 11623-11627 (1994); Becke, et al., J. Chem. Phys. 98, 5648-5652 (1993); Lee, et al., Phys. Rev. B. 37, 785-789 (1988); Becke, et al., Phys. Rev. A. 38, 3098-3100 (1988); Vosko, et al., Can. J. Phys. 58, 1200-1211 (1980); Francl, et al, J. Chem. Phys. 77, 3654-3665 (1982); Gordon, et al, Chem. Phys. Lett. 76, 163-168 (1980); Hariharan, et al., Mol. Phys. 27, 209-214 (1974); Harihara. Pc et al., Theor. Chim. Acta. 28, 213-222 (1973); Hehre, et al., J. Chem. Phys. 56, 2257-+(1972); Ditchfield, et al., J. Chem. Phys. 54, 724-+(1971)) with the SMD implicit solvation model (Marenich, et al., J. Phys. Chem. B. 113, 6378-6396 (2009)) in diethyl ether. There is only one imaginary frequency for transition state structures and no imaginary frequency for reactant, intermediates, and products. Non-covalent interactions (NCI) were calculated with the NCIPLOT program (Johnson, et al., J. Am. Chem. Soc. 132, 6498-6506 (2010)).

FIGS. 49A-49M show the optimized structures and the cartesian coordinates for the optimized structures. FIG. 49B shows the cartesian coordinate for reactant pyridine. FIG. 49C shows the cartesian coordinate for transition state 1_pyridine. FIG. 49D shows the cartesian coordinate for early intermediate_pyridine. FIG. 49E shows the cartesian coordinate for late intermediate_pyridine. FIG. 49F shows the cartesian coordinate for transition state 2_pyridine. FIG. 49G shows the cartesian coordinate for protonated product_pyridine. FIG. 49H shows the cartesian coordinate for reactant phenanthroline. FIG. 49I shows the cartesian coordinate for transition state 1_phenanthroline. FIG. 49J shows the cartesian coordinate for early intermediate_phenanthroline. FIG. 49K shows the cartesian coordinate for late-intermediate_phenanthroline. FIG. 49L shows the cartesian coordinate for transition state 2_phenanthroline. FIG. 49M shows the cartesian coordinate for protonated product_phenanthroline. FIG. 49N shows the cartesian coordinate for the final product.

(v) Additional Xenograft Models. This section describes additional xenograft models and methods that can be used to confirm the anti-cancer effects of compounds described herein. Particular dose examples are provided, however, as will be understood by one of ordinary skill in the art, optimization of particular parameters may be needed.

(vi-a) Mesothelioma. Mesothelioma tumors express high levels of heparanase and exhibit high sensitivity to treatment with heparanase-inhibiting compounds (i.e., PG545), providing a strong rational for confirming the effect of Glycopolymer on mesothelioma progression (Barash et al., J. Nat. Cancer Inst. 110:1102-1114, 2018).

Experimental design. Luciferase-labeled MSTO-211H human mesothelioma cells are inoculated (5×10⁶/0.2 ml) i.p into NOD/SCID mice. Eight days after cell inoculation, mice are randomly assigned to 2 cohorts (8 mice each) receiving: (a) vehicle; and (b) Glycopolymer (i.p, 600 μg/mouse; Daily). Tumor development is inspected (once a week) by IVIS imaging following administration of luciferin (see below).

Other models (i.e., LUC-U87 human glioma; LUC-TC-71 human Ewing's sarcoma, LUC-PANC-02 mouse pancreatic carcinoma) can be applied as well.

The injected dose (600 μg/mouse; Daily) is based on results with Roneparstat (glycol-split heparin=SST0001) administered (1 mg/mouse) twice a day (Ritchie et al., Clin Cancer Res, 2011; 17:1382-93).

(vi-b) Myeloma. Injection of myeloma cells into the tail vein of mice has been widely used to study myeloma homing and growth within the bone marrow. CAG human myeloma cells localize almost exclusively to bone following i.v. injection, thus representing an orthotopic model that mimics the human disease (Ramani et al., Oncotarget. 2016; 7:1598-607). The cells are highly aggressive in vivo, exhibit rampant metastasis and promote widespread osteolysis, thereby mimicking aggressive human disease. Thus, if a test compound is efficacious against CAG cells growing within the murine bone marrow in vivo, it has a high probability of being effective in human myeloma patients.

Experimental design. Luciferase-labeled CAG human myeloma cells (3×10⁶) are injected into the tail vein of NOD/SCID mice. 3-5 days after cell inoculation, mice are randomly assigned to 2 cohorts (8 mice each) receiving: (a) vehicle; and (b) Glycopolymer (i.p, 600 μg/mouse; Daily). Tumor development is inspected (once a week) by IVIS imaging following administration of luciferin (see below).

(vi-c) B-Lymphoma. B-lymphoma bearing mice exhibit high sensitivity to treatment with heparanase-inhibiting compounds (PG545) and neutralizing antibodies (M. Weissmann et al., PNAS, 113:704-709, 2016), providing a strong rational for confirming the effect of Glycopolymer on B-lymphoma progression.

Experimental design. Luciferase-labeled Raji lymphoma cells (5×10⁶) cells are injected into the tail vein of NOD/SCID mice. 3-5 days after cell inoculation, mice are randomly assigned to 2 cohorts (8 mice each) receiving: (a) vehicle; and (b) Glycopolymer (i.p, 600 μg/mouse; Daily). Tumor development is inspected (once a week) by IVIS imaging following administration of luciferin.

It is expected that treatment with the Glycopolymer will yield at least a partial inhibition of tumor growth. In subsequent experiments, a combined treatment with chemotherapy can be considered (e.g., cisplatin for mesothelioma, melphalan for myeloma, and daunorubicin for B-lymphoma).

IVIS imaging. Bioluminescent imaging of luciferase-expressing tumors is performed with a highly sensitive, cooled charge coupled device (CCD) camera mounted in a light-tight specimen box (IVIS; Xenogen Corp., Waltham, Mass.). Imaging is performed in real time, is non-invasive and provides quantitative data. Briefly, mice are injected intraperitoneally with D-luciferin substrate at 150 mg/kg, anesthetized and placed onto a warmed stage inside the light-tight camera box, with continuous exposure to isoflurane (EZAnesthesia, Palmer, Pa.). Light emitted from the bioluminescent cells is detected by the IVIS camera system with images quantified for tumor burden using a log-scale color range set at 5×10⁴ to 1×10⁷ and measurement of total photon counts per second (PPS) using Living Image software (Xenogen).

Pathology. At the end of the experiment (14-32 days, depending on the tumor model) mice are sacrificed and the tumors are excised, fixed and subjected to pathological examination. Briefly, tumor sections are subjected to immunostaining with a panel of antibodies routinely applied in the lab to evaluate tumor cell proliferation (Ki67, BrdU), vascular density (CD31), lymphangiogenesis (LYVE), apoptosis (tunnel), autophagy (LC3II) and phosphorylation of key signaling molecules found to be activated by heparanase (i.e., EGFR, Akt, STAT3, Src). Heparanase staining extent and cellular localization (cytoplasmic vs. nuclear) will be examined as well.

(vi) Closing Paragraphs. Unless otherwise indicated, the practice of the present disclosure can employ conventional techniques of chemistry, organic chemistry, biochemistry, analytical chemistry, and physical chemistry. These methods are described in the following publications. See, e.g., Harcourt, et al., Holt McDougal Modern Chemistry: Student Edition (2018); J. Karty, Organic Chemistry Principles and Mechanisms (2014); Nelson, et al., Lehninger Principles of Biochemistry 5th edition (2008); Skoog, et al., Fundamentals of Analytical Chemistry (8th Edition); Atkins, et al., Atkins' Physical Chemistry (11th Edition).

The term aqueous pharmaceutically acceptable carrier is a solution in which the solvent used is water. The term alcoholic pharmaceutically acceptable carrier includes low alkyl alcohols such as methanol, ethanol, isopropyl alcohol, or similar alcohol as defined by its ordinary meaning to a person skilled in the art. A vicious base pharmaceutically acceptable carrier includes a thickening agent such as a combination of a polymer, carboxyvinyl polymer or viscous polymeric liquid and polymeric micelles and a water-soluble, high molecular cellulose compound.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the ability to obtain a claimed effect according to a relevant experimental method described in the current disclosure. For example, heparin would cause a statistically significant increase in anti-coagulation activity measured by the binding affinity of heparin to antithrombin III (ATM), compared to the binding affinity of the anti-heparanase glycopolymer to ATIII. Alternatively, high concentrations of the anti-heparanase glycopolymer would cause a statistically significant decrease in binding affinity between the glycopolymer and a heparan sulfate-binding protein as measured by a solution-based BLI assay.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

“Specifically binds” refers to an association of a molecule with its cognate binding molecule with an affinity or Ka (i.e., an equilibrium association constant of a particular binding interaction with units of 1/M) equal to or greater than 10⁵ M⁻¹, while not significantly associating with any other molecules or components in a relevant environment sample. “Specifically binds” is also referred to as “binds” herein. Molecules may be classified as “high affinity” or “low affinity”. In particular embodiments, “high affinity” binding domains refer to those molecules with a Ka of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In particular embodiments, “low affinity” binding domains refer to those binding domains with a Ka of up to 10⁷ M⁻¹, up to 10⁶ M⁻¹, up to 10⁵ M⁻¹. Alternatively, affinity may be defined as an equilibrium dissociation constant (Kd) of a particular binding interaction with units of M (e.g., 10⁻⁵ M to 10⁻¹³ M). In certain embodiments, a binding domain may have “enhanced affinity,” which refers to a selected or engineered binding domains with stronger binding to a cognate binding molecule than a wild type (or parent) binding domain. For example, enhanced affinity may be due to a Ka (equilibrium association constant) for the cognate binding molecule that is higher than the reference binding domain or due to a K_{d} (dissociation constant) for the cognate binding molecule that is less than that of the reference binding domain, or due to an off-rate (Koff) for the cognate binding molecule that is less than that of the reference binding domain. A variety of assays are known for detecting binding domains that specifically bind a particular cognate binding molecule as well as determining binding affinities, such as Western blot, ELISA, and BIACORE® analysis (see also, e.g., Scatchard, et al., 1949, Ann. N.Y. Acad. Sci. 51:660; and U.S. Pat. Nos. 5,283,173, 5,468,614, or the equivalent).

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A method of treating cancer in a subject in need thereof comprising administering a therapeutically effective amount of a salt form of an anti-heparanase compound having the structure: 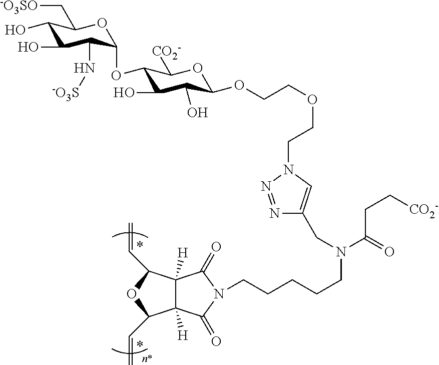
wherein n=2-100 repeating units of the structure; the ring opening bonds designated as (*) are independently single or double bonds; and the salt form is selected from a sodium salt, a calcium salt, a magnesium salt, a lithium salt, a potassium salt, a cesium salt, or a triethylammonium salt.

2. A method of claim 1 wherein the salt form is sodium salt.

3. A method of treating cancer in a subject in need thereof comprising administering a therapeutically effective amount of a salt form of an anti-heparanase compound to the subject wherein the anti-heparanase compound comprises a glycopolymer linked to a disaccharide.

4. The method of claim 3 wherein the salt form of the anti-heparanase compound has 5-12 repeating units of the glycopolymer linked to the disaccharide.

5. The method of claim 3 wherein the glycopolymer is linked to the disaccharide through nitrogen bonding.

6. The method of claim 3 wherein the disaccharide comprises a glucosamine unit sulfated at the carbon 2 and carbon 6 nitrogen positions of the disaccharide.

7. The method of claim 3 wherein the disaccharide comprises a glucosamine unit fluorinated at the carbon 2 or carbon 3 positions of the disaccharide.

8. The method of claim 3 wherein the salt form of the anti-heparanase compound is a heparan sulfate mimicking glycopolymer having the structure: 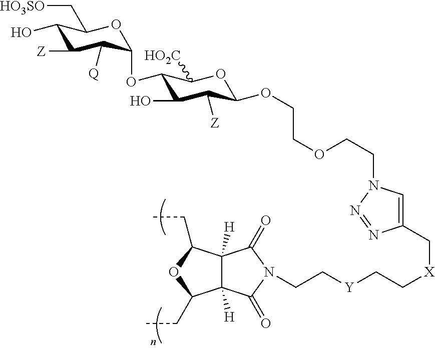
wherein:
X is —O— or 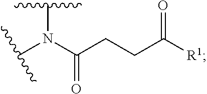
Y is —O— or —CH₂—; R¹ is OH or —N(H)-L-R^{a}; L is a linking group; R^{a} is a saccharide or disaccharide, which saccharide or disaccharide comprises a —SO₃Na group; Q is —NSO₃Na or —F; Z is either —OH or —F; the positioning of the carboxylic acid, or salt thereof, can either be axial or equatorial; and the dash bond --- is a single bond or a double bond.

9. The method of claim 8 wherein the heparan sulfate mimicking glycopolymer is a compound having the structure: 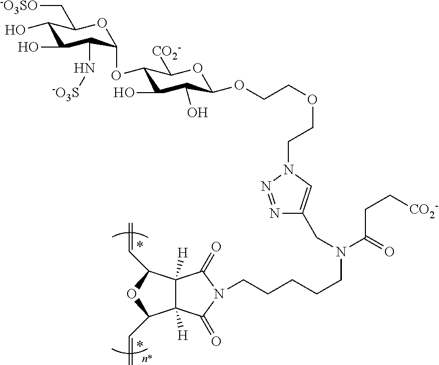
wherein the ring opening bonds designated as (*) are independently single or double bonds; and the salt form is selected from a sodium salt, a calcium salt, a magnesium salt, a lithium salt, a potassium salt, a cesium salt, or a triethylammonium salt.

10. The method of claim 8 wherein the heparan sulfate mimicking glycopolymer is a compound having the structure: 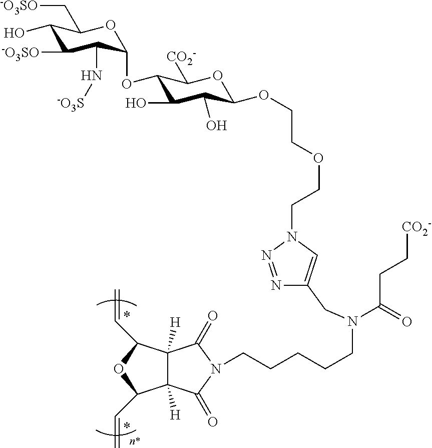
wherein the ring opening bonds designated as (*) are independently single or double bonds; and the salt form is selected from a sodium salt, a calcium salt, a magnesium salt, a lithium salt, a potassium salt, a cesium salt, or a triethylammonium salt.

11. The method of claim 8 wherein the heparan sulfate mimicking glycopolymer is a compound having the structure: 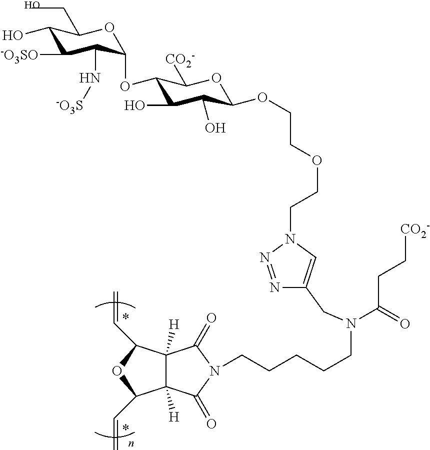
wherein the ring opening bonds designated as (*) are independently single or double bonds; and the salt form is selected from a sodium salt, a calcium salt, a magnesium salt, a lithium salt, a potassium salt, a cesium salt, or a triethylammonium salt.

12. The method of claim 8 wherein the heparan sulfate mimicking glycopolymer is a compound having the structure: 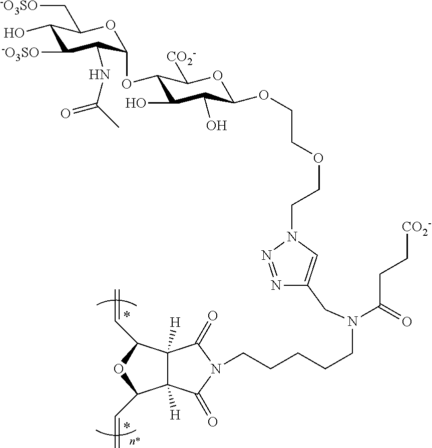
wherein the ring opening bonds designated as (*) are independently single or double bonds; and the salt form is selected from a sodium salt, a calcium salt, a magnesium salt, a lithium salt, a potassium salt, a cesium salt, or a triethylammonium salt.

13. The method of claim 8 wherein the heparan sulfate mimicking glycopolymer is a compound having the structure: 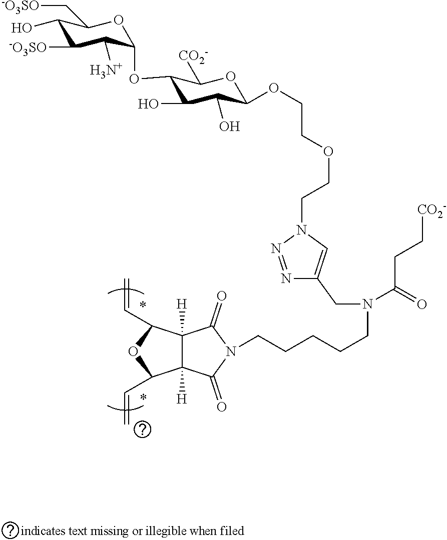
wherein the ring opening bonds designated as (*) are independently single or double bonds; and the salt form is selected from a sodium salt, a calcium salt, a magnesium salt, a lithium salt, a potassium salt, a cesium salt, or a triethylammonium salt.

14. The method of claim 8 wherein the heparan sulfate mimicking glycopolymer is a compound having the structure: 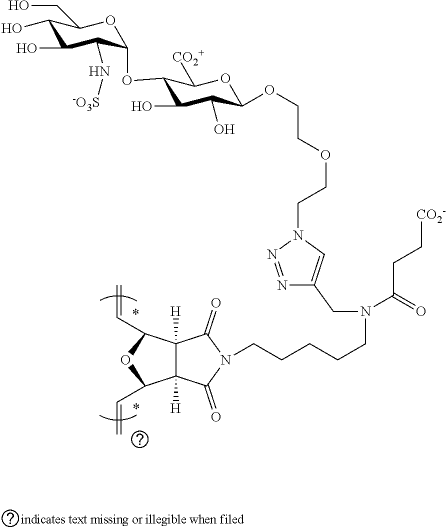
wherein the ring opening bonds designated as (*) are independently single or double bonds; and the salt form is selected from a sodium salt, a calcium salt, a magnesium salt, a lithium salt, a potassium salt, a cesium salt, or a triethylammonium salt.

15. The method of claim 8 wherein the heparan sulfate mimicking glycopolymer is a compound having the structure: 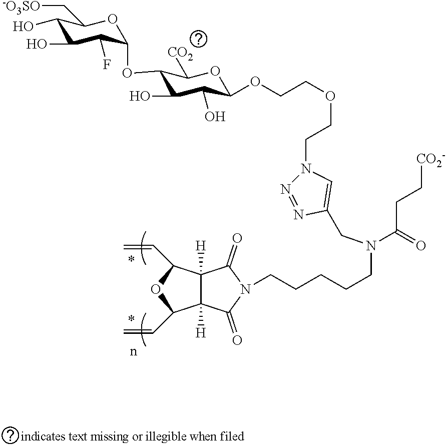
wherein the ring opening bonds designated as (*) are independently single or double bonds; and the salt form is selected from a sodium salt, a calcium salt, a magnesium salt, a lithium salt, a potassium salt, a cesium salt, or a triethylammonium salt.

16. The method of claim 8 wherein the heparan sulfate mimicking glycopolymer is a compound having the structure: 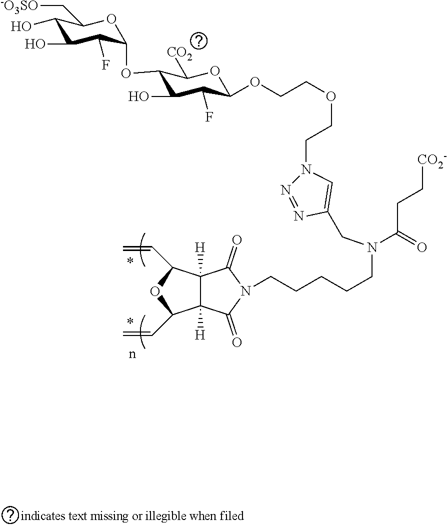
wherein the ring opening bonds designated as (*) are independently single or double bonds; and the salt form is selected from a sodium salt, a calcium salt, a magnesium salt, a lithium salt, a potassium salt, a cesium salt, or a triethylammonium salt.

17. The method of claim 8 wherein the heparan sulfate mimicking glycopolymer is a compound having the structure: 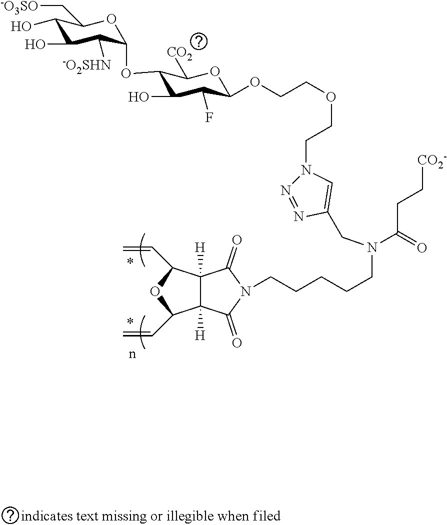
wherein the ring opening bonds designated as (*) are independently single or double bonds; and the salt form is selected from a sodium salt, a calcium salt, a magnesium salt, a lithium salt, a potassium salt, a cesium salt, or a triethylammonium salt.

18. The method of claim 8 wherein the heparan sulfate mimicking glycopolymer is a compound having the structure: 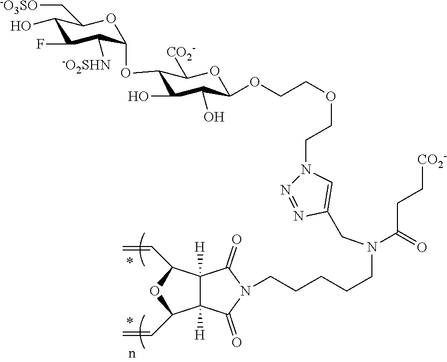
wherein the ring opening bonds designated as (*) are independently single or double bonds; and the salt form is selected from a sodium salt, a calcium salt, a magnesium salt, a lithium salt, a potassium salt, a cesium salt, or a triethylammonium salt.

19. The method of claim 3 wherein the salt form of the anti-heparanase compound has the structure: 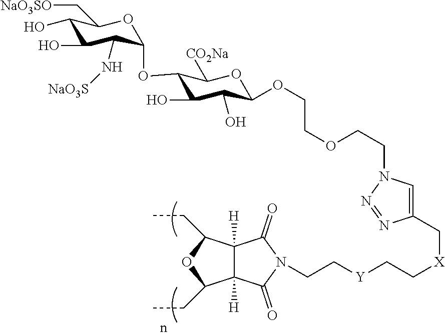
wherein:
X is —O— or 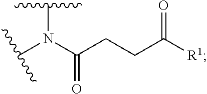
Y is —O— or —CH₂—; R¹ is OH or —N(H)-L-R^{a}; L is a linking group; R^{a} is a saccharide or disaccharide, which saccharide or disaccharide comprises a —SO₃Na group; and the dash bond --- is a single bond or a double bond.

20. The method of claim 19 wherein X is —O— and Y is —O—; or X is 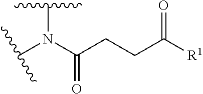
and Y is —CH₂—.

21. The method of claim 19 wherein the salt form of the anti-heparanase compound has the structure: 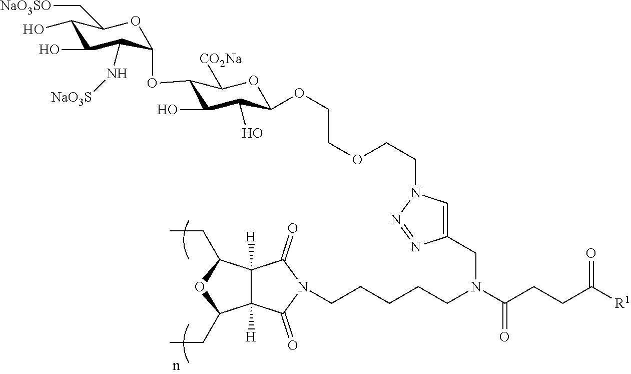
wherein R¹ is OH or —N(H)-L-R^{a}; L is a linking group; and R^{a} is a saccharide or disaccharide, which saccharide or disaccharide comprises a —SO₃Na group; and the carboxylic acid group is a salt thereof.

22. The method of claim 19 wherein the salt form of the anti-heparanase compound has the structure:

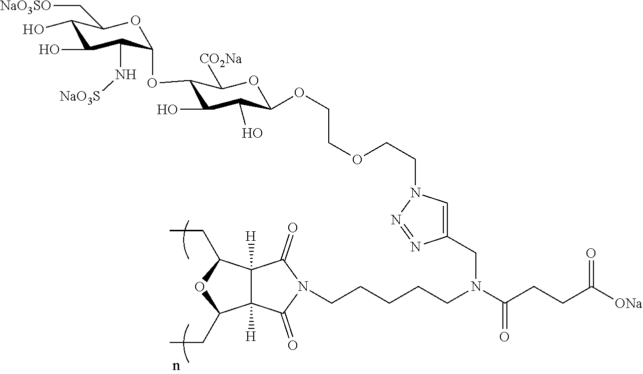

23. The method of claim 19 wherein the salt form of the anti-heparanase compound has the structure:

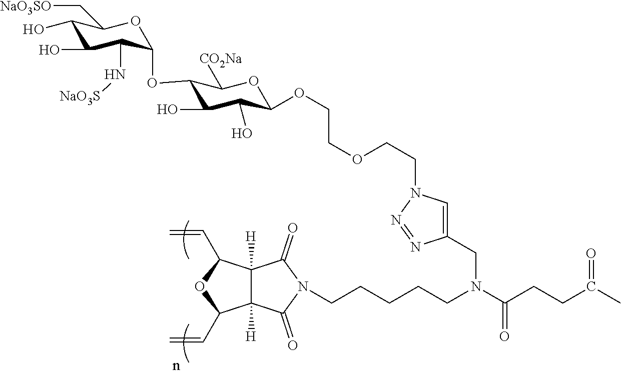

24. The method of claim 19 wherein the salt form of the anti-heparanase compound has the structure:

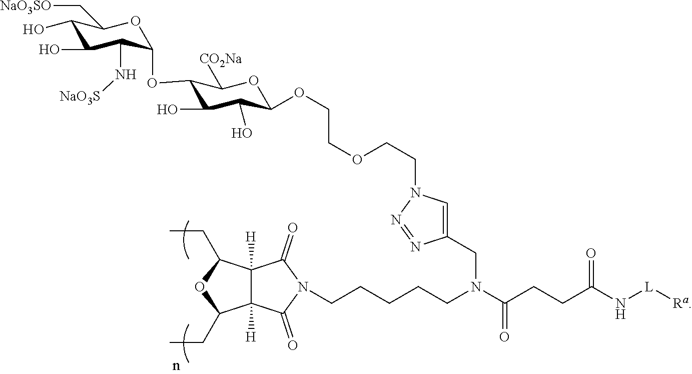

25. The method of claim 19 wherein the salt form of the anti-heparanase compound has the structure:

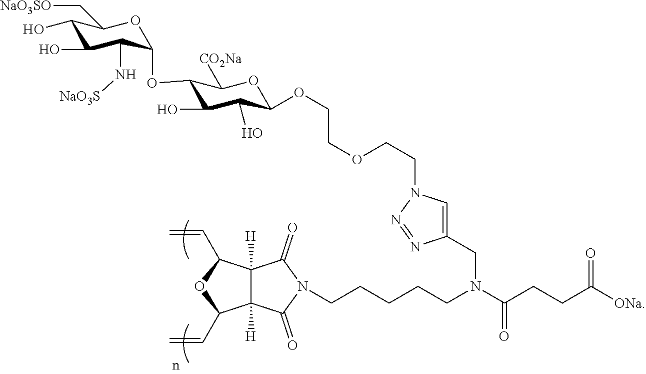

26. The method of claim 19 wherein the salt for of the anti-heparanase compound has the structure:

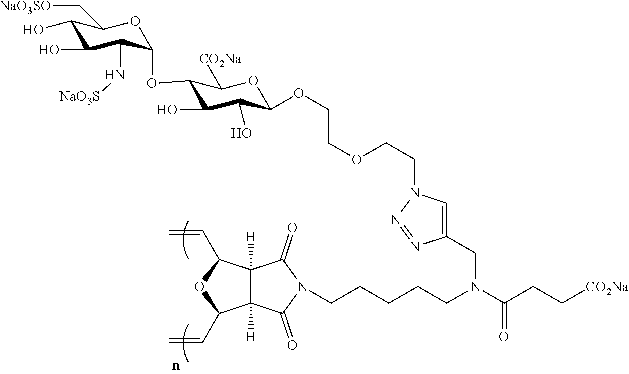

27. The method of claim 19 wherein the salt form of the anti-heparanase compound has the structure: 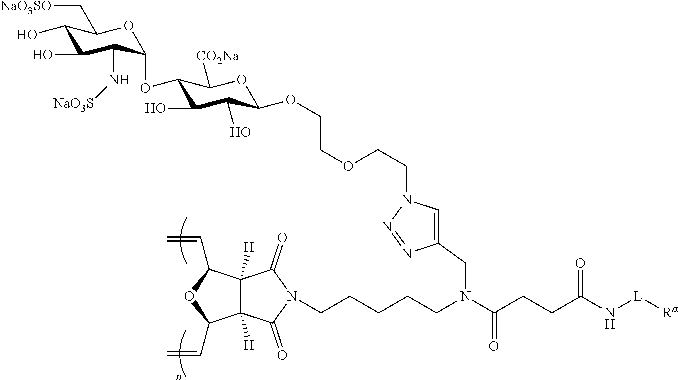
wherein L is a linking group; R^{a} is a saccharide or disaccharide, which saccharide or disaccharide comprises a —SO₃Na group.

28. The method of claim 19 wherein the salt form of the anti-heparanase compound has the structure:

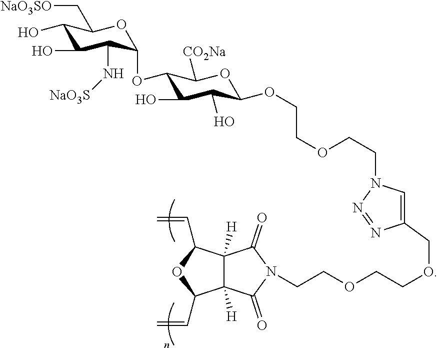

29. The method of claim 19 wherein R^{a} is selected from:

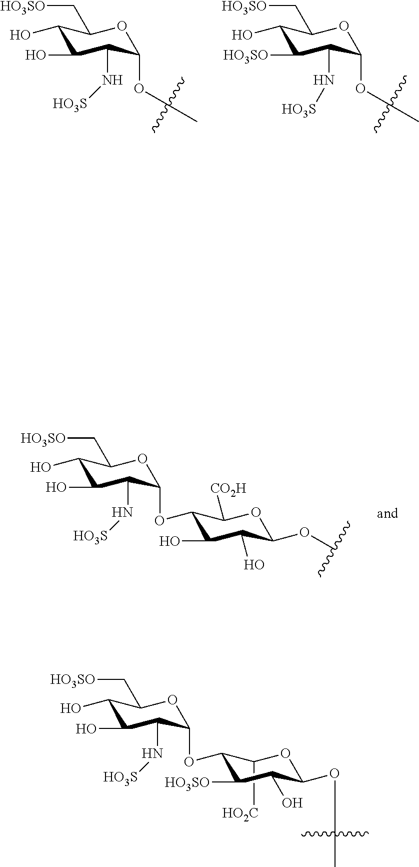

30. The method of claim 19 wherein R^{a} is selected from:

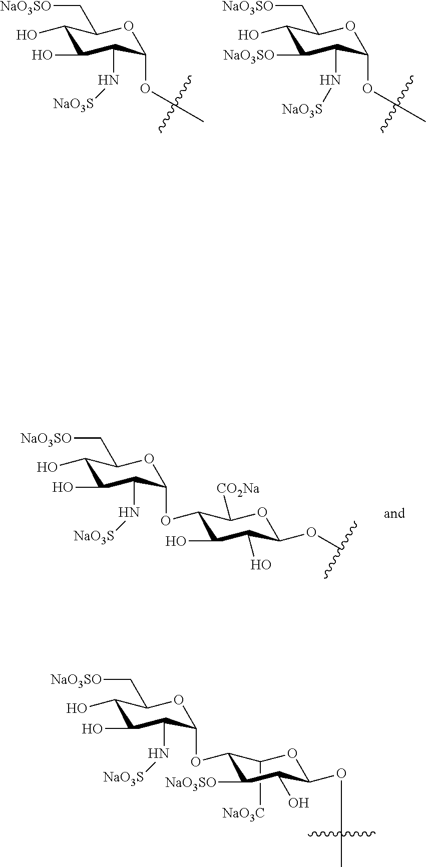

31. The method of claim 19 wherein R^{a} is:

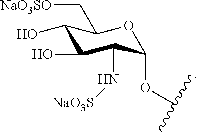

32. The method of claim 19 wherein the salt form of the anti-heparanase compound has the structure:

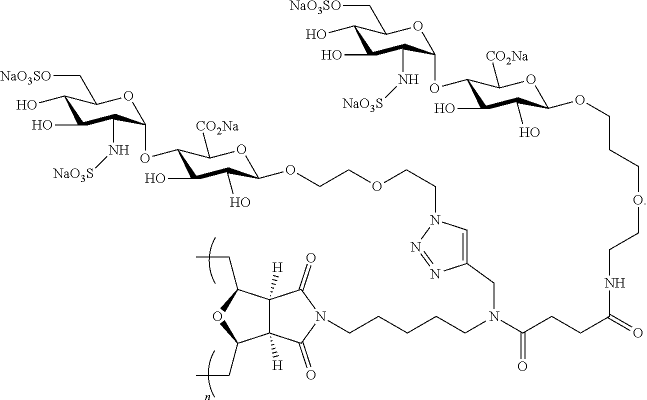

33. The method of claim 19 wherein the salt form of the anti-heparanase compound has the structure:

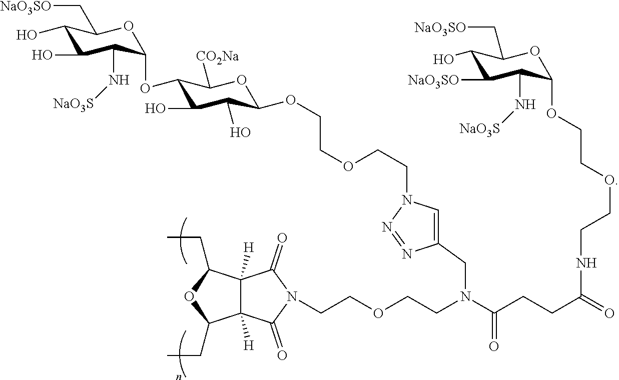

34. The method of claim 19 wherein the salt form of the anti-heparanase compound has the structure:

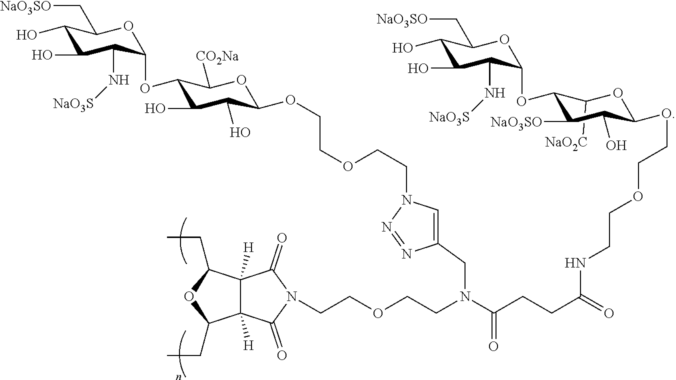

35. The method of claim 19 wherein the salt form of the anti-heparanase compound has the structure: 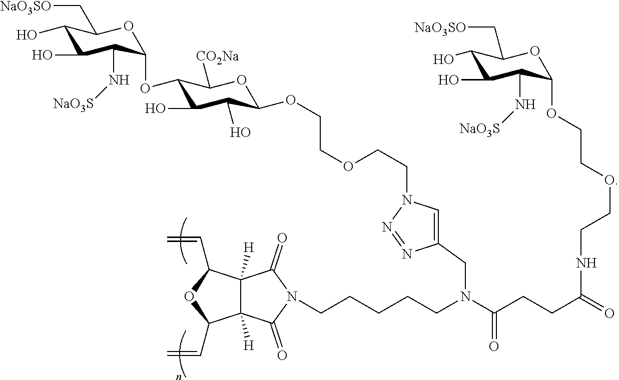
wherein n is 8.

36. The method of any one of claim 8-35 wherein n is an integer from 2-100.

37. The method of any one of claim 8-35 wherein n is an integer from 5-55.

38. The method of any one of claim 8-35 wherein n is 5, 8, 9, 12, 27, or 51.

39. A compound having the structure: 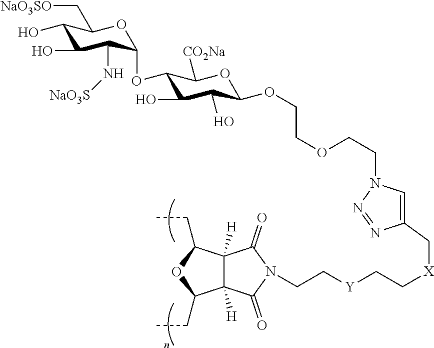
wherein:
X is —O— or 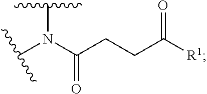
Y is —O— or —CH₂—; n=2-100 repeating units; R¹ is OH or —N(H)-L-R^{a}; L is a linking group; R^{a} is a saccharide or disaccharide, which saccharide or disaccharide comprises one or more —SO₃H groups; the carboxylic acid group is a salt thereof; and the dash bond --- is a single bond or a double bond.

40. The compound of claim 39, having the structure: 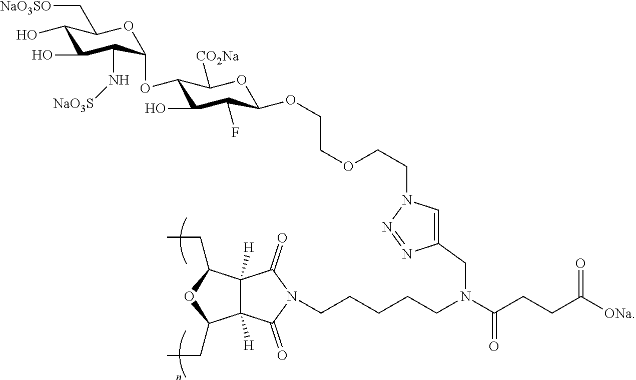
wherein: n=2-100 repeating units; and the saccharide or disaccharide further comprises one or more F⁻ groups.

41. The compound of claim 39, having the structure:

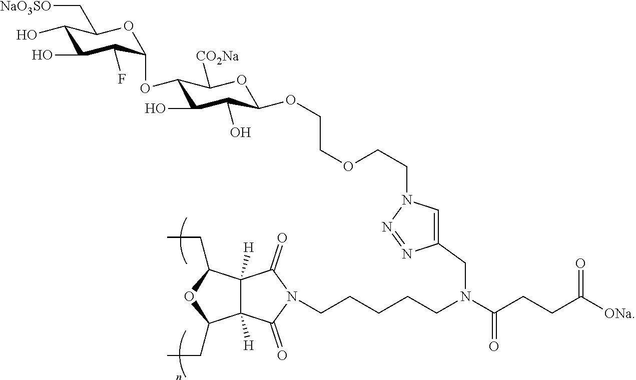

42. The compound of claim 39, having the structure:

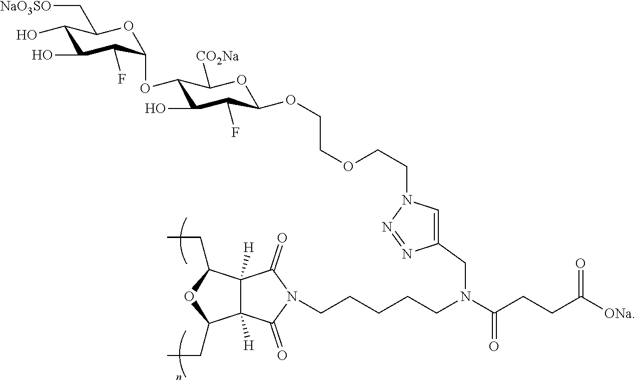

43. The compound of claim 39, having the structure:

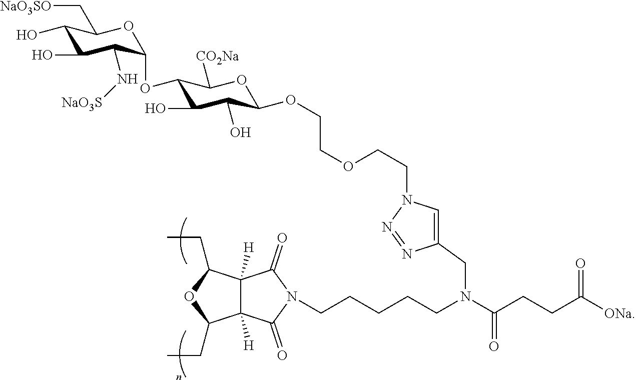

44. The compound of 40 wherein the one or more F− groups comprise axial 2-fluoro-glycoside.

45. The compound of 41 wherein the one or more F− groups comprise axial 2-fluoro-glycoside.

46. The compound of 42 wherein the one or more F− groups comprise axial 2-fluoro-glycoside.

47. The compound of 43 wherein the one or more F− groups comprise axial 2-fluoro-glycoside.

48. An anti-cancer composition comprising: (i) an anti-heparanase compound of any one of claims 1-35 and (ii) a pharmaceutically acceptable carrier.

49. The anti-cancer composition of claim 48 wherein the binding affinity of the anti-heparanase compound to FGF-1 is more than the binding affinity of heparin to FGF-1.

50. The anti-cancer composition of claim 48 wherein the binding affinity of the anti-heparanase compound to FGF-1 is at least 2000 nM more than the binding affinity of heparin to FGF-1.

51. The anti-cancer composition of claim 48 wherein the binding affinity of the anti-heparanase compound to FGF-2 is more than the binding affinity of heparin to FGF-2.

52. The anti-cancer composition of claim 48 wherein the binding affinity of the anti-heparanase compound to FGF-2 is at least 530 nM more than the binding affinity of heparin to FGF-2.

53. The anti-cancer composition of claim 48 wherein the binding affinity of the anti-heparanase compound to VEGF is more than the binding affinity of heparin to VEGF.

54. The anti-cancer composition of claim 48 wherein the binding affinity of the anti-heparanase compound to VEGF is at least 115 nM more than the binding affinity of heparin to VEGF.

55. The anti-cancer composition of claim 48 wherein the binding affinity of the anti-heparanase compound to PF4 is more than the binding affinity of heparin to PF4

56. The anti-cancer composition of claim 48 wherein the binding affinity of the anti-heparanase compound to PF4 is at least 35 nM more than the binding affinity of heparin to PF4.

57. The anti-cancer composition of claim 48 wherein the binding affinity of the anti-heparanase compound to P-Selectin is more than or equal to the binding affinity of heparin to P-Selectin

58. The anti-cancer composition of claim 48 wherein the binding affinity of the anti-heparanase compound to P-Selectin is at least −570 nM more than or equal to the binding affinity of heparin to P-Selectin.

59. The anti-cancer composition of claim 48 wherein the anti-heparanase compound has lower binding affinity to antithrombin III than heparin's binding affinity to antithrombin III.

60. The anti-cancer composition of claim 48 wherein the pharmaceutically acceptable carrier is aqueous or alcoholic and comprises a viscous base.
